(19) United States
(12) Patent Application Publication
(10) Pub. No.: US 2024/0124460 A1
(43) Pub. Date: Apr. 18, 2024

(54) METHODS AND COMPOSITIONS FOR TARGETED PROTEIN DEGRADATION

(71) Applicant: Ranok Therapeutics (Hangzhou) Co. Ltd., Hangzhou (CN)

(72) Inventors: Weiwen Ying, Lexington, MA (US); Kevin P. Foley, Waltham, MA (US); Wei Yin, Lianyungang (CN); Long Ye, Shanghai (CN); Mingkai Wang, Hangzhou (CN); Chenghao Ying, Shanghai (CN); Lingjie Zhang, Wuxi (CN)

(21) Appl. No.: 18/031,678

(22) PCT Filed: Oct. 12, 2021
(86) PCT No.: PCT/CN2021/123366

§ 371 Date: Apr. 13, 2023

(30) Foreign Application Priority Data
Oct. 14, 2020 (WO) PCT/CN2020/120911

(51) Int. Cl. C07D 487/04; A61P 35/00

(52) U.S. Cl. CPC C07D 487/04; A61P 35/00

(57) ABSTRACT

Provided are compounds of Formula (I) and pharmaceutically acceptable salts and compositions thereof, which are useful for treating cancers and related conditions.

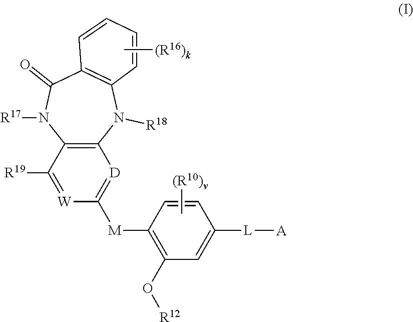

### RELATED APPLICATIONS

This application claims the benefit of priority to PCT/CN2020/120911, filed Oct. 14, 2020, the entire contents of which are incorporated herein by reference.

### BACKGROUND

Protein homeostasis, or proteostasis, refers to the ability of cells to regulate the synthesis, folding, trafficking and degradation of proteins. In particular, properly regulated protein degradation is required for the normal functioning of cells, including their proliferation, differentiation and death, and is often dysregulated in cancers and other diseases (Van Die, Chin J Cancer, 2011, 30:124-137).

The ubiquitin-proteasome system (UPS) is one of the major pathways in cells that mediates the disposal and metabolic recycling of proteins (Yu and Matouschek, Annu Rev Biophys, 2017, 46:149-173; Navon and Ciechanover, J Biol Chem, 2009, 284:33713-33718). Ubiquitin is a 76 amino acid-residue protein that is ubiquitously expressed. With respect to protein degradation by the UPS, the process of ubiquitination occurs when a ubiquitin is attached to a lysine amino acid residue in a substrate protein, which involves a series of enzymatic steps. First, ubiquitin is transferred to an E1 ubiquitin-activating enzyme. Second, activated ubiquitin is transferred from the E1 to an E2 ubiquitin-conjugating enzyme. And third, one of the several hundred different E3 ubiquitin ligase enzymes links the ubiquitin to a lysine residue in a substrate protein. Repetition of this enzymatic process results in tagging substrate proteins with polyubiquitin chains. Such ubiquitin-tagged proteins can then be delivered to the proteasome, a large multi-subunit complex that degrades proteins. The ability of some cellular chaperone proteins and chaperone complexes to direct proteins towards the UPS is facilitated by their direct interaction with E3 ubiquitin ligases (Amm et al., Biochim Biophys Acta, 2014, 1843:182-196; Taipale et al., Cell, 2012, 150:987-1001). In addition to protein degradation, the ubiquitination of proteins can also regulate other processes, such as subcellular localization, activity and protein-protein interactions.

Chemically induced, targeted protein degradation (TPD) has emerged as a new modality for small molecule drug development. A small molecule can be used to promote the interaction of a target protein or proteins with a component or components of various cellular protein degradation pathways, thereby inducing the degradation of the targeted protein or proteins as a way to treat disease.

In particular, proteolysis-targeting chimeras (PROTACs) are an example of such small molecules that purposely induce protein degradation of specific proteins by coopting the UPS (Burslem and Crews, Cell, 2020, 181:102-114; Pettersson and Crews, Drug Discov Today Technol, 2019, 31:15-27). PROTAC molecules are bifunctional small molecules that simultaneously bind to a target protein or proteins and an E3 ubiquitin ligase, creating ternary complexes in cells between the target protein(s), the PROTAC molecule and an E3 ligase protein. The induced proximity of the target protein(s) and the E3 ligase causes the ubiquitination of the target protein(s) and subsequent degradation of the target protein(s) by the proteasome. Although PROTACs that incorporate target protein binders that promiscuously bind to multiple proteins can often degrade multiple proteins, in some cases protein-protein interactions between individual targets and an E3 ligase can increase or decrease the observed potency and selectivity of degradation, for example by inhibiting formation of some ternary complexes due to charge repulsion and steric clashing between a given target protein and E3 ligase pair (Pettersson and Crews, Drug Discov Today Technol, 2019, 31:15-27; Bondeson et al., Cell Chem Biol, 2018, 25:78-87; Gadd et al., Nat Chem Biol, 2017, 13:514-521; Zengerle et al., ACS Chem Biol, 2015, 10:1770-1777).

Other methods to chemically induce TPD have also been described, such as molecular glues (Che et al., Bioog Med Chem Lett, 2018, 28:2585-2592), AUTACs, ATTECs and LYTACs (Ding et al., Trends Pharmacol Sci, 2020, 41:464-474). For example, AUTAC technology follows a similar principle of induced proximity, but targets proteins for degradation via autophagy (Daiki et al., Mol Cell, 2019, 76:797-810).

Collectively, TPD technologies have a number of advantages over conventional biochemical inhibitors (Pettersson and Crews, Drug Discov Today Technol, 2019, 31:15-27; Ding et al., Trends Pharmacol Sci, 2020, 41:464-474). For example, unlike conventional inhibitors, TPD agents work sub-stoichiometrically and can typically mediate the sequential degradation of multiple molecules of the target protein(s), often leading to greater potency than the isolated target binding moiety that they incorporate and other biochemical inhibitors. Also, since inhibition of target protein(s) function by TPD agents is principally due to degradation rather than solely biochemical inhibition, recovery of the function of target protein(s) is typically slower than is observed for biochemical inhibitors. TPD agents may also have improved target selectivity over biochemical inhibitors. Finally, TPD agents can target proteins that are not amenable to biochemical inhibition by interacting with binding pockets that do not affect the biochemical activity of the target but still permit its degradation.

However, some disadvantages are associated with current TPD technologies. These include the promiscuous degradation of the target protein(s) in many tissues and organs, not just the tissue(s) and organ(s) where the target protein(s) is involved in a disease process, which is expected to result in unwanted side effects of treatment. Also, resistance to these technologies can develop through mutations or alterations in expression of components of the UPS such as E3 ligases (Ottis et al., ACS Chem Biol, 2019, 14:2215-2223; Zhang et al., Mol Cancer Ther, 2019, 18:1302-1311), resulting in loss of therapeutic efficacy. As such, a need exists for improved/alternative methods and compositions for TPD.

It is also desirable to develop improved/alternative TPD agents that mediate the degradation of proteins involved in cancer and other diseases. Mitogen-activated protein kinases (MAPKs) regulate many different aspects of cellular function, including the growth, survival and death of cancer cells. In particular, MAPK7 (also known as ERK5), a component of the MEK5 signaling pathway, has been implicated as playing an important role in a variety of different cancer types (Hoang et al., Cancer Lett, 2017, 392:51-59; Stecca and Rovida, Int J Mol Sci, 2019, 20:1426; Pereira and Rodrigues, Trends Mol Med, 2020, 26:394-407; Tubita et al., Int J Mol Sci, 2020, 21:938). For example, in triple-negative breast (TNBC), there is a correlation between high MAPK7 expression and poor prognosis (Ortiz-Ruiz et al., Oncotarget, 2014, 5:11308-11318). Likewise, in prostate cancer, strong nuclear MAPK7 localization is an independent prognostic factor of poor disease-specific survival (McCracken et al., Oncogene, 2008, 27:2978-2988). Importantly, many of the functions of MAPK7 are not affected by selective MAPK7 biochemical inhibitors and therefore TPD approaches are required to block its function in cancer cells (Lin et al., Proc Natl Acad Sci USA, 2016, 113:11865-11870). As such MAPK7 represents an attractive drug target and it is desirable to develop agents that induce TPD of MAPK7.

### SUMMARY

The present disclosure provides tumor-targeted protein degradation chimeras, termed chaperone-mediated protein degraders (CHAMPs) comprising a first moiety that is capable of binding to a target protein (e.g., MAPK7) or proteins and a second moiety that is capable of binding a chaperone protein or proteins or protein component of chaperone complexes (e.g., HSP90). Such CHAMP compounds include those having the Formula I:

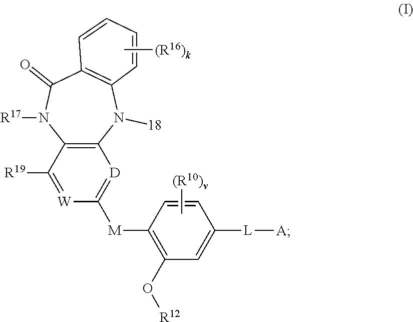

and pharmaceutically acceptable salts thereof, wherein W, D, L, A, R¹⁰, R¹², R¹⁶, R¹⁷, R¹⁸, R¹⁹, k and v are as defined herein.

Compositions comprising the disclosed compounds of Formula I as well as methods for their manufacture are also provided. In one aspect, the disclosed compounds induce targeted oncogenic protein degradation in a tumor-selective fashion and are useful in the treatment of cancer and related conditions.

### DETAILED DESCRIPTION

### 1. General Description of Compounds

Provided herein are CHAMP compounds having the Formula I:

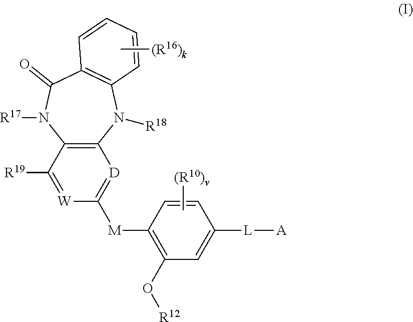

or a pharmaceutically acceptable salt thereof, wherein,
A is a chemical moiety that binds HSP90 protein;
L is a linker;
W and D are each independently N or CR⁹;
R¹⁰, R¹⁶, and R¹⁹ are each independently selected from halo, (C₁-C₆)alkyl, (C₂-C₆)alkenyl, halo(C₂-C₆)alkenyl, (C₂-C₆)alkynyl, —(C₁-C₆)alkylOR^{c}, —(C₁-C₆)alkylN(R^{d})₂, —(C₁-C₆)alkylC(O)OR^{d}, —(C₁-C₆)alkylC(O)N(R^{d})₂, —(C₁-C₆)alkylO(C₁-C₆)alkylN(R^{d})₂, —(C₁-C₆)alkylSOR^{d}, —(C₁-C₆)alkylS(O)₂R^{d}, —(C₁-C₆)alkylSON(R^{d})₂, —(C₁-C₆)alkylSO₂N(R^{d})₂, —(C₁-C₆)alkylcycloalkyl, —(C₁-C₆)alkylheterocyclyl, —(C₁-C₆)alkylheteroaryl, —(C₁-C₆)alkylaryl, —(C₁-C₆)alkoxy, halo(C₁-C₆)alkoxy, CN, aryl, heteroaryl, cycloalkyl, heterocycloalkyl, —C(O)R^{d}, —C(O)OR^{d}, —C(O)N(R^{d})₂, N(R^{d})₂, —C(O)NR^{d}(C₁-C₆)alkylN(R^{d})₂, —NR^{d}(C₁-C₆)alkylN(R^{d})₂, —NR^{d}(C₁-C₆)alkylOR^{d}, —SOR^{d}, —S(O)₂R^{d}, —SON(R^{d})₂, —SO₂N(R^{d})₂, and CN, wherein each aryl, cycloalkyl, heterocyclyl, and heteroaryl alone and in connection with —(C₁-C₆)alkylcycloalkyl, —(C₁-C₆)alkylheterocyclyl, —(C₁-C₆)alkylheteroaryl, —(C₁-C₆)alkylaryl are optionally substituted with 1 to 3 groups selected from R^{e};
M is O, S, or NR¹¹;
R¹¹, R¹⁷, R¹⁸, and R²⁰, are each independently selected from hydrogen, (C₁-C₆)alkyl, and S(O)₂(C₁-C₆)alkyl (alternatively, R¹⁷ or R¹⁸ are each independently selected from hydrogen, (C₁-C₆)alkyl, C₃-C₆ cycloalkyl, and S(O)₂(C₁-C₆)alkyl);
R¹² is hydrogen, (C₁-C₆)alkyl, halo(C₁-C₆)alkyl, —(C₁-C₆)alkylOR^{c}, S(O)₂(C₁-C₆)alkyl, aryl, heteroaryl, cycloalkyl, heterocycloalkyl, C(O)(C₁-C₆)alkyl, or —(C₁-C₆)alkylaryl, wherein each aryl, cycloalkyl, heterocyclyl, and heteroaryl alone and in connection with —(C₁-C₆)alkylaryl are optionally substituted with 1 to 3 groups selected from R^{e};
R^{c} and R^{d} are each independently selected from hydrogen, (C₁-C₆)alkyl, and halo(C₁-C₆)alkyl;
R^{e} is selected from halo, oxo, CN, NO₂, —N(R^{d})₂, —OR^{d}, —C(O)OR^{d}, (C₁-C₆)alkyl, —(C₁-C₆)alkylOR^{c}, halo(C₁-C₆)alkyl, (C₁-C₆)alkoxy, halo(C₁-C₆)alkoxy, —(C₁-C₆)alkylC(O)OR^{d}, —(C₁-C₆)alkylC(O)N(R^{d})₂, (C₂-C₆)alkenyl, halo(C₂-C₆)alkenyl, (C₂-C₆)alkynyl, —(C₁-C₆)alkylSR^{d}, —(C₁-C₆)alkylOR^{c}, —(C₁-C₆)alkylN(R^{d})₂, —C(O)N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, aryl, heteroaryl, cycloalkyl, and heterocycloalkyl; and
k and v are each independently 0, 1, 2, or 3.

### 2. Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements.

Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” are used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkenyl” means a saturated straight chain or branched non-cyclic hydrocarbon having, unless specified otherwise, from 2 to 10 carbon atoms (e.g., (C₂-C₆)alkenyl or (C₂-C₄)alkenyl) and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

As used herein, the term “cycloalkyl” means a saturated, monocyclic alkyl radical having from e.g., 3 to 10 carbon atoms (e.g., from 4 to 6 carbon atoms). Representative cycloalkyls include cyclopropyl, cyclobutyl, cyclopentyl, cyclohexyl, cycloheptyl, cyclooctyl, cyclononyl, and cyclodecanyl.

The term “oxo” refers to the group ═O.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker.

As used herein, an “haloalkoxy” is an haloalkyl group which is attached to another moiety via an oxygen linker.

As used herein, the term “alkylene” refers to an alkyl group that has two points of attachment. Straight chain alkylene groups are preferred. Non-limiting examples of alkylene groups include methylene ethylene, n-propylene, isopropylene, and the like. Alkylene groups may be optionally substituted with one or more substituents.

As used herein, the term “heterocyclyl” means a monocyclic heterocyclic ring system which is either a saturated ring or an unsaturated non-aromatic ring comprising, as size and valency permits, up to 5 heteroatoms independently selected from nitrogen, oxygen, and sulfur. The heterocycle may be attached via any heteroatom or carbon atom. Representative heterocycles include morpholinyl, thiomorpholinyl, pyrrolidinonyl, pyrrolidinyl, piperidinyl, piperazinyl, oxiranyl, oxetanyl, tetrahydrofuranyl, tetrahydropyranyl, tetrahydropyrindinyl, tetrahydropyrimidinyl, and the like.

As used herein, the term “heteroaryl” means, as the defined size permits, a monocyclic or polycyclic heteroaromatic ring comprising carbon atom ring members and one or more heteroatom ring members selected from nitrogen, oxygen, and sulfur. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. The point of attachment of a heteroaromatic or heteroaryl ring to another group may be at either a carbon atom or a heteroatom of the heteroaromatic or heteroaryl rings.

As used herein, the term “halogen” or “halo” means F, Cl, Br or I.

When a heterocyclyl or heteroaryl, group contains a nitrogen atom, it may be substituted or unsubstituted as valency permits.

The term “linker” or “tether,” used interchangeably, refers to a chemical moiety that joins two other moieties (e.g., a first binding moiety and a second binding moiety). A linker can covalently join a first binding moiety and a second binding moiety. In one aspect, the linker is uncleavable in vivo. In one aspect, the linker comprises one or more cyclic ring systems. In another aspect, the linker comprises an alkyl chain optionally substituted by and/or interrupted with one or more chemical groups. In one aspect, the linker comprises optimal spatial and chemical properties to effectuate optimal therapeutic activity. In one aspect, the linker does not interfere with the ability of the first binding moiety and/or the second binding moiety to bind their respective targets (e.g., HSP90 and MAPK7). In one aspect, the linker alters the ability of the first binding moiety and/or the second binding moiety to bind their respective targets (e.g., HSP90 and MAPK7).

The term “MAPK7” refers to the protein product of the mitogen-activated protein kinase 7 gene, also known as the extracellular-signal-regulated kinase 5 or ERK5 gene.

The term “HSP90” refers collectively, individually or in various combinations to the protein products of members of the heat shock protein 90 (90 kDa) gene family, including: HSP90AA1 (HSP90-alpha or HSP90α), HSP90AB1 (HSP90-beta or HSP90β), HSP90B1 (GRP94) and TRAP1.

When used in connection to describe a chemical group that may have multiple points of attachment, a hyphen (-) designates the point of attachment of that group to the variable to which it is defined. For example, —NR^{a}R^{b} and —C(O)NR^{a}(C₁₋₄alkylene)NR^{a}R mean that the point of attachment for these groups occur on the nitrogen atom and carbon atom respectively.

A hash bond as in “ [symbol] ” represents the point at which the depicted group is attached to the defined variable.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the other stereoisomers. For example, when a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. In another preferred embodiment, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

In a first embodiment, provided is a compound of the Formula I:

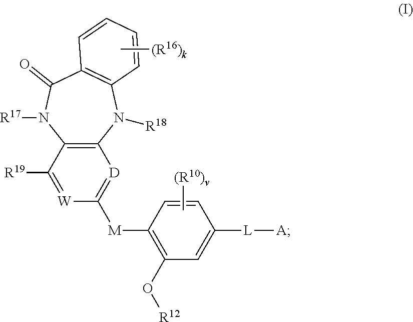

or a pharmaceutically acceptable salt thereof, wherein the variables are as described above.

In a second embodiment, A is selected from

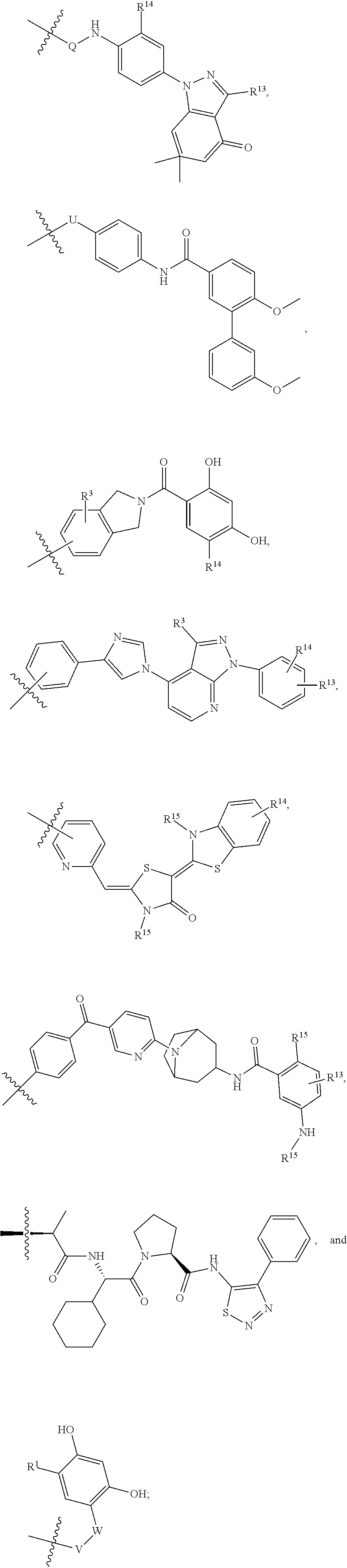

wherein
Q and U are each independently selected from phenyl, heteroaryl, heterocyclyl, and cycloalkyl, each of which being optionally substituted with 1 to 3 groups selected from R².
R¹³ and R¹⁴ are each independently selected from hydrogen, halo, —CN, (C₁-C₄)alkyl, halo(C₁-C₄)alkyl, and —C(O)NR^{a}R^{b};
R¹⁵ is hydrogen, (C₁-C₄)alkyl, or halo(C₁-C₄)alkyl;
W is 5- or 6-membered heteroaryl optionally substituted with 1 to 3 groups selected from R²;
V is phenyl or 5- to 9-membered heteroaryl optionally substituted with 1 to 3 groups selected from R³;
R¹ is halo, (C₁-C₄)alkyl, halo(C₁-C₄)alkyl, (C₁-C₄)alkoxy, or halo(C₁-C₄)alkoxy;
R² is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{a}, —OR^{a}, —C(O)R^{a}, —C(O)OR^{a}, —C(O)NR^{a}R^{b}, —C(O)NR^{a}(C₁₋₄alkylene)OR^{a}, —C(O)NR^{a}(C₁₋₄alkylene)NR^{a}R^{b}, —C(O)NR^{a}(C₁₋₄alkylene)OR, —NR^{a}R^{b}, —O(C₁₋₄alkylene)NR^{a}R^{b}, —C₁₋₄alkylNR^{a}R^{b}, —SR^{a}, —S(O)R^{a}, —S(O)₂R^{a}, —S(O)NR^{a}R^{b}, —SO₂NR^{a}R^{b}, —NR^{a}(C₁₋₄alkyl)OR^{a}, —SH, —S(C₁₋₄alkyl), —NR^{a}(C₁₋₄alkyl)NR^{a}R^{b}, —C₁₋₆alkylC(O)NR^{a}R^{b}, —O(C₁₋₄alkylene)NR^{a}C(O)(C₁₋₄alkylene)NR^{a}R^{b}, phenyl or 5- to 7-membered heteroaryl, wherein said phenyl and 5- to 7-membered heteroaryl are each optionally and independently substituted with 1 to 3 groups selected from R⁴;
R^{a} and R^{b} are each independently selected from hydrogen and (C₁-C₄)alkyl, wherein said (C₁-C₄)alkyl is optionally substituted with one or more halo or a 3- to 7-membered heterocyclyl, or both (alternatively, the 3- to 7-membered heterocyclyl may be optionally substituted e.g., with 1 to 3 groups selected from halo, (C₁-C₄)alkyl, halo(C₁-C₄)alkyl, (C₁-C₄)alkoxy, halo(C₁-C₄)alkoxy, and —OSO₂(C₁-C₃)alkyl; and
R³ and R⁴ are each independently halo, —NR^{a}R^{b}, (C₁-C₄)alkyl, halo(C₁-C₄)alkyl, (C₁-C₄)alkoxy, or halo(C₁-C₄)alkoxy, wherein the remaining variables are as described for Formula I. Alternatively, as part of a second embodiment, A is selected from

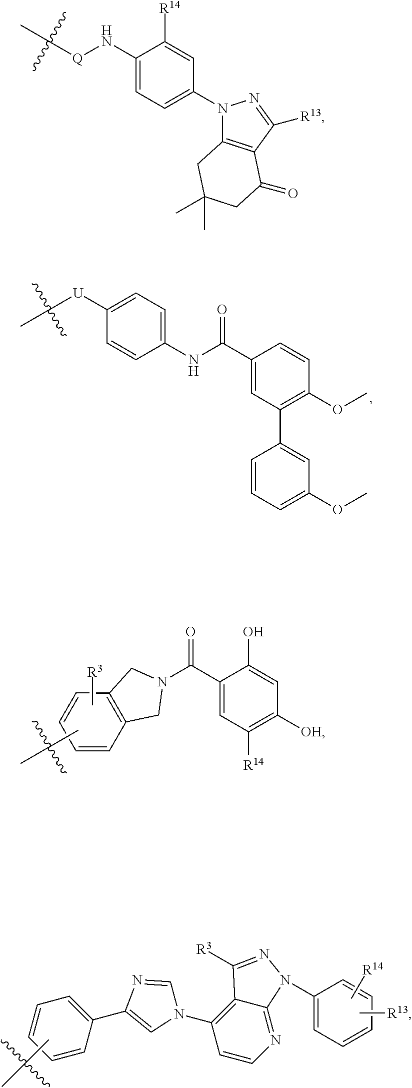

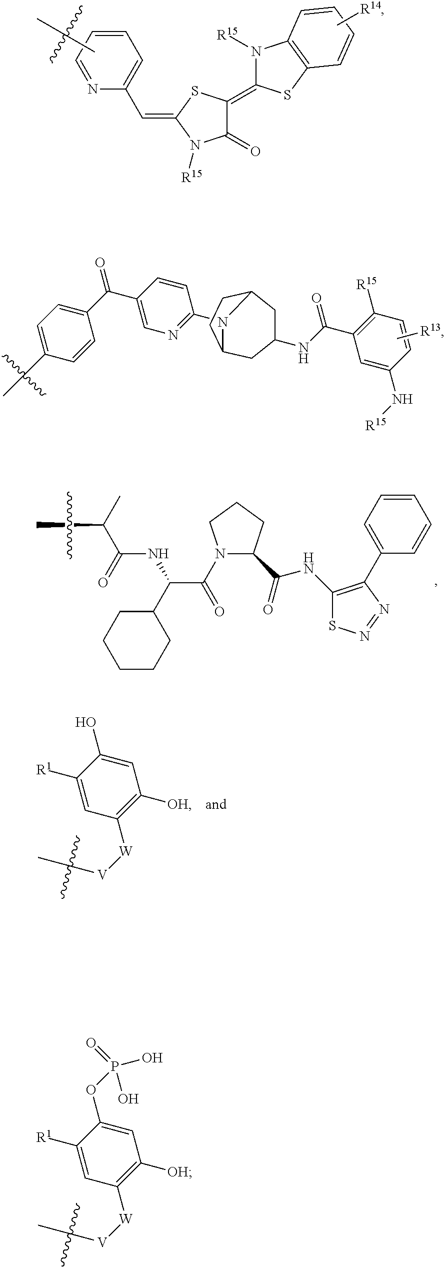

wherein
Q and U are each independently selected from phenyl, heteroaryl, heterocyclyl, and cycloalkyl, each of which being optionally substituted with 1 to 3 groups selected from R²
R¹³ and R¹⁴ are each independently selected from hydrogen, halo, —CN, (C₁-C₄)alkyl, halo(C₁-C₄)alkyl, and —C(O)NR^{a}R^{b};
R¹⁵ is hydrogen, (C₁-C₄)alkyl, or halo(C₁-C₄)alkyl;
W is 5- or 6-membered heteroaryl optionally substituted with 1 to 3 groups selected from R²;
V is phenyl or 5- to 9-membered heteroaryl optionally substituted with 1 to 3 groups selected from R³;
R¹ is halo, (C₁-C₄)alkyl, halo(C₁-C₄)alkyl, (C₁-C₄)alkoxy, or halo(C₁-C₄)alkoxy;
R² is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{a}, —OR^{a}, —C(O)R^{a}, —C(O)OR^{a}, —C(O)NR^{a}R^{b}, —C(O)NR^{a}(C₁₋₄alkylene)OR^{a}, —C(O)NR^{a}(C₁₋₄alkylene)NR^{a}R^{b}, —C(O)NR^{a}(C₁₋₄alkylene)OR, —NR^{a}R^{b}, —O(C₁₋₄alkylene)NR^{a}R^{b}, —C₁₋₄alkylNR^{a}R^{b}, —SR^{a}, —S(O)R^{a}, —S(O)₂R^{a}, S(O)NR^{a}R^{b}, —SO₂NR^{a}R^{b}, —NR^{a}(C₁₋₄alkyl)OR^{a}, —SH, —S(C₁₋₄alkyl), —NR^{a}(C₁₋₄alkyl)NR^{a}R^{b}, —C₁₋₆alkylC(O)NR^{a}R^{b}, —O(C₁₋₄alkylene)NR^{a}C(O)(C₁₋₄alkylene)NR^{a}R^{b}, phenyl or 5- to 7-membered heteroaryl, wherein said phenyl and 5- to 7-membered heteroaryl are each optionally and independently substituted with 1 to 3 groups selected from R⁴;
R^{a} and R^{b} are each independently selected from hydrogen and (C₁-C₄)alkyl, wherein said (C₁-C₄)alkyl is optionally substituted with one or more halo or a 3- to 7-membered heterocyclyl, or both (alternatively, the 3- to 7-membered heterocyclyl may be optionally substituted e.g., with 1 to 3 groups selected from halo, (C₁-C₄)alkyl, halo(C₁-C₄)alkyl, (C₁-C₄)alkoxy, halo(C₁-C₄)alkoxy, and —OSO₂(C₁-C₃)alkyl; and
R³ and R⁴ are each independently halo, —NR^{a}R^{b}, (C₁-C₄)alkyl, halo(C₁-C₄)alkyl, (C₁-C₄)alkoxy, or halo(C₁-C₄)alkoxy, wherein the remaining variables are as described for Formula I.

In another alternative, as part of a second embodiment, A is

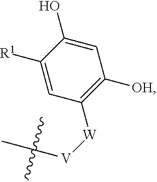

wherein the remaining variables are as described above and for Formula I. In another alternative, A is

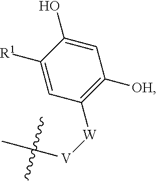

wherein the remaining variables are as described above and for Formula I.

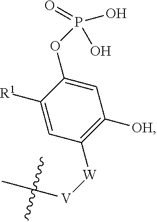

In a third embodiment, the compound of Formula I is of the Formula:

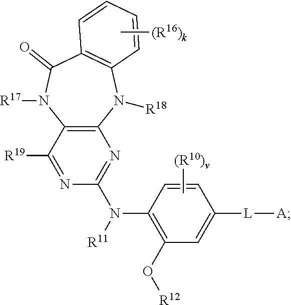

or a pharmaceutically acceptable salt thereof, wherein the variables are as described for Formula I or the second embodiment.

In a fourth embodiment, the compound of Formula I is of the Formula:

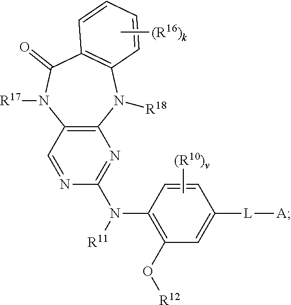

or a pharmaceutically acceptable salt thereof, wherein the variables are as described for Formula I or the second embodiment.

In a fifth embodiment, k is 0, wherein the remaining variables are as described above for Formula I or the first, second, third, or fourth embodiment.

In a sixth embodiment, v is 0, wherein the remaining variables are as described above for Formula I or the first, second, third, fourth, or fifth embodiment.

In a seventh embodiment, R¹¹ is hydrogen, wherein the remaining variables are as described above for Formula I or the first, second, third, fourth, fifth, or sixth embodiment.

In an eighth embodiment, R¹⁷ is (C₁-C₆)alkyl, wherein the remaining variables are as described above for Formula I or the first, second, third, fourth, fifth, sixth, or seventh embodiment. Alternatively, as part of an eighth embodiment, R¹⁷ is methyl, wherein the remaining variables are as described above for Formula I or the first, second, third, fourth, fifth, sixth, or seventh embodiment.

In an ninth embodiment, R¹² is (C₁-C₆)alkyl, wherein the remaining variables are as described above for Formula I or the first, second, third, fourth, fifth, sixth, seventh, or eighth embodiment. Alternatively, as part of an ninth embodiment, R¹² is ethyl, wherein the remaining variables are as described above for Formula I or the first, second, third, fourth, fifth, sixth, seventh, or eighth embodiment.

In a tenth embodiment, R¹⁸ is (C₁-C₃)alkyl or S(O)₂(C₁-C₃)alkyl, wherein the remaining variables are as described above for Formula I or the first, second, third, fourth, fifth, sixth, seventh, eighth, or ninth embodiment. Alternatively, as part of a tenth embodiment, R¹⁸ is S(O)₂Me, wherein the remaining variables are as described above for Formula I or the first, second, third, fourth, fifth, sixth, seventh, eighth, or ninth embodiment.

In an eleventh embodiment, A is selected from

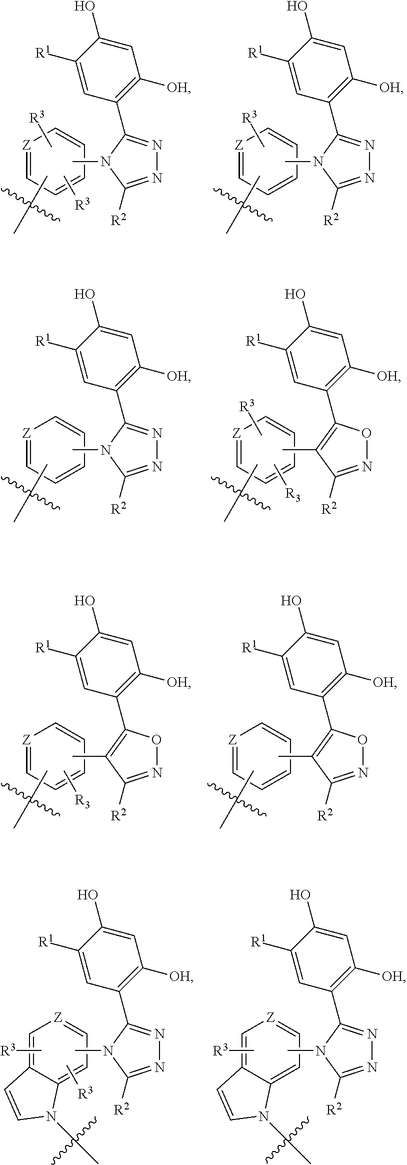

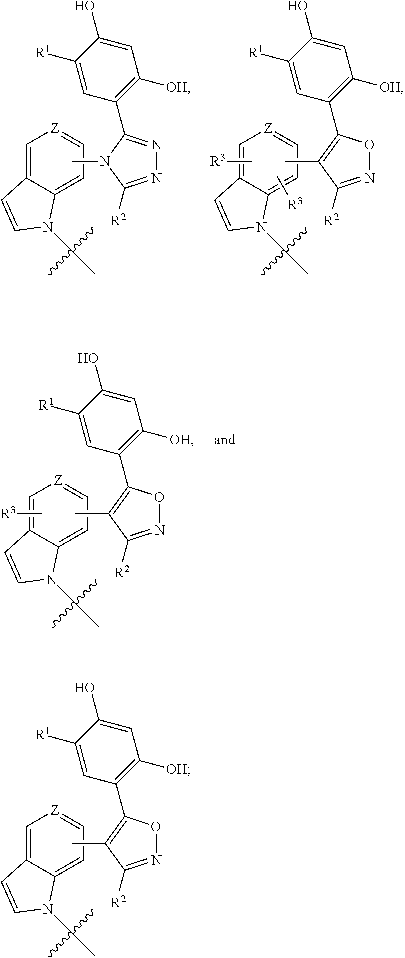

and Z is N or CH, wherein the remaining variables are as described above for Formula I or the first, second, third, fourth, fifth, sixth, seventh, eighth, ninth, or tenth embodiment. Alternatively, as part of an eleventh embodiment, A is selected from the structures above and Z is CH, wherein the remaining variables are as described above for Formula I or the first, second, third, fourth, fifth, sixth, seventh, eighth, ninth, or tenth embodiment.

In an twelfth embodiment, R³ is (C₁-C₄)alkyl or halo, wherein the remaining variables are as described above for Formula I or the first, second, third, fourth, fifth, sixth, seventh, eighth, ninth, tenth, or eleventh embodiment.

In a thirteenth embodiment, A is selected from

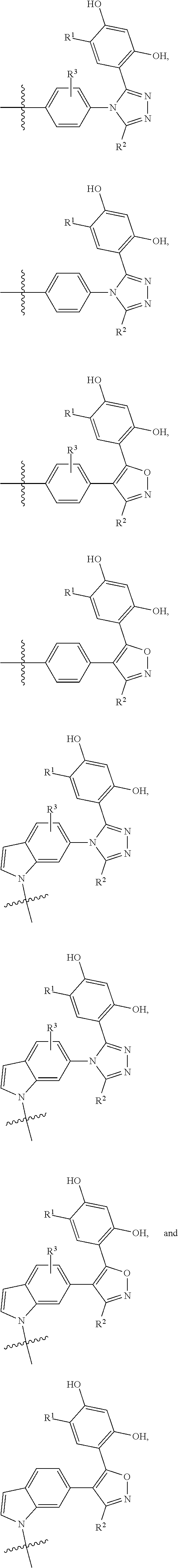

wherein the remaining variables are as described above for Formula I or the first, second, third, fourth, fifth, sixth, seventh, eighth, ninth, tenth, eleventh, or twelfth embodiment. Alternatively, as part of a thirteenth embodiment, A is selected from

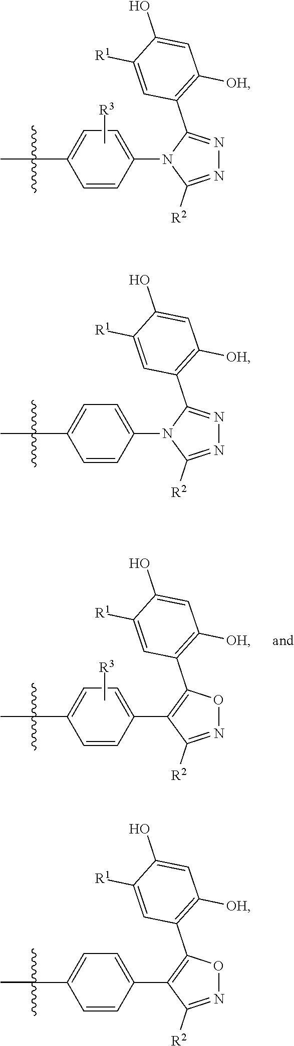

wherein the remaining variables are as described above for Formula I or the first, second, third, fourth, fifth, sixth, seventh, eighth, ninth, tenth, eleventh, or twelfth embodiment. In another alternative, as part of a thirteenth embodiment, A is selected from

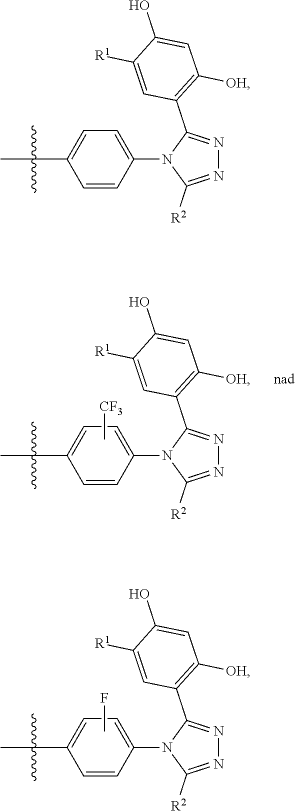

wherein the remaining variables are as described above for Formula I or the first, second, third, fourth, fifth, sixth, seventh, eighth, ninth, tenth, eleventh, or twelfth embodiment. In another alternative, as part of a thirteenth embodiment, A is

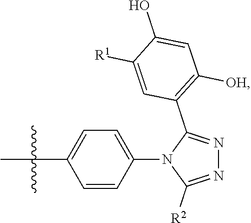

wherein the remaining variables are as described above for Formula I or the first, second, third, fourth, fifth, sixth, seventh, eighth, ninth, tenth, eleventh, or twelfth embodiment.

In a fourteenth embodiment, R¹ is halo or (C₁-C₄)alkyl, wherein the remaining variables are as described above for Formula I or the first, second, third, fourth, fifth, sixth, seventh, eighth, ninth, tenth, eleventh, twelfth, or thirteenth embodiment. Alternatively, as part of a fourteenth embodiment, R¹ is chloro, isopropyl, methyl, propyl, or ethyl, wherein the remaining variables are as described above for Formula I or the first, second, third, fourth, fifth, sixth, seventh, eighth, ninth, tenth, eleventh, twelfth, or thirteenth embodiment. In another alternative, as part of a fourteenth embodiment, R¹ is isopropyl or ethyl, wherein the remaining variables are as described above for Formula I or the first, second, third, fourth, fifth, sixth, seventh, eighth, ninth, tenth, eleventh, twelfth, or thirteenth embodiment.

In a fifteenth embodiment, R² is —OR^{a}, —SR^{a}, —C(O)NR^{a}R^{b}, or —C(O)NR^{a}(C₁₋₄alkylene)NR^{a}R^{b}, wherein the remaining variables are as described above for Formula I or the first, second, third, fourth, fifth, sixth, seventh, eighth, ninth, tenth, eleventh, twelfth, thirteenth, or fourteenth embodiment.

In a sixteenth embodiment, R^{a} and R^{b} are each independently selected from hydrogen and (C₁-C₄)alkyl, wherein said (C₁-C₄)alkyl is optionally substituted with 1 to 3 halo or a 6-membered heterocyclyl, wherein the remaining variables are as described above for Formula I or the first, second, third, fourth, fifth, sixth, seventh, eighth, ninth, tenth, eleventh, twelfth, thirteenth, fourteenth, or fifteenth embodiment.

In a seventeenth embodiment, R² is OH, —C(O)NHCH₂CF₃, —C(O)NHCH₂CH₃, —C(O)NHCH(CH₃)₂, —C(O)NH(CH₂CH₃)₂, —C(O)NHCH(CH₃)CF₃, —C(O)NHcyclopropyl, —C(O)NHmethylcyclopropyl, C(O)NH₂, or —C(O)NH(CH₂)₂piperidinyl, wherein the remaining variables are as described above for Formula I or the first, second, third, fourth, fifth, sixth, seventh, eighth, ninth, tenth, eleventh, twelfth, thirteenth, fourteenth, fifteenth, or sixteenth embodiment. Alternatively, as part of a seventeenth embodiment, R² is —C(O)NHCH₂CF₃ or OH, wherein the remaining variables are as described above for Formula I or the first, second, third, fourth, fifth, sixth, seventh, eighth, ninth, tenth, eleventh, twelfth, thirteenth, fourteenth, fifteenth, or sixteenth embodiment. In another alternative, as part of a seventeenth embodiment, R² is OH, wherein the remaining variables are as described above for Formula I or the first, second, third, fourth, fifth, sixth, seventh, eighth, ninth, tenth, eleventh, twelfth, thirteenth, fourteenth, fifteenth, or sixteenth embodiment.

In an eighteenth embodiment, L is selected from -Het¹-X¹—, -Het¹-, -Het¹-Het²-X¹—, -Het¹-Het²-, —NR^{d}—(CH₂)ₘ—X³—NR^{c}—(CH₂)ₘ-Het¹-X¹-Het²-X²—, —NR^{c}—(CH₂)ₘ-Het¹-X¹-Het²-X²—, -Het¹-X¹-Het²-X²—, O—(CH₂)ₘ—NR^{c}—X¹—(CH₂)ₘ—NR^{d}—, —X¹—NR^{c}—X²—O—(CH₂)ₘ—NR^{d}—, —X¹-Het¹-X²-Het²-(CH₂)ₘO—, O-Het¹-, O-Het¹-X¹—, —X¹(OCH₂CH₂)ₙ—NR^{c}—, —(CH₂)ₘNR^{c}—, —(CH₂)ₘ—, —O—, X¹NR^{c}—, —NR^{c}—(CH₂)ₘ—X¹-Het¹-X²—, —NR^{d}—(CH₂)ₘ—X³—NR^{c}—(CH₂)ₘ-Het¹-X¹-Het²-X²—, O-Het¹-X¹—(CH₂)ₘ—NR^{d}—, —X¹—NR¹—X²—(CH₂)ₘ—NR^{d}—, X¹-Het¹-X²—NR^{c}—X³-Het²-(OCH₂CH₂)ₙ—(CH₂)ₘ—NR^{d}—(CH₂)ₘ—, —NR^{d}—(CH₂)ₘ—X¹—NR^{c}—(CH₂CH₂O)ₙ—, —NR^{c}—(CH₂)ₘ—X¹—NR^{c}—(CH₂)ₚ—, X¹-Het-X²—NR^{c}—X³-Het²-(OCH₂CH₂)ₙ—NR^{d}—(CH₂)ₘ—, —NR^{c}—(CH₂)ₘ—X¹-Het¹-X²-Het²-X³—, O—X¹-Het¹-, —O(CH₂)ₘ—X¹-Het¹-X²-Het²-X³—, —O(CH₂)ₘ—X¹—NR^{c}—(CH₂)ₚ-Het¹-X²-Het²-X³—, O—(CH₂)ₘ—NR^{c}—, O—X¹-Het¹-X²—, —X¹—NR^{c}—(CH₂)ₘ-Het¹-X²-Het²-X³—(CH₂)ₚ—NR^{d}—(CH₂)ₚ—, —NR^{c}—(CH₂)ₘ—X¹—(CH)CH₃-Het¹-X²-Het³-X³—, —NR^{c}—(CH₂)ₘ—X¹—(CH₂)ₚ-Het¹-X²-Het²-X³—, —NR^{c}—(CH₂)ₘ—X¹—NR^{d}—(CH₂)ₚ-Het¹-X²-Het²-X³—, —NR^{c}—(CH₂)ₘ—NR^{d}—X¹-Het¹-X²—, Het¹-X¹-Het²-X²—, -Het¹-X¹-Het²-X²—O—, —O(CH₂)ₘ-Het¹-(CH₂)ₚ—O(CH₂)ₘ—NR^{c}—X²—, —O(CH₂)ₘ-Het¹-(CH₂)ₚ—O(CH₂)ₘ—NR^{c}—X²—, -Het¹-O—(CH₂)ₘ—X¹-Het²-X²—, -Het¹-O—(CH₂)ₘ—X¹—NR^{c}—(CH₂CH₂O)ₙ(CH₂)ₘ-Het²-X²—, -Het¹-X¹—NR^{c}—(CH₂)ₘ—, -Het¹-X¹-Het²-Het³-X²—, -Het¹-X¹—NR^{c}—(CH₂CH₂O)ₙ(CH₂)ₘ—, -Het¹-X¹—NR^{c}—(CH₂CH₂O)ₙHet²-(CH₂)ₘ—X²—, -Het¹-X¹—NR^{c}—(CH₂CH₂O)ₙ—, -Het¹-X¹—NR^{c}—(CH₂)ₘ-Het²-X²-Het³-(CH₂)ₘ—, -Het¹-X¹-Het²-(CH₂)ₘ-Het³-X²—, -Het¹-X¹-Het²-, -Het¹-X¹—NR^{c}—, -Het¹-X¹—NR^{c}—(CH₂)ₘ-Phe-X²-Het²-(CH₂)ₘ—, -Het¹-X¹-Het²-Het³-, -Het¹-X¹-Het²-(CH₂)ₘ-Het³-X²—(CH₂)ₚ—NR^{c}—(CH₂)ₘ—, -Het¹-X¹-Het²-(CH₂)ₘ-Het³-(CH₂)ₘO—, -Het¹-X¹-Het²-(CH₂)ₘ-Het³-(CH₂)ₚ—NR^{c}—(CH₂)ₘ—, -Het¹-X¹-Het²-(CH₂CH₂O)ₙ—, -Het¹-X¹—(CH₂)ₘ-Het²-X²—, —(CH₂CH₂O)ₒ—(CH₂)ₚ-Het¹-X¹-Het²-(CH₂CH₂O)ₙ, —(CH₂CH₂O)ₙ—(CH₂)ₘ-Het¹-X¹-Het²-X², -Het¹-X¹-Phe-X²—NR^{c}—X³—, —(CH₂CH₂O)ₒ—(CH₂)ₚ-Het¹-X¹-Phe-X²—NR^{c}—(CH₂CH₂O)ₙ—, —(CH₂CH₂O)ₙ—(CH₂)ₘ—NR^{c}-Phe-X¹—, —(CH₂CH₂O)ₒ—(CH₂)ₚ—NR^{c}-Phe-(CH₂CH₂O)ₙ—, —(CH₂CH₂O)ₒ—(CH₂)ₚ—NR^{c}—(CH₂CH₂O)ₙ—(CH₂)ₘ—, —(CH₂CH₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, —(CH₂CH₂O)ₒ—(CH₂)ₚ—NR^{c}—(CH₂CH₂O)ₙ—(CH₂)ₘ-Het¹-X¹-Het²-X²—, —(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ, —NR^{c}—(CH₂CH₂O)ₙ—(CH₂)ₘ-Phe- NH—X¹-Het¹-X², —NR^{c}—(CH₂CH₂O)ₙ—(CH₂)ₘ-Phe-NH—X¹-Het¹-X²—(CH₂CH₂O)ₒ, —(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ—(CH₂)ₚ—, —(CH₂CH₂O)ₒ—(CH₂)ₚ—NR^{c}—(CH₂CH₂)ₙ—(CH₂)ₘ-Het¹-X¹—, —(CH₂CH₂O)ₒ—(CH₂)ₚ—NR^{c}—(CH₂CH₂O)ₙ—(CH₂)ₘ-Het¹-X¹—(CH₂CH₂O)ₙ—, —(CH₂CH₂O)ₙ—(CH₂)ₘ—NR^{c}—(CH₂)ₘ—C(O)—NR^{d}-Het¹-X¹-Het²-(CH₂CH₂O)ₒ—(CH₂)ₚ, or —NR^{c}—(CH₂)ₘ—C(O)—NR^{d}—(CH₂)ₘ-Het¹-X¹-Het²-X²—, C(O)O—X¹-Het¹-(CH₂CH₂O)ₒ—(CH₂)ₘ—NR^{c}—, -Het¹-(CH₂)ₘ-Het²-, -Het¹-X¹-Het²-(CH₂)ₚ—O—(CH₂)ₘ—, O(CH₂)ₘC(O), —OC(O)—NR^{c}—(CH₂)ₘ—NR^{d}—, —OC(O)—NR^{c}—(CH₂)ₘO—(CH₂)ₘ—NR^{d}—, OC(O)Het¹, —OC(O)—NR^{c}—(CH₂CH₂O)ₒ—NR^{d}—, OC(O)Het¹-Het²-, —OC(O)—NR^{c}—(CH₂)ₘC(O)-Het¹-X¹-Het²-, O—(CH₂)ₘ-Het¹-, and O—(CH₂)ₘ-Het¹-X¹-Het²;
Het¹, Het², and Het³ are each independently phenyl, a 4- to 6-membered heterocyclyl, 5- to 7-membered heteroaryl, or a 4- to 6-membered cycloalkyl, each of which are optionally substituted with (C₁-C₄)alkyl;
X¹, X², and X³, are each independently C(O) or (CH₂)ᵣ; and
m, n, o, p, q and r are each independently integers selected from 0, 1, 2, 3, 4, 5, and 6, wherein the remaining variables are as described above for Formula I or the first, second, third, fourth, fifth, sixth, seventh, eighth, ninth, tenth, eleventh, twelfth, thirteenth, fourteenth, fifteenth, sixteenth, or seventeenth embodiment. Alternatively, as part of an eighteenth embodiment, L is selected from Het¹-X¹—, Het¹-X¹-Het²-X²—, Het¹-O—(CH₂)ₘ—X¹-Het²-X²—, Het¹-O—(CH₂)ₘX¹—NR^{c}—(CH₂CH₂O)ₙ(CH₂)ₘ-Het²-X²—, Het¹-X¹—NR^{c}—(CH₂)ₘ—, Het¹-X¹-Het²-Het³-X²—, Het¹-X¹—NR^{c}—(CH₂CH₂O)ₙ(CH₂)ₘ—, Het¹-X¹—NR^{c}—(CH₂CH₂O)ₙHet²-(CH₂)ₘ—X², Het¹-X¹—NR^{c}—(CH₂CH₂O)ₙ—, Het¹-X¹—NR^{c}—(CH₂)ₘ-Het²-X²-Het³-(CH₂)ₘ—, Het¹-X¹-Het²-(CH₂)ₘ-Het³-X²—, Het¹-X¹-Het²-, Het¹-X¹—NR^{c}—, Het¹-X¹—NR^{c}—(CH₂)ₘ-Phe-X²-Het²-(CH₂)ₘ—, Het¹-X¹-Het²-Het³-, Het¹-X¹-Het²-(CH₂)ₘ-Het³-X²—(CH₂)ₚ—NR^{c}—(CH₂)ₘ—, Het¹-X¹-Het²-(CH₂)ₘ-Het³-(CH₂)ₘ—O—, Het¹-X¹-Het²-(CH₂)ₘ-Het³-(CH₂)ₚ—NR^{c}—(CH₂)ₘ—, Het¹-X¹-Het²-(CH₂CH₂O)ₙ—, Het¹-X¹—(CH₂)ₘ-Het²-X²—, —(CH₂CH₂O)ₒ—(CH₂)ₚ-Het¹-X¹-Het²-(CH₂CH₂O)ₙ, —(CH₂CH₂O)ₙ—(CH₂)ₘ-Het¹-X¹-Het²-X², Het¹-X¹-Phe-X²—NR^{c}—X³—, —(CH₂CH₂O)ₒ—(CH₂)ₚ-Het¹-X¹-Phe-X²—NR^{c}—(CH₂CH₂O)ₙ—, —(CH₂CH₂O)ₙ—(CH₂)ₘ—NR^{c}-Phe-X¹—, —(CH₂CH₂O)ₒ—(CH₂)ₚ—NR^{c}-Phe-(CH₂CH₂O)ₙ—, —(CH₂CH₂O)ₒ—(CH₂)ₚ—NR^{c}—(CH₂CH₂O)ₙ—(CH₂)ₘ—, (CH₂CH₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, —(CH₂CH₂O)ₒ—(CH₂)ₚ—NR^{c}—(CH₂CH₂O)ₙ—(CH₂)ₘ-Het¹-X¹-Het²- X²—, —(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ, NR^{c}—(CH₂CH₂O)ₙ—(CH₂)ₘ-Phe-NH—X¹-Het¹-X², NR^{c}—(CH₂CH₂O)ₙ—(CH₂)ₘ-Phe-NH—X¹-Het¹-X²—(CH₂CH₂O)ₒ, —(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ—(CH₂)ₚ—, —(CH₂CH₂O)ₒ—(CH₂)ₚ—NR^{c}—(CH₂CH₂O)ₙ—(CH₂)ₘ-Het¹-X¹—, —(CH₂CH₂O)ₒ—(CH₂)ₚ—NR^{c}—(CH₂CH₂O)ₙ—(CH₂)ₘ-Het¹-X¹—(CH₂CH₂O)ₙ—, —(CH₂CH₂O)ₙ—(CH₂)ₘ—NR^{c}—(CH₂)ₘ—C(O)—NR^{d}-Het¹-X¹-Het²-(CH₂CH₂O)ₒ—(CH₂)ₚ, and NR^{c}—(CH₂)ₘ—C(O)—NR^{d}—(CH₂)ₘ-Het¹-X¹-Het²-X², wherein the remaining variables are as described above for Formula I or the first, second, third, fourth, fifth, sixth, seventh, eighth, ninth, tenth, eleventh, twelfth, thirteenth, fourteenth, fifteenth, sixteenth, or seventeenth embodiment. Alternatively, as part of an eighteenth embodiment, L is Het¹-X¹—*, Het¹-X¹-Het²-X²—*, Het¹-O—(CH₂)ₘ—X¹-Het²-X²—*, Het¹-O—(CH₂)ₘX¹—NR^{c}—(CH₂CH₂O)ₙ(CH₂)ₘ-Het²-X²—*, Het¹-X¹—NR^{c}—(CH₂)ₘ—*, Het¹-X¹-Het²-Het³-X²—*, Het¹-X¹—NR^{c}—(CH₂CH₂O)ₙ(CH₂)ₘ—*, Het¹-X¹—NR^{c}—(CH₂CH₂O)ₙHet²-(CH₂)ₘ—X²*, Het¹-X¹—NR^{c}—(CH₂CH₂O)ₙ—*, Het¹-X¹—NR^{c}—(CH₂)ₘ-Het²-X²-Het³-(CH₂)ₘ—*, Het¹-X¹-Het²-(CH₂)ₘ-Het³-X²—*, Het¹-X¹-Het²-*, Het¹-X¹—NR^{c}—*, Het¹-X¹—NR^{c}—(CH₂)ₘ-Phe-X²-Het²-(CH₂)ₘ—*, Het¹-X¹-Het²-Het³-*, Het-X¹-Het²-(CH₂)ₘ-Het³-X²—(CH₂)ₚ—NR^{c}—(CH₂)ₘ—*, Het¹-X¹-Het²-(CH₂)ₘ-Het³-(CH₂)ₘ—O—*, Het¹-X¹-Het²-(CH₂)ₘ-Het³-(CH₂)ₚ—NR^{c}—(CH₂)ₘ—*, Het¹-X¹-Het²-(CH₂CH₂O)ₙ—*, Het¹-X¹—(CH₂)ₘ-Het²-X²—*, —(CH₂CH₂O)ₒ—(CH₂)ₚ-Het¹-X¹-Het²-(CH₂CH₂O)ₙ*, —(CH₂CH₂O)ₙ—(CH₂)ₘ-Het¹-X¹-Het²-X²*, Het¹-X¹-Phe-X²—NR^{c}—X³—*, —(CH₂CH₂O)ₒ—(CH₂)ₚ-Het¹-X¹-Phe-X²—NR^{c}—(CH₂CH₂O)ₙ—*, —(CH₂CH₂O)ₙ—(CH₂)ₘ—NR^{c}-Phe-X¹—*, —(CH₂CH₂O)ₒ—(CH₂)ₚ—NR^{c}-Phe-(CH₂CH₂O)ₙ—*, —(CH₂CH₂O)ₒ—(CH₂)ₚ—NR^{c}—(CH₂CH₂O)ₙ—(CH₂)ₘ—*, (CH₂CH₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—*, —(CH₂CH₂O)ₒ—(CH₂)ₚ—NR^{c}—(CH₂CH₂O)ₙ—(CH₂)ₘ-Het¹-X¹-Het²-X²—*, —(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ*, NR^{c}—(CH₂CH₂O)ₙ—(CH₂)ₘ-Phe-NH—X¹-Het¹-X²*, NR^{c}—(CH₂CH₂O)ₙ—(CH₂)ₘ-Phe-NH—X¹-Het¹-X²—(CH₂CH₂O)ₒ*, —(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ—(CH₂)ₚ—*, —(CH₂CH₂O)ₒ—(CH₂)ₚ—NR^{c}—(CH₂CH₂)ₙ—(CH₂)ₘ-Het¹-X¹—*, —(CH₂CH₂O)ₒ—(CH₂)ₚ—NR^{c}—(CH₂CH₂)ₙ—(CH₂)ₘ-Het¹-X¹—(CH₂CH₂O)ₙ—*, —(CH₂CH₂O)ₙ—(CH₂)ₘ—NR^{c}—(CH₂)ₘ—C(O)—NR^{d}-Het¹-X¹-Het²-(CH₂CH₂O)ₒ—(CH₂)ₚ*, or NR^{c}—(CH₂)ₘ—C(O)—NR^{d}—(CH₂)ₘ-Het¹-X¹-Het²-X²*, wherein * indicates the point of attachment to A and wherein the remaining variables are as described above for Formula I or the first, second, third, fourth, fifth, sixth, seventh, eighth, ninth, tenth, eleventh, twelfth, thirteenth, fourteenth, fifteenth, sixteenth, or seventeenth embodiment. Alternatively, as part of an eighteenth embodiment, Het¹, Het², and Het³ are each independently phenyl, a 7- to 9-membered heterocyclyl, 5- to 7-membered heteroaryl, or a 4- to 6-membered cycloalkyl, each of which are optionally substituted with (C₁-C₄)alkyl, wherein the remaining variables are as described above for Formula I or the first, second, third, fourth, fifth, sixth, seventh, eighth, ninth, tenth, eleventh, twelfth, thirteenth, fourteenth, fifteenth, sixteenth, or seventeenth embodiment.

In a nineteenth embodiment, L is Het¹-X¹-Het²-X²—*, Het¹-O—(CH₂)ₘ—X¹-Het²-X²—*, Het-O—(CH₂)ₘX¹—NR^{c}—(CH₂CH₂O)ₙ(CH₂)ₘ-Het²-X²—*, Het¹-X¹—NR^{c}—(CH₂)ₘ—*, Het¹-X¹-Het²-Het³-X²—*, Het¹-X¹—NR^{c}—(CH₂CH₂O)ₙ(CH₂)ₘ—*, Het¹-X¹—NR^{c}—(CH₂CH₂O)ₙHet²-(CH₂)ₘ—X²*, Het¹-X¹—NR^{c}—(CH₂CH₂O)ₙ—*, Het¹-X¹—NR^{c}—(CH₂)ₘ-Het²-X²-Het³-(CH₂)ₘ—*, Het¹-X¹-Het²-(CH₂)ₘ-Het³-X²—*, Het¹-X¹-Het²-*, Het¹-X¹—NR^{c}—*, Het¹-X¹—NR^{c}—(CH₂)ₘ-Phe-X²-Het²-(CH₂)ₘ—*, Het¹-X¹-Het²-Het³-*, Het-X¹-Het²-(CH₂)ₘ-Het³-X²—(CH₂)ₚ—NR^{c}—(CH₂)ₘ—*, Het¹-X¹-Het²-(CH₂)ₘ-Het³-(CH₂)ₘ—O—*, Het¹-X¹-Het²-(CH₂)ₘ-Het³-(CH₂)ₚ—NR^{c}—(CH₂)ₘ—*, or Het¹-X¹-Het²-(CH₂CH₂O)ₙ—*, wherein the remaining variables are as described above for Formula I or the first, second, third, fourth, fifth, sixth, seventh, eighth, ninth, tenth, eleventh, twelfth, thirteenth, fourteenth, fifteenth, sixteenth, seventeenth, or eighteenth embodiment. Alternatively, as part of a nineteenth embodiment, L is Het¹-X¹-Het²-X²—*, Het¹-X¹—NR^{c}—(CH₂)ₘ—*, Het¹-X¹-Het²-Het³-X²—*, or Het¹-X¹-Het²-(CH₂)ₘ-Het³-X²—*, wherein the remaining variables are as described above for Formula I or the first, second, third, fourth, fifth, sixth, seventh, eighth, ninth, tenth, eleventh, twelfth, thirteenth, fourteenth, fifteenth, sixteenth, seventeenth, or eighteenth embodiment. In another alternative, as part of a nineteenth embodiment, L is Het¹-X¹—NR^{c}—(CH₂)ₘ—* or Het¹-X¹-Het²-(CH₂)ₘ-Het³-X²—*, wherein the remaining variables are as described above for Formula I or the first, second, third, fourth, fifth, sixth, seventh, eighth, ninth, tenth, eleventh, twelfth, thirteenth, fourteenth, fifteenth, sixteenth, seventeenth, or eighteenth embodiment. In yet another alternative, as part of an nineteenth embodiment, L is Het¹-X¹-Het²-(CH₂)ₘ-Het³-X²—*, wherein the remaining variables are as described above for Formula I or the first, second, third, fourth, fifth, sixth, seventh, eighth, ninth, tenth, eleventh, twelfth, thirteenth, fourteenth, fifteenth, sixteenth, seventeenth, or eighteenth embodiment.

In a twentieth embodiment, Het and Het² as described herein (e.g., as in the eighteenth or nineteenth embodiment), are each independently phenyl or a 4- to 6-membered heterocyclyl. Alternatively, as part of a twentieth embodiment, Het¹ and Het² as described herein (e.g., as in the eighteenth or nineteenth embodiment), are each independently piperidinyl, phenyl, pyridinyl, piperazinyl, or pyrrolidinyl.

In a twenty-first embodiment, m, n, o, p, q and r as described herein (e.g., as in the eighteenth or nineteenth embodiment, or as applied to the twentieth embodiment), are each independently integers selected from 0, 1, 2, and 3.

In a twenty-second embodiment, L is selected from

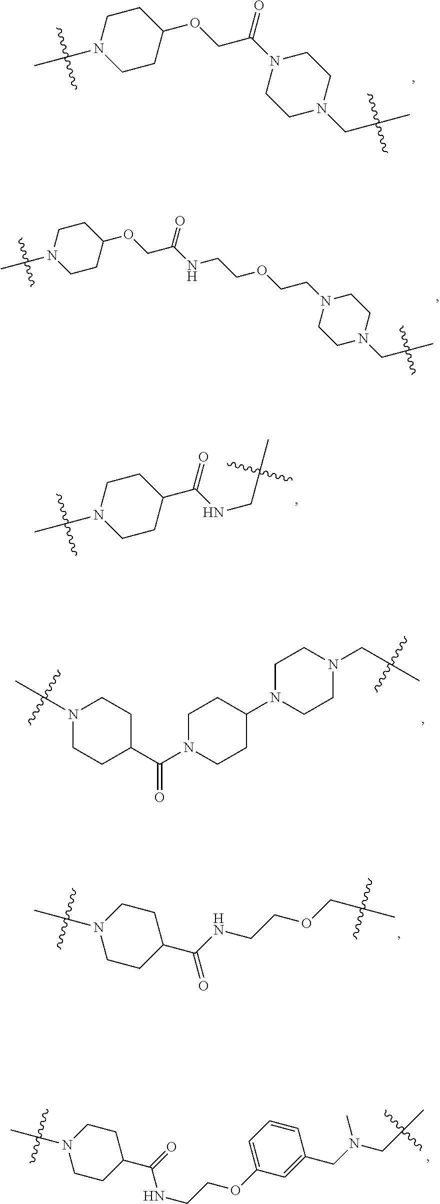

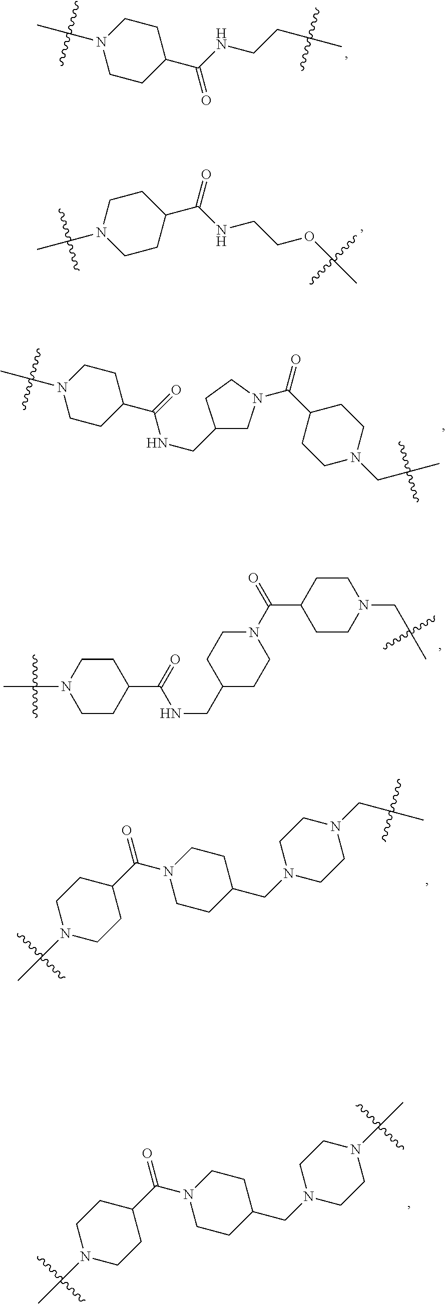 [SMILES: *CCNC(=O)C1CCN(*)CC1 ; *OCCNC(=O)C1CCN(*)CC1 ; *CN1CCC(C(=O)N2CCC(CNC(=O)C3CCN(*)CC3)C2)CC1 ; *CN1CCC(C(=O)N2CCC(CNC(=O)C3CCN(*)CC3)CC2)CC1 ; *CN1CCN(CC2CCN(C(=O)C3CCN(*)CC3)CC2)CC1 ; *N1CCC(C(=O)N2CCC(CN3CCN(*)CC3)CC2)CC1]

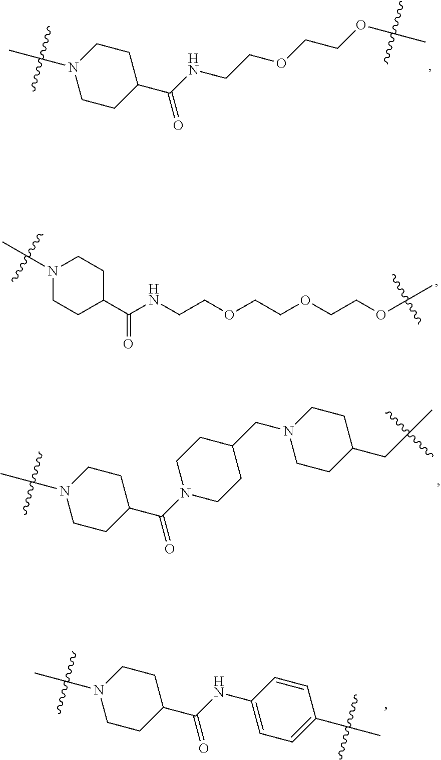 [SMILES: *OCCOCCNC(=O)C1CCN(*)CC1 ; *OCCOCCOCCNC(=O)C1CCN(*)CC1 ; *CC1CCN(CC2CCN(C(=O)C3CCN(*)CC3)CC2)CC1 ; *c1ccc(NC(=O)C2CCN(*)CC2)cc1]

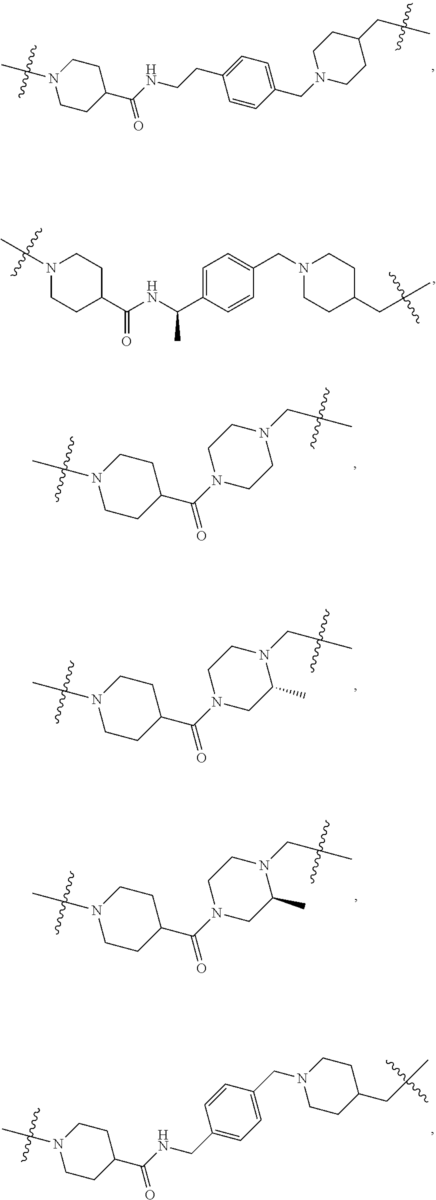 [SMILES: *CC1CCN(Cc2ccc(CCNC(=O)C3CCN(*)CC3)cc2)CC1 ; *CC1CCN(Cc2ccc([C@@H](C)NC(=O)C3CCN(*)CC3)cc2)CC1 ; *CN1CCN(C(=O)C2CCN(*)CC2)CC1 ; *CN1CCN(C(=O)C2CCN(*)CC2)C[C@H]1C ; *CN1CCN(C(=O)C2CCN(*)CC2)C[C@@H]1C ; *CC1CCN(Cc2ccc(CNC(=O)C3CCN(*)CC3)cc2)CC1]

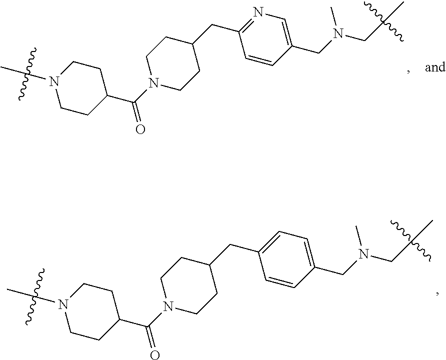 [SMILES: *CN(C)Cc1ccc(CC2CCN(C(=O)C3CCN(*)CC3)CC2)nc1 ; *CN(C)Cc1ccc(CC2CCN(C(=O)C3CCN(*)CC3)CC2)cc1]

wherein the remaining variables are as described above for Formula I or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, or twenty-first embodiment. Alternatively, as part of a twenty-second embodiment, L is selected from

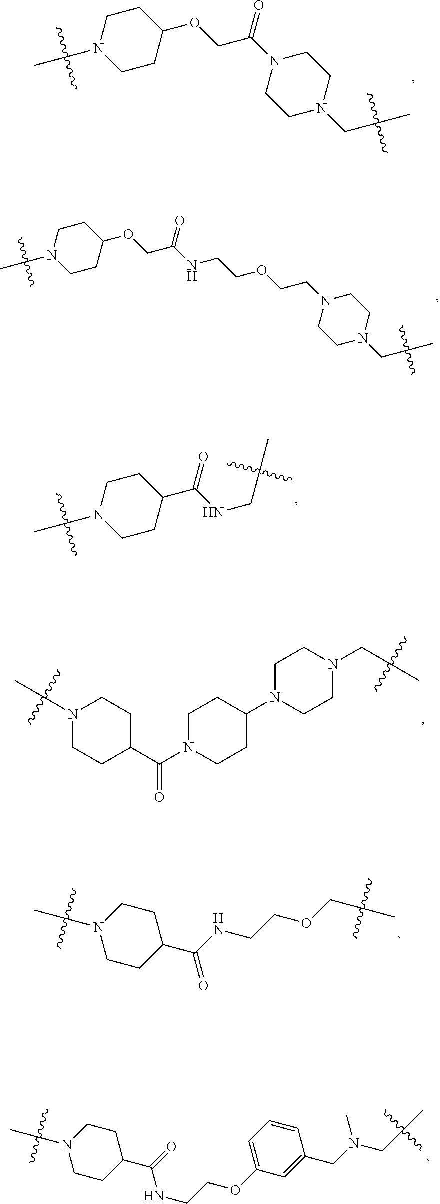

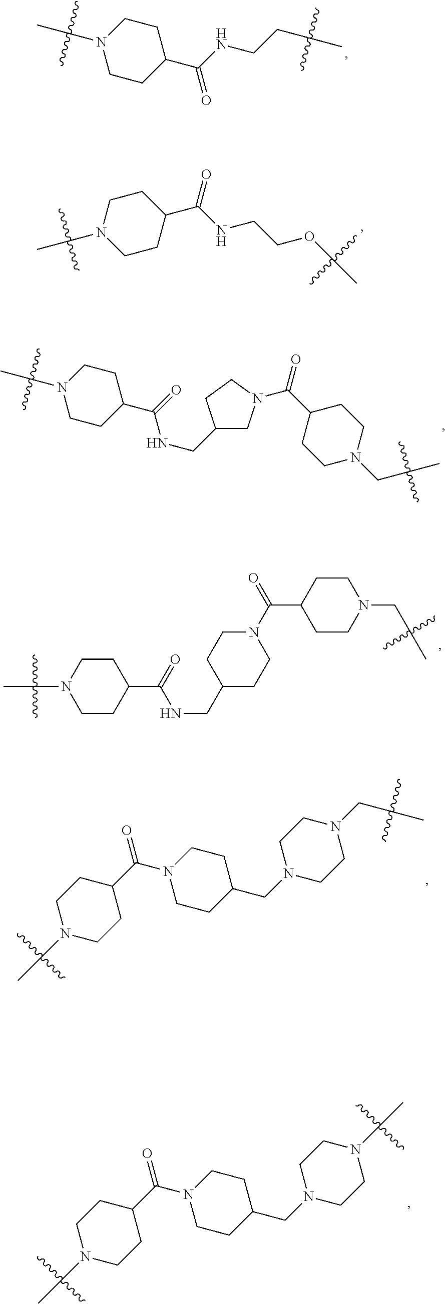 [SMILES: *CCNC(=O)C1CCN(*)CC1 ; *OCCNC(=O)C1CCN(*)CC1 ; *CN1CCC(C(=O)N2CCC(CNC(=O)C3CCN(*)CC3)C2)CC1 ; *CN1CCC(C(=O)N2CCC(CNC(=O)C3CCN(*)CC3)CC2)CC1 ; *CN1CCN(CC2CCN(C(=O)C3CCN(*)CC3)CC2)CC1 ; *N1CCC(C(=O)N2CCC(CN3CCN(*)CC3)CC2)CC1]

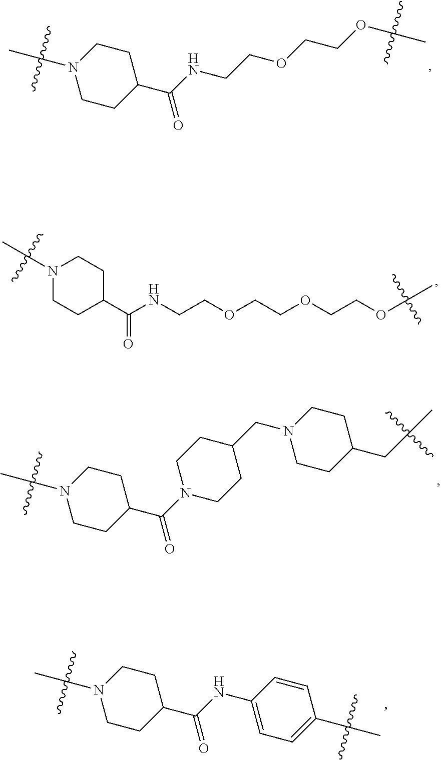 [SMILES: *OCCOCCNC(=O)C1CCN(*)CC1 ; *OCCOCCOCCNC(=O)C1CCN(*)CC1 ; *CC1CCN(CC2CCN(C(=O)C3CCN(*)CC3)CC2)CC1 ; *c1ccc(NC(=O)C2CCN(*)CC2)cc1]

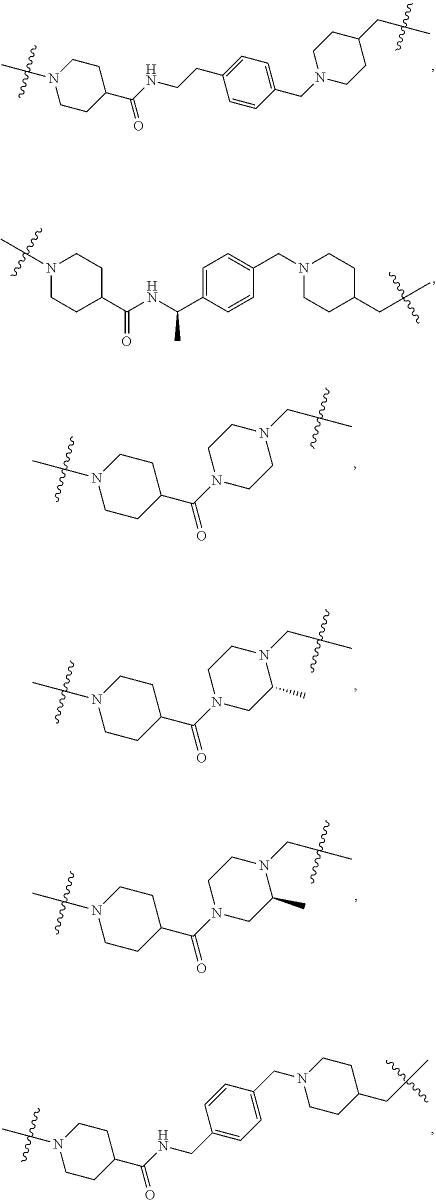 [SMILES: *CC1CCN(Cc2ccc(CCNC(=O)C3CCN(*)CC3)cc2)CC1 ; *CC1CCN(Cc2ccc([C@@H](C)NC(=O)C3CCN(*)CC3)cc2)CC1 ; *CN1CCN(C(=O)C2CCN(*)CC2)CC1 ; *CN1CCN(C(=O)C2CCN(*)CC2)C[C@H]1C ; *CN1CCN(C(=O)C2CCN(*)CC2)C[C@@H]1C ; *CC1CCN(Cc2ccc(CNC(=O)C3CCN(*)CC3)cc2)CC1]

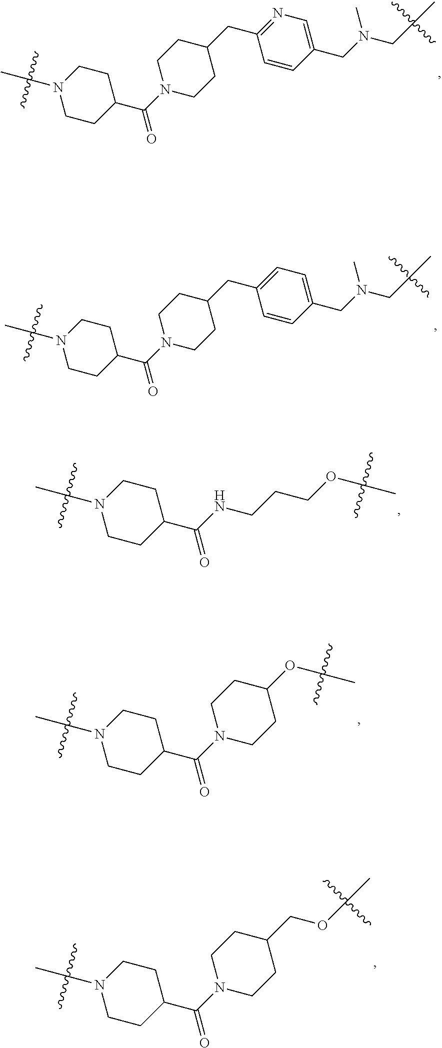 [SMILES: *CN(C)Cc1ccc(CC2CCN(C(=O)C3CCN(*)CC3)CC2)nc1 ; *CN(C)Cc1ccc(CC2CCN(C(=O)C3CCN(*)CC3)CC2)cc1 ; *OCCCNC(=O)C1CCN(*)CC1 ; *OC1CCN(C(=O)C2CCN(*)CC2)CC1 ; *OCC1CCN(C(=O)C2CCN(*)CC2)CC1]

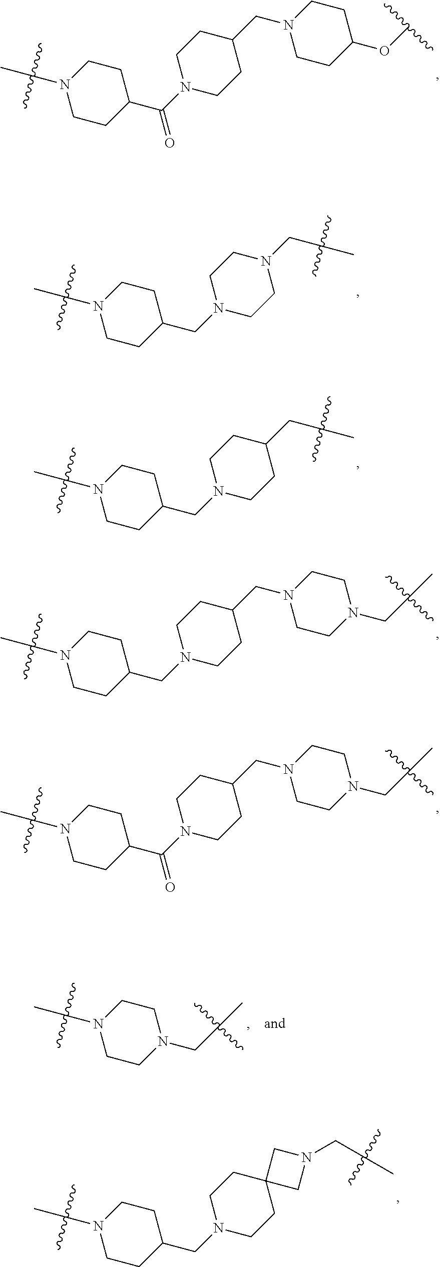

wherein the remaining variables are as described above for Formula I or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, or twenty-first embodiment. Alternatively, as part of a twenty-second embodiment, L is selected from

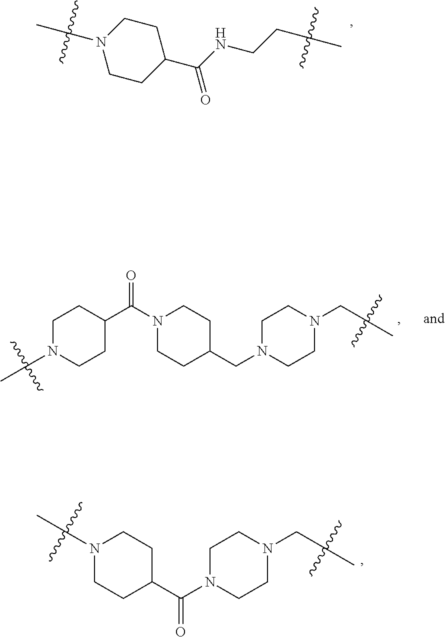 [SMILES: *CCNC(=O)C1CCN(*)CC1 ; *CN1CCN(CC2CCN(C(=O)C3CCN(*)CC3)CC2)CC1 ; *CN1CCN(C(=O)C2CCN(*)CC2)CC1]

wherein the remaining variables are as described above for Formula I or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, or twenty-first embodiment.

Specific compounds are provided in the exemplification section below and are included as part of the invention. Salts and free bases of these compounds are also included.

### 4. Uses, Formulation and Administration

Compounds and compositions described herein are generally useful as anticancer therapies. In one aspect, the disclosed compounds and compositions behave as tumor-targeted, chaperone-mediated protein degraders (CHAMPs) in which one portion of the compounds is responsible for binding MAPK7 and the other portion is responsible for binding to HSP90 or other chaperone proteins or protein components of chaperone complexes (e.g., members of the HSP70 family). Their mechanisms of action include, but are not limited to, degrading MAPK7 and/or other related members of the mitogen activated protein kinase (MAPK) protein family, and thereby impeding down-stream signals that may result in inhibition of cancer cell growth and/or induction of cancer cell death or other MAPK7 or MAPK functions. In one aspect, the disclosed compounds effectuate the degradation of MAPK7.

In one aspect, the disclosed compounds and compositions include chaperone or chaperone complex binders that have a range of different binding affinities. In different embodiments, it is desirable to use a high-affinity binder, a moderate-affinity binder or a low-affinity binder. Since a HSP90-binding moiety that interacts with the N-terminal ATP-binding pocket of HSP90 may inhibit HSP90 activity and induce the degradation of HSP90 client proteins (Schopf et al., Nat Rev Mol Cell Biol, 2017, 18:345-360), some CHAMP molecules may not only induce the degradation of the desired target protein or proteins (which may or may not be HSP90 client proteins), but also simultaneously induce the degradation of HSP90 client proteins. EGFR and ERBB2 (HER2) are two such HSP90 client proteins (Xu et al., J Biol Chem, 2001, 276:3702-3708). Such combinations of degradation activities may increase the biological activity of CHAMP molecules over that of other TPD technologies directed towards the same target(s) and may evade mechanisms of resistance to MAPK7 inhibitors and degraders such as that mediated by HSP90 client protein(s).

In one aspect, the disclosed compounds and compositions behave as tumor-targeted CHAMPs in which one portion of the compounds is responsible for binding MAPK7 and the other portion is responsible for binding to HSP90 or other chaperone proteins or protein components of chaperone complexes (e.g., members of the HSP70 family). In one aspect, the disclosed compounds and compositions have prolonged pharmacokinetic exposures in cancer cells and tumors relative to normal cells, tissues and organs (Kamal et al., Nature, 2003, 425:407-410; Vilenchik et al., Chem Biol, 2004, 11:787-797). In one aspect, the disclosed compounds have increased therapeutic indexes relative to other MAPK7 inhibitors.

Thus, provided herein are methods of treating conditions which are responsive to the degradation of MAPK7 comprising administering to a subject in need thereof, a therapeutically effective amount of one or more compounds or compositions described herein. Also provided is the use of one or more compounds or compositions described herein in the manufacture of a medicament for treating conditions which are responsive to the degradation of MAPK7. Further provided is the use of a compound or composition described herein for treating conditions which are responsive to the degradation of MAPK7.

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphangioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### EXEMPLIFICATION

### Example 1: Compound 043 Synthesis

A representative synthesis scheme for compound 043 is shown in below. Specific synthesis routes of intermediates are also shown.

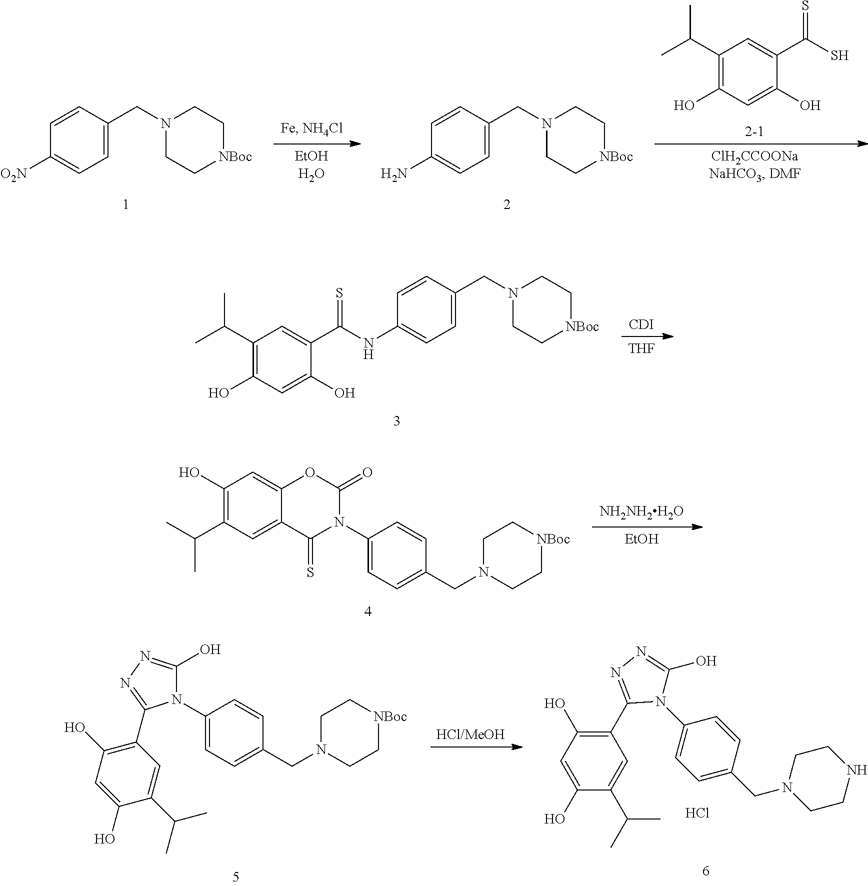

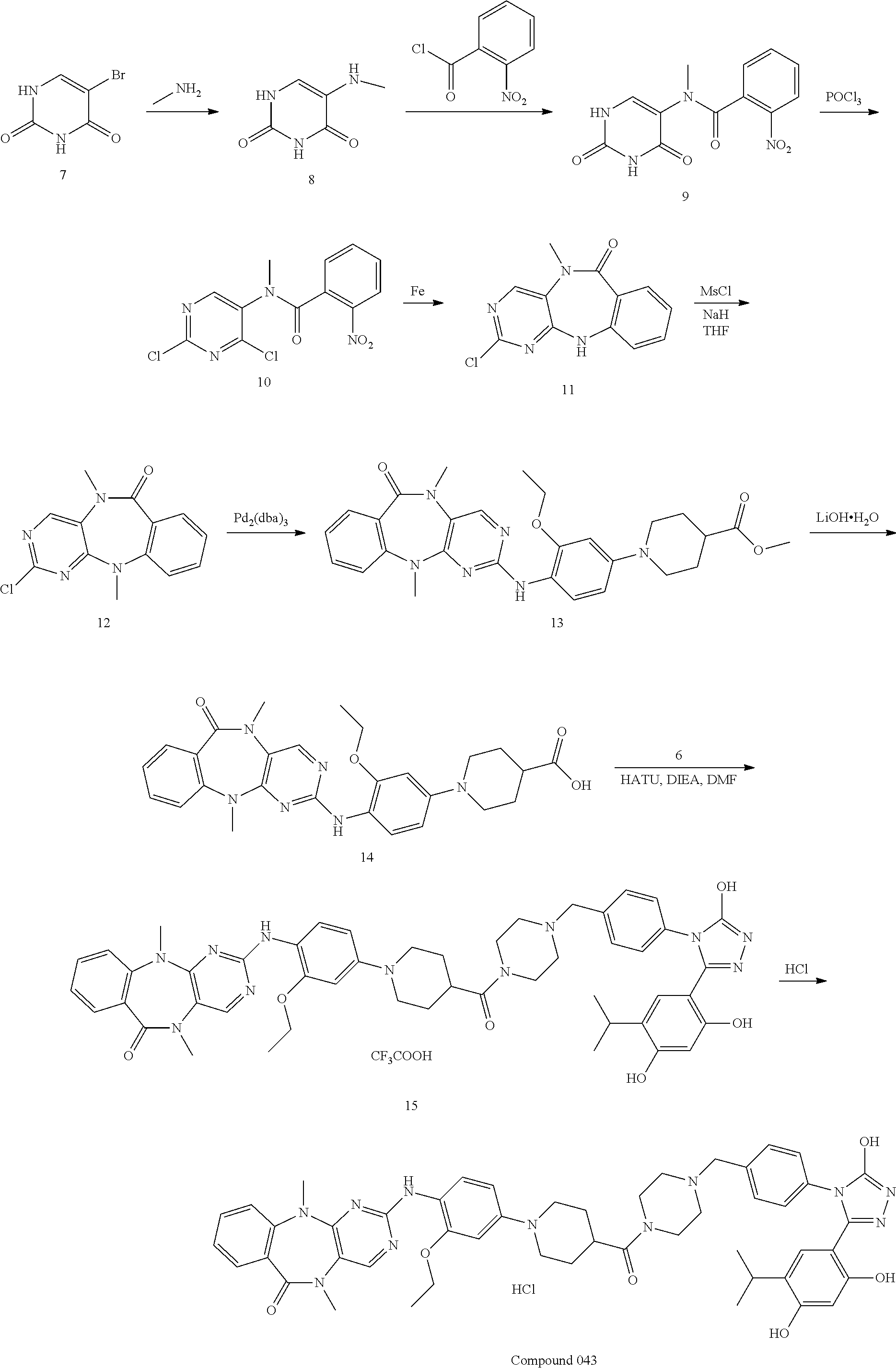

### Intermediate 2:

### tert-butyl 4-(4-aminobenzyl)piperazine-1-carboxylate

To a solution of compound 1 (2.0 g, 15.6 mmol) in EtOH (15 mL) and H₂O (5 mL) was added Fe power (1.72 g, 77.9 mmol) and NH₄Cl (3.4 g, 105.5 mmol). The resulting mixture was heated to 80° C. for 2 hours. The reaction solution was cooled to room temperature and filtered. The filtrate was poured into aq. NaHCO₃ solution, extracted with EtOAc (20 mL*3). The combine organic layers were washed with brine, dried over Na₂SO₄ and concentrated to give intermediate 2 (1.81 g, yield 100%) as a white solid.

### Intermediate 3:

### tert-butyl 4-(4-(2,4-dihydroxy-5-isopropylphenylthioamido)benzyl)piperazine-1-carboxylate

The solution of compound 2-1 (1.45 g, 6.30 mmol), ClCH₂COONa (1.09 g, 9.53 mmol) and NaHCO₃ (1.60 g, 19.1 mmol) in DMF (10 mL) was stirred for at 30° C. 3 hours. Compound 2 (1.85 g, 6.3 mmol) was added to the mixture. After the resulting mixture was heated at 80° C. for 4 hours, the reaction mixture was poured into ice-water and extracted with EtOAc (15 mL*3). The combine organic layers was washed with brine, dried over Na₂SO₄ and filtered. The filtrate was concentrated and purified by SGC eluted with DCM:MeOH=20:1 to give intermediate 3 (2.1 g, yield 70%) as a yellow oil.

### Intermediate 4:

### tert-butyl 4-(4-(7-hydroxy-6-isopropyl-2-oxo-4-thioxo-2H-benzo[e][1,3]oxazin-3(4H)-yl)benzyl)piperazine-1-carboxylate

The solution of intermediate 3 (2.1 g 4.3 mmol) and CDI (1.40 g, 8.6 mmol) in THF (15 mL) was stirred at room temperature for 4 hours. The reaction solution was poured into brine (25 mL) and extracted with EtOAc (25 mL*2). The combine organic layers was washed with brine, dried over Na₂SO₄ and concentrated to give intermediate 4 (2.7 g, crude) which was used for further reaction without purification.

### Intermediate 5:

### tert-butyl 4-(4-(3-(2,4-dihydroxy-5-isopropylphenyl)-5-hydroxy-4H-1,2,4-triazol-4-yl)benzyl)piperazine-1-carboxylate

To a solution of intermediate 4 (2.7 g, crude) in EtOH (6 mL) was added NH₂NH₂·H₂O (253 mg, 7.9 mmol). The resulting mixture was stirred at room temperature overnight. The precipitated solid was filtered to give intermediate 5 (1.3 g, yield 48.5%) as a white solid.

### Intermediate 6:

### 4-(5-hydroxy-4-(4-(piperazin-1-ylmethyl)phenyl)-4H-1,2,4-triazol-3-yl)-6-isopropylbenzene-1,3-diol, Hydrochloride

The solution of intermediate 5 (1.3 g, 2.50 mmol) in HCl/MeOH (3 N, 15 mL) was stirred at room temperature for 16 hours. The reaction solution was concentrated to give intermediate 6 (1.02 g, yield 98%) as a white solid.

### Intermediate 8:

### 5-(methylamino)pyrimidine-2,4(1H,3H)-dione

To a 500-mL three-neck round bottom flask filled with a condenser, 5-bromopyrimidine-2,4(1H,3H)-dione (40 g, 0.2 mol) and methanamine (30% (wt/vol) aqueous solution, 400 mL) were added. The reaction mixture was stirred and heated at 80° C. for 3.5 h. At 25° C., the reaction mixture was acidified to pH ˜4.5 with diluted HCl aqueous solution. The generated light yellow precipitates were filtered and washed with water, and then dried in vacuo to provide intermediate 8 (25 g, 89% yield) as a light yellow solid.

### Intermediate 9:

### N-(2,4-dioxo-1,2,3,4-tetrahydropyrimidin-5-yl)-N-methyl-2-nitrobenzamide

To a solution of intermediate 8 (30 g, 0.21 mol) in tetrahydrofuran (THF) (300 mL), NaOH aqueous solution (2.5 N, 300 mL, 0.75 mol) was added at 0° C. 2-Nitrobenzoyl chloride (61 g, 0.32 mol) was added slowly. The generated clear brown solution was stirred at 0° C. for 40 min and then at room temperature for 4.5 h. The reaction mixture was acidified by diluted HCl aqueous solution. The generated light yellow solid was filtered, and the cake was washed with water and then dried in vacuo to provide intermediate 9 (32 g, 52% yield) as a light yellow solid.

### Intermediate 10:

### N-(2,4-dichloropyrimidin-5-yl)-N-methyl-2-nitrobenzamide

A solution of intermediate 9 (32 g, 0.11 mol) and N,N-dimethylaniline (12 g, 0.1 mol) in phosphorus oxychloride (205 mL) was heated at 100° C. overnight. The solvent was removed by rotavapor, and the residue was dried in vacuo to provide the crude product intermediate 10, which was used for the next step reaction without further purification.

### Intermediate 11:

### 2-chloro-5-methyl-5,11-dihydro-6H-benzo[e]pyrimido[5,4-b][1,4]diazepin-6-one

The crude intermediate 10 (36 g, 0.11 mol) was dissolved in acetic acid (400 mL), and then iron (37 g, 0.66 mol) was added at 25° C. with rigorous stirring. The mixture was heated at 60° C. for 5 h. Water (100 mL) and ethanol (10 mL) were added and the reaction mixture was stirred for 30 min. The precipitates were filtered and extracted between EtOAc and water. The combined EtOAc phase was dried over sodium sulfate and then concentrated to produce the crude intermediate 11 which was purified by SGC to give intermediate 11 (20 g, 70% yield for two steps) as a white solid.

### Intermediate 12:

### 2-chloro-5,11-dimethyl-5,11-dihydro-6H-benzo[e]pyrimido[5,4-b][1,4]diazepin-6-one

To a solution of intermediate 11 (5 g, 19.18 mmol) was dissolved in DMF (50 mL), and then NaH (60% in mineral oil, 1.53 g, 38 mmol) was added at 0° C. with stirring. The mixture was stirred at r.t. for 2 h. Water was added and extracted with EtOAc (50 mL*3). The combined EtOAc phase was dried over sodium sulfate and then concentrated to produce the crude intermediate 12 which was purified by SGC to give intermediate 12 (4.8 g, 91% yield) as white solid.

### Intermediate 13:

### methyl 1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxylate

To a solution of intermediate 12 (5 g, 18.2 mmol) in dioxane (50 mL) were added methyl 1-(4-amino-3-ethoxyphenyl)piperidine-4-carboxylate (5.07 g, 18.2 mmol), K₂CO₃ (7.55 g, 54.6 mmol), X-Phos (1.7 g) and Pd₂(dba)₃ (1.6 g). The mixture was stirred at reflux overnight under Ar atmosphere. Water was added and extracted with EtOAc (50 mL*3). The combined EtOAc phase was dried over sodium sulfate and then concentrated to produce the crude intermediate 13 which was purified by SGC to give intermediate 13 (4.0 g, 43% yield) as white solid.

### Intermediate 14:

### 1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxylic Acid

To a solution of intermediate 13 (2 g, 3.87 mmol) in H₂O/MeOH/THF (10 mL/5 mL/5 mL) was added LiOH—H₂O (0.42 g, 10 mmol). The mixture was stirred at r.t. overnight. Water was added and extracted with EtOAc (50 mL*3). 1N HCl was added and the pH was adjusted to 4. Solid precipitated were collected by filtration and dried to give compound intermediate 14 (1.5 g, 87.7% yield) as white solid.

### Compound 043:

### 2-((4-(4-(4-(4-(3-(2,4-dihydroxy-5-isopropylphenyl)-5-hydroxy-4H-1,2,4-triazol-4-yl)benzyl)piperazine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, Hydrochloride

To a solution of intermediate 6 (340 mg, 0.7 mmol), HATU (290 mg, 0.77 mmol) and DIEA (450 mg, 3.48 mmol) in DMF (8 mL) was added intermediate 14 (350 mg, 0.7 mmol). The resulting mixture was stirred at room temperature for 2 hours. The mixture was purified by prep-HPLC to give intermediate 15 (TFA salt) as a white solid. It was added to NaHCO₃ solution and EtOAc was added then extracted. Organic phase was dried and concentrated. H₂O (10 mL) and CH₃CN (1 mL) were added to the residue followed by 3N HCl (0.17 mL). It was lyophilized to give compound 043 (230 mg) as yellow solid.

¹H NMR (400 MHz, DMSO-d₆): δ 13.02 (s, 1H), 11.92 (m, 2H), 9.94-9.24 (m, 1H), 8.49 (d, J=28.4 Hz, 2H), 8.26 (d, J=8.4 Hz, 1H), 7.72-7.45 (m, 6H), 7.30-7.19 (m, 4H), 6.91 (s, 1H), 6.36 (s, 1H), 4.49-4.16 (m, 6H), 3.76-3.53 (m, 5H), 3.41 (s, 3H), 3.37 (s, 3H), 3.29 (d, J=8.4 Hz, 2H), 3.23-3.12 (m, 2H), 3.10-2.89 (m, 3H), 2.29 (s, 2H), 1.94 (s, 2H), 1.40 (t, J=7.2 Hz, 3H), 1.03 (d, J=6.8 Hz, 6H). LCMS (ESI): R_{T}=1.080 min, m/z. found 894.3 [M−HCl+H]⁺.

### Example 2: Compound 065 Synthesis

A representative synthesis scheme for compound 065 is shown in below. Specific synthesis routes of intermediates are also shown.

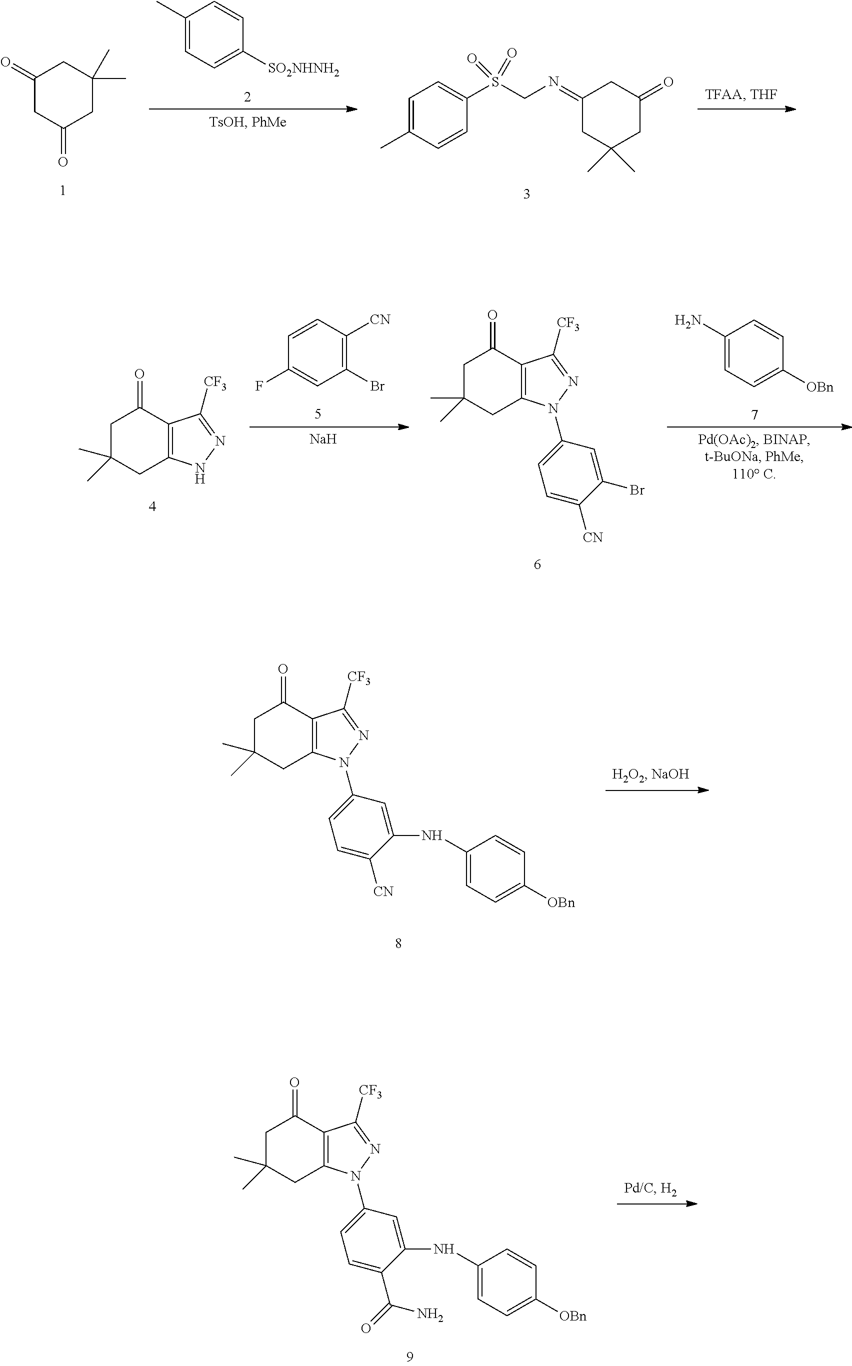

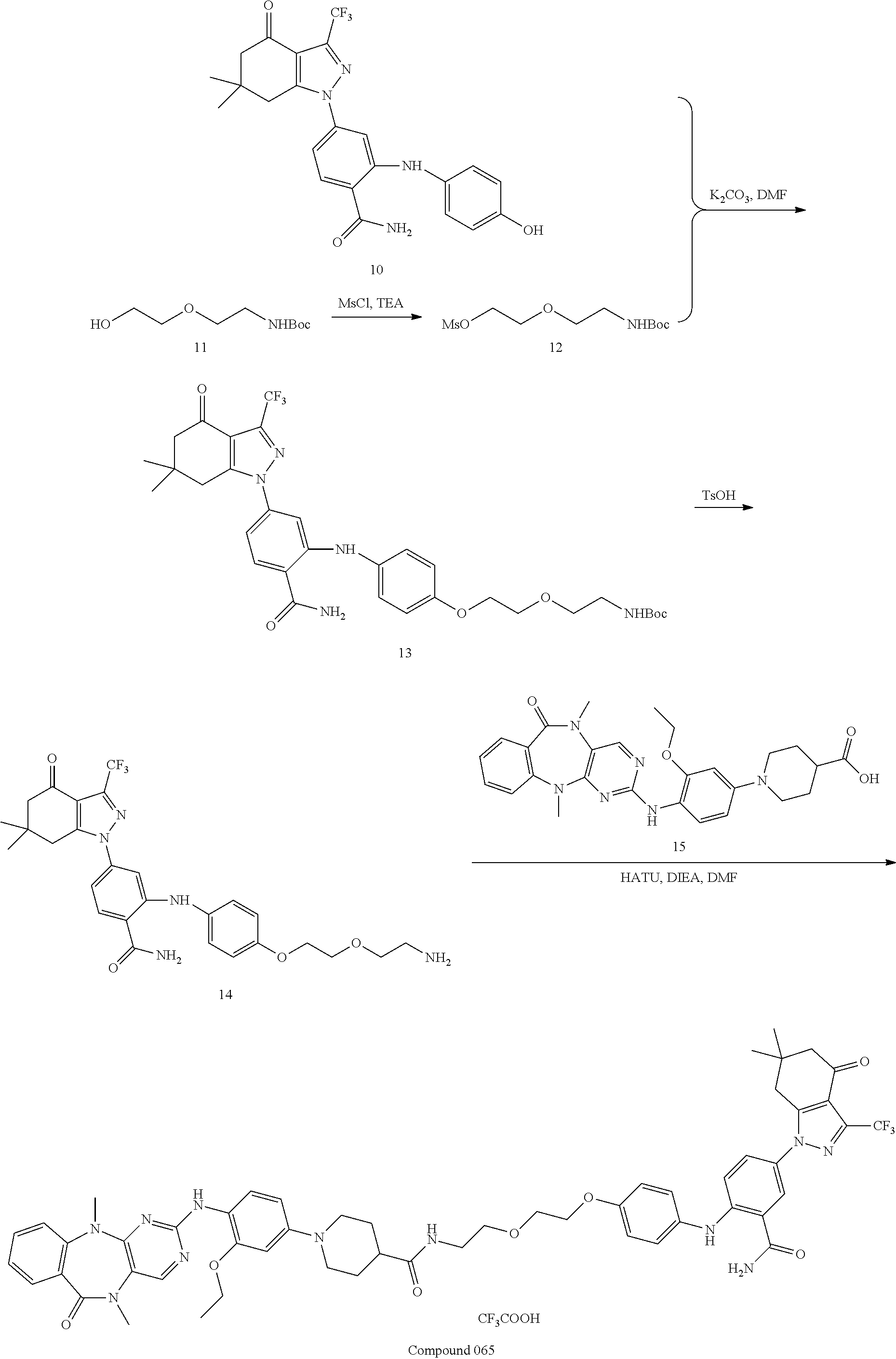

### Intermediate 3:

### (E)-N′-(3,3-dimethyl-5-oxocyclohexylidene)-4-methylbenzenesulfonohydrazide

A mixture of 1 (200 g, 1426.72 mmol), 2 (265.71 g, 1426.7167 mmol) and p-Toluene sulfonic acid (24.54 g, 142.67 mmol) in toluene (8 L) was heated to 120° C. After 1 h, the mixture was cooled and followed by the addition of toluene (1.2 L). The mixture was then reflux for 1 h. The reaction was cooled to ambient temperature. The precipitated solids was collected by filtration, washed three times with ether and dried under vacuum to give intermediate 3 (360 g, 1167.30 mmol, 81.82%). LCMS: m/z 309 [M+H]⁺.

### Intermediate 4:

### 6,6-dimethyl-3-(trifluoromethyl)-1,5,6,7-tetrahydro-4H-indazol-4-one

To a suspension of 3 (360 g, 1167.30 mmol) and TEA (486.67 mL, 3501.33 mmol) in THF (3 L) was added trifluoroacetyl 2,2,2-trifluoroacetate (243.51 mL, 1750.67 mmol) at 0° C. The resulting reaction was heated to 55° C. for 3 h, the reaction mixture was cooled to ambient temperature. To the mixture was added Methanol (1.4 L) and 1 N NaOH (1.4 L). After stirring for 3 h, the reaction mixture was diluted with saturated ammonium chloride (3 L), extracted with ethyl acetate three times, the combine organic layers was washed with brine, dried over sodium sulfate, and concentrated in vacuum. The residue was purified by column chromatography to give the intermediate 4 (160 g, 689.05 mmol, 59.04%). LCMS: m/z 233 [M+H]⁺.

### Intermediate 6:

### 2-bromo-4-(6,6-dimethyl-4-oxo-3-(trifluoromethyl)-4,5,6,7-tetrahydro-1H-indazol-1-yl)benzonitrile

NaH (15.50 g, 645.98 mmol) was added to a solution of 4 (150 g, 645.98 mmol) in DMSO (2 L) at room temperature. After 15 min, 2-bromo-4-fluorobenzonitrile (129.20 g, 645.98 mmol) was added as solid. The reaction mixture was heated at 45° C. overnight. The mixture was cooled to room temperature and quenched with saturated aqueous NH₄Cl. The mixture was diluted with water and extracted with EtOAc. The combined organic layers were washed with brine, dried over anhydrous sodium sulfate and concentrated in vacuum. The residue was purified by column chromatography to give intermediate 6 (180 g, 436.67 mmol, 67.59%). LCMS: m/z 412 [M+H]⁺.

### Intermediate 8:

### 2-((4-(benzyloxy)phenyl)amino)-4-(6,6-dimethyl-4-oxo-3-(trifluoromethyl)-4,5,6,7-tetrahydro-1H-indazol-1-yl)benzonitrile

To a solution of 6 (50 g, 121.30 mmol) in toluene (500 mL) was added 7 (24.17 g, 121.30 mmol) and Cs₂CO₃ (79.04 g, 242.59 mmol). Then BINAP (15.10 g, 24.26 mmol) and Pd(OAc)₂ (2.74 g, 12.13 mmol) was added successively under nitrogen protection. The mixture reaction was heated to 120° C. for 3 h. After which in was filtered, the filtrate was concentrated in vacuum, the residue was purified by silica gel chromatography to give the intermediate 8 (40 g, 75.39 mmol, 62.16%). LCMS: m/z 531 [M+H]⁺.

### Intermediate 9:

### 2-((4-(benzyloxy)phenyl)amino)-4-(6,6-dimethyl-4-oxo-3-(trifluoromethyl)-4,5,6,7-tetrahydro-1H-indazol-1-yl)benzamide

To a solution of 8 (40 g, 75.39 mmol) in EtOH (400 mL) and DMSO (100 mL) was added 1 N NaOH (226.18 mL, 226.18 mmol) and H₂O₂ (25.63 g, 226.18 mmol) dropwise successively at 0° C. Then the mixture was stirred at RT for 2 h before diluting with water, it was extracted with EtOAc, washed with brine, dried over sodium sulfate. The organic layer was concentrated in vacuum, the residue was purified by silica gel column to give the intermediate 9 (35 g, 63.80 mmol, 84.63%). LCMS: m/z 549 [M+H]⁺.

### Intermediate 10:

### 4-(6,6-dimethyl-4-oxo-3-(trifluoromethyl)-4,5,6,7-tetrahydro-1H-indazol-1-yl)-2-((4-hydroxyphenyl)amino)benzamide

To a solution of 9 (35 g, 63.80 mmol) in MeOH (400 mL) was added Pd/C 10% (6.7 g, 6.38 mmol), the mixture was stirred at RT overnight with H₂ existence. After which it was filtered, washed with EA followed by DCM, the filler was concentrated in vacuum to give intermediate 10 (26 g, 56.71 mmol, 88.89%) as a solid. LCMS: m/z 459 [M+H]⁺.

### Intermediate 12:

### 2-(2-((tert-butoxycarbonyl)amino)ethoxy)ethyl Methanesulfonate

To a solution of 11 (1 g, 4.87 mmol) in THF (15 mL) was added TEA (2.03 mL, 14.62 mmol) at 0° C., then MsCl (0.57 mL, 7.31 mmol) was added to the mixture reaction dropwised and the temperature was controlled below 5° C. After it was stirred at RT for 2 h before diluting with water. Then it was extracted with EA, wash with saturated brine, dried over sodium sulfate, concentrated in vacuum to give the intermediate 12 (1.3 g, 4.59 mmol, 94.17%). LCMS: m/z 284 [M+H]⁺.

### Intermediate 13:

### tert-butyl(2-(2-(4-((2-carbamoyl-5-(6,6-dimethyl-4-oxo-3-(trifluoromethyl)-4,5,6,7-tetrahydro-1H-indazol-1-yl)phenyl)amino)phenoxy)ethoxy)ethyl)carbamate

To a solution of 12 (1.3 g, 4.59 mmol) in DMF (20 mL) was added 10 (1.89 g, 4.13 mmol) and K₂CO₃ (1.90 g, 13.76 mmol), the mixture was stirred at 90° C. overnight. Then water was added, it was extracted with EA, washed with saturated brine, dried over sodium sulfate, concentrated in vacuum, the residue was purified by silica gel column to give the intermediate (1.6 g, 2.48 mmol, 54.01%) as a solid. LCMS: 646 [M+H]⁺.

### Intermediate 14:

### 2-((4-(2-(2-aminoethoxy)ethoxy)phenyl)amino)-4-(6,6-dimethyl-4-oxo-3-(trifluoromethyl)-4,5,6,7-tetrahydro-1H-indazol-1-yl)benzamide

To a solution of 13 (1.6 g, 2.48 mmol) in acetonitrile (25 mL) was added TsOH (1.41 g, 7.43 mmol), the mixture was stirred at 50° C. for 2 h, saturated NaHCO₃ solution was added to pH 8-9, then it was extracted with EA, washed with saturated brine, dried over sodium sulfate, concentrated in vacuum to give the intermediate 14 without further purification as a yellow solid. LCMS: 546 [M+H]⁺. H NMR (400 MHz, DMSO-d₆): δ 10.08 (s, 1H), 8.16 (s, 1H), 7.88 (d, J=8.4 Hz, 1H), 7.57 (s, 1H), 7.21 (d, J=8.4 Hz, 2H), 7.01 (s, 1H), 6.94 (t, J=9.6 Hz, 3H), 4.07 (t, J=4.6 Hz, 2H), 3.70 (d, J=9.0 Hz, 1H), 3.41 (t, J=5.8 Hz, 3H), 2.89 (s, 2H), 2.65 (t, J=5.8 Hz, 2H), 2.41 (s, 2H), 1.02 (s, 6H).

### Compound 065:

### N-(2-(2-(4-((2-carbamoyl-4-(6,6-dimethyl-4-oxo-3-(trifluoromethyl)-4,5,6,7-tetrahydro-1H-indazol-1-yl)phenyl)amino)phenoxy)ethoxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, Trifluoroacetic Acid

To a solution of intermediate 14 (340 mg, 0.7 mmol), HATU (290 mg, 0.77 mmol) and DIEA (450 mg, 3.48 mmol) in DMF (8 mL) was added intermediate 15 (350 mg, 0.7 mmol). The resulting mixture was stirred at room temperature for 2 hours. The mixture was purified by prep-HPLC to give compound 067 (TFA salt) as a white solid. (230 mg) as yellow solid. ¹H NMR (400 MHz, DMSO-d₆): δ 10.12 (s, 1H), 8.37 (s, 1H), 8.20-8.05 (m, 3H), 7.90 (d, J=8.4 Hz, 1H), 7.69 (dd, J=7.6, 1.6 Hz, 1H), 7.64-7.47 (m, 2H), 7.26-7.16 (m, 4H), 7.03-6.93 (m, 5H), 4.55-4.28 m, 4H), 4.16-4.08 (m, 5H), 3.74 (t, J=4.4 Hz, 2H), 3.64 (d, J=12.2 Hz, 2H), 3.51 (t, J=5.6 Hz, 2H), 3.39 (s, 3H), 3.32 (s, 3H), 3.26 (dd, J=11.4, 5.8 Hz, 2H), 2.91 (s, 2H), 2.42 (s, 3H), 1.88 (s, 3H), 1.35 (t, J=6.8 Hz, 3H), 1.03 (s, 6H). LCMS (ESI): R_{T}=1.500 min, m/z. found 1030.4 [M-CF₃COOH+H]⁺.

### Example 3: Compound 143 Synthesis

A representative synthesis scheme for compound 143 is shown in below. Specific synthesis routes of intermediates are also shown.

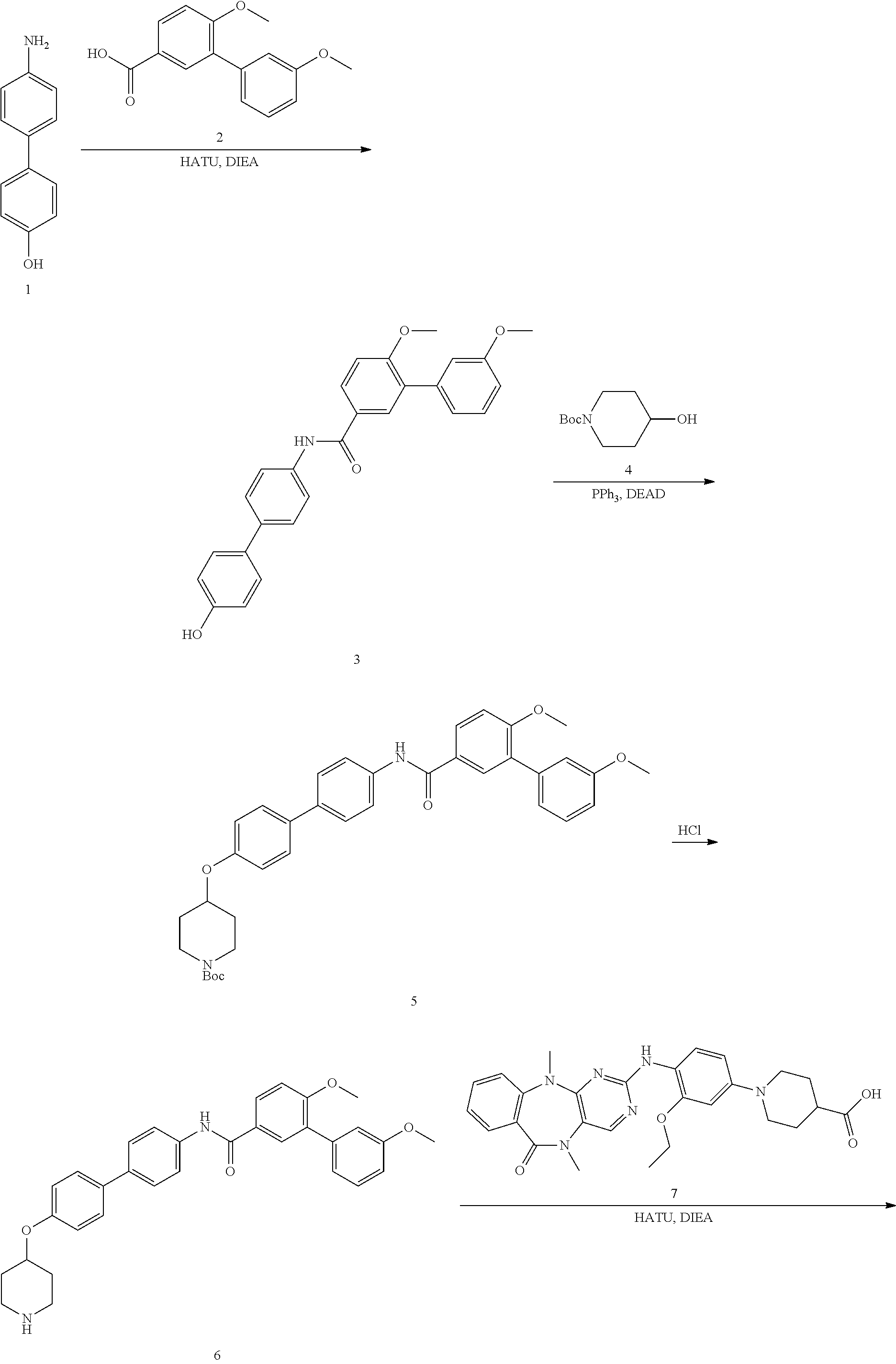

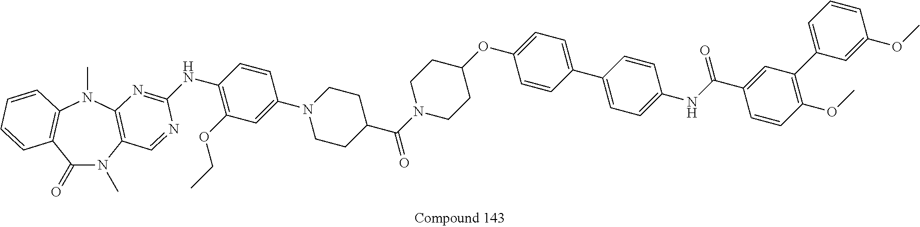 [SMILES: CCOc1cc(N2CCC(C(=O)N3CCC(Oc4ccc(-c5ccc(NC(=O)c6ccc(OC)c(-c7cccc(OC)c7)c6)cc5)cc4)CC3)CC2)ccc1Nc1ncc2c(n1)N(C)c1ccccc1C(=O)N2C]

### Intermediate 3:

To a solution of compound 1 (3.0 g, 16.20 mmol) and compound 2 (4.6 g, 17.82 mmol) in DMF (50 mL) was added DIEA (8.37 g, 64.79 mmol), followed by addition of HATU (6.77 g, 17.82 mmol). The mixture was stirred at room temperature for overnight. LC-MS indicated the reaction was completed. The reaction mixture was diluted with water (100 mL), extracted with EA (150 mL×2). The combined organic layer was washed saturated aqueous solution of NaHCO₃, concentrated under vacuum, the crude was purified by SGC eluted with DCM:MeOH=50:1 to give compound 3 (2.7 g, yield 39%) as a brown solid.

### Intermediate 5:

To a solution of compound 3 (200 mg, 0.47 mmol), compound 4 (189.2 mg, 0.94 mmol) and PPh₃ (369.9 mg, 1.41 mmol) in dry THF (9 mL). The mixture was stirred at room temperature for 15 min under Ar atmosphere. The DEAD (245.6 mg, 1.41 mmol) was added. Then the reaction mixture was heated to 65° C. and stirred for overnight under Ar atmosphere. LC-MS indicated the reaction was completed. The reaction mixture was diluted with water (30 mL), extracted with EA (50 mL×2). The combined organic layer was concentrated under vacuum and purified by SGC eluted with PE:EA=3:1 to give compound 5 (152 mg, yield 53%) as a yellow solid.

### Intermediate 6:

The solution of compound 3 (152 mg, 0.25 mmol) in MeOH (2 mL) was added HCl/dioxane (2 mL). The mixture was stirred at room temperature for 1 h, LC-MS indicated the reaction was completed. The reaction mixture was concentrated under vacuum to give intermediate 6 (124 mg, yield 97%) as a yellow solid.

### Compound 143:

### N-(4′-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)oxy)-[1,1′-biphenyl]-4-yl)-3′,6-dimethoxy-[1,1′-biphenyl]-3-carboxamide, Trifluoroacetic Acid

To a solution of compound 6 (35.79 mg, 0.066 mmol) and compound 7 (30 mg, 0.06 mmol) in DMF (2 mL) was added DIEA (30.86 mg, 0.239 mmol), followed by addition of HATU (24.97 mg, 0.066 mmol). The mixture was stirred at room temperature for 1 h. LC-MS indicated the reaction was completed. The reaction mixture was purified by pre-HPLC (TFA) to give compound 143 (18.92 mg, yield 32%) as a yellow solid. ¹H NMR (400 MHz, DMSO-d₆): δ 10.22 (s, 1H), 8.39 (s, 1H), 8.25-7.95 (m, 4H), 7.85 (d, J=8.4 Hz, 2H), 7.73-7.49 (m, 6H), 7.40-6.95 (m, 11H), 4.75-4.66 (m, 2H), 4.19-4.11 (m, 2H), 3.94-3.78 (m, 8H), 3.71-3.63 (m, 2H), 3.54-3.45 (m, 1H), 3.40 (s, 4H), 3.33 (s, 4H), 3.09-2.95 (m, 1H), 2.10-1.85 (m, 6H), 1.74-1.50 (m, 2H), 1.37 (t, J=6.8 Hz, 3H), 1.27-1.21 (m, 1H). LCMS (ESI): R_{T}=1.778 min, m/z. found 993.1 [M-CF₃COOH+H]⁺.

Additional compounds were made according to the general procedure and scheme noted in the Examples including the following:

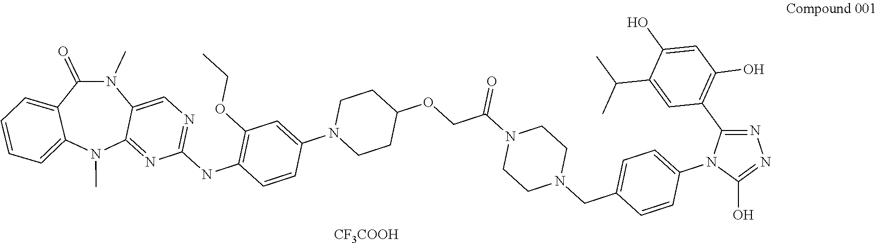 [SMILES: CCOc1cc(N2CCC(OCC(=O)N3CCN(Cc4ccc(-n5c(O)nnc5-c5cc(C(C)C)c(O)cc5O)cc4)CC3)CC2)ccc1Nc1ncc2c(n1)N(C)c1ccccc1C(=O)N2C ; "CF3COOH"]

2-((4-(4-(2-(4-(4-(3-(2,4-dihydroxy-5-isopropylphenyl)-5-hydroxy-4H-1,2,4-triazol-4-yl)benzyl)piperazin-1-yl)-2-oxoethoxy)piperidin-1-yl)-2-ethoxyphenyl)amino)-5,11-dimethyl-5H-benzo[e]pyrimido[5,4-b][1,4]diazepin-6(11H)-one, trifluoroacetic acid. ¹H NMR (400 MHz, DMSO-d₆): δ 11.98 (s, 1H), 9.63 (s, 1H), 9.36 (s, 1H), 8.20-8.19 (m, 1H), 7.66 (s, 1H), 7.51-7.50 (m, 2H), 7.23-7.21 (m, 5H), 6.88 (s, 1H), 6.63-6.62 (m, 2H), 6.25 (s, 1H), 5.33 (d, J=4.7 Hz, 1H), 4.34 (s, 3H), 3.99 (s, 3H), 3.26-3.20 (m, 12H), 2.95-2.93 (m, 5H), 1.95-1.93 (m, 4H), 1.49-1.47 (m, 2H), 1.23-1.20 (m, 5H), 0.85 (d, J=6.8 Hz, 6H). LCMS (ESI): R_{T}=1.047 min, m/z. found 925.3 [M-CF₃COOH+H]⁺.

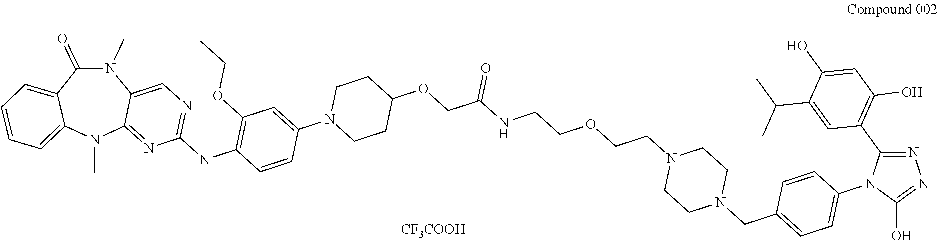 [SMILES: CCOc1cc(N2CCC(OCC(=O)NCCOCCN3CCN(Cc4ccc(-n5c(O)nnc5-c5cc(C(C)C)c(O)cc5O)cc4)CC3)CC2)ccc1Nc1ncc2c(n1)N(C)c1ccccc1C(=O)N2C ; "CF3COOH"]

N-(2-(2-(4-(4-(3-(2,4-dihydroxy-5-isopropylphenyl)-5-hydroxy-4H-1,2,4-triazol-4-yl)benzyl)piperazin-1-yl)ethoxy)ethyl)-2-((1-(4-((5,11-dimethyl-6-oxo-6,11-dihydro-5H-benzo[e]pyrimido[5,4-b][1,4]diazepin-2-yl)amino)-3-ethoxyphenyl)piperidin-4-yl)oxy)acetamide, trifluoroacetic acid. ¹H NMR (400 MHz, DMSO-d₆): δ 11.95 (s, 1H), 9.63 (s, 1H), 9.39 (s, 1H), 8.19 (s, 1H), 7.96 (s, 1H), 7.66 (d, J=7.5 Hz, 1H), 7.48 (s, 1H), 7.38-7.10 (m, 6H), 6.87-6.60 (m, 3H), 6.26 (s, 1H), 5.32 (d, J=4.7 Hz, 1H), 4.00 (s, 3H), 3.88-3.49 (m, 8H), 3.30 (m, 11H), 3.08-2.77 (m, 7H), 1.96 (m, 3H), 1.51 (d, J=47.6 Hz, 3H), 1.19 (m, 7H), 0.97 (d, J=6.8 Hz, 6H). LCMS (ESI): R_{T}=1.061 min, m/z. found 1012.0 [M-CF₃COOH+H]⁺.

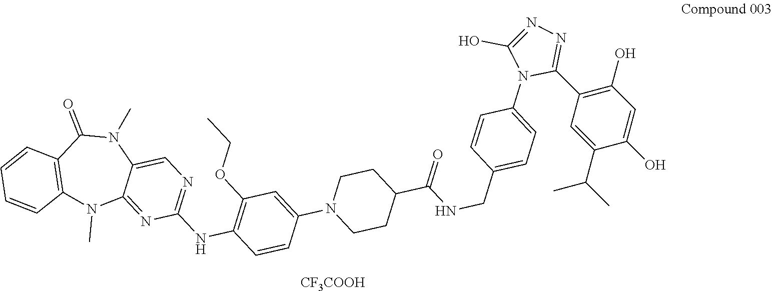

N-(4-(3-(2,4-dihydroxy-5-isopropylphenyl)-5-hydroxy-4H-1,2,4-triazol-4-yl)benzyl)-1-(3-ethoxy-4-((11-methyl-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 11.90 (s, 1H), 9.58 (s, 1H), 9.37 (s, 1H), 8.53-8.43 (m, 2H), 8.11-8.00 (m, 2H), 7.77-7.43 (m, 2H), 7.19-7.11 (m, 6H), 6.86 (s, 1H), 6.24 (s, 1H), 4.28 (d, J=5.3 Hz, 2H), 4.13 (d, J=6.2 Hz, 2H), 3.67-3.50 (m, 5H), 3.39 (s, 3H), 3.32 (s, 3H), 3.00-2.95 (m, 1H), 1.92-1.80 (m, 3H), 1.36-1.34 (m, 3H), 1.19-1.15 (m, 2H), 1.01 (d, J=6.9 Hz, 6H). LC-MS (ESI): R_{T}=1.161 min, m/z. found 825.5 [M-CF₃COOH+H]⁺.

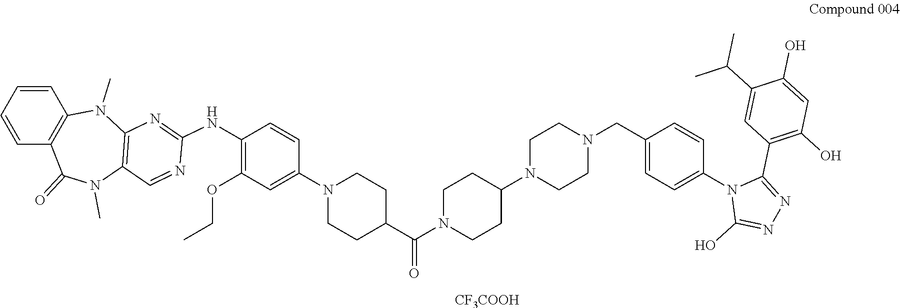 [SMILES: CCOc1cc(N2CCC(C(=O)N3CCC(N4CCN(Cc5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; "CF3COOH"]

2-((4-(4-(4-(4-(4-(3-(2,4-dihydroxy-5-isopropylphenyl)-5-hydroxy-4H-1,2,4-triazol-4-yl)benzyl)piperazin-1-yl)piperidine-1-carbonyl)piperidin-1-yl)-2-ethoxyphenyl)amino)-5,11-dimethyl-5H-benzo[e]pyrimido[5,4-b][1,4]diazepin-6(11H)-one, trifluoroacetic acid. ¹H NMR (400 MHz, DMSO-d₆): δ 9.60 (s, 1H), 9.41 (s, 1H), 8.31 (s, 1H), 7.87 (s, 1H), 7.78 (d, J=8.7 Hz, 1H), 7.67 (d, J=7.5 Hz, 1H), 7.49 (t, J=7.8 Hz, 1H), 7.20-7.11 (m, 7H), 6.77 (s, 1H), 6.61 (s, 1H), 6.49 (d, J=9.5 Hz, 1H), 6.26 (s, 1H), 4.38-4.30 (m, 1H), 4.09-4.03 (m, 4H), 3.67-3.44 (m, 2H), 3.42-4.40 (m, 2H), 3.38-3.35 (m, 2H), 3.28-3.20 (m, 2H), 2.96-2.90 (m, 2H), 2.73-2.63 (m, 5H), 2.33 (s, 3H), 1.79-1.67 (m, 5H), 1.29 (t, J=6.9 Hz, 5H), 1.25-1.17 (m, 2H), 0.94 (d, J=6.9 Hz, 6H). LC-MS (ESI): R_{T}=1.154 min, m/z. found 977.3 [M-CF₃COOH+H]⁺.

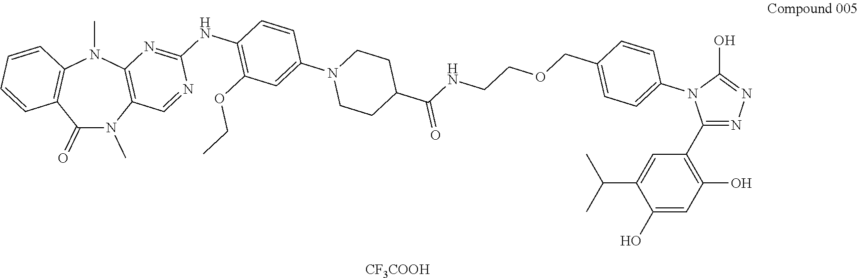 [SMILES: CCOc1cc(N2CCC(C(=O)NCCOCc3ccc(-n4c(O)nnc4-c4cc(C(C)C)c(O)cc4O)cc3)CC2)ccc1Nc1ncc2c(n1)N(C)c1ccccc1C(=O)N2C ; "CF3COOH"]

N-(2-((4-(3-(2,4-dihydroxy-5-isopropylphenyl)-5-hydroxy-4H-1,2,4-triazol-4-yl)benzyl)oxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, CD₃OD): δ 8.54 (d, J=8.7 Hz, 1H), 8.31 (s, 1H), 7.75 (d, J=6.8 Hz, 1H), 7.51 (t, J=7.2 Hz, 1H), 7.44 (d, J=8.2 Hz, 2H), 7.33-7.13 (m, 6H), 6.74 (s, 1H), 6.25 (s, 1H), 4.56 (s, 2H), 4.24 (d, J=6.6 Hz, 2H), 3.81-3.53 (m, 6H), 3.46-3.44 (m, 8H), 3.01 (dt, J=13.7, 6.8 Hz, 1H), 2.70-2.65 (m, 1H), 2.15-2.08 (m, 4H), 1.51 (t, J=6.9 Hz, 3H), 0.92 (d, J=6.9 Hz, 6H). LCMS (ESI): R_{T}=1.39 min, m/z. found 869.5 [M-CF₃COOH+H]⁺.

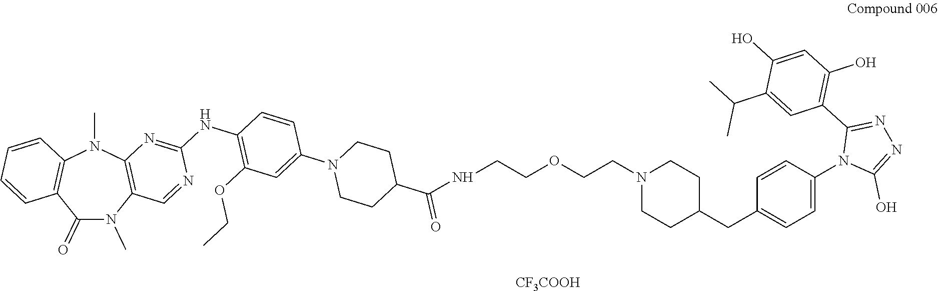 [SMILES: CCOc1cc(N2CCC(C(=O)NCCOCCN3CCC(Cc4ccc(-n5c(O)nnc5-c5cc(C(C)C)c(O)cc5O)cc4)CC3)CC2)ccc1Nc1ncc2c(n1)N(C)c1ccccc1C(=O)N2C ; "CF3COOH"]

N-((2-(4-(4-(3-(2,4-dihydroxy-5-isopropylphenyl)-5-hydroxy-4H-1,2,4-triazol-4-yl)benzyl)piperidin-1-yl)ethoxy)methyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.92 (s, 1H), 9.60 (s, 1H), 9.40 (s, 1H), 9.07 (s, 1H), 8.33 (s, 1H), 7.94-7.80 (m, 3H), 7.68 (d, J=7.6 Hz, 1H), 7.48 (d, J=6.9 Hz, 1H), 7.16-7.09 (m, 6H), 6.75 (s, 1H), 6.26 (s, 1H), 4.08-4.07 (m, 3H), 3.69-3.56 (m, 5H), 3.40-3.35 (m, 5H), 3.26-3.20 (m, 9H), 2.98-2.87 (m, 4H), 1.75-1.72 (m, 8H), 1.42-1.40 (m, 2H), 1.30-1.28 (m, 4H), 0.92 (d, J=6.9 Hz, 6H). LC-MS (ESI): R_{T}=1.148 min, m/z. found 981.0 [M-CF₃COOH+H]⁺.

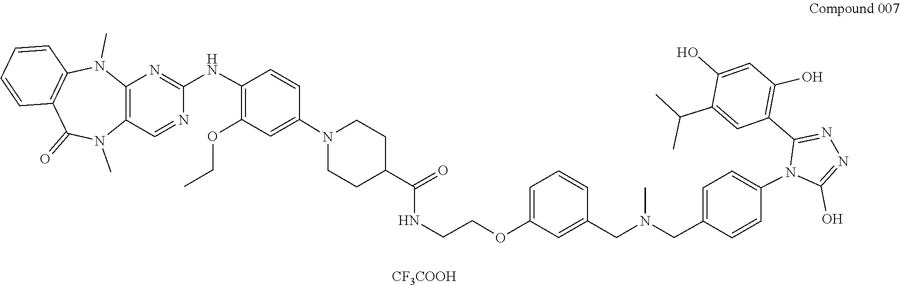 [SMILES: CCOc1cc(N2CCC(C(=O)NCCOc3cccc(CN(C)Cc4ccc(-n5c(O)nnc5-c5cc(C(C)C)c(O)cc5O)cc4)c3)CC2)ccc1Nc1ncc2c(n1)N(C)c1ccccc1C(=O)N2C ; "CF3COOH"]

N-(2-(3-(((4-(3-(2,4-dihydroxy-5-isopropylphenyl)-5-hydroxy-4H-1,2,4-triazol-4-yl)benzyl)(methyl)amino)methyl)phenoxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.98 (s, 1H), 9.64-9.50 (m, 2H), 8.43-8.09 (m, 3H), 7.68 (d, J=7.8 Hz, 1H), 7.62-7.47 (m, 3H), 7.38 (t, J=7.8 Hz, 1H), 7.28-7.07 (m, 8H), 6.90 (s, 1H), 6.26 (s, 1H), 4.42-4.00 (m, 16H), 3.64-3.62 (m, 2H), 3.39-3.32 (m, 8H), 3.05-2.92 (m, 1H), 2.31-2.30 (m, 1H), 1.92-1.90 (m, 4H), 1.35-1.30 (m, 3H), 1.01 (d, J=6.8 Hz, 6H). LCMS (ESI): R_{T}1.333 min, m/z. found 988.4 [M-CF₃COOH+H]⁺.

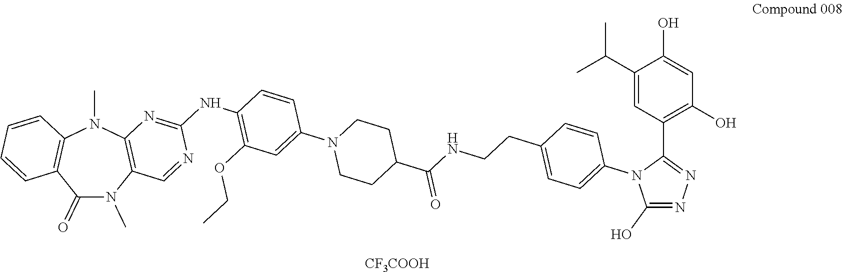 [SMILES: CCOc1cc(N2CCC(C(=O)NCCc3ccc(-n4c(O)nnc4-c4cc(C(C)C)c(O)cc4O)cc3)CC2)ccc1Nc1ncc2c(n1)N(C)c1ccccc1C(=O)N2C ; "CF3COOH"]

N-(4-(3-(2,4-dihydroxy-5-isopropylphenyl)-5-hydroxy-4H-1,2,4-triazol-4-yl)phenethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.91 (s, 1H), 9.60 (s, 1H), 9.38 (s, 1H), 8.38 (s, 1H), 8.16-8.07 (m, 2H), 7.69 (dd, J=7.7, 1.4 Hz, 1H), 7.51 (t, J=7.0 Hz, 1H), 7.26-7.09 (m, 7H), 6.81 (s, 1H), 6.26 (s, 1H), 4.16-4.12 (m, 2H), 3.66-3.62 (m, 2H), 3.32-3.26 (m, 9H), 3.00-2.98 (m, 1H), 2.74-2.71 (m, 2H), 2.50-2.40 (m, 2H), 1.90-1.85 (m, 4H), 1.36 (t, J=6.8 Hz, 3H), 0.98 (d, J=6.9 Hz, 6H). LCMS (ESI): R_{T}=1.513 min, m/z. found 839.8 [M−CF₃COOH+H]⁺.

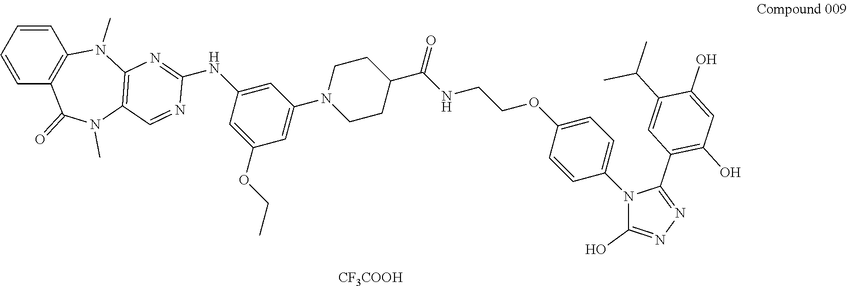 [SMILES: CCOc1cc(Nc2ncc3c(n2)N(C)c2ccccc2C(=O)N3C)cc(N2CCC(C(=O)NCCOc3ccc(-n4c(O)nnc4-c4cc(C(C)C)c(O)cc4O)cc3)CC2)c1 ; "CF3COOH"]

N-(2-(4-(3-(2,4-dihydroxy-5-isopropylphenyl)-5-hydroxy-4H-1,2,4-triazol-4-yl)phenoxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.85 (s, 1H), 9.71-9.43 (m, 2H), 8.35 (s 1H), 8.15-8.09 (m, 2H), 7.70-7.67 (m, 1H), 7.51-7.42 (m, 1H), 7.30-7.06 (m, 5H), 6.94 (d, J=8.9 Hz, 2H), 6.82 (s, 1H), 6.24 (s, 1H), 4.13 (q, J=6.8 Hz, 2H), 4.03-3.84 (m, 2H), 3.65 (d, J=11.6 Hz, 2H), 3.52-3.18 (m, 10H), 2.98-2.96 (m, 1H), 2.46-2.36 (m, 2H), 1.89-1.80 (m, 3H), 1.36-1.33 (m, 6H), 0.98 (d, J=6.9 Hz, 6H). LCMS (ESI): R_{T}=1.39 min, m/z. found 855.5 [M−CH₃COOH+H]⁺.

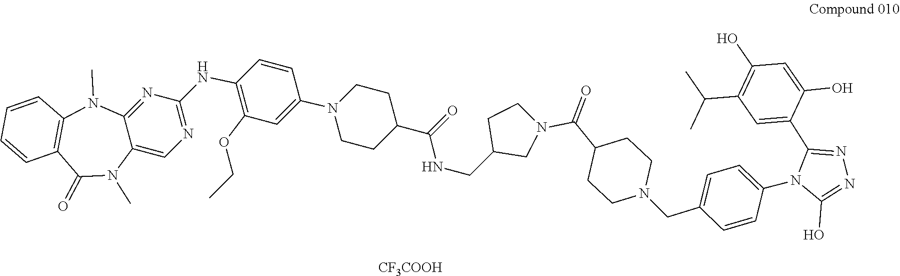 [SMILES: CCOc1cc(N2CCC(C(=O)NCC3CCN(C(=O)C4CCN(Cc5ccc(-n6c(O)nnc6-c6cc(C(C)C)c(O)cc6O)cc5)CC4)C3)CC2)ccc1Nc1ncc2c(n1)N(C)c1ccccc1C(=O)N2C ; "CF3COOH"]

N-((1-(1-(4-(3-(2,4-dihydroxy-5-isopropylphenyl)-5-hydroxy-4H-1,2,4-triazol-4-yl)benzyl)piperidine-4-carbonyl)pyrrolidin-3-yl)methyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.94 (s, 1H), 9.45 (s, 2H), 8.26-8.25 (m, 2H), 7.91-7.90 (m, 3H), 7.68 (s, 1H), 7.49 (s, 1H), 7.33-7.07 (m, 5H), 6.76 (s, 1H), 6.56-6.53 (m, 2H), 6.27 (s, 1H), 5.32 (s, 1H), 4.08 (s, 2H), 3.64 (s, 3H), 3.38 (s, 3H), 3.28 (s, 3H), 2.79 (s, 8H), 2.33 (s, 4H), 1.93 (s, 4H), 1.66-1.61 (m, 8H), 1.26-1.20 (m, 7H). LCMS (ESI): R_{T}=1.018 min, m/z. found 1019.3 [M−CF₃COOH+H]⁺.

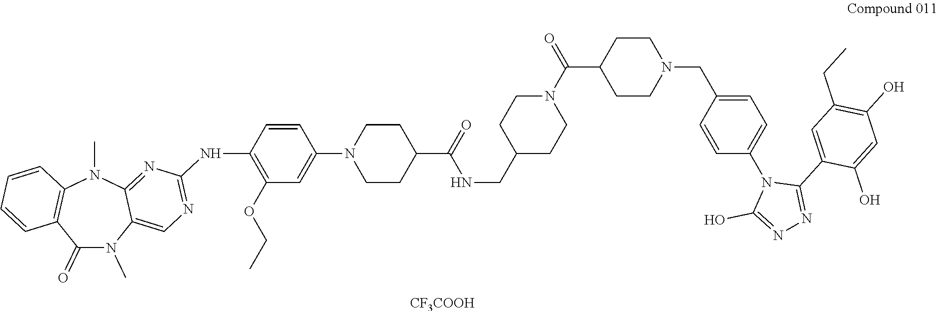 [SMILES: CCOc1cc(N2CCC(C(=O)NCC3CCN(C(=O)C4CCN(Cc5ccc(-n6c(O)nnc6-c6cc(CC)c(O)cc6O)cc5)CC4)CC3)CC2)ccc1Nc1ncc2c(n1)N(C)c1ccccc1C(=O)N2C ; "CF3COOH"]

1-(4-((5,11-dimethyl-6-oxo-6,11-dihydro-5H-benzo[e]pyrimido[5,4-b][1,4]diazepin-2-yl)amino)-3-ethoxyphenyl)-N-((1-(1-(4-(3-(5-ethyl-2,4-dihydroxyphenyl)-5-hydroxy-4H-1,2,4-triazol-4-yl)benzyl)piperidine-4-carbonyl)piperidin-4-yl)methyl)piperidine-4-carboxamide, trifluoroacetic acid. ¹H NMR (400 MHz, DMSO): δ 11.98 (s, 1H), 9.60 (s, 1H), 8.38 (s, 1H), 8.22 (s, 1H), 8.06-8.05 (m, 2H), 7.69 (d, J=7.1 Hz, 1H), 7.50-7.48 (m, 3H), 7.31-7.15 (m, 4H), 7.04-7.01 (m, 2H), 6.91 (s, 1H), 6.24 (s, 1H), 4.22-4.20 (m, 8H), 3.97 (s, 1H), 3.67 (d, J=11.1 Hz, 2H), 3.40 (s, 3H), 3.33 (s, 3H), 3.20-3.18 (m, 2H), 2.97 (s, 6H), 2.38-2.36 (m, 2H), 1.98-1.58 (m, 12H), 1.36 (t, J=6.9 Hz, 3H), 1.23 (s, 3H), 1.01 (t, J=7.4 Hz, 3H). LCMS (ESI): R_{T}=1.005 min, m/z. found 1019.3 [M−CF₃COOH+H]⁺.

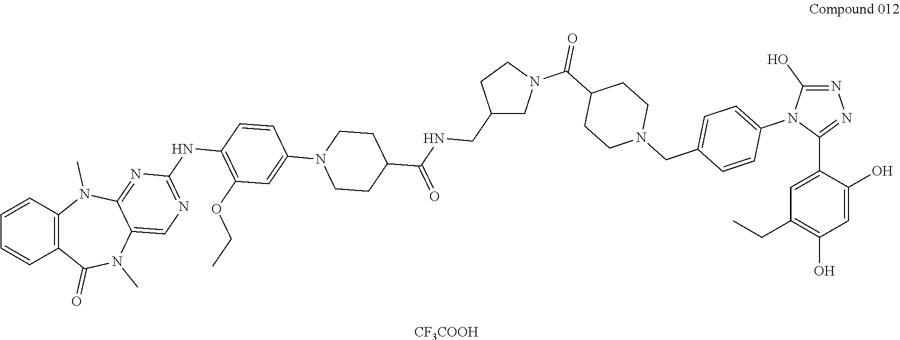 [SMILES: CCOc1cc(N2CCC(C(=O)NCC3CCN(C(=O)C4CCN(Cc5ccc(-n6c(O)nnc6-c6cc(CC)c(O)cc6O)cc5)CC4)C3)CC2)ccc1Nc1ncc2c(n1)N(C)c1ccccc1C(=O)N2C ; "CF3COOH"]

1-(4-((5,11-dimethyl-6-oxo-6,11-dihydro-5H-benzo[e]pyrimido[5,4-b][1,4]diazepin-2-yl)amino)-3-ethoxyphenyl)-N-((1-(1-(4-(3-(5-ethyl-2,4-dihydroxyphenyl)-5-hydroxy-4H-1,2,4-triazol-4-yl)benzyl)piperidine-4-carbonyl)pyrrolidin-3-yl)methyl)piperidine-4-carboxamide, trifluoroacetic acid. ¹H NMR (400 MHz, DMSO): δ 12.00 (s, 1H), 9.50 (m, 3H), 8.37 (s, 1H), 8.10 (m, 3H), 7.69 (dd, J=7.7, 1.4 Hz, 1H), 7.50 (m, 3H), 7.30-7.13 (m, 4H), 6.96 (d, J=41.6 Hz, 3H), 6.24 (s, 1H), 4.27 (s, 2H), 4.13 (d, J=6.9 Hz, 2H), 3.36 (m, 10H), 3.20-2.85 (m, 8H), 2.67 (s, 1H), 2.35-2.32 (m, 4H), 2.04-1.75 (m, 9H), 1.35 (t, J=6.8 Hz, 3H), 1.23 (s, 2H), 1.01 (dd, J=8.3, 6.5 Hz, 3H). LCMS (ESI): R_{T}=0.999 min, m/z. found 1005.3 [M−CH₃COOH+H]⁺.

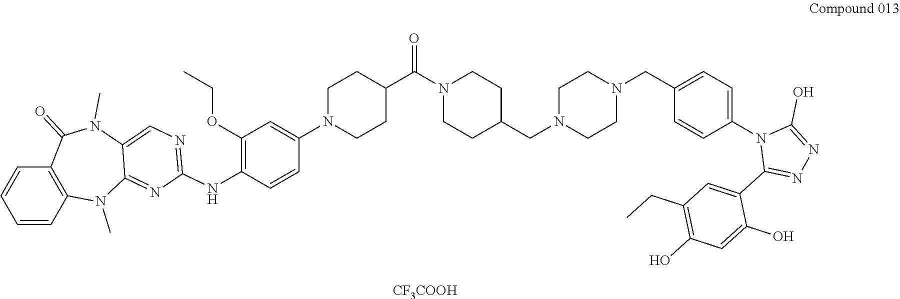 [SMILES: CCOc1cc(N2CCC(C(=O)N3CCC(CN4CCN(Cc5ccc(-n6c(O)nnc6-c6cc(CC)c(O)cc6O)cc5)CC4)CC3)CC2)ccc1Nc1ncc2c(n1)N(C)c1ccccc1C(=O)N2C ; "CF3COOH"]

2-((2-ethoxy-4-(4-(4-((4-(4-(3-(5-ethyl-2,4-dihydroxyphenyl)-5-hydroxy-4H-1,2,4-triazol-4-yl)benzyl)piperazin-1-yl)methyl)piperidine-1-carbonyl)piperidin-1-yl)phenyl)amino)-5,11-dimethyl-5H-benzo[e]pyrimido[5,4-b][1,4]diazepin-6(11H)-one, trifluoroacetic acid. ¹H NMR (400 MHz, DMSO): δ 11.96 (s, 1H), 8.39 (s, 1H), 8.20 (m, 2H), 7.69 (dd, J=7.7, 1.6 Hz, 1H), 7.52 (t, J=7.0 Hz, 1H), 7.43 (s, 1H), 7.42 (d, J=8.2 Hz, 2H), 7.29-7.14 (m, 5H), 7.03 (s, 1H), 6.89 (s, 1H), 6.25 (s, 1H), 4.40 (d, J=12.7 Hz, 1H), 4.23-3.88 (m, 5H), 3.66 (d, J=11.0 Hz, 2H), 3.40 (s, 3H), 3.34 (s, 3H), 3.01 (m, 9H), 2.62-2.53 (m, 1H), 2.37 (q, J=7.4 Hz, 3H), 1.86 (m, 8H), 1.37 (t, J=6.9 Hz, 3H), 1.27-1.11 (m, 2H), 1.00 (t, J=7.5 Hz, 4H). LCMS (ESI): R_{T}=1.078 min, m/z. found 977.3 [M−CF₃COOH+H]⁺.

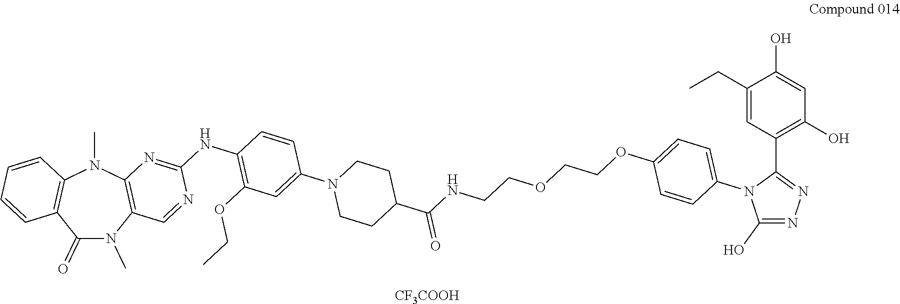 [SMILES: CCOc1cc(N2CCC(C(=O)NCCOCCOc3ccc(-n4c(O)nnc4-c4cc(CC)c(O)cc4O)cc3)CC2)ccc1Nc1ncc2c(n1)N(C)c1ccccc1C(=O)N2C ; "CF3COOH"]

1-(4-((5,11-dimethyl-6-oxo-6,11-dihydro-5H-benzo[e]pyrimido[5,4-b][1,4]diazepin-2-yl)amino)-3-ethoxyphenyl)-N-(2-(2-(4-(3-(5-ethyl-2,4-dihydroxyphenyl)-5-hydroxy-4H-1,2,4-triazol-4-yl)phenoxy)ethoxy)ethyl)piperidine-4-carboxamide, trifluoroacetic acid. ¹H NMR (400 MHz, DMSO-d₆): δ 11.84 (s, 1H), 9.55 (s, 1H), 9.34 (s, 1H), 8.37 (s, 1H), 8.09-8.06 (m, 3H), 7.69 (dd, J=7.8, 1.6 Hz, 1H), 7.54-7.48 (m, 1H), 7.25 (d, J=8.4 Hz, 1H), 7.18 (t, J=7.4 Hz, 1H), 7.08 (d, J=8.9 Hz, 2H), 6.92 (d, J=9.0 Hz, 2H), 6.85 (s, 1H), 6.24 (s, 1H), 4.17-4.04 (m, 4H), 3.76-3.69 (m, 2H), 3.65 (d, J=11.6 Hz, 2H), 3.49 (t, J=5.7 Hz, 2H), 3.39 (s, 3H), 3.32 (s, 3H), 3.28-3.22 (m, 2H), 2.35 (m, 3H), 2.00-1.99 (m, 1H), 1.88 (s, 4H), 1.35 (t, J=6.7 Hz, 3H), 1.23 (s, 3H), 0.99 (t, J=7.5 Hz, 3H). LCMS (ESI): R_{T}=1.157 min, m/z. found 885.2 [M-CF₃COOH+H]⁺.

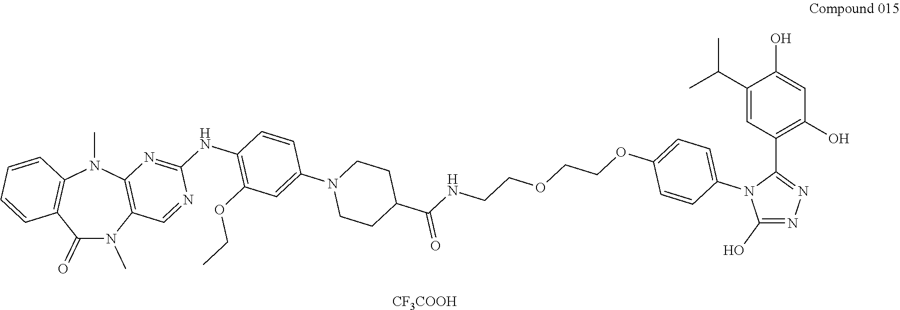 [SMILES: CCOc1cc(N2CCC(C(=O)NCCOCCOc3ccc(-n4c(O)nnc4-c4cc(C(C)C)c(O)cc4O)cc3)CC2)ccc1Nc1ncc2c(n1)N(C)c1ccccc1C(=O)N2C ; "CF3COOH"]

N-(2-(2-(4-(3-(2,4-dihydroxy-5-isopropylphenyl)-5-hydroxy-4H-1,2,4-triazol-4-yl)phenoxy)ethoxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO): δ 11.86 (s, 1H), 9.58 (s, 1H), 9.38 (s, 1H), 8.37 (s, 1H), 8.09 (m, 3H), 7.69 (dd, J=7.7, 1.6 Hz, 1H), 7.50 (d, J=6.9 Hz, 1H), 7.25 (d, J=8.3 Hz, 1H), 7.18 (t, J=7.3 Hz, 1H), 7.10 (d, J=8.9 Hz, 2H), 6.93 (d, J=9.0 Hz, 2H), 6.81 (s, 1H), 6.25 (s, 1H), 4.10-4.08 (m, 5H), 3.72 (s, 2H), 3.65 (d, J=11.2 Hz, 2H), 3.49 (t, J=5.8 Hz, 2H), 3.39 (s, 3H), 3.32 (s, 3H), 3.25 (d, J=5.8 Hz, 2H), 3.01-2.96 (m, 1H), 2.00 (d, J=7.5 Hz, 1H), 1.87 (s, 4H), 1.35 (t, J=6.9 Hz, 3H), 1.24 (s, 3H), 0.98 (d, J=6.9 Hz, 6H). LCMS (ESI): R_{T}=1.192 min, m/z. found 899.3 [M-CF₃COOH+H]⁺.

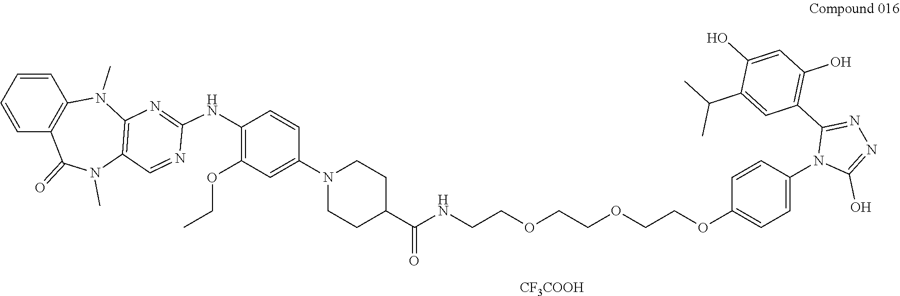 [SMILES: CCOc1cc(N2CCC(C(=O)NCCOCCOCCOc3ccc(-n4c(O)nnc4-c4cc(C(C)C)c(O)cc4O)cc3)CC2)ccc1Nc1ncc2c(n1)N(C)c1ccccc1C(=O)N2C ; "CF3COOH"]

N-(2-(2-(2-(4-(3-(2,4-dihydroxy-5-isopropylphenyl)-5-hydroxy-4H-1,2,4-triazol-4-yl)phenoxy)ethoxy)ethoxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.86 (s, 1H), 9.58 (s, 1H), 9.38 (s, 1H), 8.37 (s, 1H), 8.09-8.06 (m, 3H), 7.69 (d, J=7.7 Hz, 1H), 7.51 (t, J=7.1 Hz, 1H), 7.28-7.15 (m, 3H), 7.09 (d, J=8.9 Hz, 2H), 6.93 (d, J=8.8 Hz, 2H), 6.81 (s, 1H), 6.25 (s, 1H), 4.10-4.09 (m, 4H), 3.63-3.61 (m, 9H), 3.42-3.40 (m, 5H), 3.32 (s, 3H), 3.23 (d, J=5.5 Hz, 2H), 3.01-2.95 (m, 1H), 2.04-1.81 (m, 5H), 1.35 (t, J=6.9 Hz, 3H), 1.24 (s, 2H), 0.98 (d, J=6.9 Hz, 6H). LCMS (ESI): R_{T}=1.186 min, m/z. found 943.2 [M-CF₃COOH+H]⁺.

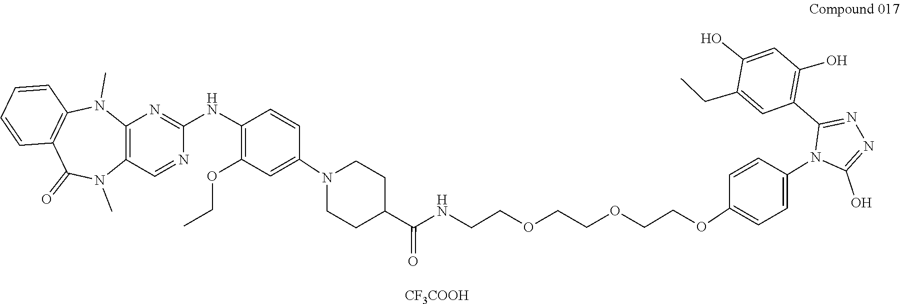 [SMILES: CCOc1cc(N2CCC(C(=O)NCCOCCOCCOc3ccc(-n4c(O)nnc4-c4cc(CC)c(O)cc4O)cc3)CC2)ccc1Nc1ncc2c(n1)N(C)c1ccccc1C(=O)N2C ; "CF3COOH"]

1-(4-((5,11-dimethyl-6-oxo-6,11-dihydro-5H-benzo[e]pyrimido[5,4-b][1,4]diazepin-2-yl)amino)-3-ethoxyphenyl)-N-(2-(2-(2-(4-(3-(5-ethyl-2,4-dihydroxyphenyl)-5-hydroxy-4H-1,2,4-triazol-4-yl)phenoxy)ethoxy)ethoxy)ethyl)piperidine-4-carboxamide, trifluoroacetic acid. ¹H NMR (400 MHz, DMSO-d₆): δ 11.83 (s, 1H), 9.54 (s, 1H), 9.33 (s, 1H), 8.37 (s, 1H), 8.01 (s, 2H), 7.69 (dd, J=7.7, 1.6 Hz, 1H), 7.51 (t, J=6.9 Hz, 1H), 7.25 (d, J=8.4 Hz, 1H), 7.18 (t, J=7.4 Hz, 1H), 7.07 (d, J=8.9 Hz, 2H), 6.91 (d, J=9.0 Hz, 2H), 6.85 (s, 1H), 6.24 (s, 1H), 4.13 (d, J=6.8 Hz, 2H), 4.08-4.04 (m, 2H), 3.75-3.52 (m, 9H), 3.45-3.38 (m, 5H), 3.32 (s, 3H), 3.22 (d, J=5.7 Hz, 2H), 2.35 (q, J=7.6 Hz, 2H), 2.02-1.84 (m, 4H), 1.38-1.22 (m, 8H), 0.99 (t, J=7.5 Hz, 3H). LCMS (ESI): R_{T}=1.159 min, m/z. found 929.2 [M-CF₃COOH+H]⁺.

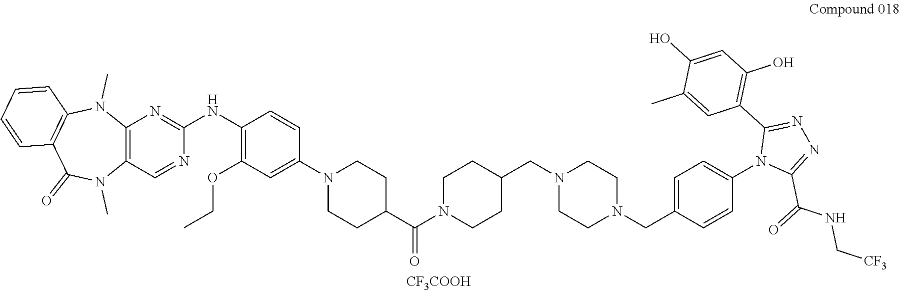 [SMILES: CCOc1cc(N2CCC(C(=O)N3CCC(CN4CCN(Cc5ccc(-n6c(C(=O)NCC(F)(F)F)nnc6-c6cc(C)c(O)cc6O)cc5)CC4)CC3)CC2)ccc1Nc1ncc2c(n1)N(C)c1ccccc1C(=O)N2C ; "CF3COOH"]

5-(2,4-dihydroxy-5-methylphenyl)-4-(4-((4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperazin-1-yl)methyl)phenyl)-N-(2,2,2-trifluoroethyl)-4H-1,2,4-triazole-3-carboxamide, trifluoroacetic acid. ¹H NMR (400 MHz, DMSO-d₆): δ 9.71-9.63 (m, 3H), 8.36 (s, 1H), 8.09-8.01 (m, 2H), 7.69 (dd, J=1.2 Hz, 1H), 7.53-7.16 (m, 8H), 6.72 (s, 2H), 6.29 (s, 1H), 4.42-4.39 (m, 1H), 4.13-4.11 (m, 3H), 4.01-3.92 (m, 6H), 3.68-3.65 (m, 2H), 3.39 (s, 4H), 3.31 (s, 4H), 3.10-2.86 (m, 7H), 2.67-2.50 (m, 2H), 2.05-1.96 (m, 2H), 1.87 (s, 3H), 1.78 (s, 6H), 1.34 (t, J=6.8 Hz, 3H), 1.23 (s, 2H), 1.17-0.98 (m, 2H). LCMS (ESI): R_{T}=1.123 min, m/z. found 1072.3 [M-CF₃COOH+H]⁺.

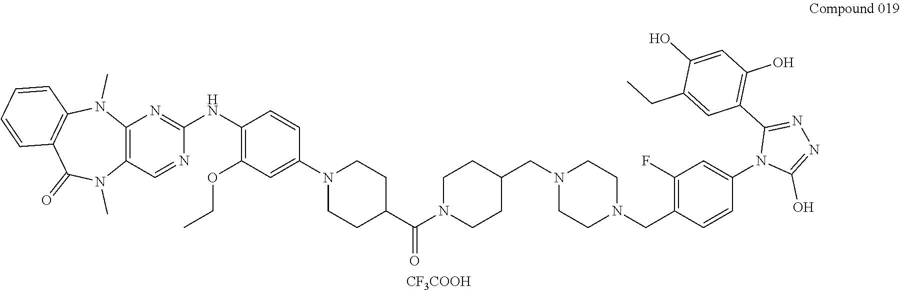 [SMILES: CCOc1cc(N2CCC(C(=O)N3CCC(CN4CCN(Cc5ccc(-n6c(O)nnc6-c6cc(CC)c(O)cc6O)cc5F)CC4)CC3)CC2)ccc1Nc1ncc2c(n1)N(C)c1ccccc1C(=O)N2C ; "CF3COOH"]

2-((2-ethoxy-4-(4-(4-((4-(4-(3-(5-ethyl-2,4-dihydroxyphenyl)-5-hydroxy-4H-1,2,4-triazol-4-yl)-2-fluorobenzyl)piperazin-1-yl)methyl)piperidine-1-carbonyl)piperidin-1-yl)phenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 12.00 (s, 1H), 9.64-9.40 (m, 2H), 8.37 (s, 1H), 8.15-8.05 (m, 2H), 7.69 (d, J=7.6 Hz, 1H), 7.53-7.49 (m, 1H), 7.44-7.40 (m, 1H), 7.26-6.94 (m, 6H), 6.26 (s, 1H), 4.57-4.37 (m, 5H), 4.16-4.11 (m, 3H), 4.01-3.98 (m, 1H), 3.76-3.64 (m, 3H), 3.39 (s, 4H), 3.32 (s, 4H), 3.07-2.94 (m, 7H), 2.42-2.33 (s, 4H), 2.02-1.77 (m, 8H), 1.37-1.33 (m, 3H), 1.23-1.08 (m, 2H), 1.05-1.01 (m, 4H). LCMS (ESI): R_{T}=1.033 min, m/z. found 995.3 [M-CF₃COOH+H]⁺.

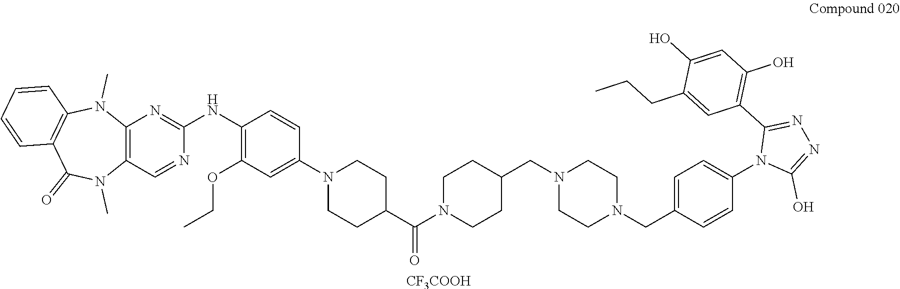 [SMILES: CCCc1cc(-c2nnc(O)n2-c2ccc(CN3CCN(CC4CCN(C(=O)C5CCN(c6ccc(Nc7ncc8c(n7)N(C)c7ccccc7C(=O)N8C)c(OCC)c6)CC5)CC4)CC3)cc2)c(O)cc1O ; "CF3COOH"]

2-((4-(4-(4-((4-(4-(3-(2,4-dihydroxy-5-propylphenyl)-5-hydroxy-4H-1,2,4-triazol-4-yl)benzyl)piperazin-1-yl)methyl)piperidine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, CD₃OD): δ 8.23 (s, 1H), 8.10 (d, J=8.8 Hz, 1H), 7.76-7.70 (m, 1H), 7.53-7.48 (m, 1H), 7.42 (d, J=8.4 Hz, 2H), 7.26-7.14 (m, 4H), 6.72-6.59 (m, 3H), 6.23 (s, 1H), 5.36-5.31 (m, 1H), 4.12 (m, 6.9 Hz, 2H), 3.65 (m, 4H), 3.47 (s, 3H), 3.41 (s, 3H), 3.13 (s, 1H), 2.81-2.58 (m, 10H), 2.32 (m, 6.9 Hz, 4H), 2.18 (d, J=7.7 Hz, 1H), 2.03 (d, J=5.9 Hz, 1H), 1.94-1.85 (m, 4H), 1.81 (d, J=11.5 Hz, 2H), 1.60 (s, 2H), 1.45-1.37 (m, 6H), 0.90 (t, J=6.7 Hz, 3H), 0.79 (t, J=7.4 Hz, 3H). LCMS (ESI): R_{T}=1.033 min, m/z. found 991.3[M-CF₃COOH+H]⁺.

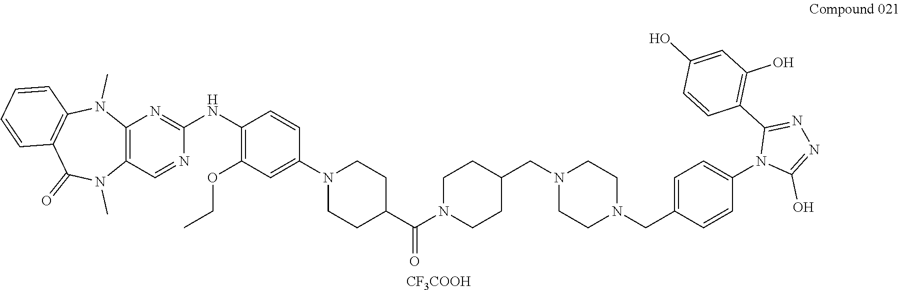 [SMILES: CCOc1cc(N2CCC(C(=O)N3CCC(CN4CCN(Cc5ccc(-n6c(O)nnc6-c6ccc(O)cc6O)cc5)CC4)CC3)CC2)ccc1Nc1ncc2c(n1)N(C)c1ccccc1C(=O)N2C ; "CF3COOH"]

2-((4-(4-(4-((4-(4-(3-(2,4-dihydroxyphenyl)-5-hydroxy-4H-1,2,4-triazol-4-yl)benzyl)piperazin-1-yl)methyl)piperidine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, CD₃OD): δ 8.56 (d, J=8.7 Hz, 1H), 8.33 (s, 1H), 7.76 (m, 1.5 Hz, 1H), 7.56-7.50 (m, 1H), 7.46 (d, J=8.4 Hz, 2H), 7.30-7.19 (m, 6H), 7.03 (d, J=8.5 Hz, 1H), 6.26 (m, 1H), 6.17 (d, J=2.2 Hz, 1H), 4.57 (m, 1H), 4.25 (m, 6.8 Hz, 2H), 4.14 (m, 1H), 3.97 (s, 2H), 3.82-3.66 (m, 5H), 3.47 (m, 6H), 3.13 (m, 12H), 2.80 (m, 2H), 2.71 (m, 1H), 2.15 (m, 6H), 1.90 (m, 2H), 1.51 (t, J=7.0 Hz, 3H). LCMS (ESI): R_{T}=0.973 min, m/z. found 949.2[M-CF₃COOH+H]⁺.

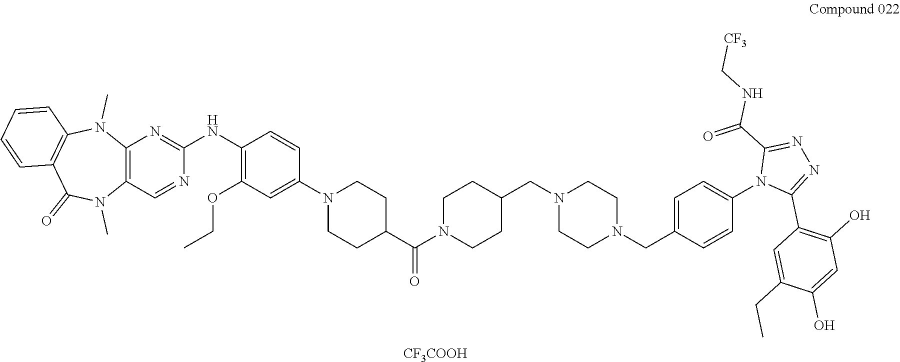 [SMILES: CCOc1cc(N2CCC(C(=O)N3CCC(CN4CCN(Cc5ccc(-n6c(C(=O)NCC(F)(F)F)nnc6-c6cc(CC)c(O)cc6O)cc5)CC4)CC3)CC2)ccc1Nc1ncc2c(n1)N(C)c1ccccc1C(=O)N2C ; "CF3COOH"]

4-(4-((4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperazin-1-yl)methyl)phenyl)-5-(5-ethyl-2,4-dihydroxyphenyl)-N-(2,2,2-trifluoroethyl)-4H-1,2,4-triazole-3-carboxamide, trifluoroacetic acid. ¹H NMR (400 MHz, DMSO-d₆): δ 9.67 (m, 2H), 8.37 (s, 1H), 8.09 (m, 2H), 7.69 (m, 1.5 Hz, 1H), 7.51 (m, 1H), 7.39 (m, 7.8 Hz, 4H), 7.27-7.14 (m, 2H), 6.98 (s, 1H), 6.68 (s, 1H), 6.31 (s, 1H), 4.40 (m, 1H), 4.13 (m, 2H), 4.05-3.88 (m, 4H), 3.66 (m, 2H), 3.39 (s, 3H), 3.32 (s, 3H), 3.14-2.72 (m, 9H), 2.58 (s, 1H), 2.34-2.22 (m, 3H), 1.90 (m, 9H), 1.35 (t, J=6.9 Hz, 3H), 1.25-0.98 (m, 4H), 0.89 (m, 4H). LCMS (ESI): R_{T}=1.083 min, m/z. found 1086.3 [M-CF₃COOH+H]⁺.

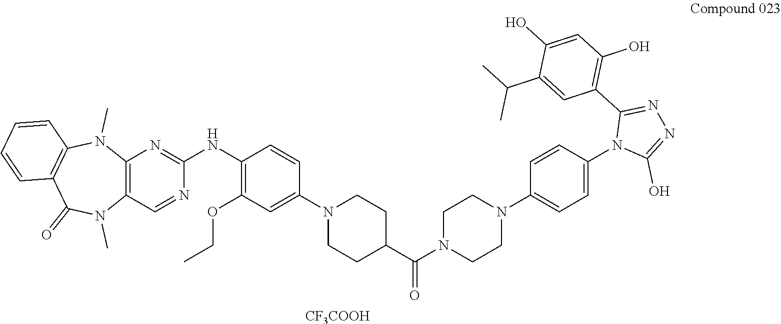 [SMILES: CCOc1cc(N2CCC(C(=O)N3CCN(c4ccc(-n5c(O)nnc5-c5cc(C(C)C)c(O)cc5O)cc4)CC3)CC2)ccc1Nc1ncc2c(n1)N(C)c1ccccc1C(=O)N2C ; "CF3COOH"]

2-((4-(4-(4-(4-(3-(2,4-dihydroxy-5-isopropylphenyl)-5-hydroxy-4H-1,2,4-triazol-4-yl)phenyl)piperazine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.92 (s, 1H), 9.66 (s, 1H), 9.51 (s, 1H), 8.44 (s, 1H), 8.23 (s, 1H), 8.18 (s, 1H), 7.76-7.74 (m, 1H), 7.60-7.55 (m, 1H), 7.32 (d, J=8.4 Hz, 1H), 7.27-7.22 (m, 1H), 7.13-7.01 (m, 5H), 6.85 (s, 1H), 6.33 (s, 1H), 4.24-4.18 (m, 2H), 3.85-3.68 (m, 9H), 3.46 (s, 4H), 3.39 (s, 4H), 3.32-3.27 (m, 4H), 3.09-3.02 (m, 2H), 1.97 (s, 3H), 1.42 (t, J=6.8 Hz, 3H), 1.02 (d, J=6.8 Hz, 6H). LCMS (ESI): R_{T}=1.268 min, m/z. found 880.2 [M-CF₃COOH+H]⁺.

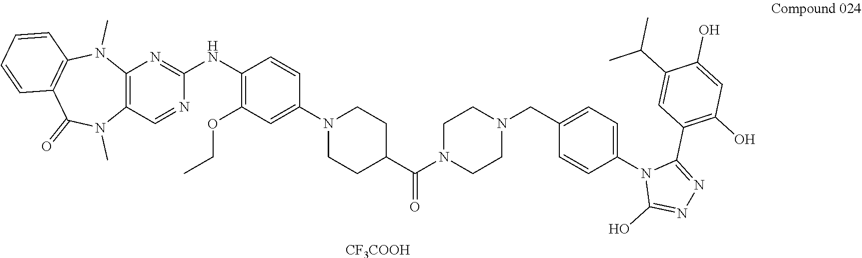 [SMILES: CCOc1cc(N2CCC(C(=O)N3CCN(Cc4ccc(-n5c(O)nnc5-c5cc(C(C)C)c(O)cc5O)cc4)CC3)CC2)ccc1Nc1ncc2c(n1)N(C)c1ccccc1C(=O)N2C ; "CF3COOH"]

2-((4-(4-(4-(4-(3-(2,4-dihydroxy-5-isopropylphenyl)-5-hydroxy-4H-1,2,4-triazol-4-yl)benzyl)piperazine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆) δ 12.00 (s, 1H), 10.11 (s, 1H), 9.66 (s, 1H), 9.37 (s, 1H), 8.35 (s, 1H), 8.09 (s, 1H), 7.95 (s, 1H), 7.69 (m, 1.5 Hz, 1H), 7.51 (t, J=8.5 Hz, 3H), 7.33-7.13 (m, 4H), 6.89 (s, 1H), 6.76 (s, 1H), 6.26 (s, 1H), 4.34 (m, 3H), 4.11 (m, 6.7 Hz, 3H), 3.73 (s, 3H), 3.35 (m, 8H), 3.15-2.85 (m, 7H), 1.81 (s, 4H), 1.33 (t, J=6.9 Hz, 3H), 1.02 (d, J=6.9 Hz, 6H). LCMS (ESI): R_{T}=1.083 min, m/z. found 894.2 [M-CF₃COOH+H]⁺.

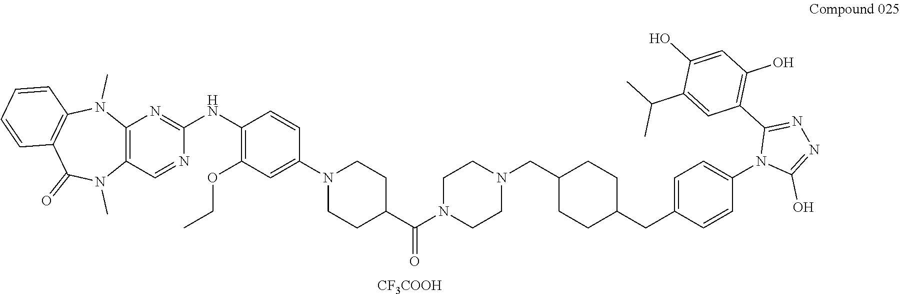 [SMILES: CCOc1cc(N2CCC(C(=O)N3CCN(CC4CCC(Cc5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; "CF3COOH"]

2-((4-(4-(4-((4-(4-(3-(2,4-dihydroxy-5-isopropylphenyl)-5-hydroxy-4H-1,2,4-triazol-4-yl)benzyl)piperidin-1-yl)methyl)piperidine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.92 (s, 1H), 9.62 (s, 1H), 9.41 (s, 1H), 8.87 (s, 1H), 8.35 (s, 1H), 8.16-7.85 (m, 2H), 7.68 (d, J=7.6 Hz, 1H), 7.56-7.46 (m, 1H), 7.28-7.09 (m, 7H), 6.76 (s, 1H), 6.27 (s, 1H), 4.44-4.34 (m, 2H), 4.16-3.93 (m, 6H), 3.70-3.61 (m, 2H), 3.54-3.44 (m, 2H), 3.39 (s, 3H), 3.30 (s, 3H), 3.22-2.64 (m, 10H), 2.12-1.91 (m, 2H), 1.81-1.70 (m, 8H), 1.53-1.40 (m, 2H), 1.36-1.29 (m, 3H), 0.94 (d, J=6.8 Hz, 6H). LCMS (ESI): R_{T}=1.009 min, m/z. found 990.4 [M-CF₃COOH+H]⁺.

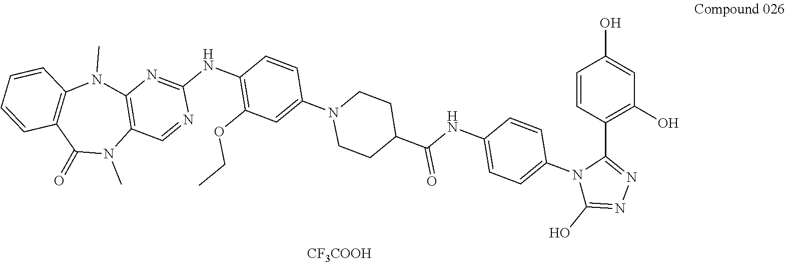 [SMILES: CCOc1cc(N2CCC(C(=O)Nc3ccc(-n4c(O)nnc4-c4ccc(O)cc4O)cc3)CC2)ccc1Nc1ncc2c(n1)N(C)c1ccccc1C(=O)N2C ; "CF3COOH"]

N-(4-(3-(2,4-dihydroxyphenyl)-5-hydroxy-4H-1,2,4-triazol-4-yl)benzyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.89 (s, 1H), 9.59 (s, 2H), 8.49-8.37 (m, 2H), 8.13-8.02 (m, 2H), 7.69 (dd, J=7.6, 1.3 Hz, 1H), 7.53-7.49 (m, 1H), 7.26-7.04 (m, 8H), 6.24-6.17 (m, 2H), 4.32 (s, 4H), 4.28 (d, J=5.1 Hz, 2H), 4.13 (dd, J=12.1, 8.6 Hz, 2H), 3.67 (d, J=12.2 Hz, 2H), 3.39 (s, 3H), 3.32 (s, 3H), 1.93 (s, 4H), 1.35 (t, J=6.8 Hz, 3H). LCMS (ESI): R_{T}=1.063 min, m/z. found 783.3 [M-CF₃COOH+H]⁺.

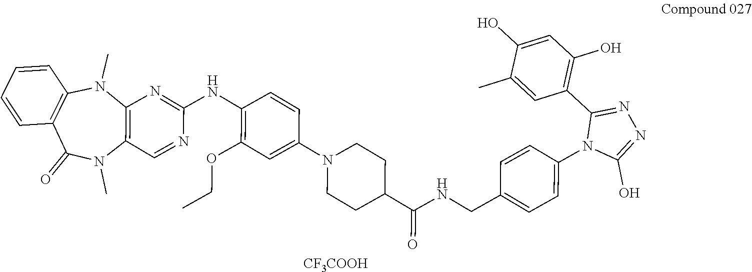 [SMILES: CCOc1cc(N2CCC(C(=O)NCc3ccc(-n4c(O)nnc4-c4cc(C)c(O)cc4O)cc3)CC2)ccc1Nc1ncc2c(n1)N(C)c1ccccc1C(=O)N2C ; "CF3COOH"]

N-(4-(3-(2,4-dihydroxy-5-methylphenyl)-5-hydroxy-4H-1,2,4-triazol-4-yl)benzyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.86 (s, 1H), 9.64-9.24 (m, 2H), 8.54-8.32 (m, 2H), 8.19-7.99 (m, 1H), 7.74-7.63 (m, 1H), 7.57-7.46 (m, 1H), 7.28-7.09 (m, 6H), 6.93 (s, 1H), 6.23 (s, 1H), 5.35-4.45 (m, 3H), 4.28 (d, J=5.2 Hz, 2H), 4.19-4.09 (m, 2H), 3.71-3.62 (m, 2H), 3.39 (s, 3H), 3.32 (s, 3H), 2.02-1.76 (m, 8H), 1.40-1.29 (m, 3H). LCMS (ESI): R_{T}=1.094 min, m/z. found 797.4 [M-CF₃COOH+H]⁺.

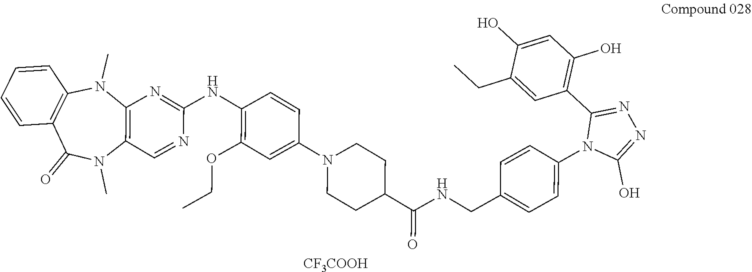 [SMILES: CCOc1cc(N2CCC(C(=O)NCc3ccc(-n4c(O)nnc4-c4cc(CC)c(O)cc4O)cc3)CC2)ccc1Nc1ncc2c(n1)N(C)c1ccccc1C(=O)N2C ; "CF3COOH"]

1-(4-((5,11-dimethyl-6-oxo-6,11-dihydro-5H-benzo[e]pyrimido[5,4-b][1,4]diazepin-2-yl)amino)-3-ethoxyphenyl)-N-(4-(3-(5-ethyl-2,4-dihydroxyphenyl)-5-hydroxy-4H-1,2,4-triazol-4-yl)benzyl)piperidine-4-carboxamide, trifluoroacetic acid. ¹H NMR (400 MHz, DMSO-d₆): δ 11.96-11.84 (m, 1H), 9.58 (s, 1H), 9.39 (s, 1H), 8.51 (s, 1H), 8.43-7.32 (m, 1H), 8.13 (s, 2H), 7.72 (s, 1H), 7.53 (s, 1H), 7.36-7.08 (m, 7H), 6.97-6.87 (m, 1H), 6.31-6.21 (m, 1H), 4.32 (s, 3H), 4.15 (s, 3H), 3.69 (s, 2H), 3.48-3.27 (m, 7H), 2.44-2.33 (m, 3H), 1.93 (s, 4H), 1.37 (s, 3H), 1.12-0.96 (m, 3H). LCMS (ESI): R_{T}=1.137 min, m/z. found 811.7 [M-CF₃COOH+H]⁺.

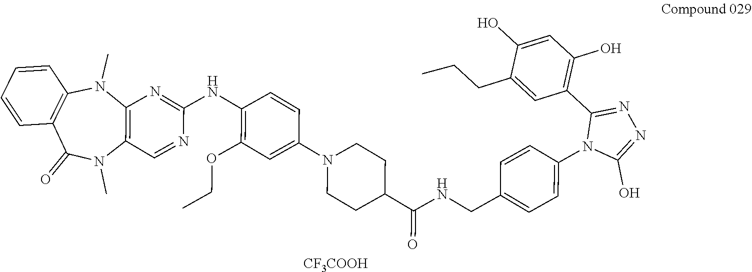 [SMILES: CCCc1cc(-c2nnc(O)n2-c2ccc(CNC(=O)C3CCN(c4ccc(Nc5ncc6c(n5)N(C)c5ccccc5C(=O)N6C)c(OCC)c4)CC3)cc2)c(O)cc1O ; "CF3COOH"]

N-(4-(3-(2,4-dihydroxy-5-propylphenyl)-5-hydroxy-4H-1,2,4-triazol-4-yl)benzyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.87 (s, 1H), 9.60-9.25 (m, 2H), 8.56-8.32 (m, 2H), 8.12 (s, 1H), 7.68 (d, J=8.0 Hz, 1H), 7.55-7.47 (m, 1H), 7.30-7.06 (m 7H), 6.86 (s, 1H), 6.24 (s, 1H), 4.92-4.35 (m, 5H), 4.28 (d, J=5.2 Hz, 2H), 4.17-4.08 (m, 2H), 3.72-3.61 (m, 2H), 3.44-3.26 (m, 6H), 2.35-2.28 (m, 3H), 1.93 (s, 4H), 1.49-1.30 (m, 5H), 0.80 (t, J=7.2 Hz, 3H). LCMS (ESI): R_{T}=1.174 min, m/z. found 825.3 [M-CF₃COOH+H]⁺.

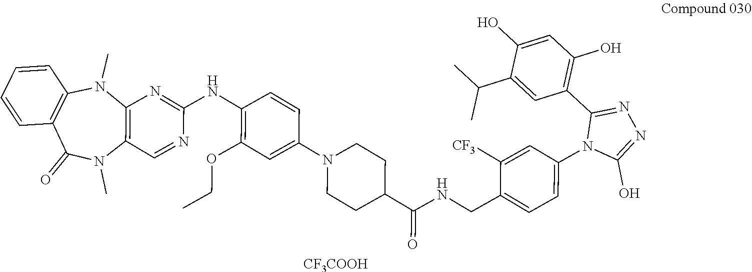 [SMILES: CCOc1cc(N2CCC(C(=O)NCc3ccc(-n4c(O)nnc4-c4cc(C(C)C)c(O)cc4O)cc3C(F)(F)F)CC2)ccc1Nc1ncc2c(n1)N(C)c1ccccc1C(=O)N2C ; "CF3COOH"]

N-(4-(3-(2,4-dihydroxy-5-isopropylphenyl)-5-hydroxy-4H-1,2,4-triazol-4-yl)-2-(trifluoromethyl)benzyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 12.02 (s, 1H), 9.61-6.37 (m, 2H), 8.58 (s, 1H), 8.37 (s, 1H), 8.15-8.05 (m, 2H), 7.69 (d, J=7.6 Hz, 1H), 7.51-7.46 (m, 4H), 7.26-7.16 (m, 2H), 6.99 (s, 2H), 6.24 (s, 1H), 5.04-4.51 (m, 4H), 4.44 (d, J=4.4 Hz, 2H), 4.14 (dd, J=11.9, 5.4 Hz, 2H), 3.68 (d, J=11.2 Hz, 2H), 3.39 (s, 3H), 3.32 (s, 3H), 3.07-3.01 (m, 1H), 2.10-1.76 (m, 4H), 1.35 (t, J=6.5 Hz, 3H), 1.06 (d, J=6.8 Hz, 6H). LCMS (ESI): R_{T}=1.253 min, m/z. found 893.7 [M-CF₃COOH+H]⁺.

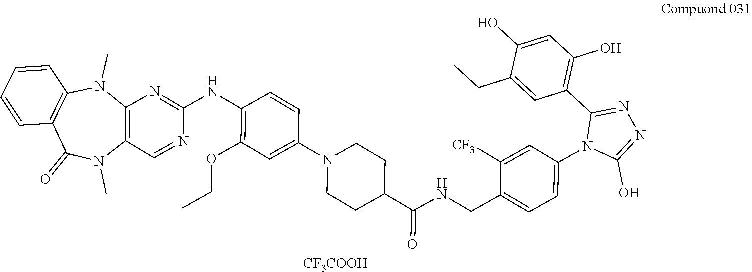 [SMILES: CCOc1cc(N2CCC(C(=O)NCc3ccc(-n4c(O)nnc4-c4cc(CC)c(O)cc4O)cc3C(F)(F)F)CC2)ccc1Nc1ncc2c(n1)N(C)c1ccccc1C(=O)N2C ; "CF3COOH"]

1-(4-((5,11-dimethyl-6-oxo-6,11-dihydro-5H-benzo[e]pyrimido[5,4-b][1,4]diazepin-2-yl)amino)-3-ethoxyphenyl)-N-(4-(3-(5-ethyl-2,4-dihydroxyphenyl)-5-hydroxy-4H-1,2,4-triazol-4-yl)-2-(trifluoromethyl)benzyl)piperidine-4-carboxamide, trifluoroacetic acid. ¹H NMR (400 MHz, DMSO-d₆): δ 12.01 (s, 1H), 9.59 (s, 1H), 9.37 (s, 1H), 8.54 (s, 1H), 8.36 (s, 1H), 8.17-7.96 (m, 2H), 7.69 (d, J=7.2 Hz, 1H), 7.54-7.43 (m, 4H), 7.26-7.16 (m, 2H), 7.01 (s, 1H), 6.24 (s, 1H), 4.71-4.27 (m, 6H), 4.12 (dd, J=14.0, 6.7 Hz, 2H), 3.68 (d, J=12.0 Hz, 2H), 3.35 (d, J=30.0 Hz, 6H), 2.44-2.33 (m, 4H), 2.05-1.76 (m, 3H), 1.34 (t, J=6.4 Hz, 3H), 1.26-1.14 (m, 1H), 1.05 (t, J=7.6 Hz, 3H). LCMS (ESI): R_{T}=1.167 min, m/z. found 879.7 [M-CF₃COOH+H]⁺.

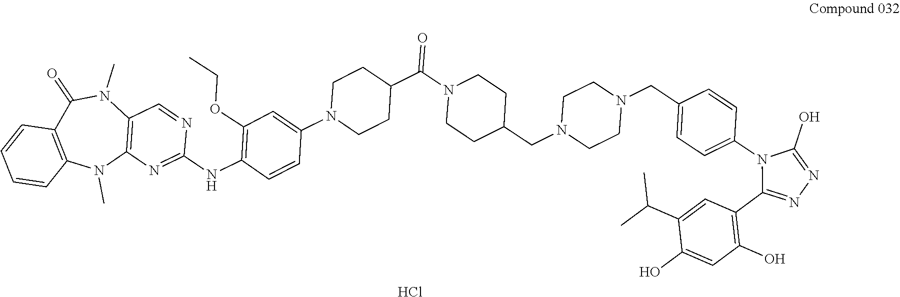 [SMILES: CCOc1cc(N2CCC(C(=O)N3CCC(CN4CCN(Cc5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; Cl]

2-((4-(4-(4-((4-(4-(3-(2,4-dihydroxy-5-isopropylphenyl)-5-hydroxy-4H-1,2,4-triazol-4-yl)benzyl)piperazin-1-yl)methyl)piperidine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, hydrochloride. ¹H NMR (400 MHz, DMSO-d₆): δ 11.95 (s, 1H), 9.50 (d, J=95.9 Hz, 2H), 8.36 (s, 1H), 8.20-7.94 (m, 2H), 7.69 (d, J=7.6 Hz, 1H), 7.51-7.38 (m, 3H), 7.26-7.16 (m, 4H), 7.10-6.87 (m, 1H), 6.82 (s, 1H), 6.26 (s, 1H), 5.29-4.66 (m, 4H), 4.39 (d, J=12.0 Hz, 1H), 4.13 (q, J=6.8 Hz, 2H), 3.99 (d, J=9.6 Hz, 1H), 3.66 (d, J=12.0 Hz, 2H), 3.39 (s, 3H), 3.32 (s, 4H), 3.18-2.81 (m, 8H), 2.45-2.30 (m, 2H), 2.06-1.92 (m, 1H), 1.90-1.64 (m, 7H), 1.34 (t, J=6.7 Hz, 3H), 1.27-1.20 (m, 1H), 1.17-1.06 (m, 1H), 0.98 (d, J=6.8 Hz, 9H). LCMS (ESI): R_{T}=1.060 min, m/z. found 991.4 [M−HCl+H]⁺.

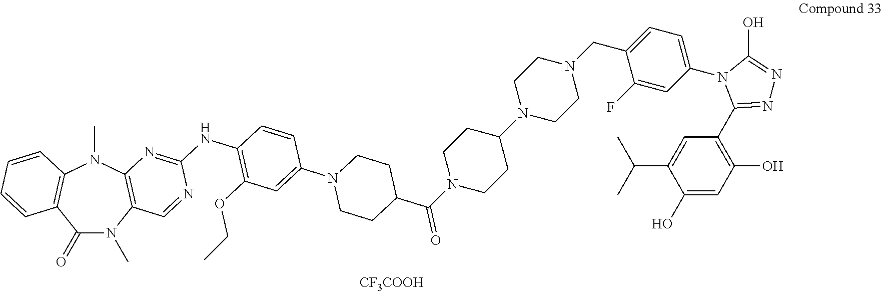 [SMILES: CCOc1cc(N2CCC(C(=O)N3CCC(N4CCN(Cc5ccc(-n6c(O)nnc6-c6cc(C(C)C)c(O)cc6O)cc5F)CC4)CC3)CC2)ccc1Nc1ncc2c(n1)N(C)c1ccccc1C(=O)N2C ; "CF3COOH"]

2-((4-(4-(4-(4-(4-(3-(2,4-dihydroxy-5-isopropylphenyl)-5-hydroxy-4H-1,2,4-triazol-4-yl)-2-fluorobenzyl)piperazin-1-yl)piperidine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.99 (s, 1H), 9.64 (s, 1H), 9.39 (s, 1H), 8.36 (s, 1H), 8.09 (s, 3H), 7.69 (d, J=7.5 Hz, 1H), 7.54-7.48 (m, 1H), 7.47-7.39 (m, 1H), 7.28-7.09 (m, 4H), 7.01 (d, J=9.1 Hz, 1H), 6.91 (s, 1H), 6.26 (s, 1H), 4.52 (s, 1H), 4.12 (d, J=6.3 Hz, 4H), 3.67 (m, 6H), 3.39 (s, 6H), 3.31 (s, 3H), 3.16-2.92 (m, 8H), 2.08 (m, 2H), 1.80 (s, 4H), 1.51 (s, 2H), 1.34 (m, 4H), 1.02 (d, J=6.8 Hz, 6H). LCMS (ESI): R_{T}=1.180 min, m/z. found 996.0 [M-CF₃COOH+H]⁺.

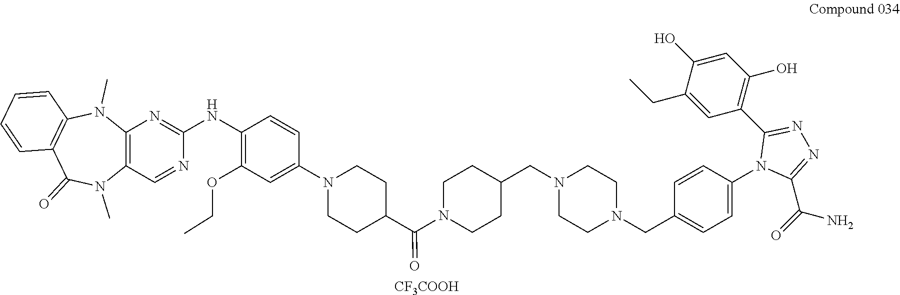 [SMILES: CCOc1cc(N2CCC(C(=O)N3CCC(CN4CCN(Cc5ccc(-n6c(C(N)=O)nnc6-c6cc(CC)c(O)cc6O)cc5)CC4)CC3)CC2)ccc1Nc1ncc2c(n1)N(C)c1ccccc1C(=O)N2C ; "CF3COOH"]

4-(4-((4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperazin-1-yl)methyl)phenyl)-5-(5-ethyl-2,4-dihydroxyphenyl)-4H-1,2,4-triazole-3-carboxamide, trifluoroacetic acid. ¹H NMR (400 MHz, DMSO-d₆): δ 9.64 (s, 1H), 8.36-8.32 (m, 2H), 8.09-8.08 (m, 2H), 7.68-7.73 (m, 2H), 7.53-7.16 (m, 8H), 6.91-6.75 (m, 1H), 6.62 (s, 1H), 6.31 (s, 1H), 4.39-4.38 (m, 1H), 4.15-4.10 (m, 2H), 4.05-3.95 (m, 1H), 3.68-3.65 (m, 2H), 3.39 (s, 4H), 3.32 (s, 4H), 3.11-2.89 (m, 6H), 2.53-2.35 (m, 5H), 2.41-2.22 (m, 3H), 1.80-1.73 (m, 8H), 1.34 (m, 4H), 1.02 (m, 2H), 0.85-0.84 (m, 4H). LCMS (ESI): R_{T}=1.060 min, m/z. found 1004.4 [M-CF₃COOH+H]⁺.

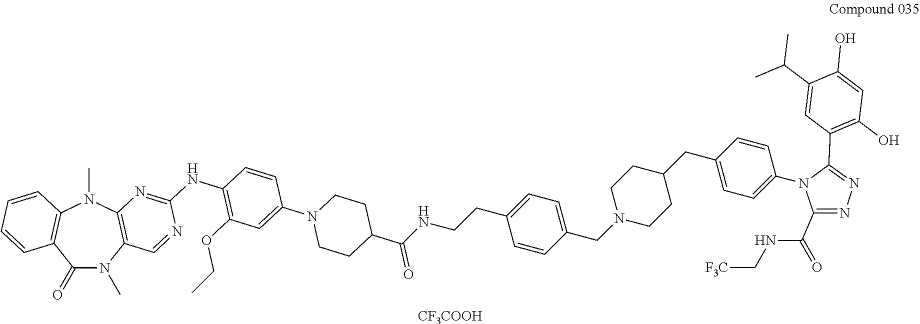 [SMILES: CCOc1cc(N2CCC(C(=O)NCCc3ccc(CN4CCC(Cc5ccc(-n6c(C(=O)NCC(F)(F)F)nnc6-c6cc(C(C)C)c(O)cc6O)cc5)CC4)cc3)CC2)ccc1Nc1ncc2c(n1)N(C)c1ccccc1C(=O)N2C ; "CF3COOH"]

N-(4-((4-(4-(3-(2,4-dihydroxy-5-isopropylphenyl)-5-((2,2,2-trifluoroethyl)carbamoyl)-4H-1,2,4-triazol-4-yl)benzyl)piperidin-1-yl)methyl)phenethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 9.78 (s, 1H), 9.61 (m, 1H), 8.33 (s, 1H), 7.95 (s, 3H), 7.68-7.67 (d, J=7.5 Hz, 1H), 7.50-7.17 (m, 13H), 6.59 (s, 1H), 6.34 (s, 1H), 4.24-4.23 (m, 2H), 4.11-4.07 (m, 4H), 3.99-3.90 (m, 4H), 3.65-3.62 (m, 3H), 3.38-3.29 (m, 9H), 2.91-2.74 (m, 5H), 2.55-2.54 (m, 1H), 2.33 (m, 1H), 2.01 (m, 1H), 1.77-1.70 (m, 5H), 1.33-1.24 (m, 5H), 0.82-0.81 (d, J=6.8 Hz, 6H). LCMS (ESI): R_{T}=1.230 min, m/z. found 1135.3 [M-CF₃COOH+H]⁺.

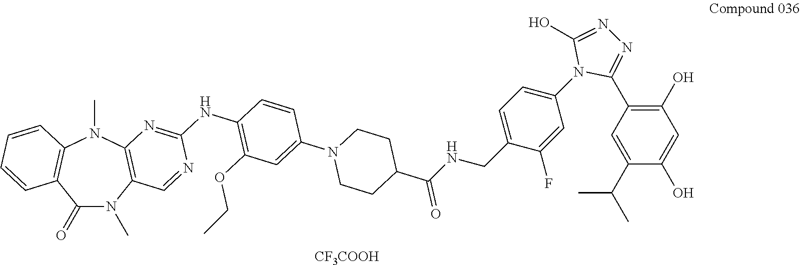 [SMILES: CCOc1cc(N2CCC(C(=O)NCc3ccc(-n4c(O)nnc4-c4cc(C(C)C)c(O)cc4O)cc3F)CC2)ccc1Nc1ncc2c(n1)N(C)c1ccccc1C(=O)N2C ; "CF3COOH"]

N-(4-(3-(2,4-dihydroxy-5-isopropylphenyl)-5-hydroxy-4H-1,2,4-triazol-4-yl)-2-fluorobenzyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.96 (s, 1H), 9.61 (s, 1H), 9.42 (s, 1H), 8.49 (s, 1H), 8.37 (s, 1H), 8.28-7.99 (m, 2H), 7.70-7.67 (m, 1H), 7.53-7.49 (m, 1H), 7.29-6.93 (m, 9H), 6.25 (s, 1H), 4.31 (d, J=4.4 Hz, 2H), 4.16-4.11 (m, 2H), 3.67 (d, J=12 Hz, 2H), 3.39 (s, 3H), 3.32 (s, 3H), 3.04-3.00 (m, 2H), 1.99-1.78 (m, 4H), 1.35 (t, J=7.2 Hz, 3H), 1.24 (s, 2H), 1.04 (d, J=7.2 Hz, 6H). LCMS (ESI): R_{T}=1.366 min, m/z. found 843.8 [M-CF₃COOH+H]⁺.

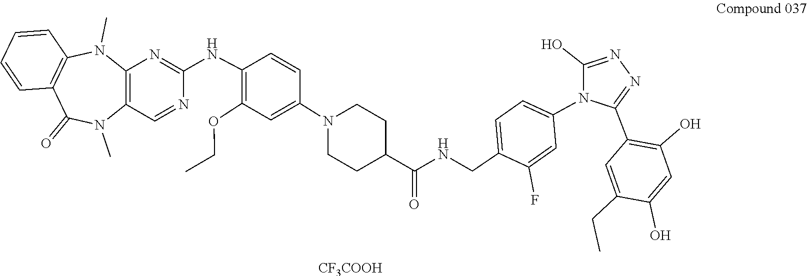 [SMILES: CCOc1cc(N2CCC(C(=O)NCc3ccc(-n4c(O)nnc4-c4cc(CC)c(O)cc4O)cc3F)CC2)ccc1Nc1ncc2c(n1)N(C)c1ccccc1C(=O)N2C ; "CF3COOH"]

1-(4-((5,11-dimethyl-6-oxo-6,11-dihydro-5H-benzo[e]pyrimido[5,4-b][1,4]diazepin-2-yl)amino)-3-ethoxyphenyl)-N-(4-(3-(5-ethyl-2,4-dihydroxyphenyl)-5-hydroxy-4H-1,2,4-triazol-4-yl)-2-fluorobenzyl)piperidine-4-carboxamide, trifluoroacetic acid. ¹H NMR (400 MHz, DMSO-d₆): δ 11.95 (s, 1H), 9.59 (s, 1H), 9.42 (s, 1H), 8.47 (s, 1H), 8.37 (s, 1H), 8.20-8.01 (m, 2H), 7.70-7.67 (m, 1H), 7.53-7.49 (m, 1H), 7.27-6.95 (m, 8H), 6.25 (s, 1H), 4.30 (d, J=5.2 Hz, 2H), 4.16-4.11 (m, 2H), 3.67 (d, J=12 Hz, 2H), 3.39 (s, 3H), 3.32 (s, 3H), 2.43-2.37 (m, 3H), 1.96-1.73 (m, 4H), 1.35 (t, J=6.8 Hz, 3H), 1.24 (s, 2H), 1.04 (d, J=7.6 Hz, 4H). LCMS (ESI): R_{T}=1.327 min, m/z. found 829.8 [M-CF₃COOH+H]⁺.

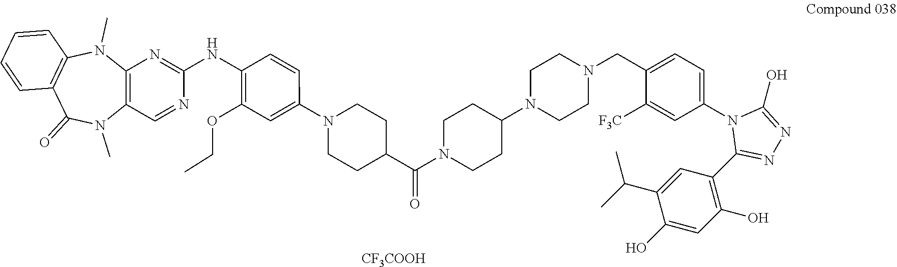 [SMILES: CCOc1cc(N2CCC(C(=O)N3CCC(N4CCN(Cc5ccc(-n6c(O)nnc6-c6cc(C(C)C)c(O)cc6O)cc5C(F)(F)F)CC4)CC3)CC2)ccc1Nc1ncc2c(n1)N(C)c1ccccc1C(=O)N2C ; "CF3COOH"]

2-((4-(4-(4-(4-(4-(3-(2,4-dihydroxy-5-isopropylphenyl)-5-hydroxy-4H-1,2,4-triazol-4-yl)-2-(trifluoromethyl)benzyl)piperazin-1-yl)piperidine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 12.02 (s, 1H), 9.60 (s, 1H), 9.36 (s, 1H), 8.31 (s, 1H), 7.86-7.65 (m, 5H), 7.49-7.42 (m, 3H), 7.24-7.14 (m, 2H), 6.90 (s, 1H), 6.61 (d, J=2.8 Hz, 1H), 6.51-6.47 (m, 1H), 6.25 (s, 1H), 4.42-4.38 (m, 1H), 4.08-3.98 (m, 3H), 3.67-3.55 (m, 5H), 3.78 (s, 3H), 3.26 (s, 3H), 3.03-2.99 (m, 2H), 2.73-2.54 (m, 4H), 2.51 (s, 2H), 2.39-2.35 (m, 4H), 1.85-1.62 (m, 6H), 1.29 (t, J=6.8 Hz, 4H), 1.24-1.15 (m, 3H), 1.01 (d, J=7.2 Hz, 6H). LCMS (ESI): R_{T}=1.367 min, m/z. found 1046.0 [M-CF₃COOH+H]⁺.

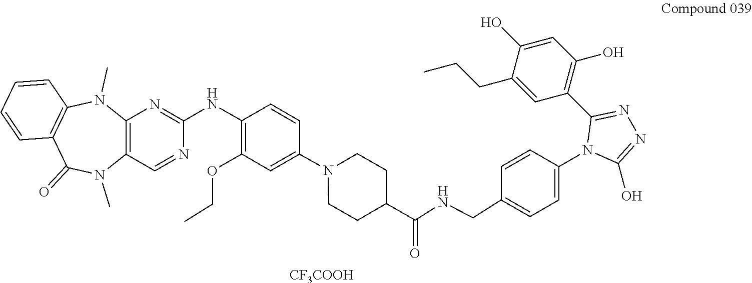 [SMILES: CCCc1cc(-c2nnc(O)n2-c2ccc(CNC(=O)C3CCN(c4ccc(Nc5ncc6c(n5)N(C)c5ccccc5C(=O)N6C)c(OCC)c4)CC3)cc2)c(O)cc1O ; "CF3COOH"]

N-(4-(3-(2,4-dihydroxy-5-propylphenyl)-5-hydroxy-4H-1,2,4-triazol-4-yl)benzyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.87 (s, 1H), 9.58-9.26 (m, 2H), 8.56-8.34 (m, 2H), 8.30-7.95 (m, 2H), 7.69 (dd, J=7.6 Hz, 1.2 Hz, 1H), 7.55-7.47 (m, 1H), 7.28-7.08 (m, 7H), 6.86 (s, 1H), 6.24 (s, 1H), 4.31-4.24 (m, 4H), 4.18-4.11 (m, 4H), 3.71-3.61 (m, 2H), 3.44-3.26 (m, 7H), 2.37-2.29 (m, 2H), 1.97 (s, 4H), 1.48-1.32 (m, 5H), 0.83-0.77 (m, 3H). LCMS (ESI): R_{T}=1.240 min, m/z. found 825.3 [M-CF₃COOH+H]⁺.

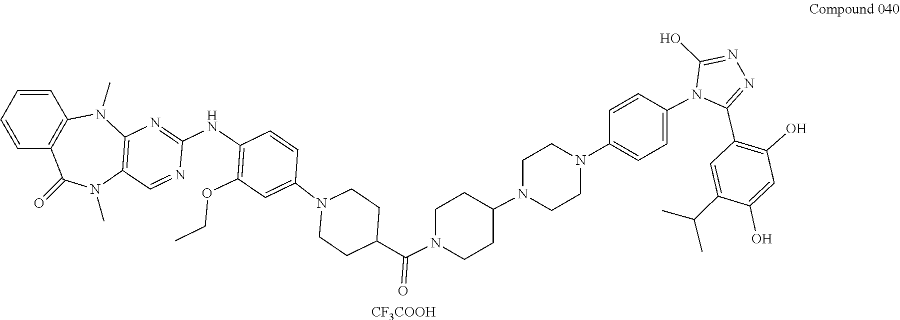 [SMILES: CCOc1cc(N2CCC(C(=O)N3CCC(N4CCN(c5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; "CF3COOH"]

2-((4-(4-(4-(4-(4-(3-(2,4-dihydroxy-5-isopropylphenyl)-5-hydroxy-4H-1,2,4-triazol-4-yl)phenyl)piperazin-1-yl)piperidine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.87 (s, 1H), 9.74-9.36 (m, 3H), 8.35 (s, 1H), 8.14-7.84 (m, 2H), 7.68 (dd, J=7.6 Hz, 1.6 Hz, 1H), 7.54-7.47 (m, 1H), 7.26-7.00 (m, 6H), 6.90-6.65 (m, 2H), 6.27 (s, 1H), 4.67-4.47 (m, 3H), 4.24-4.08 (m, 4H), 3.94-3.84 (m, 2H), 3.74-3.50 (m, 6H), 3.39 (s, 3H), 3.31 (s, 3H), 3.23-3.09 (m, 3H), 3.02-2.91 (m, 4H), 2.22-2.08 (m, 2H), 1.79 (s, 4H), 1.66-1.43 (m, 2H), 1.37-1.30 (m, 3H), 0.96 (d, J=6.8 Hz, 6H). LCMS (ESI): R_{T}=1.206 min, m/z. found 964.0 [M-CF₃COOH+H]⁺.

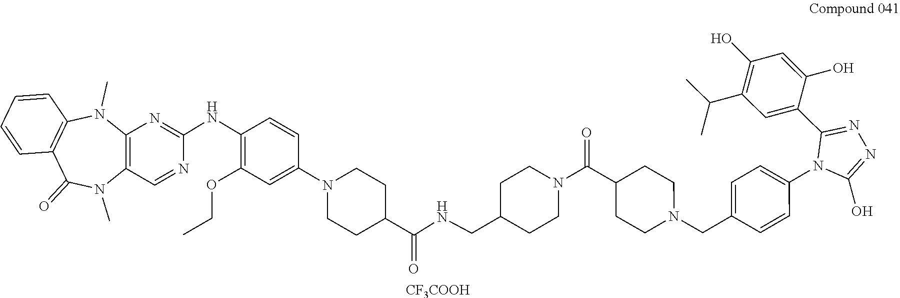 [SMILES: CCOc1cc(N2CCC(C(=O)NCC3CCN(C(=O)C4CCN(Cc5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; "CF3COOH"]

N-((1-(1-(4-(3-(2,4-dihydroxy-5-isopropylphenyl)-5-hydroxy-4H-1,2,4-triazol-4-yl)benzyl)piperidine-4-carbonyl)piperidin-4-yl)methyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.91 (s, 1H), 9.59 (s, 1H), 9.41 (s, 1H), 8.31 (s, 1H), 7.89-7.77 (m, 3H), 7.70-7.64 (m, 1H), 7.54-7.45 (m, 1H), 7.29 (d, J=8.0 Hz, 2H), 7.23 (d, J=8.4 Hz, 1H), 7.19-7.10 (m, 3H), 6.75 (s, 1H), 6.63-6.59 (m, 1H), 6.52-6.47 (m, 1H), 6.26 (s, 1H), 4.41-4.32 (m, 1H), 4.12-4.02 (m, 2H), 3.96-3.85 (m, 1H), 3.71-3.60 (m, 2H), 3.42 (s, 2H), 3.38 (s, 3H), 3.28 (s, 3H), 3.02-2.89 (m, 4H), 2.84-2.74 (m, 2H), 2.71-2.56 (m, 3H), 2.29-2.21 (m, 1H), 2.03-1.93 (m, 2H), 1.85-1.41 (m, 14H), 1.29 (t, J=6.8 Hz, 3H), 0.93 (d, J=6.8 Hz, 6H). LCMS (ESI): R_{T}=1.035 min, m/z. found 1033.4 [M-CF₃COOH+H]⁺.

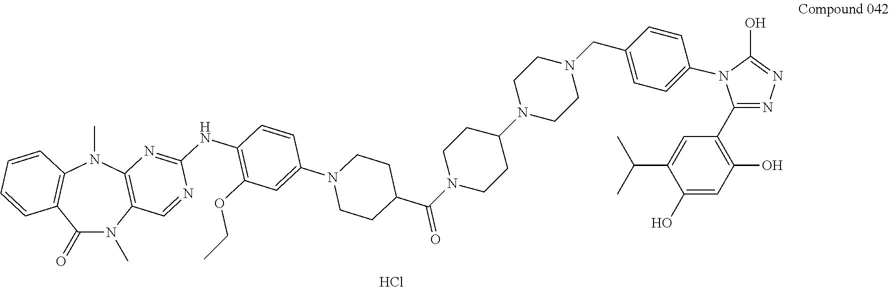 [SMILES: CCOc1cc(N2CCC(C(=O)N3CCC(N4CCN(Cc5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; Cl]

2-((4-(4-(4-(4-(4-(3-(2,4-dihydroxy-5-isopropylphenyl)-5-hydroxy-4H-1,2,4-triazol-4-yl)benzyl)piperazin-1-yl)piperidine-1-carbonyl)piperidin-1-yl)-2-ethoxyphenyl)amino)-5,11-dimethyl-5H-benzo[e]pyrimido[5,4-b][1,4]diazepin-6(11H)-one, hydrochloride. ¹H NMR (400 MHz, DMSO-d₆): δ 11.98 (s, 1H), 9.70 (s, 1H), 9.29 (s, 1H), 8.44 (s, 1H), 8.36 (s, 1H), 8.26 (s, 1H), 7.74-7.58 (m, 4H), 7.55-7.49 (m, 1H), 7.44 (s, 1H), 7.31-7.17 (m, 4H), 6.93 (s, 1H), 6.34 (s, 1H), 4.59-4.53 (m, 1H), 4.45-4.33 (m, 2H), 4.23-4.12 (m, 3H), 3.80-3.45 (m, 12H), 3.40 (s, 3H), 3.36 (s, 3H), 3.20-3.08 (m, 2H), 3.05-2.99 (m, 1H), 2.70-2.54 (m, 2H), 2.35-2.10 (m, 4H), 1.98-1.82 (m, 2H), 1.76-1.64 (m, 1H), 1.61-1.49 (m, 1H), 1.40 (t, J=6.8 Hz, 3H), 1.04 (d, J=6.8 Hz, 6H). LC-MS(ESI): R_{T}=1.031 min, m/z. found 977.3 [M−HCl+H]⁺.

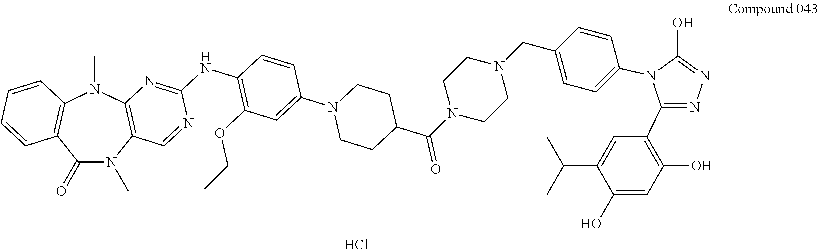 [SMILES: CCOc1cc(N2CCC(C(=O)N3CCN(Cc4ccc(-n5c(O)nnc5-c5cc(C(C)C)c(O)cc5O)cc4)CC3)CC2)ccc1Nc1ncc2c(n1)N(C)c1ccccc1C(=O)N2C ; Cl]

2-((4-(4-(4-(4-(3-(2,4-dihydroxy-5-isopropylphenyl)-5-hydroxy-4H-1,2,4-triazol-4-yl)benzyl)piperazine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, hydrochloride. ¹H NMR (400 MHz, DMSO-d₆): δ 13.02 (s, 1H), 11.92 (d, J=50.0 Hz, 2H), 9.94-9.24 (m, 1H), 8.49 (d, J=28.4 Hz, 2H), 8.26 (d, J=8.4 Hz, 1H), 7.72-7.45 (m, 6H), 7.30-7.19 (m, 4H), 6.91 (s, 1H), 6.36 (s, 1H), 4.49-4.16 (m, 6H), 3.76-3.53 (m, 5H), 3.41 (s, 3H), 3.37 (s, 3H), 3.29 (d, J=8.4 Hz, 2H), 3.23-3.12 (m, 2H), 3.10-2.89 (m, 3H), 2.29 (s, 2H), 1.94 (s, 2H), 1.40 (t, J=7.2 Hz, 3H), 1.03 (d, J=6.8 Hz, 6H). LCMS (ESI): R_{T}=1.080 min, m/z. found 894.3 [M−HCl+H]⁺.

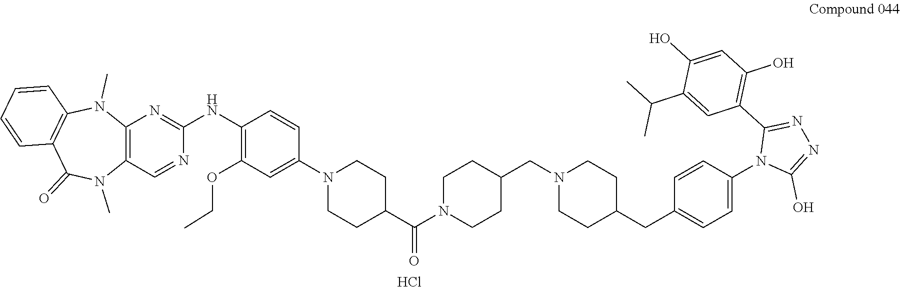 [SMILES: CCOc1cc(N2CCC(C(=O)N3CCC(CN4CCC(Cc5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; Cl]

2-((4-(4-(4-((4-(4-(3-(2,4-dihydroxy-5-isopropylphenyl)-5-hydroxy-4H-1,2,4-triazol-4-yl)benzyl)piperidin-1-yl)methyl)piperidine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, hydrochloride. H NMR (400 MHz, DMSO-d₆): δ 11.93 (s, 1H), 9.64 (m, 3H), 8.42 (s, 1H), 8.28 (s, 2H), 7.70 (d, J=7.7 Hz, 1H), 7.52 (t, J=7.5 Hz, 2H), 7.27 (d, J=8.3 Hz, 1H), 7.19 (t, J=7.8 Hz, 3H), 7.12 (d, J=8.3 Hz, 2H), 6.77 (d, J=4.5 Hz, 1H), 6.31 (s, 1H), 4.37 (s, 2H), 4.21-4.15 (m, 4H), 3.99 (s, 1H), 3.59 (s, 3H), 3.45 (s, 2H), 3.40 (s, 3H), 3.35 (s, 3H), 3.10 (s, 3H), 3.01-2.89 (m, 3H), 2.81 (s, 2H), 2.68-2.54 (m, 3H), 2.10 (s, 2H), 1.87 (m, 3H), 1.69 (s, 3H), 1.39 (t, J=6.8 Hz, 3H), 1.24 (s, 1H), 1.17 (s, 1H), 1.04 (s, 1H), 0.97-0.93 (m, 6H). LCMS (ESI): R_{T}=1.100 min, m/z. found 990.4 [M−HCl+H]⁺.

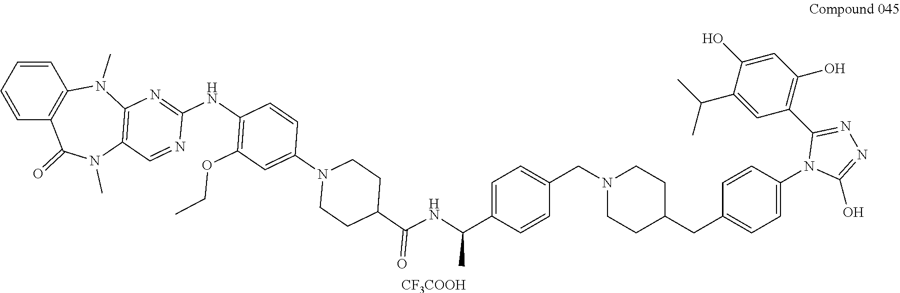 [SMILES: CCOc1cc(N2CCC(C(=O)N[C@H](C)c3ccc(CN4CCC(Cc5ccc(-n6c(O)nnc6-c6cc(C(C)C)c(O)cc6O)cc5)CC4)cc3)CC2)ccc1Nc1ncc2c(n1)N(C)c1ccccc1C(=O)N2C ; "CF3COOH"]

(R)—N-(1-(4-((4-(4-(3-(2,4-dihydroxy-5-isopropylphenyl)-5-hydroxy-4H-1,2,4-triazol-4-yl)benzyl)piperidin-1-yl)methyl)phenyl)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 11.92 (s, 1H), 9.62 (s, 1H), 9.39 (s, 1H), 8.42-8.35 (m, 2H), 8.07-7.95 (m, 2H), 7.68 (dd, J=8.0, 1.2 Hz, 1H), 7.55-7.34 (m, 5H), 7.25-7.09 (m, 6H), 6.92-6.67 (m, 2H), 6.26 (d, J=3.6 Hz, 1H), 4.97-4.93 (m, 1H), 4.37-4.08 (m, 4H), 3.70-3.65 (m, 2H), 3.39 (s, 3H), 3.35-3.31 (m, 5H), 3.14-2.66 (m, 4H), 2.48-2.30 (s, 4H), 2.02-1.63 (m, 7H), 1.41-1.24 (m, 9H), 0.94 (d, J=6.8 Hz, 6H). LCMS (ESI): R_{T}=1.345 min, m/z. found 1027.0 [M-CF₃COOH+H]⁺.

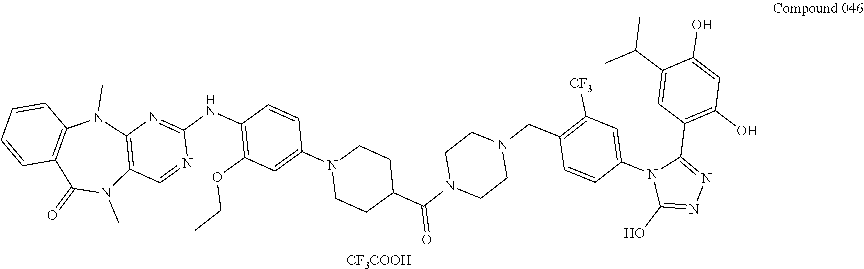 [SMILES: CCOc1cc(N2CCC(C(=O)N3CCN(Cc4ccc(-n5c(O)nnc5-c5cc(C(C)C)c(O)cc5O)cc4C(F)(F)F)CC3)CC2)ccc1Nc1ncc2c(n1)N(C)c1ccccc1C(=O)N2C ; "CF3COOH"]

2-((4-(4-(4-(4-(3-(2,4-dihydroxy-5-isopropylphenyl)-5-hydroxy-4H-1,2,4-triazol-4-yl)-2-(trifluoromethyl)benzyl)piperazine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 12.07 (s, 1H), 9.65 (s, 1H), 9.38 (s, 1H), 8.36 (s, 1H), 8.23-7.95 (m, 2H), 7.85 (d, J=7.6 Hz, 1H), 7.71-7.49 (m, 4H), 7.26-7.16 (m, 2H), 7.01-6.89 (m, 2H), 6.25 (s, 1H), 4.72-4.54 (m, 4H), 4.13 (q, J=6.8 Hz, 4H), 3.67 (d, J=10.8 Hz, 4H), 3.36 (d, J=29.2 Hz, 8H), 3.12-2.81 (m, 4H), 1.85 (s, 4H), 1.35 (t, J=6.8 Hz, 3H), 1.04 (d, J=6.8 Hz, 7H). LCMS (ESI): R_{T}=1.320 min, m/z. found 962.3 [M-CF₃COOH+H]⁺.

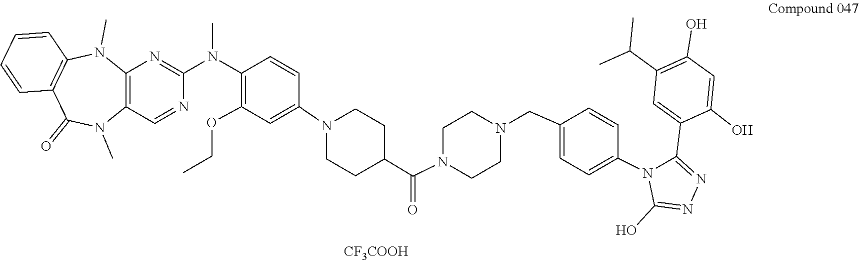 [SMILES: CCOc1cc(N2CCC(C(=O)N3CCN(Cc4ccc(-n5c(O)nnc5-c5cc(C(C)C)c(O)cc5O)cc4)CC3)CC2)ccc1N(C)c1ncc2c(n1)N(C)c1ccccc1C(=O)N2C ; "CF3COOH"]

2-((4-(4-(4-(4-(3-(2,4-dihydroxy-5-isopropylphenyl)-5-hydroxy-4H-1,2,4-triazol-4-yl)benzyl)piperazine-1-carbonyl)piperidin-1-yl)-2-ethoxyphenyl)(methyl)amino)-5,11-dimethyl-5,11-dihydro-6H-benzo[e]pyrimido[5,4-b][1,4]diazepin-6-one, trifluoroacetic acid. ¹H NMR (400 MHz, CD₃OD): δ 7.96 (s, 1H), 7.79-7.76 (m, 2H), 7.60-7.55 (m, 3H), 7.40 (d, J=8.4 Hz, 2H), 7.26-7.21 (m, 4H), 6.89-6.76 (m, 3H), 6.22 (s, 1H), 4.39 (m, 3H), 4.08-4.03 (m, 3H), 3.96-3.80 (m, 4H), 3.49-3.43 (m, 9H), 3.11-3.05 (m, 4H), 1.99-1.91 (s, 4H), 1.32-1.41 (m, 5H), 1.21 (t, J=7.6 Hz, 3H), 1.02 (d, J=7.6 Hz, 6H). LCMS (ESI): R_{T}=1.043 min, m/z. found 908.5 [M-CF₃COOH+H]⁺.

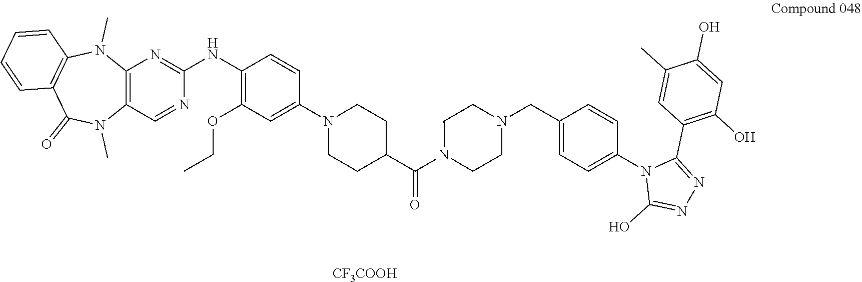 [SMILES: CCOc1cc(N2CCC(C(=O)N3CCN(Cc4ccc(-n5c(O)nnc5-c5cc(C)c(O)cc5O)cc4)CC3)CC2)ccc1Nc1ncc2c(n1)N(C)c1ccccc1C(=O)N2C ; "CF3COOH"]

2-((4-(4-(4-(4-(3-(2,4-dihydroxy-5-methylphenyl)-5-hydroxy-4H-1,2,4-triazol-4-yl)benzyl)piperazine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.89 (s, 1H), 9.57 (s, 1H), 9.32 (s, 1H), 8.31 (s, 1H), 7.86 (s, 1H), 7.78 (d, J=8.8 Hz, 1H), 7.68-7.65 (m, 1H), 7.51-7.46 (m, 1H), 7.30-7.11 (m, 6H), 6.90 (s, 1H), 6.61 (d, J=2.0 Hz, 1H), 6.50-6.47 (m, 1H), 6.23 (s, 1H), 4.09-4.03 (m, 2H), 3.67-3.64 (m, 2H), 3.53-3.47 (m, 6H), 3.38 (s, 3H), 3.31 (s, 1H), 3.28 (s, 3H), 2.73-2.67 (m, 3H), 2.37-2.28 (m, 4H), 1.95 (s, 3H), 1.67-1.71 (m, 4H), 1.29 (t, J=7.2 Hz, 3H). LCMS (ESI): R_{T}=1.041 min, m/z. found 866.3 [M-CF₃COOH+H]⁺.

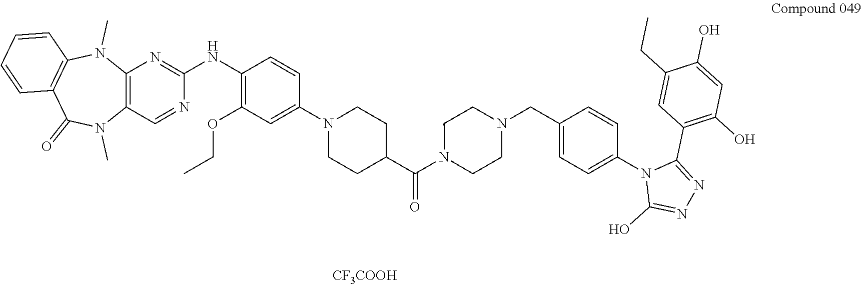 [SMILES: CCOc1cc(N2CCC(C(=O)N3CCN(Cc4ccc(-n5c(O)nnc5-c5cc(CC)c(O)cc5O)cc4)CC3)CC2)ccc1Nc1ncc2c(n1)N(C)c1ccccc1C(=O)N2C ; "CF3COOH"]

2-((2-ethoxy-4-(4-(4-(4-(3-(5-ethyl-2,4-dihydroxyphenyl)-5-hydroxy-4H-1,2,4-triazol-4-yl)benzyl)piperazine-1-carbonyl)piperidin-1-yl)phenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.98 (s, 1H), 10.05 (s, 1H), 9.62 (d, J=3.2 Hz, 1H), 9.34 (d, J=4.0 Hz, 1H), 8.35 (s, 1H), 8.10-8.18 (m, 2H), 7.70-7.67 (m, 1H), 7.53-7.47 (m, 3H), 7.29-7.10 (m, 5H), 6.98-6.68 (m, 3H), 6.24 (s, 1H), 4.34 (s, 1H), 4.30 (s, 2H), 4.14-4.08 (m, 3H), 3.70-3.66 (m, 2H), 3.39 (s, 4H), 3.31 (s, 4H), 3.13-2.90 (m, 5H), 2.45-2.33 (m, 2H), 1.82 (s, 5H), 1.33 (t, J=7.6 Hz, 3H), 1.02 (t, J=7.8 Hz, 4H). LCMS (ESI): R_{T}=1.076 min, m/z. found 880.3 [M-CF₃COOH+H]⁺.

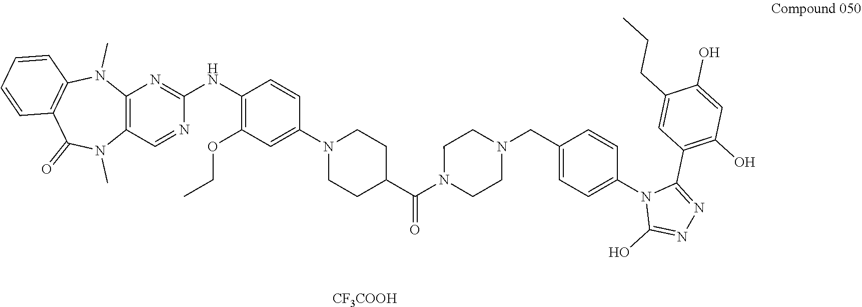 [SMILES: CCCc1cc(-c2nnc(O)n2-c2ccc(CN3CCN(C(=O)C4CCN(c5ccc(Nc6ncc7c(n6)N(C)c6ccccc6C(=O)N7C)c(OCC)c5)CC4)CC3)cc2)c(O)cc1O ; "CF3COOH"]

2-((4-(4-(4-(4-(3-(2,4-dihydroxy-5-propylphenyl)-5-hydroxy-4H-1,2,4-triazol-4-yl)benzyl)piperazine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.89 (s, 1H), 9.52 (s, 1H), 9.35 (s, 1H), 8.31 (s, 1H), 7.86 (s, 1H), 7.79 (d, J=8.8 Hz, 1H), 7.67 (dd, J=7.6, 1.6 Hz, 1H), 7.52-7.46 (m, 1H), 7.31-7.10 (m, 6H), 6.79 (s, 1H), 6.61 (d, J=2.0 Hz, 1H), 6.49 (dd, J=8.8, 2.0 Hz, 1H), 6.25 (s, 1H), 4.11-4.03 (m, 2H), 3.70-3.62 (m, 2H), 3.53 (s, 2H), 3.47 (s, 3H), 3.38 (s, 3H), 3.31-3.25 (m, 4H), 2.79-2.62 (m, 3H), 2.41-2.25 (m, 7H), 1.72-1.63 (m, 4H), 1.44-1.35 (m, 2H), 1.32-1.26 (m, 3H), 0.80-0.74 (m, 3H). LCMS (ESI): R_{T}=1.198 min, m/z. found 894.9 [M-CF₃COOH+H]⁺.

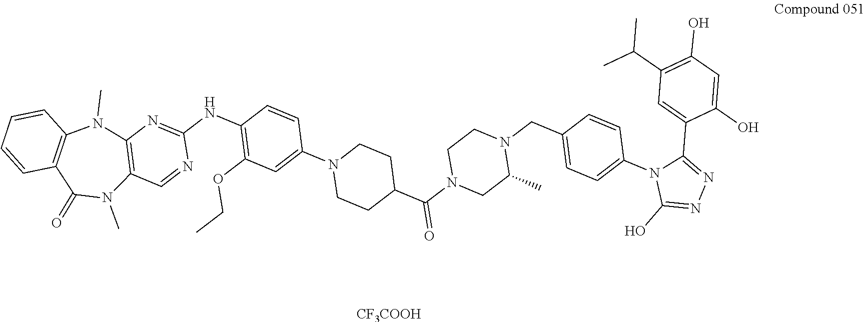 [SMILES: CCOc1cc(N2CCC(C(=O)N3CCN(Cc4ccc(-n5c(O)nnc5-c5cc(C(C)C)c(O)cc5O)cc4)[C@H](C)C3)CC2)ccc1Nc1ncc2c(n1)N(C)c1ccccc1C(=O)N2C ; "CF3COOH"]

(R)-2-((4-(4-(4-(4-(3-(2,4-dihydroxy-5-isopropylphenyl)-5-hydroxy-4H-1,2,4-triazol-4-yl)benzyl)-3-methylpiperazine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.98 (s, 1H), 9.62 (s, 1H), 9.34 (s, 1H), 8.33 (s, 1H), 8.05-7.82 (m, 1H), 7.71-7.65 (m, 1H), 7.59-7.43 (m, 3H), 7.33-6.85 (m, 6H), 6.78-6.54 (m, 1H), 6.25 (s, 1H), 4.13-4.05 (m, 5H), 3.73-3.63 (m, 4H), 3.41-3.27 (m, 9H), 3.10-2.75 (m, 7H), 1.83-1.66 (m, 4H), 1.52-1.28 (m, 7H), 1.06-0.97 (m, 7H). LCMS (ESI): R_{T}=1.042 min, m/z. found 908.3 [M-CF₃COOH+H]⁺.

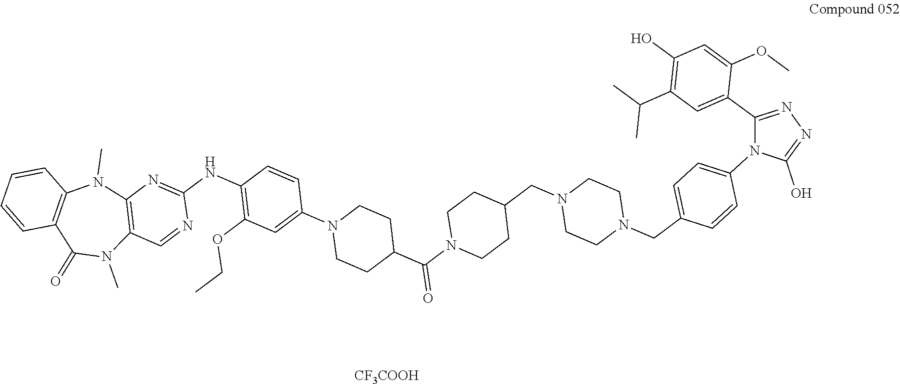 [SMILES: CCOc1cc(N2CCC(C(=O)N3CCC(CN4CCN(Cc5ccc(-n6c(O)nnc6-c6cc(C(C)C)c(O)cc6OC)cc5)CC4)CC3)CC2)ccc1Nc1ncc2c(n1)N(C)c1ccccc1C(=O)N2C ; "CF3COOH"]

2-((2-ethoxy-4-(4-(4-((4-(4-(3-hydroxy-5-(4-hydroxy-5-isopropyl-2-methoxyphenyl)-4H-1,2,4-triazol-4-yl)benzyl)piperazin-1-yl)methyl)piperidine-1-carbonyl)piperidin-1-yl)phenyl)amino)-5,11-dimethyl-5,11-dihydro-6H-benzo[e]pyrimido[5,4-b][1,4]diazepin-6-one, trifluoroacetic acid. ¹H NMR (400 MHz, CD₃OD): δ 8.23 (s, 1H), 8.10 (d, J=8.8 Hz, 1H), 7.75-7.71 (m, 1H), 7.53-7.47 (m, 1H), 7.35 (d, J=8.0 Hz, 2H), 7.25-7.11 (m, 5H), 6.69-6.59 (m, 2H), 6.24 (s, 1H), 5.36-5.31 (m, 1H), 4.60 (s, 3H), 4.57-4.50 (m, 1H), 4.16-4.04 (m, 3H), 3.67-3.56 (m, 4H), 3.47 (s, 3H), 3.40 (s, 3H), 3.21-3.11 (m, 3H), 2.83-2.72 (m, 3H), 2.63-2.48 (m, 7H), 2.33-2.26 (m, 2H), 2.21-2.15 (m, 1H), 2.06-1.99 (m, 1H), 1.82-1.78 (m, 7H), 1.43 (t, J=6.8 Hz, 3H), 1.16 (d, J=6.8 Hz, 6H). LCMS (ESI): R_{T}=1.163 min, m/z. found 1005.4 [M-CF₃COOH+H]⁺.

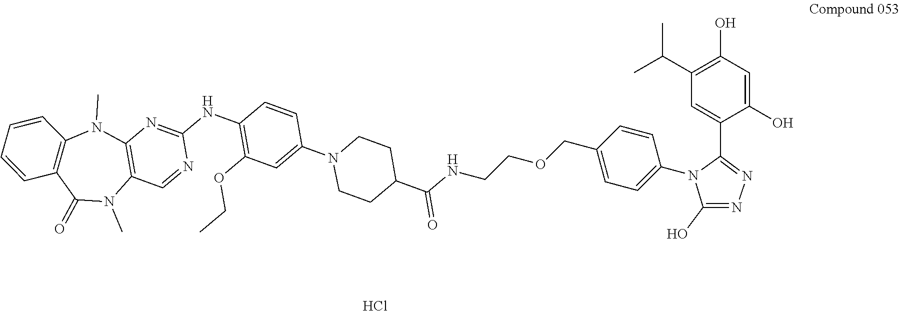 [SMILES: CCOc1cc(N2CCC(C(=O)NCCOCc3ccc(-n4c(O)nnc4-c4cc(C(C)C)c(O)cc4O)cc3)CC2)ccc1Nc1ncc2c(n1)N(C)c1ccccc1C(=O)N2C ; Cl]

N-(2-((4-(3-(2,4-dihydroxy-5-isopropylphenyl)-5-hydroxy-4H-1,2,4-triazol-4-yl)benzyl)oxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, hydrochloride. ¹H NMR (400 MHz, DMSO): δ 11.92 (s, 1H), 9.60 (s, 1H), 9.36 (d, J=2.4 Hz, 1H), 8.41 (d, J=4.8 Hz, 1H), 8.20-8.13 (m, 2H), 7.70-7.68 (m, 1H), 7.52-7.50 (m, 1H), 7.34-7.14 (m, 7H), 6.85 (s, 1H), 6.25 (s, 1H), 4.47 (d, J=6.0 Hz, 2H), 4.18-4.13 (m, 2H), 3.64-3.55 (m, 8H), 3.54-3.45 (m, 3H), 3.43 (s, 3H), 3.40 (s, 3H), 3.31-3.27 (m, 2H), 3.01-2.97 (m, 1H), 2.04-1.92 (m, 2H), 1.38-1.33 (m, 4H), 0.98 (d, J=7.6 Hz, 6H). LCMS (ESI): R_{T}=1.341 min, m/z. found 870.0 [M−HCl+H]⁺.

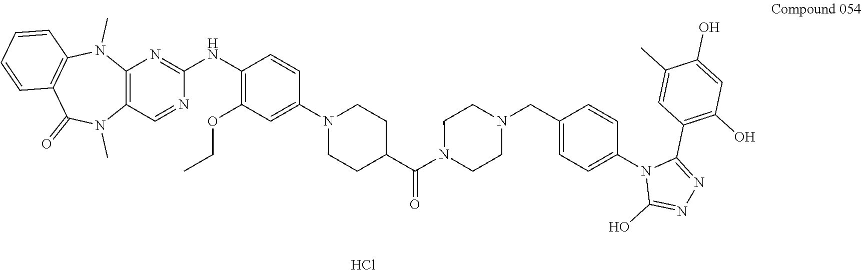 [SMILES: CCOc1cc(N2CCC(C(=O)N3CCN(Cc4ccc(-n5c(O)nnc5-c5cc(C)c(O)cc5O)cc4)CC3)CC2)ccc1Nc1ncc2c(n1)N(C)c1ccccc1C(=O)N2C ; Cl]

2-((4-(4-(4-(4-(3-(2,4-dihydroxy-5-isopropylphenyl)-5-hydroxy-4H-1,2,4-triazol-4-yl)benzyl)piperazine-1-carbonyl)piperidin-1-yl)-2-ethoxyphenyl)(methyl)amino)-5,11-dimethyl-5,11-dihydro-6H-benzo[e]pyrimido[5,4-b][1,4]diazepin-6-one, hydrochloride. ¹H NMR (400 MHz, DMSO-d₆): δ 11.98 (s, 1H), 11.33-10.48 (m, 1H), 9.64 (s, 1H), 9.41 (s, 1H), 8.20 (s, 1H), 7.68-7.47 (m, 5H), 7.28-7.14 (m, 6H), 6.89 (s, 2H), 6.28 (s, 1H), 4.48-4.46 (m, 1H), 4.33-4.30 (m, 2H), 4.20 (d, J=3.2 Hz, 1H), 4.06-3.91 (m, 4H), 3.64-3.48 (m, 3H), 3.34-3.31 (m, 9H), 3.23-2.85 (m, 5H), 1.88-1.80 (m, 4H), 1.09-0.97 (m, 10H). LCMS (ESI): R_{T}=1.084 min, m/z. found 908.4 [M−HCl+H]⁺.

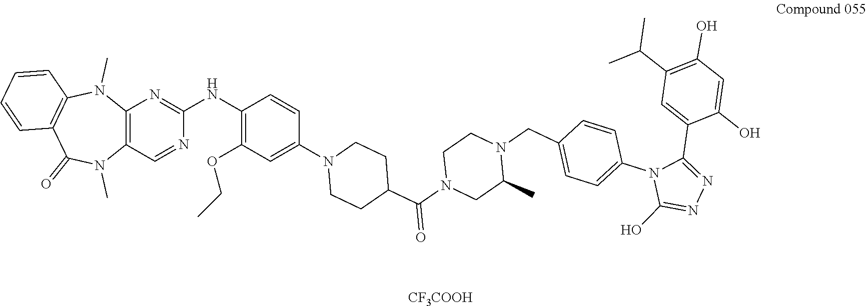 [SMILES: CCOc1cc(N2CCC(C(=O)N3CCN(Cc4ccc(-n5c(O)nnc5-c5cc(C(C)C)c(O)cc5O)cc4)[C@@H](C)C3)CC2)ccc1Nc1ncc2c(n1)N(C)c1ccccc1C(=O)N2C ; "CF3COOH"]

(S)-2-((4-(4-(4-(4-(3-(2,4-dihydroxy-5-isopropylphenyl)-5-hydroxy-4H-1,2,4-triazol-4-yl)benzyl)-3-methylpiperazine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.93 (s, 1H), 9.60 (s, 1H), 9.41 (s, 1H), 8.31 (s, 1H), 7.87-7.13 (m, 11H), 6.78-6.48 (m, 3H), 6.27 (s, 1H), 4.07 (q, J=6.8 Hz, 2H), 3.92-3.62 (m, 5H), 3.38 (s, 3H), 3.33 (s, 3H), 3.28 (s, 1H), 3.10-2.91 (m, 2H), 2.87-2.61 (m, 3H), 2.45-2.32 (m, 1H), 2.15-1.94 (m, 1H), 1.77-1.59 (m, 4H), 1.31-1.23 (m, 5H), 1.07 (dd, J=31.7, 5.4 Hz, 2H), 0.95 (d, J=6.0 Hz, 6H). LCMS (ESI): R_{T}=1.060 min, m/z. found 908.4 [M-CF₃COOH+H]⁺.

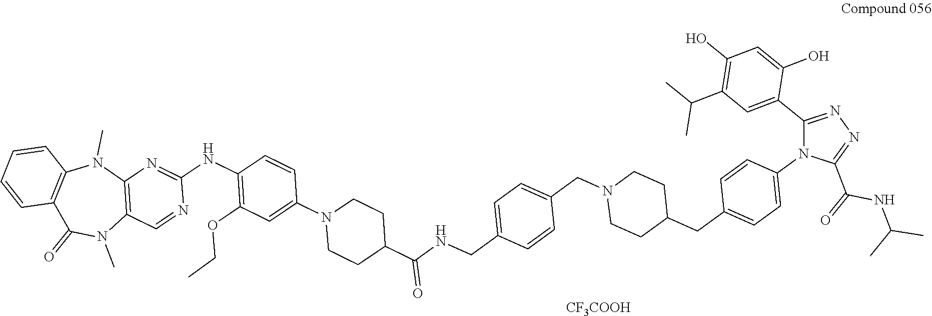 [SMILES: CCOc1cc(N2CCC(C(=O)NCc3ccc(CN4CCC(Cc5ccc(-n6c(C(=O)NC(C)C)nnc6-c6cc(C(C)C)c(O)cc6O)cc5)CC4)cc3)CC2)ccc1Nc1ncc2c(n1)N(C)c1ccccc1C(=O)N2C ; "CF3COOH"]

N-(4-((4-(4-(3-(2,4-dihydroxy-5-isopropylphenyl)-5-(isopropylcarbamoyl)-4H-1,2,4-triazol-4-yl)benzyl)piperidin-1-yl)methyl)benzyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 10.62 (d, J=24 Hz, 1H), 9.79 (d, J=15.6 Hz, 1H), 8.76 (d, J=8.8 Hz, 1H), 8.40-8.32 (m, 2H), 7.87 (s, 1H), 7.80 (d, J=8.8 Hz, 1H), 7.67 (dd, J=7.7, 1.6 Hz, 1H), 7.51-7.4 (m, 1H), 7.35-7.14 (m, 10H), 6.63-6.37 (m, 2H), 6.51 (dd, J=8.8, 1.9 Hz, 1H), 6.35 (s, 1H), 4.27-4.05 (m, 5H), 3.95-3.86 (m, 1H), 3.68 (d, J=12.0 Hz, 2H), 3.38 (s, 3H), 3.28 (s, 3H), 2.96-2.72 (m, 3H), 2.70-2.59 (m, 2H), 2.58-2.52 (m, 2H), 2.39-2.27 (m, 2H), 2.04-1.92 (m, 1H), 1.85-1.66 (m, 5H), 1.65-1.52 (m, 3H), 1.31-1.23 (m, 6H), 1.11-1.06 (m, 5H), 0.80 (d, J=6.9 Hz, 6H). LCMS (ESI): R_{T}=1.330 min, m/z. found 1082.2 [M-CF₃COOH+H]⁺.

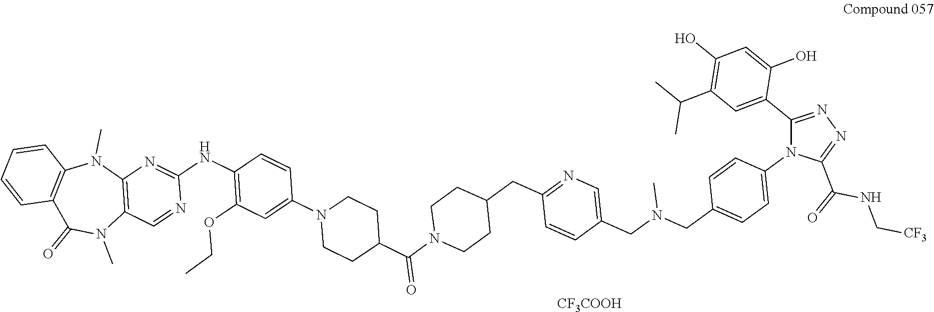 [SMILES: CCOc1cc(N2CCC(C(=O)N3CCC(Cc4ccc(CN(C)Cc5ccc(-n6c(C(=O)NCC(F)(F)F)nnc6-c6cc(C(C)C)c(O)cc6O)cc5)cn4)CC3)CC2)ccc1Nc1ncc2c(n1)N(C)c1ccccc1C(=O)N2C ; "CF3COOH"]

5-(2,4-dihydroxy-5-isopropylphenyl)-4-(4-((((6-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)pyridin-3-yl)methyl)(methyl)amino)methyl)phenyl)-N-(2,2,2-trifluoroethyl)-4H-1,2,4-triazole-3-carboxamide, trifluoroacetic acid. ¹H NMR (400 MHz, DMSO-d₆): δ 10.03 (s, 1H), 9.87-9.64 (m, 2H), 8.64 (s, 1H), 8.36 (s, 1H), 8.12-7.87 (m, 3H), 7.6 (dd, J=7.8, 1.5 Hz, 1H), 7.67-7.40 (m, 7H), 7.27-7.16 (m, 2H), 6.99-6.76 (m, 2H), 6.29 (s, 1H), 4.56-4.36 (m, 6H), 4.12 (dd, J=13.7, 6.0 Hz, 3H), 4.01-3.92 (m, 4H), 3.66 (d, J=11.2 Hz, 2H), 3.39 (s, 3H), 3.31 (s, 3H), 3.09-2.86 (m, 3H), 2.81-2.86 (m, 2H), 2.57-2.51 (m, 3H), 2.11-1.97 (m, 1H), 1.94-1.80 (m, 4H), 1.72-1.55 (m, 2H), 1.34 (t, J=6.4 Hz, 3H), 1.26-1.01 (m, 2H), 0.91 (d, J=6.6 Hz, 6H). LCMS (ESI): R_{T}=1.140 min, m/z. found 1136.4 [M-CF₃COOH+H]⁺.

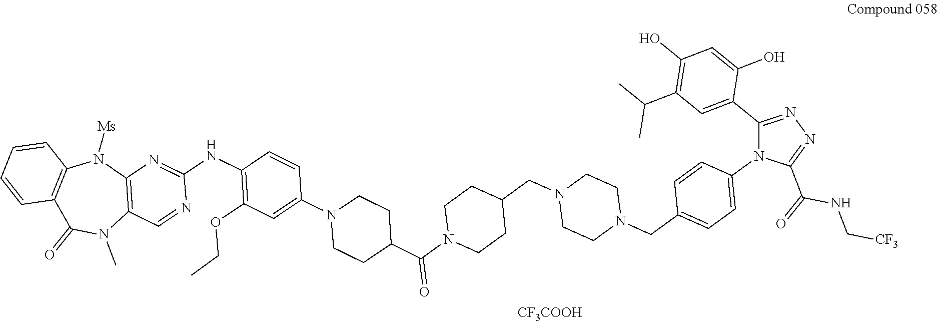 [SMILES: CCOc1cc(N2CCC(C(=O)N3CCC(CN4CCN(Cc5ccc(-n6c(C(=O)NCC(F)(F)F)nnc6-c6cc(C(C)C)c(O)cc6O)cc5)CC4)CC3)CC2)ccc1Nc1ncc2c(n1)N(S(C)(=O)=O)c1ccccc1C(=O)N2C ; "CF3COOH"]

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(2,2,2-trifluoroethyl)-4H-1,2,4-triazole-3-carboxamide, trifluoroacetic acid. ¹H NMR (400 MHz, DMSO-d₆): δ 9.67-9.63 (m, 2H), 8.85 (s, 1H), 8.64 (s, 1H), 7.76-7.74 (d, J=7.7 Hz, 1H), 7.60-7.59 (d, J=6.9 Hz, 2H), 7.48-7.34 (m, 6H), 6.87-6.68 (m, 3H), 6.31 (s, 1H), 4.42-4.27 (m, 3H), 4.14-4.10 (m, 8H), 3.69 (m, 6H), 3.46 (s, 3H), 3.09-2.86 (m, 9H), 2.57-2.47 (m, 2H), 2.30-2.24 (m, 2H), 2.01 (s, 1H), 1.77-1.71 (m, 6H), 1.19 (t, J=6.9 Hz, 3H), 1.18-0.90 (m, 2H), 0.89 (t, J=7.4 Hz, 3H). LCMS (ESI): R_{T}=1.200 min, m/z. found 1149.9 [M-CF₃COOH+H]⁺.

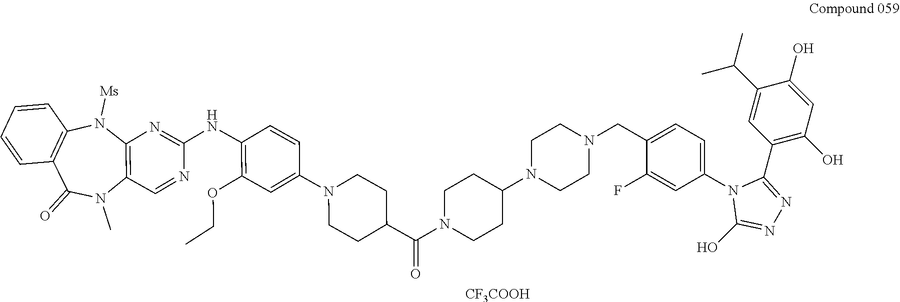 [SMILES: CCOc1cc(N2CCC(C(=O)N3CCC(N4CCN(Cc5ccc(-n6c(O)nnc6-c6cc(C(C)C)c(O)cc6O)cc5F)CC4)CC3)CC2)ccc1Nc1ncc2c(n1)N(S(C)(=O)=O)c1ccccc1C(=O)N2C ; "CF3COOH"]

2-((4-(4-(4-(4-(4-(3-(2,4-dihydroxy-5-isopropylphenyl)-5-hydroxy-4H-1,2,4-triazol-4-yl)-2-fluorobenzyl)piperazin-1-yl)piperidine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 12.00 (s, 1H), 9.70 (s, 1H), 9.14 (s, 1H), 8.84 (s, 1H), 8.63 (s, 1H), 7.76-7.74 (m, 1H), 7.60 (m, 2H), 7.59-7.40 (m, 3H), 7.14-7.11 (m, 1H), 7.02-6.99 (m, 1H), 6.91 (s, 1H), 6.26 (s, 1H), 4.55-4.52 (m, 2H), 4.05-4.02 (m, 3H), 3.69-3.60 (m, 8H), 3.46-3.41 (m, 3H), 3.16-2.84 (m, 9H), 2.06 (m, 2H), 1.74 (s, 3H), 1.28-1.17 (m, 8H), 1.20 (t, J=6.4 Hz, 3H), 1.01 (d, J=6.9 Hz, 6H). LCMS (ESI): R_{T}=1.210 min, m/z. found 1058.9 [M-CF₃COOH+H]⁺.

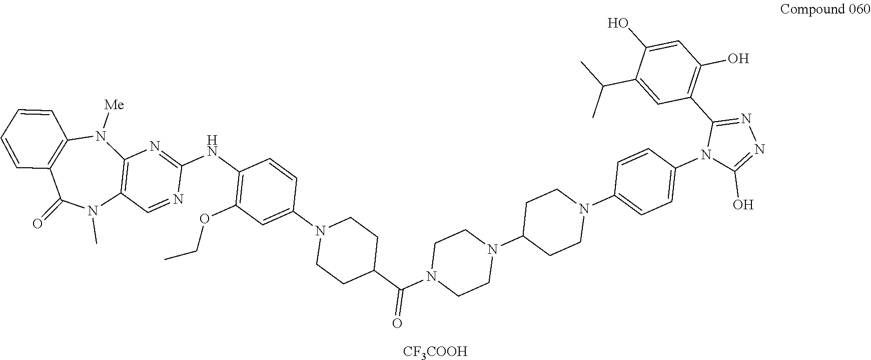 [SMILES: CCOc1cc(N2CCC(C(=O)N3CCN(C4CCN(c5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; "CF3COOH"]

2-((4-(4-(4-(4-(4-(3-(2,4-dihydroxy-5-isopropylphenyl)-5-hydroxy-4H-1,2,4-triazol-4-yl)phenyl)piperazin-1-yl)piperidine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.88 (s, 1H), 9.62 (m, 2H), 9.43 (s, 1H), 8.82 (s, 1H), 8.62 (s, 1H), 7.76-7.74 (m, 1H), 7.59-7.58 (m, 2H), 7.47-7.45 (m, 2H), 7.10-7.00 (m, 4H), 6.78-6.52 (m, 2H), 6.27 (s, 1H), 4.60-4.50 (m, 1H), 4.25-3.54 (m, 15H), 3.46 (s, 3H), 3.21-2.75 (m, 8H), 2.15 (m, 2H), 1.73-1.45 (m, 6H), 1.19 (t, J=6.4 Hz, 3H), 0.94 (d, J=6.9 Hz, 6H). LCMS (ESI): R_{T}=1.150 min, m/z. found 1028.1 [M-CF₃COOH+H]⁺.

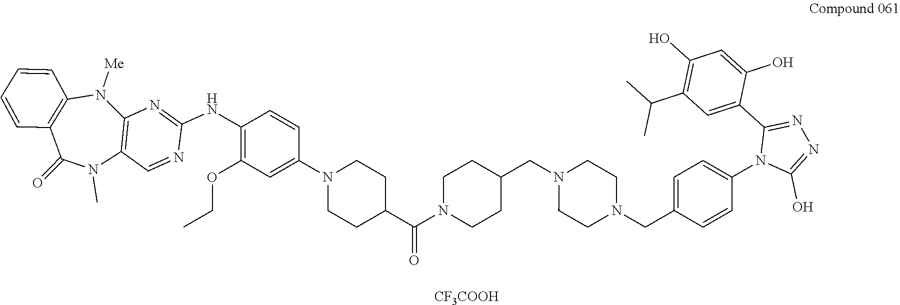 [SMILES: CCOc1cc(N2CCC(C(=O)N3CCC(CN4CCN(Cc5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; "CF3COOH"]

2-((4-(4-(4-((4-(4-(3-(2,4-dihydroxy-5-isopropylphenyl)-5-hydroxy-4H-1,2,4-triazol-4-yl)benzyl)piperazin-1-yl)methyl)piperidine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.97 (s, 1H), 9.62 (s, 1H), 9.43 (s, 1H), 8.88 (s, 1H), 8.64 (s, 1H), 7.76 (d, J=7.8 Hz, 1H), 7.60-7.20 (m, 8H), 6.78-6.68 (m, 3H), 6.26 (s, 1H), 4.43-4.31 (m, 1H), 4.07-3.99 (m, 4H), 3.90-3.80 (s, 3H), 3.70 (s, 8H), 3.46 (s, 4H), 3.06-2.81 (m, 11H), 2.02-1.90 (m, 1H), 1.88-1.72 (m, 6H), 1.23-1.18 (m, 4H), 0.98 (d, J=7.6 Hz, 7H). LCMS (ESI): R_{T}1.043 min, m/z. found 1054.9 [M-CF₃COOH+H]⁺.

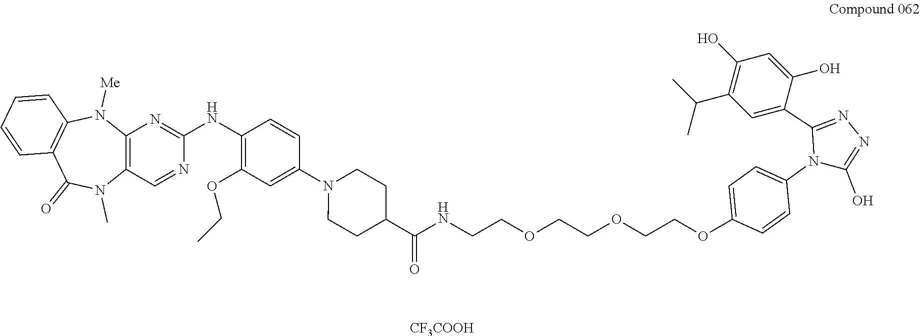 [SMILES: CCOc1cc(N2CCC(C(=O)NCCOCCOCCOc3ccc(-n4c(O)nnc4-c4cc(C(C)C)c(O)cc4O)cc3)CC2)ccc1Nc1ncc2c(n1)N(C)c1ccccc1C(=O)N2C ; "CF3COOH"]

N-(2-(2-(2-(4-(3-(2,4-dihydroxy-5-isopropylphenyl)-5-hydroxy-4H-1,2,4-triazol-4-yl)phenoxy)ethoxy)ethoxy)ethyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 11.88 (s, 1H), 9.66 (s, 1H), 9.43 (s, 1H), 8.88 (s, 1H), 8.65 (s, 1H), 8.01-7.96 (m, 1H), 7.76 (d, J=7.8 Hz, 1H), 7.60 (d, J=3.6 Hz, 2H), 7.48-7.45 (m, 2H), 7.09 (d, J=9.2 Hz, 1H), 6.95-6.72 (m, 5H), 6.26 (s, 1H), 4.08-4.03 (m, 4H), 3.74-3.66 (m, 9H), 3.47-3.40 (m, 10H), 3.23-3.17 (m, 4H), 3.00-2.96 (m, 2H), 2.42-2.33 (m, 1H), 1.82 (s, 4H), 1.23-1.18 (m, 4H), 0.98 (d, J=6.8 Hz, 7H). LCMS (ESI): R_{T}=1.253 min, m/z. found 1006.9 [M-CF₃COOH+H]⁺.

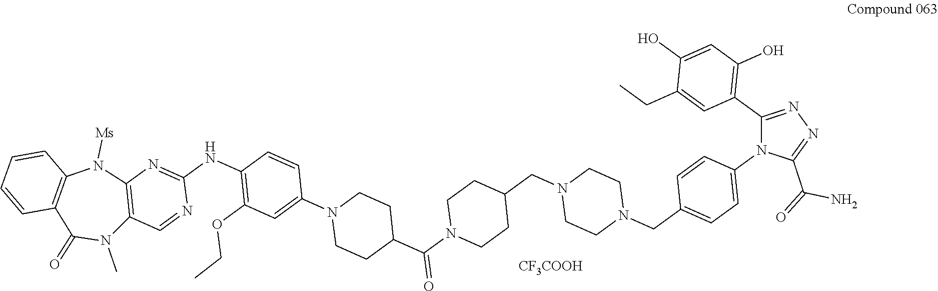

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-4H-1,2,4-triazole-3-carboxamide, trifluoroacetic acid. ¹H NMR (400 MHz, DMSO-d₆): δ 10.39 (s, 1H), 9.71 (s, 1H), 8.76 (s, 1H), 8.60 (s, 1H), 8.28 (s, 1H), 7.74 (d, J=7.2 Hz, 2H), 7.59-7.57 (m, 2H), 7.47-7.43 (m, 1H), 7.35-7.25 (m, 5H), 6.59-6.47 (m, 3H), 6.32 (s, 1H), 4.41-4.37 (m, 1H), 4.03-3.96 (m, 3H), 3.72-3.63 (m, 5H), 3.49-3.45 (m, 6H), 3.03-2.94 (m, 1H), 2.77-2.70 (m, 4H), 2.39-2.27 (m, 7H), 2.24-2.08 (m, 5H), 1.78-1.67 (m, 7H), 1.17-1.13 (m, 3H), 1.03-0.95 (m, 1H), 0.83 (t, J=7.2 Hz, 4H). LCMS (ESI): R_{T}1.118 min, m/z. found 1069.2 [M-CF₃COOH+H]⁺.

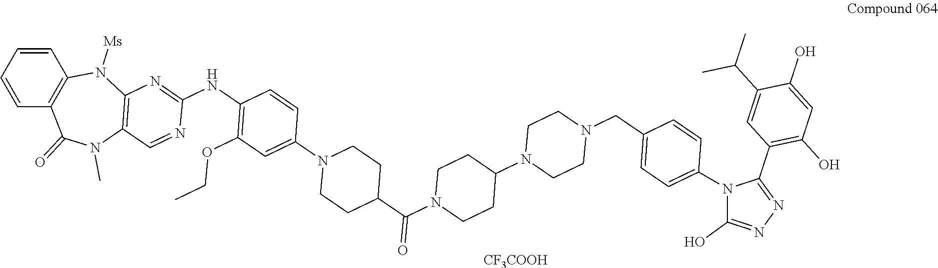 [SMILES: CCOc1cc(N2CCC(C(=O)N3CCC(N4CCN(Cc5ccc(-n6c(O)nnc6-c6cc(C(C)C)c(O)cc6O)cc5)CC4)CC3)CC2)ccc1Nc1ncc2c(n1)N(S(C)(=O)=O)c1ccccc1C(=O)N2C ; "CF3COOH"]

2-((4-(4-(4-(4-(4-(3-(2,4-dihydroxy-5-isopropylphenyl)-5-hydroxy-4H-1,2,4-triazol-4-yl)benzyl)piperazin-1-yl)piperidine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.93 (s, 1H), 9.60 (s, 1H), 9.41 (s, 1H), 8.76 (s, 1H), 8.60 (s, 1H), 7.74 (d, J=7.2 Hz, 1H), 7.64-7.55 (m, 2H), 7.47-7.43 (m, 1H), 7.29-7.24 (m, 3H), 7.12 (d, J=8.4 Hz, 2H), 6.77 (s, 1H), 6.59-6.47 (m, 2H), 6.26 (s, 1H), 4.39 (d, J=12.8 Hz, 1H), 4.06-3.97 (m, 3H), 3.70 (d, J=11.6 Hz, 5H), 3.44 (d, J=12.4 Hz, 6H), 3.34-3.30 (m, 4H), 3.05-2.93 (m, 2H), 2.82-2.66 (m, 4H), 2.46-2.23 (m, 4H), 1.86-1.58 (m, 6H), 1.38-1.13 (m, 5H), 0.94 (d, J=6.8 Hz, 6H). LCMS (ESI): R_{T}=1.150 min, m/z. found 1042.2 [M-CF₃COOH+H]⁺.

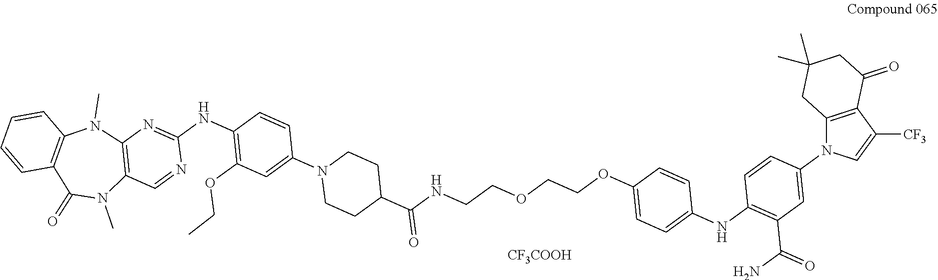 [SMILES: CCOc1cc(N2CCC(C(=O)NCCOCCOc3ccc(Nc4ccc(-n5cc(C(F)(F)F)c6c5CC(C)(C)CC6=O)cc4C(N)=O)cc3)CC2)ccc1Nc1ncc2c(n1)N(C)c1ccccc1C(=O)N2C ; "CF3COOH"]

N-(2-(2-(4-((2-carbamoyl-4-(6,6-dimethyl-4-oxo-3-(trifluoromethyl)-4,5,6,7-tetrahydro-1H-indazol-1-yl)phenyl)amino)phenoxy)ethoxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 10.12 (s, 1H), 8.37 (s, 1H), 8.20-8.05 (m, 3H), 7.90 (d, J=8.4 Hz, 1H), 7.69 (dd, J=7.6, 1.6 Hz, 1H), 7.64-7.47 (m, 2H), 7.26-7.16 (m, 4H), 7.03-6.93 (m, 5H), 4.55-4.28 m, 4H), 4.16-4.08 (m, 5H), 3.74 (t, J=4.4 Hz, 2H), 3.64 (d, J=12.2 Hz, 2H), 3.51 (t, J=5.6 Hz, 2H), 3.39 (s, 3H), 3.32 (s, 3H), 3.26 (dd, J=11.4, 5.8 Hz, 2H), 2.91 (s, 2H), 2.42 (s, 3H), 1.88 (s, 3H), 1.35 (t, J=6.8 Hz, 3H), 1.03 (s, 6H). LCMS (ESI): R_{T}=1.500 min, m/z. found 1030.4 [M-CF₃COOH+H]⁺.

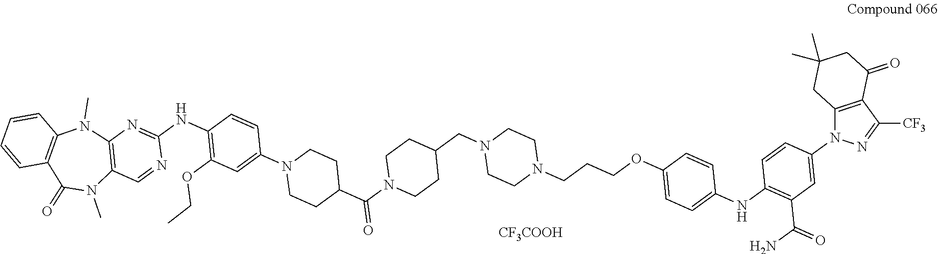 [SMILES: CCOc1cc(N2CCC(C(=O)N3CCC(CN4CCN(CCCOc5ccc(Nc6ccc(-n7nc(C(F)(F)F)c8c7CC(C)(C)CC8=O)cc6C(N)=O)cc5)CC4)CC3)CC2)ccc1Nc1ncc2c(n1)N(C)c1ccccc1C(=O)N2C ; "CF3COOH"]

(4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperazin-1-yl)propoxy)phenyl)amino)benzamide, trifluoroacetic acid. ¹H NMR (400 MHz, DMSO-d₆): δ 10.13 (s, 1H), 8.36 (s, 1H), 8.21 (s, 1H), 8.16-8.07 (m, 1H), 7.91 (d, J=8.4 Hz, 1H), 7.70-7.49 (m, 3H), 7.26-7.16 (m, 4H), 7.04-6.94 (m, 5H), 4.41 (d, J=16.4 Hz, 2H), 4.22-3.83 (m, 18H), 3.67 (d, J=10.4 Hz, 3H), 3.39 (s, 3H), 3.32 (s, 3H), 2.92 (s, 3H), 2.43 (s, 3H), 2.15-2.04 (m, 2H), 1.97-1.67 (m, 8H), 1.34 (t, J=6.9 Hz, 3H), 1.13-0.93 (m, 9H). LCMS (ESI): R_{T}=1.180 min, m/z. found 1166.5 [M-CF₃COOH+H]⁺.

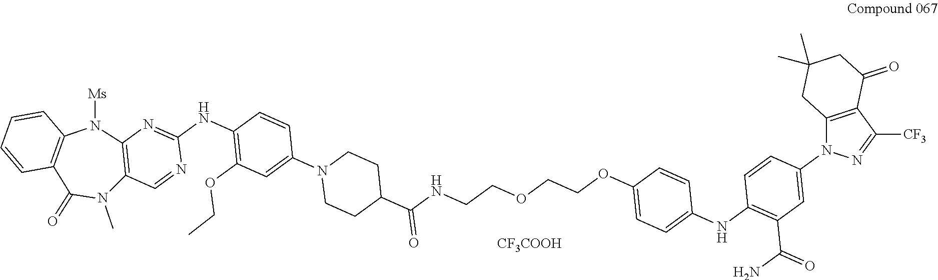 [SMILES: CCOc1cc(N2CCC(C(=O)NCCOCCOc3ccc(Nc4ccc(-n5nc(C(F)(F)F)c6c5CC(C)(C)CC6=O)cc4C(N)=O)cc3)CC2)ccc1Nc1ncc2c(n1)N(S(C)(=O)=O)c1ccccc1C(=O)N2C ; "CF3COOH"]

N-(2-(2-(4-((2-carbamoyl-4-(6,6-dimethyl-4-oxo-3-(trifluoromethyl)-4,5,6,7-tetrahydro-1H-indazol-1-yl)phenyl)amino)phenoxy)ethoxy)ethyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 11.12 (s, 1H), 8.85 (s, 1H), 8.64 (s, 1H), 8.19 (s, 1H), 8.01 (s, 1H), 7.91-7.89 (m, 1H), 7.76-7.74 (m, 1H), 7.59-7.58 (m, 3H), 7.49-7.44 (m, 2H), 7.25-7.22 (m, 2H), 7.03-6.93 (m, 5H), 4.10-3.96 (m, 5H), 3.75-3.65 (m, 8H), 3.51-3.46 (m, 6H), 3.28-3.23 (m, 2H), 2.91 (s, 3H), 2.43 (s, 3H), 1.85-1.72 (m, 4H), 1.21-1.18 (m, 3H), 1.03 (s, 6H). LCMS (ESI): R_{T}=1.500 min, m/z. found 1094.3 [M-CF₃COOH+H]⁺.

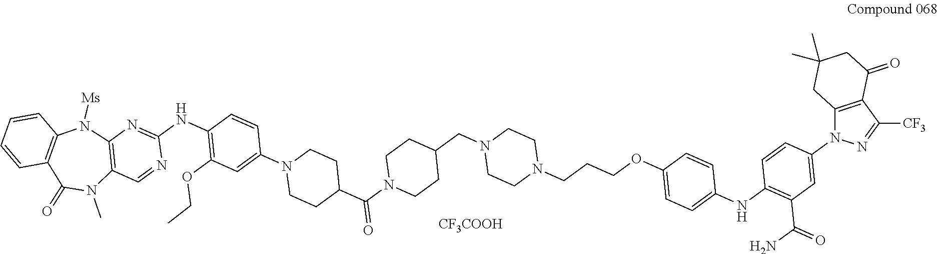

5-(6,6-dimethyl-4-oxo-3-(trifluoromethyl)-4,5,6,7-tetrahydro-1H-indazol-1-yl)-2-((4-(3-(4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)propoxy)phenyl)amino)benzamide, trifluoroacetic acid. ¹H NMR (400 MHz, DMSO-d₆): δ 10.13 (s, 1H), 8.85 (s, 1H), 8.63 (s, 1H), 8.21 (s, 1H), 7.91 (d, J=8.5 Hz, 1H), 7.75 (d, J=7.8 Hz, 1H), 7.59 (d, J=3.8 Hz, 3H), 7.50-7.43 (m, 1H), 7.25 (d, J=8.9 Hz, 2H), 7.04 (d, J=2.0 Hz, 1H), 7.00-6.91 (m, 3H), 4.41 (m, 2H), 4.11-3.95 (m, 13H), 3.70 (s, 6H), 3.46 (s, 3H), 3.11 (m, 8H), 2.92 (s, 3H), 2.89-2.79 (m, 2H), 2.43 (s, 2H), 2.00 (m, 4H), 1.76 (s, 6H), 1.19 (t, J=6.7 Hz, 3H), 1.04 (s, 6H). LCMS (ESI): R_{T}=1.160 min, m/z. found 616.1 [½M-CF₃COOH+H]⁺.

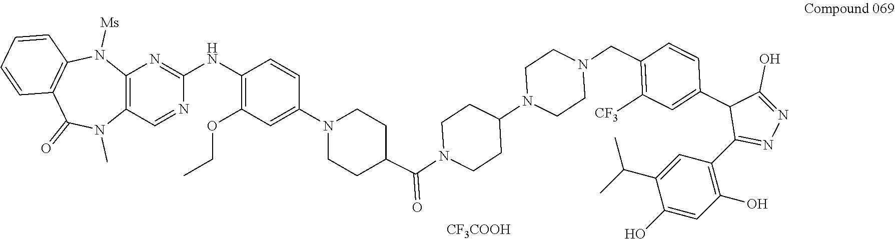 [SMILES: CCOc1cc(N2CCC(C(=O)N3CCC(N4CCN(Cc5ccc(C6C(O)=NN=C6c6cc(C(C)C)c(O)cc6O)cc5C(F)(F)F)CC4)CC3)CC2)ccc1Nc1ncc2c(n1)N(S(C)(=O)=O)c1ccccc1C(=O)N2C ; "CF3COOH"]

2-((4-(4-(4-(4-(4-(3-(2,4-dihydroxy-5-isopropylphenyl)-5-hydroxy-4H-1,2,4-triazol-4-yl)-2-(trifluoromethyl)benzyl)piperazin-1-yl)piperidine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 12.04 (s, 1H), 9.69-9.35 (m, 3H), 8.84 (s, 1H), 8.63 (s, 1H), 7.75 (d, J=7.5 Hz, 2H), 7.62-7.43 (m, 5H), 6.93 (s, 1H), 6.72 (m, 2H), 6.25 (s, 1H), 4.55 (m, 1H), 4.14-4.02 (m, 8H), 3.70 (s, 6H), 3.46 (s, 6H), 3.15-2.83 (m, 9H), 2.10 (s, 2H), 1.76 (s, 4H), 1.19 (t, J=6.7 Hz, 3H), 1.02 (d, J=6.9 Hz, 6H). LCMS (ESI): R_{T}=1.100 min, m/z. found 1109.3 [M-CF₃COOH+H]⁺.

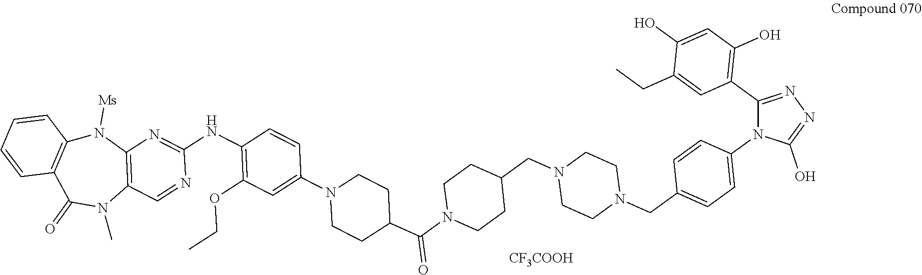

2-((2-ethoxy-4-(4-(4-((4-(4-(3-(5-ethyl-2,4-dihydroxyphenyl)-5-hydroxy-4H-1,2,4-triazol-4-yl)benzyl)piperazin-1-yl)methyl)piperidine-1-carbonyl)piperidin-1-yl)phenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.94 (s, 1H), 9.62-9.33 (m, 2H), 8.85 (s, 1H), 8.64 (s, 1H), 7.75 (d, J=7.6 Hz, 1H), 7.60-7.20 (m, 8H), 6.87 (d, J=4.4 Hz, 1H), 6.86-6.64 (m, 1H), 6.24 (s, 1H), 4.44-4.34 (m, 4H), 4.13-3.98 (m, 5H), 3.72-3.65 (m, 6H), 3.46-3.42 (m, 5H), 3.16-2.81 (m, 8H), 2.43-2.31 (m, 4H), 1.97-1.93 (m, 1H), 1.78-1.68 (m, 6H), 1.19 (d, J=6.8 Hz, 4H), 0.98 (d, J=7.6 Hz, 5H). LCMS (ESI): R_{T}=1.004 min, m/z. found 1041.4 [M-CF₃COOH+H]⁺.

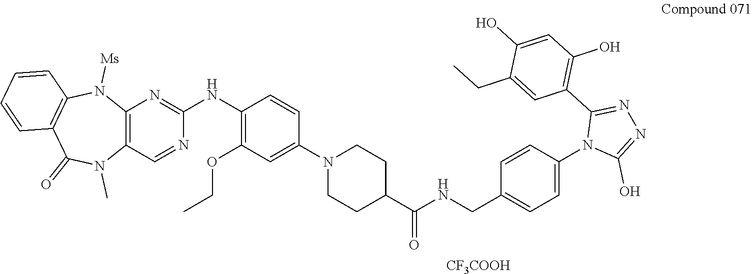 [SMILES: CCOc1cc(N2CCC(C(=O)NCc3ccc(-n4c(O)nnc4-c4cc(CC)c(O)cc4O)cc3)CC2)ccc1Nc1ncc2c(n1)N(S(C)(=O)=O)c1ccccc1C(=O)N2C ; "CF3COOH"]

1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)-N-(4-(3-(5-ethyl-2,4-dihydroxyphenyl)-5-hydroxy-4H-1,2,4-triazol-4-yl)benzyl)piperidine-4-carboxamide, trifluoroacetic acid. ¹H NMR (400 MHz, DMSO-d₆): δ 11.89 (s, 1H), 9.55 (s, 1H), 9.35 (d, J=2.4 Hz, 1H), 8.85 (s, 1H), 8.64 (s, 1H), 8.45 (s, 1H), 7.75 (d, J=7.6 Hz, 1H), 7.59 (d, J=3.6 Hz, 2H), 7.48-7.43 (m, 2H), 7.22-7.10 (m, 4H), 6.90 (s, 1H), 7.84-6.70 (m, 1H), 6.23 (s, 1H), 4.27 (d, J=5.6 Hz, 2H), 4.07-4.00 (m, 2H), 3.84-5.54 (m, 7H), 3.46 (s, 3H), 3.05-3.78 (m, 2H), 2.45-2.34 (m, 3H), 1.87-1.79 (m, 4H), 1.19 (d, J=6.4 Hz, 3H), 1.01 (d, J=7.2 Hz, 3H). LCMS (ESI): R_{T}=1.205 min, m/z. found 875.2 [M-CF₃COOH+H]⁺.

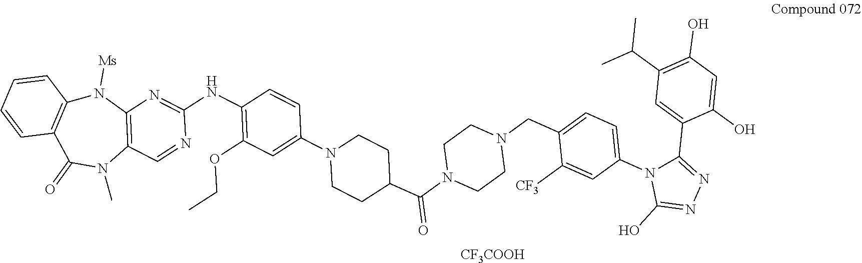 [SMILES: CCOc1cc(N2CCC(C(=O)N3CCN(Cc4ccc(-n5c(O)nnc5-c5cc(C(C)C)c(O)cc5O)cc4C(F)(F)F)CC3)CC2)ccc1Nc1ncc2c(n1)N(S(C)(=O)=O)c1ccccc1C(=O)N2C ; "CF3COOH"]

2-((4-(4-(4-(4-(3-(2,4-dihydroxy-5-isopropylphenyl)-5-hydroxy-4H-1,2,4-triazol-4-yl)-2-(trifluoromethyl)benzyl)piperazine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 12.08 (s, 1H), 9.66 (s, 1H), 9.39 (s, 1H), 8.85 (s, 1H), 8.63 (s, 1H), 7.86-7.74 (m, 2H), 7.60-7.44 (s, 6H), 6.97 (s, 1H), 6.78-6.63 (m, 2H), 6.25 (s, 1H), 4.08-4.00 (m, 2H), 3.73-3.65 (m, 7H), 3.46-3.36 (m, 10H), 3.07-2.73 (m, 5H), 1.76 (s, 4H), 1.23-1.16 (m, 4H), 1.04 (d, J=7.2 Hz, 6H). LCMS (ESI): R_{T}=1.314 min, m/z. found 1025.9 [M-CF₃COOH+H]⁺.

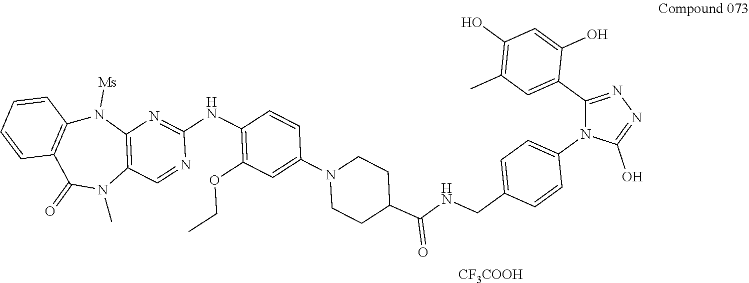 [SMILES: CCOc1cc(N2CCC(C(=O)NCc3ccc(-n4c(O)nnc4-c4cc(C)c(O)cc4O)cc3)CC2)ccc1Nc1ncc2c(n1)N(S(C)(=O)=O)c1ccccc1C(=O)N2C ; "CF3COOH"]

N-(4-(3-(2,4-dihydroxy-5-methylphenyl)-5-hydroxy-4H-1,2,4-triazol-4-yl)benzyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 11.87 (s, 1H), 9.57 (s, 1H), 9.33 (s, 1H), 8.84 (s, 1H), 8.63 (s, 1H), 8.48-8.40 (m, 1H), 7.75 (d, J=7.6 Hz, 1H), 7.59 (d, J=4.0 Hz, 2H), 7.52-7.39 (m, 2H), 7.23-7.09 (m, 4H), 6.94 (s, 1H), 6.85-6.60 m, 2H), 6.23 (s, 1H), 4.30-4.24 (m, 2H), 4.10-3.99 (m, 2H), 3.75-3.64 (m, 6H), 3.46 (s, 3H), 3.02-2.82 (m, 2H), 2.44-2.38 (m, 1H), 1.97 (s, 3H), 1.92-1.74 (m, 4H), 1.22-1.15 (m, 3H). LCMS (ESI): RT=1.063 min, m/z. found 861.7 [M-CF₃COOH+H]⁺.

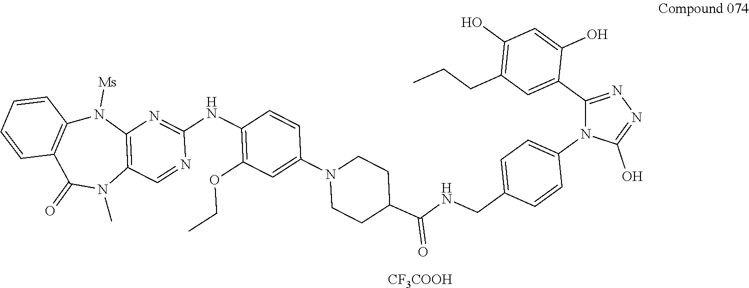 [SMILES: CCCc1cc(-c2nnc(O)n2-c2ccc(CNC(=O)C3CCN(c4ccc(Nc5ncc6c(n5)N(S(C)(=O)=O)c5ccccc5C(=O)N6C)c(OCC)c4)CC3)cc2)c(O)cc1O ; "CF3COOH"]

N-(4-(3-(2,4-dihydroxy-5-propylphenyl)-5-hydroxy-4H-1,2,4-triazol-4-yl)benzyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 11.88 (s, 1H), 9.60-9.30 (m, 2H), 8.84 (s, 1H), 8.64 (s, 1H), 8.48-8.39 (m, 1H), 7.75 (d, J=7.6 Hz, 1H), 7.59 (d, J=3.6 Hz, 2H), 7.50-7.39 (m, 2H), 7.25-7.07 (m, 5H), 6.89-6.61 (m, 3H), 6.23 (s, 1H), 4.27 (d, J=5.7 Hz, 2H), 4.10-4.00 (m, 2H), 3.70 (s, 5H), 3.46 (s, 4H), 2.36-2.29 (m, 2H), 2.04-1.76 (m, 5H), 1.47-1.38 (m, 2H), 1.24-1.16 (m, 6H), 0.82-0.77 (m, 3H). LCMS (ESI): R_{T}=1.157 min, m/z. found 889.6 [M-CF₃COOH+H]⁺.

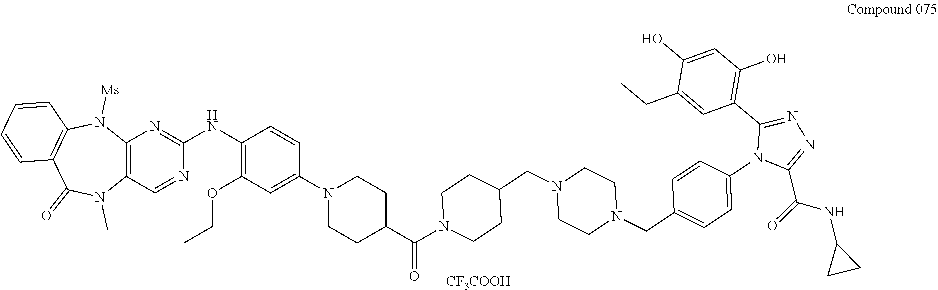 [SMILES: CCOc1cc(N2CCC(C(=O)N3CCC(CN4CCN(Cc5ccc(-n6c(C(=O)NC7CC7)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

N-cyclopropyl-4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-4H-1,2,4-triazole-3-carboxamide, trifluoroacetic acid. ¹H NMR (400 MHz, DMSO-d₆): δ 9.72 (s, 1H), 9.07 (d, J=4.8 Hz, 1H), 8.86 (s, 1H), 8.64 (s, 1H), 7.75 (d, J=7.6 Hz, 1H), 7.62-7.56 (m, 3H), 7.50-7.30 (m, 6H), 6.92-6.60 (m, 3H), 6.30 (s, 1H), 4.44-4.36 (m, 3H), 4.09-3.97 (m, 7H), 3.73-3.64 (m, 7H), 3.46 (s, 5H), 3.04-2.88 (m, 6H), 2.77-2.65 (m, 3H), 2.61-2.54 (m, 2H), 2.30-2.23 (m, 2H), 2.06-1.94 (m, 1H), 1.84-1.69 (m, 7H), 1.26-1.16 (m, 4H), 1.14-0.96 (m, 2H), 0.91-0.85 (m, 3H), 0.67-0.55 (m, 4H). LCMS (ESI): R_{T}=1.032 min, m/z. found 1108.3 [M-CF₃COOH+H]⁺.

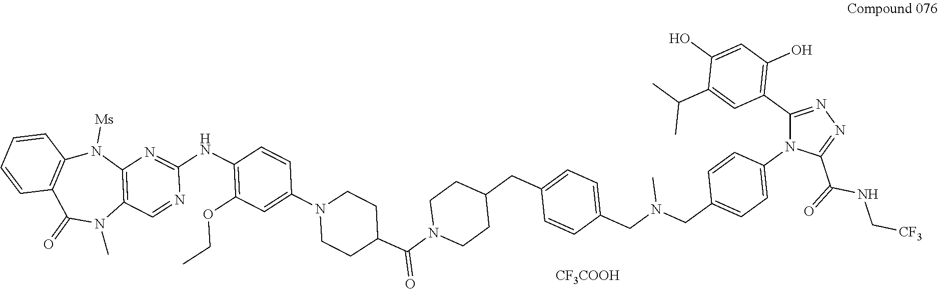 [SMILES: CCOc1cc(N2CCC(C(=O)N3CCC(Cc4ccc(CN(C)Cc5ccc(-n6c(C(=O)NCC(F)(F)F)nnc6-c6cc(C(C)C)c(O)cc6O)cc5)cc4)CC3)CC2)ccc1Nc1ncc2c(n1)N(S(C)(=O)=O)c1ccccc1C(=O)N2C ; "CF3COOH"]

5-(2,4-dihydroxy-5-isopropylphenyl)-4-(4-(((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)benzyl)(methyl)amino)methyl)phenyl)-N-(2,2,2-trifluoroethyl)-4H-1,2,4-triazole-3-carboxamide, trifluoroacetic acid. ¹H NMR (400 MHz, DMSO-d₆): δ 10.03-9.65 (m, 4H), 8.83 (s, 1H), 8.63 (s, 1H), 7.75 (d, J=7.2 Hz, 1H), 7.64-7.26 (m, 13H), 6.84-6.54 (m, 3H), 6.30 (s, 1H), 4.53-4.44 (m, 1H), 4.38 (d, J=14.1 Hz, 2H), 4.27-4.16 (m, 3H), 4.00-3.94 (m, 6H), 3.69 (s, 6H), 3.46 (s, 3H), 3.03-2.83 (m, 4H), 2.59-2.55 (m, 2H), 1.83-1.64 (m, 6H), 1.23-1.16 (m, 5H), 1.05-0.97 (m, 1H), 0.91 (d, J=6.4 Hz, 6H). LCMS (ESI): R_{T}=1.200 min, m/z. found 600.5 [M/2-CF₃COOH+H]⁺.

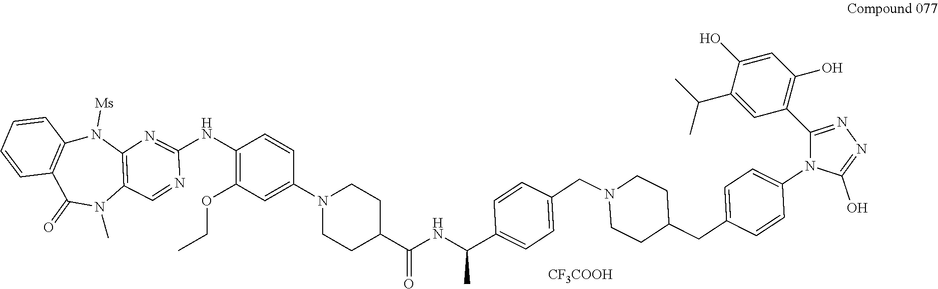

(R)—N-(1-(4-((4-(4-(3-(2,4-dihydroxy-5-isopropylphenyl)-5-hydroxy-4H-1,2,4-triazol-4-yl)benzyl)piperidin-1-yl)methyl)phenyl)ethyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 11.92 (s, 1H), 9.61 (s, 1H), 9.47-9.30 (m, 2H), 8.81 (s, 1H), 8.62 (s, 1H), 8.37 (d, J=7.6 Hz, 1H), 7.75 (d, J=7.6 Hz, 1H), 7.59 (d, J=3.6 Hz, 2H), 7.48-7.37 (m, 7H), 7.24-7.09 (m, 4H), 6.84-6.51 (m, 3H), 6.26 (d, J=3.2 Hz, 1H), 4.98-4.91 (m, 1H), 4.22 (d, J=2.9 Hz, 2H), 4.05-4.01 (m, 2H), 3.71-3.67 (m, 6H), 3.46 (s, 3H), 3.33 (d, J=13.3 Hz, 1H), 3.18-3.07 (m, 1H), 3.00-2.79 (m, 4H), 1.81-1.67 (m, 8H), 1.42-1.34 (m, 5H), 1.17 (t, J=6.8 Hz, 3H), 0.94 (d, J=6.8 Hz, 6H). LCMS (ESI): R_{T}=1.140 min, m/z. found 1089.8 [M-CF₃COOH+H]⁺.

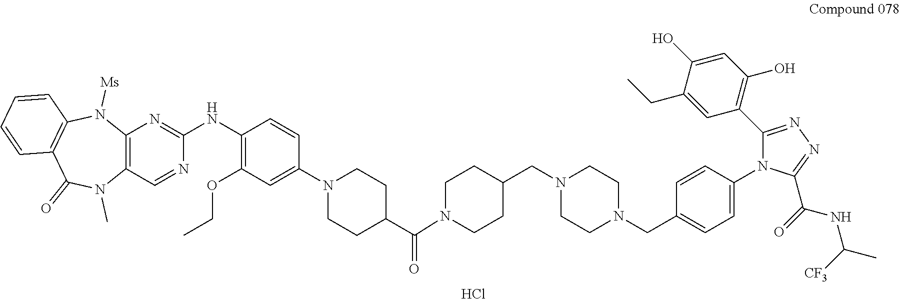 [SMILES: CCOc1cc(N2CCC(C(=O)N3CCC(CN4CCN(Cc5ccc(-n6c(C(=O)NC(C)C(F)(F)F)nnc6-c6cc(CC)c(O)cc6O)cc5)CC4)CC3)CC2)ccc1Nc1ncc2c(n1)N(S(C)(=O)=O)c1ccccc1C(=O)N2C ; Cl]

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, hydrochloride. ¹H NMR (400 MHz, DMSO-d₆): δ 9.73-9.61 (m, 2H), 8.85 (s, 1H), 8.64 (s, 1H), 7.75 (d, J=7.6 Hz, 1H), 7.59 (d, J=3.6 Hz, 2H), 7.49-7.32 (m, 6H), 6.84-6.63 (m, 2H), 6.30 (s, 1H), 4.68-4.60 (m, 1H), 4.44-4.36 (m, 2H), 4.33-4.14 (m, 6H), 4.08-0.399 (m, 4H), 3.69 (s, 5H), 3.46 (s, 3H), 3.20-2.77 (m, 10H), 2.61-2.56 (m, 1H), 2.28 (q, J=7.6 Hz, 2H), 2.08-1.95 (m, 1H), 1.85-1.67 (m, 6H), 1.34 (d, J=7.0 Hz, 3H), 1.19 (t, J=6.8 Hz, 3H), 1.14-0.98 (m, 2H), 0.90 (t, J=7.5 Hz, 3H). LCMS (ESI): R_{T}=1.100 min, m/z. found 1164.3 [M−HCl+H]⁺.

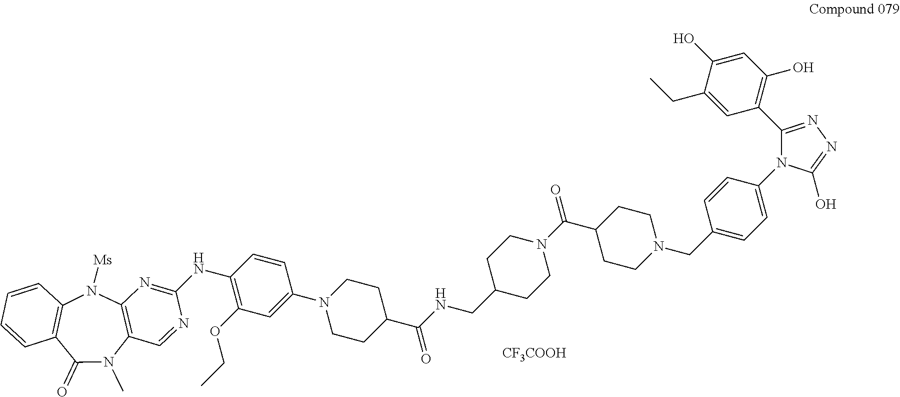 [SMILES: CCOc1cc(N2CCC(C(=O)NCC3CCN(C(=O)C4CCN(Cc5ccc(-n6c(O)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)-N-((1-(1-(4-(3-(5-ethyl-2,4-dihydroxyphenyl)-5-hydroxy-4H-1,2,4-triazol-4-yl)benzyl)piperidine-4-carbonyl)piperidin-4-yl)methyl)piperidine-4-carboxamide, trifluoroacetic acid. ¹H NMR (400 MHz, DMSO-d₆): δ 11.97 (s, 1H), 9.60 (s, 1H), 9.46 (s, 1H), 9.32 (s, 1H), 8.80 (s, 1H), 8.62 (s, 1H), 7.89 (s, 1H), 7.75 (d, J=8.0 Hz, 1H), 7.60-7.44 (m, 4H), 7.38-7.16 (m, 3H), 6.91 (s, 1H), 6.67 (s, 2H), 6.23 (s, 1H), 4.30 (m, 3H), 4.07-4.00 (m, 2H), 3.70 (m, 7H), 3.46 (s, 3H), 3.28 (m, 4H), 2.93 (s, 7H), 2.37 (m, 7.3 Hz, 3H), 1.87-1.56 (m, 10H), 1.29-1.08 (m, 5H), 1.01 (t, J=7.5 Hz, 3H). LCMS (ESI): R_{T}=1.000 min, m/z. found 1083.3 [M-CF₃COOH+H]⁺.

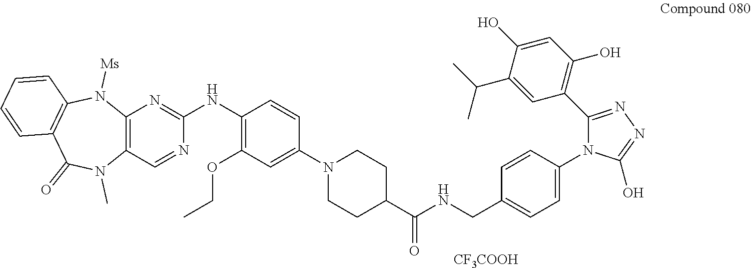 [SMILES: CCOc1cc(N2CCC(C(=O)NCc3ccc(-n4c(O)nnc4-c4cc(C(C)C)c(O)cc4O)cc3)CC2)ccc1Nc1ncc2c(n1)N(S(C)(=O)=O)c1ccccc1C(=O)N2C ; "CF3COOH"]

N-(4-(3-(2,4-dihydroxy-5-isopropylphenyl)-5-hydroxy-4H-1,2,4-triazol-4-yl)benzyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 11.90 (s, 1H), 9.58-9.36 (m, 2H), 8.86 (s, 1H), 8.64 (s, 1H), 8.46 (s, 1H), 7.75 (d, J=7.6 Hz, 1H), 7.59 (d, J=3.6 Hz, 2H), 7.48-7.44 (m, 2H), 7.24-7.11 (m, 4H), 6.88-6.69 (m, 3H), 6.24 (s, 1H), 4.28 (d, J=5.6 Hz, 2H), 4.23-3.97 (m, 5H), 3.70 (s, 4H), 3.46 (s, 3H), 3.03-2.96 (m, 2H), 1.88 (s, 4H), 1.20 (t, J=6.8 Hz, 3H), 1.01 (d, J=6.8 Hz, 7H). LCMS (ESI): R_{T}=1.225 min, m/z. found 889.2 [M-CF₃COOH+H]⁺.

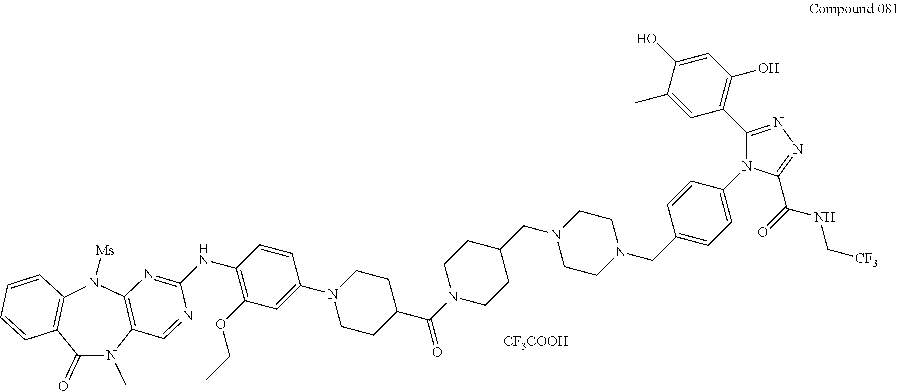

5-(2,4-dihydroxy-5-methylphenyl)-4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-N-(2,2,2-trifluoroethyl)-4H-1,2,4-triazole-3-carboxamide, trifluoroacetic acid. ¹H NMR (400 MHz, DMSO-d₆): δ 9.93-9.50 (m, 3H), 8.86 (s, 1H), 8.64 (s, 1H), 7.76 (d, J=7.6 Hz, 1H), 7.59 (d, J=3.6 Hz, 2H), 7.52-7.32 (m, 6H), 6.97-6.59 (m, 3H), 6.29 (s, 1H), 4.49-4.36 (m, 6H), 4.05-3.93 (m, 6H), 3.70 (s, 5H), 3.46 (s, 3H), 3.14-2.83 (m, 8H), 2.06-1.96 (m, 1H), 1.87 (s, 3H), 1.84-1.71 (m, 6H), 1.25-1.10 (m, 6H), 1.05-0.95 (m, 1H), 0.85-0.71 (m, 1H). LCMS (ESI): R_{T}=1.060 min, m/z. found 1136.3 [M-CF₃COOH+H]⁺.

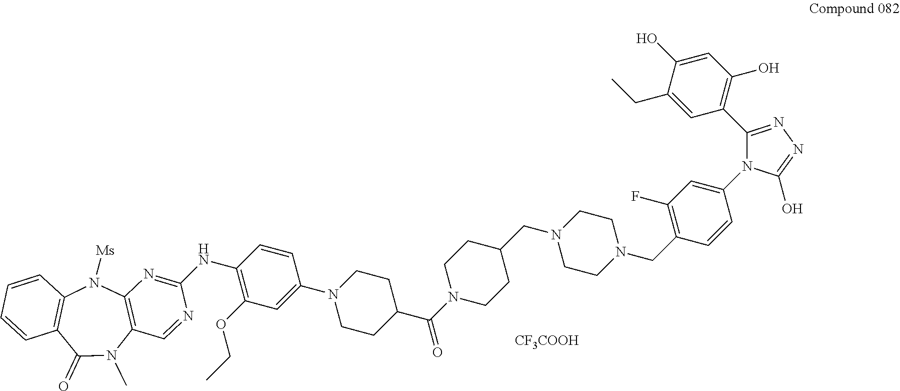

2-((2-ethoxy-4-(4-(4-((4-(4-(3-(5-ethyl-2,4-dihydroxyphenyl)-5-hydroxy-4H-1,2,4-triazol-4-yl)-2-fluorobenzyl)piperazin-1-yl)methyl)piperidine-1-carbonyl)piperidin-1-yl)phenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.96 (s, 1H), 9.59 (s, 1H), 9.37 (s, 1H), 8.74 (s, 1H), 8.60 (s, 1H), 7.74 (d, J=7.2 Hz, 1H), 7.58 (d, J=3.2 Hz, 2H), 7.47-7.25 (s, 3H), 7.05-6.89 (m, 3H), 6.59-6.47 (m, 2H), 6.25 (s, 1H), 4.38-4.29 (m, 1H), 4.03-3.96 (m, 3H), 3.68-3.65 (m, 5H), 3.47-3.45 (m, 6H), 3.08-2.96 (m, 1H), 2.81-2.66 (m, 3H), 2.65-2.55 (m, 3H), 2.40-2.32 (m, 8H), 2.10 (d, J=6.8 Hz, 2H), 1.75-1.66 (m, 7H), 1.15 (t, J=7.2 Hz, 3H), 1.01 (t, J=7.6 Hz, 4H), 0.96-0.85 (m, 1H). LCMS (ESI): R_{T}=1.000 min, m/z. found 1059.3 [M-CF₃COOH+H]⁺.

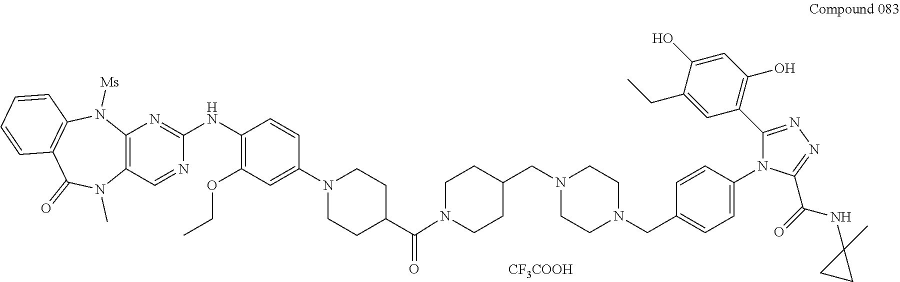

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(1-methylcyclopropyl)-4H-1,2,4-triazole-3-carboxamide, trifluoroacetic acid. ¹H NMR (400 MHz, DMSO-d₆): δ 9.72 (s, 1H), 9.14 (s, 1H), 8.83 (s, 1H), 8.63 (s, 1H), 7.75 (d, J=8.0 Hz, 2H), 7.62-7.30 (m, 10H), 6.81-6.62 (m, 3H), 6.30 (s, 1H), 4.47-4.45 (m, 2H), 4.13-4.01 (m, 5H), 3.69 (s, 7H), 3.50-3.42 (m, 7H), 3.14-3.06 (m, 3H), 2.96-2.80 (m, 2H), 2.29-2.23 (s, 2H), 2.11-1.98 (m, 1H), 1.85-1.70 (m, 8H), 1.25-1.23 (m, 3H), 1.19-1.13 (m, 3H), 1.03-0.98 (m, 1H), 0.87 (t, J=7.2 Hz, 4H), 0.67 (s, 2H), 0.53 (s, 2H). LCMS (ESI): R_{T}=1.058 min, m/z. found 1122.3 [M-CF₃COOH+H]⁺.

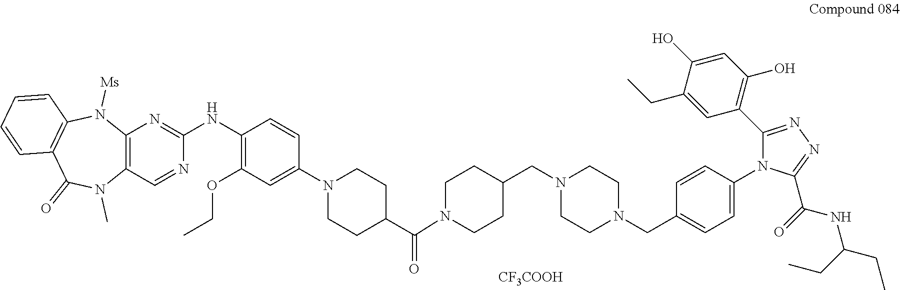 [SMILES: CCOc1cc(N2CCC(C(=O)N3CCC(CN4CCN(Cc5ccc(-n6c(C(=O)NC(CC)CC)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(pentan-3-yl)-4H-1,2,4-triazole-3-carboxamide, trifluoroacetic acid. ¹H NMR (400 MHz, DMSO-d₆): δ 9.72 (s, 1H), 8.85 (s, 1H), 8.66-8.63 (m, 2H), 7.75 (d, J=8.0 Hz, 1H), 7.59 (d, J=3.6 Hz, 2H), 7.48-7.31 (m, 6H), 6.81-6.55 (m, 3H), 6.31 (s, 1H), 4.42-4.40 (m, 2H), 4.33-4.25 (m, 4H), 4.17-4.00 (m, 6H), 3.69 (s, 5H), 3.57-3.55 (m, 1H), 3.46 (s, 5H), 3.06-2.87 (m, 9H), 2.30-2.24 (m, 2H), 2.05-1.96 (m, 1H), 1.88-1.76 (m, 6H), 1.46-1.40 (m, 4H), 1.21 (t, J=6.8 Hz, 3H), 1.15-0.97 (m, 2H), 0.90 (t, J=7.6 Hz, 3H), 0.79 (t, J=6.8 Hz, 6H). LCMS (ESI): R_{T}=1.099 min, m/z. found 1138.4 [M-CF₃COOH+H]⁺.

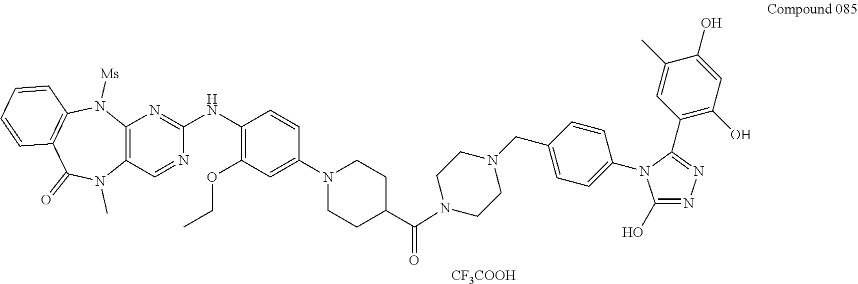 [SMILES: CCOc1cc(N2CCC(C(=O)N3CCN(Cc4ccc(-n5c(O)nnc5-c5cc(C)c(O)cc5O)cc4)CC3)CC2)ccc1Nc1ncc2c(n1)N(S(C)(=O)=O)c1ccccc1C(=O)N2C ; "CF3COOH"]

2-((4-(4-(4-(4-(3-(2,4-dihydroxy-5-methylphenyl)-5-hydroxy-4H-1,2,4-triazol-4-yl)benzyl)piperazine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.96 (s, 1H), 9.94 (s, 1H), 9.61 (s, 1H), 9.30 (s, 1H), 8.78 (s, 1H), 8.61 (s, 1H), 7.75 (d, J=7.6 Hz, 1H), 7.61-7.43 (m, 5H), 7.37-7.25 (m, 3H), 6.97 (s, 1H), 6.71-6.51 (m, 2H), 6.23 (s, 1H), 4.54-4.19 (m, 4H), 4.10-3.97 (m, 4H), 3.74-3.66 (m, 7H), 3.46 (s, 4H), 3.37-3.26 (m, 2H), 3.12-2.78 (m, 5H), 1.98 (s, 3H), 1.80-1.65 (m, 4H), 1.17 (t, J=6.8 Hz, 3H). LCMS (ESI): R_{T}=1.000 min, m/z. found 930.3 [M-CF₃COOH+H]⁺.

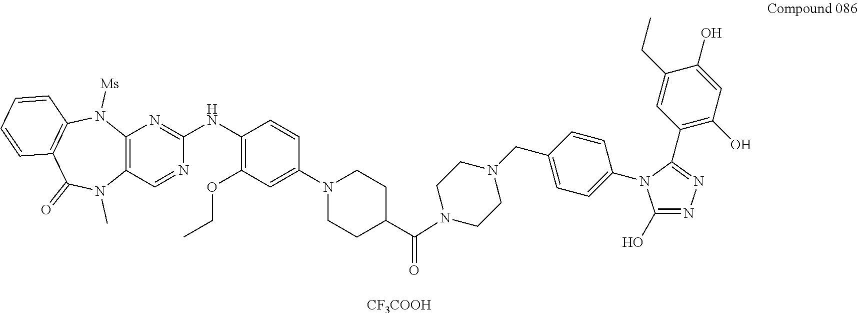 [SMILES: CCOc1cc(N2CCC(C(=O)N3CCN(Cc4ccc(-n5c(O)nnc5-c5cc(CC)c(O)cc5O)cc4)CC3)CC2)ccc1Nc1ncc2c(n1)N(S(C)(=O)=O)c1ccccc1C(=O)N2C ; "CF3COOH"]

2-((2-ethoxy-4-(4-(4-(4-(3-(5-ethyl-2,4-dihydroxyphenyl)-5-hydroxy-4H-1,2,4-triazol-4-yl)benzyl)piperazine-1-carbonyl)piperidin-1-yl)phenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.90 (s, 1H), 9.55 (s, 1H), 9.35 (s, 1H), 8.74 (s, 1H), 8.60 (s, 1H), 7.74 (d, J=7.6 Hz, 1H), 7.62-7.54 (m, 2H), 7.48-7.41 (m, 1H), 7.33-7.11 (m, 5H), 6.83 (s, 1H), 6.62-6.45 (m, 2H), 6.25 (s, 1H), 4.08-3.95 (m, 2H), 3.74-3.62 (m, 5H), 3.56-3.44 (m, 9H), 2.82-2.68 (m, 3H), 2.41-2.27 (m, 6H), 1.72-1.63 (m, 4H), 1.17-1.12 (m, 3H), 1.00-0.94 (m, 3H). LCMS (ESI): R_{T}=1.036 min, m/z. found 944.2 [M-CF₃COOH+H]⁺.

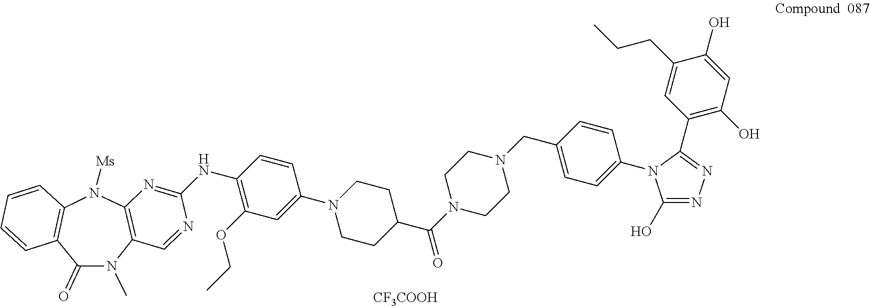 [SMILES: CCCc1cc(-c2nnc(O)n2-c2ccc(CN3CCN(C(=O)C4CCN(c5ccc(Nc6ncc7c(n6)N(S(C)(=O)=O)c6ccccc6C(=O)N7C)c(OCC)c5)CC4)CC3)cc2)c(O)cc1O ; "CF3COOH"]

2-((4-(4-(4-(4-(3-(2,4-dihydroxy-5-propylphenyl)-5-hydroxy-4H-1,2,4-triazol-4-yl)benzyl)piperazine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.89 (s, 1H), 9.54-9.33 (m, 2H), 8.76-8.58 (m, 2H), 7.74 (d, J=7.6 Hz, 1H), 7.61-7.42 (m, 3H), 7.31-7.10 (m, 5H), 6.79 (s, 1H), 6.62-6.46 (m, 2H), 6.25 (s, 1H), 4.08-3.96 (m, 2H), 3.75-3.64 (m, 5H), 3.57-3.44 (m, 9H), 2.82-2.66 (m, 3H), 2.42-2.27 (m, 6H), 1.73-1.62 (m, 4H), 1.43-1.33 (m, 2H), 1.15 (t, J=6.8 Hz, 3H), 0.77 (t, J=7.2 Hz, 3H). LCMS (ESI): R_{T}=1.069 min, m/z. found 958.3 [M-CF₃COOH+H]⁺.

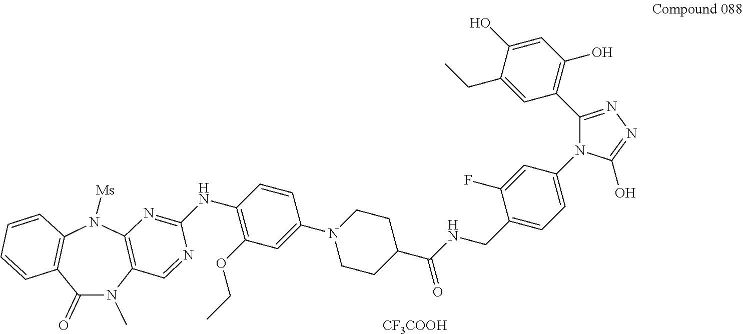 [SMILES: CCOc1cc(N2CCC(C(=O)NCc3ccc(-n4c(O)nnc4-c4cc(CC)c(O)cc4O)cc3F)CC2)ccc1Nc1ncc2c(n1)N(S(C)(=O)=O)c1ccccc1C(=O)N2C ; "CF3COOH"]

1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)-N-(4-(3-(5-ethyl-2,4-dihydroxyphenyl)-5-hydroxy-4H-1,2,4-triazol-4-yl)-2-fluorobenzyl)piperidine-4-carboxamide, trifluoroacetic acid. ¹H NMR (400 MHz, DMSO-d₆): δ 11.96 (s, 1H), 9.59 (s, 1H), 9.39 (s, 1H), 8.85 (s, 1H), 8.64 (s, 1H), 8.44 (s, 1H), 7.75 (d, J=7.6 Hz, 1H), 7.59 (d, J=4.0 Hz, 2H), 7.48-7.44 (m, 2H), 7.25 (t, J=8.4 Hz, 1H), 7.06 (dd, J=10.8, 1.5 Hz, 1H), 6.99-6.92 (m, 2H), 6.91-6.66 (m, 2H), 6.25 (s, 1H), 4.30 (d, J=5.4 Hz, 2H), 4.09-4.01 (m, 3H), 3.70 (s, 4H), 3.46 (s, 3H), 2.97 (s, 2H), 2.40 (q, J=7.6 Hz, 3H), 1.94-1.74 (m, 4H), 1.20 (t, J=6.7 Hz, 3H), 1.04 (t, J=7.6 Hz, 3H). LCMS (ESI): R_{T} 1.290 min, m/z. found 892.8 [M-CF₃COOH+H]⁺.

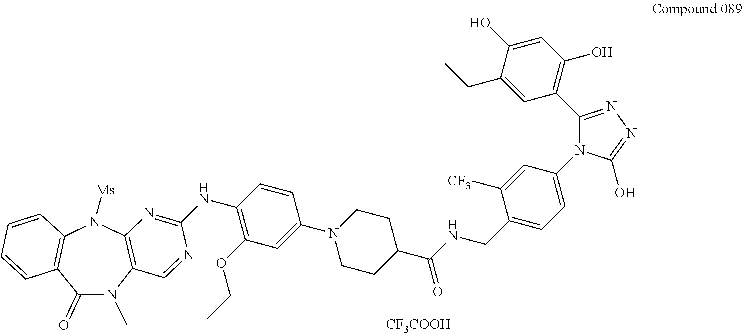 [SMILES: CCOc1cc(N2CCC(C(=O)NCc3ccc(-n4c(O)nnc4-c4cc(CC)c(O)cc4O)cc3C(F)(F)F)CC2)ccc1Nc1ncc2c(n1)N(S(C)(=O)=O)c1ccccc1C(=O)N2C ; "CF3COOH"]

1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)-N-(4-(3-(5-ethyl-2,4-dihydroxyphenyl)-5-hydroxy-4H-1,2,4-triazol-4-yl)-2-(trifluoromethyl)benzyl)piperidine-4-carboxamide, trifluoroacetic acid. ¹H NMR (400 MHz, DMSO-d₆): δ 12.02 (s, 1H), 9.60 (s, 1H), 9.38 (s, 1H), 8.86 (s, 1H), 8.64 (s, 1H), 8.54 (s, 1H), 7.76 (d, J=7.6 Hz, 1H), 7.63-7.39 (m, 7H), 7.01 (s, 1H), 6.96-6.64 (m, 2H), 6.24 (s, 1H), 4.44 (d, J=5.2 Hz, 2H), 4.08-4.03 (m, 3H), 3.70 (s, 5H), 3.47 (s, 3H), 3.17-2.85 (m, 2H), 2.41 (q, J=7.6 Hz, 2H), 2.00-1.75 (m, 4H), 1.20 (t, J=6.8 Hz, 3H), 1.05 (t, J=7.6 Hz, 3H). LCMS (ESI): R_{T}=1.370 min, m/z. found 942.7 [M-CF₃COOH+H]⁺.

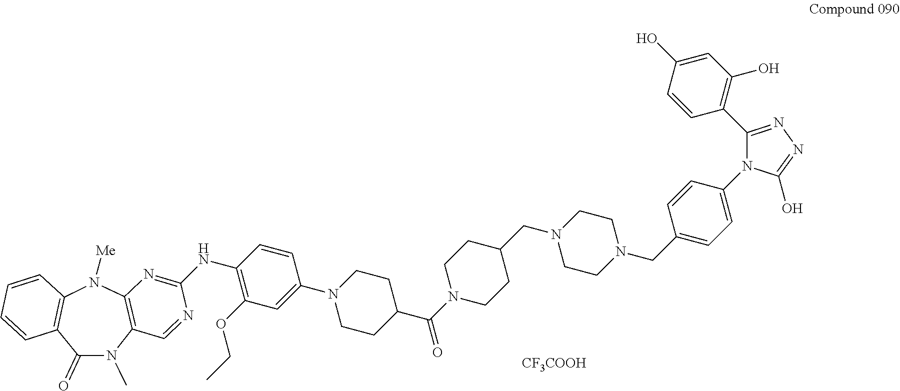 [SMILES: CCOc1cc(N2CCC(C(=O)N3CCC(CN4CCN(Cc5ccc(-n6c(O)nnc6-c6ccc(O)cc6O)cc5)CC4)CC3)CC2)ccc1Nc1ncc2c(n1)N(C)c1ccccc1C(=O)N2C ; "CF3COOH"]

2-((4-(4-(4-((4-(4-(3-(2,4-dihydroxyphenyl)-5-hydroxy-4H-1,2,4-triazol-4-yl)benzyl)piperazin-1-yl)methyl)piperidine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.89 (s, 1H), 9.59 (d, J=24.4 Hz, 2H), 8.67 (d, J=56.0 Hz, 2H), 7.74 (d, J=7.6 Hz, 1H), 7.63-7.54 (m, 2H), 7.50-7.41 (m, 1H), 7.32-7.18 (m, 3H), 7.15-6.97 (m, 3H), 6.64-6.43 (m, 2H), 6.30-6.11 (m, 2H), 4.37 (d, J=12.7 Hz, 1H), 4.08-3.92 (m, 3H), 3.80-3.59 (m, 5H), 3.43 (d, J=14.0 Hz, 5H), 3.37-3.34 (m, 2H), 3.11-2.94 (m, 1H), 2.84-2.64 (m, 3H), 2.44-2.24 (m, 7H), 2.11 (d, J=6.8 Hz, 2H), 1.82-1.57 (m, 7H), 1.15 (t, J=6.8 Hz, 3H), 1.06-0.79 (m, 2H). LCMS (ESI): R_{T}=1.040 min, m/z. found 1012.8 [M-CF₃COOH+H]⁺.

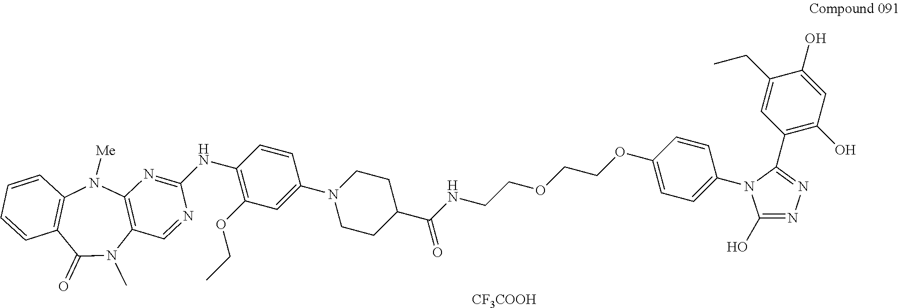 [SMILES: CCOc1cc(N2CCC(C(=O)NCCOCCOc3ccc(-n4c(O)nnc4-c4cc(CC)c(O)cc4O)cc3)CC2)ccc1Nc1ncc2c(n1)N(C)c1ccccc1C(=O)N2C ; "CF3COOH"]

1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)-N-(2-(2-(4-(3-(5-ethyl-2,4-dihydroxyphenyl)-5-hydroxy-4H-1,2,4-triazol-4-yl)phenoxy)ethoxy)ethyl)piperidine-4-carboxamide, trifluoroacetic acid. ¹H NMR (400 MHz, DMSO-d₆): δ 11.84 (s, 1H), 9.57-9.33 (m, 2H), 8.85 (s, 1H), 8.64 (s, 1H), 7.98 (s, 1H), 7.75 (d, J=7.6 Hz, 1H), 7.59 (d, J=3.6 Hz, 2H), 7.48-7.44 (m, 2H), 7.08 (d, J=7.2 Hz, 2H), 6.93-6.68 (m, 5H), 6.25 (s, 1H), 4.16-3.99 (m, 5H), 3.77-3.66 (m, 7H), 3.50-3.41 (m, 5H), 3.25-3.22 (m, 2H), 3.04-2.88 (m, 2H), 2.38-2.32 (m, 3H), 1.78 (s, 4H), 1.24-1.17 (m, 3H), 1.00 (d, J=7.2 Hz, 3H). LCMS (ESI): R_{T}=1.223 min, m/z. found 948.8 [M-CF₃COOH+H]⁺.

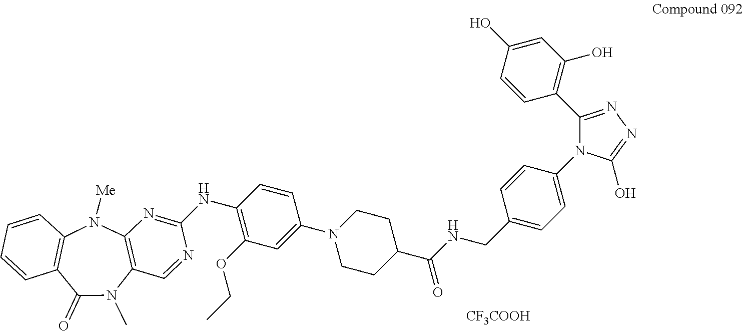 [SMILES: CCOc1cc(N2CCC(C(=O)NCc3ccc(-n4c(O)nnc4-c4ccc(O)cc4O)cc3)CC2)ccc1Nc1ncc2c(n1)N(C)c1ccccc1C(=O)N2C ; "CF3COOH"]

N-(4-(3-(2,4-dihydroxyphenyl)-5-hydroxy-4H-1,2,4-triazol-4-yl)benzyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 11.89 (s, 1H), 9.62-9.58 (m, 2H), 8.83 (d, J=4.8 Hz, 1H), 8.63 (s, 1H), 8.42 (d, J=3.6 Hz, 1H), 7.75 (d, J=8.0 Hz, 1H), 7.59 (d, J=4.0 Hz, 2H), 7.48-7.43 (m, 2H), 7.21-7.03 (m, 5H), 6.87-6.55 (m, 1H), 6.24-6.16 (m, 2H), 4.27 (d, J=6.0 Hz, 2H), 4.08-4.00 (m, 2H), 3.95-3.55 (m, 8H), 3.46 (s, 3H), 3.12-2.64 (m, 2H), 1.86-1.77 (m, 4H), 1.19 (d, J=6.8 Hz, 3H). LCMS (ESI): R_{T}=1.108 min, m/z. found 847.2 [M-CF₃COOH+H]⁺.

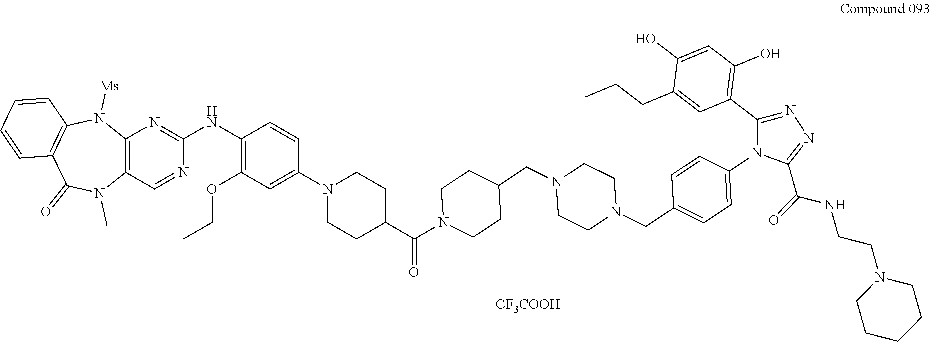 [SMILES: CCCc1cc(-c2nnc(C(=O)NCCN3CCCCC3)n2-c2ccc(CN3CCN(CC4CCN(C(=O)C5CCN(c6ccc(Nc7ncc8c(n7)N(S(C)(=O)=O)c7ccccc7C(=O)N8C)c(OCC)c6)CC5)CC4)CC3)cc2)c(O)cc1O ; "CF3COOH"]

5-(2,4-dihydroxy-5-propylphenyl)-4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-N-(2-(piperidin-1-yl)ethyl)-4H-1,2,4-triazole-3-carboxamide, trifluoroacetic acid. ¹H NMR (400 MHz, DMSO-d₆): δ 9.25 (s, 1H), 9.28-9.21 (m, 1H), 9.08 (s, 1H), 8.84 (s, 1H), 8.63 (s, 1H), 7.75 (d, J=7.6 Hz, 1H), 7.59 (d, J=4.0 Hz, 2H), 7.47-7.30 (m, 6H), 6.72-6.65 (m, 2H), 6.30 (s, 1H), 4.44-4.33 (m, 3H), 4.07-4.02 (m, 4H), 3.69 (s, 6H), 3.56-3.49 (m, 5H), 3.46 (s, 4H), 3.20 (d, J=5.2 Hz, 2H), 3.06-2.83 (m, 12H), 2.61-2.53 (m, 1H), 2.24 (t, J=7.2 Hz, 2H), 2.11-1.92 (m, 1H), 1.83-1.55 (m, 12H), 1.35-1.29 (m, 1H), 1.20-0.99 (m, 5H), 0.74 (t, J=7.2 Hz, 6H). LCMS (ESI): R_{T}=0.936 min, m/z. found 597.6 [M/2-CF₃COOH+H]⁺.

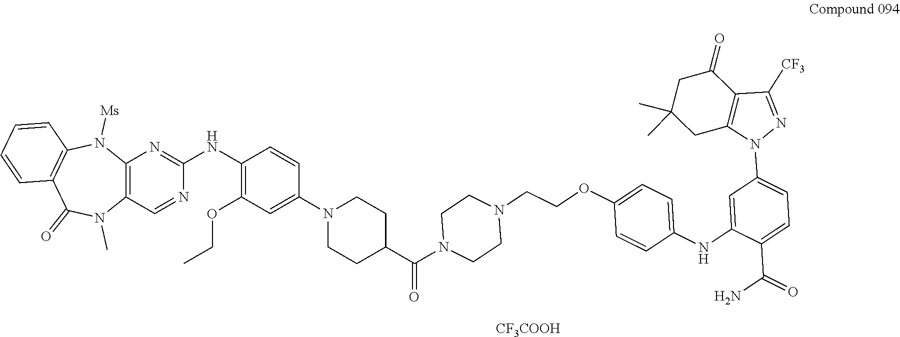 [SMILES: CCOc1cc(N2CCC(C(=O)N3CCN(CCOc4ccc(Nc5cc(-n6nc(C(F)(F)F)c7c6CC(C)(C)CC7=O)ccc5C(N)=O)cc4)CC3)CC2)ccc1Nc1ncc2c(n1)N(S(C)(=O)=O)c1ccccc1C(=O)N2C ; "CF3COOH"]

4-(6,6-dimethyl-4-oxo-3-(trifluoromethyl)-4,5,6,7-tetrahydro-1H-indazol-1-yl)-2-((4-(2-(4-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperazin-1-yl)ethoxy)phenyl)amino)benzamide, trifluoroacetic acid. ¹H NMR (400 MHz, DMSO-d₆): δ 10.16 (s, 2H), 8.82 (s, 1H), 8.63 (s, 1H), 8.21 (s, 1H), 7.91 (d, J=8.4 Hz, 1H), 7.75 (d, J=7.6 Hz, 1H), 7.65-7.56 (m, 3H), 7.51-7.36 (m, 2H), 7.29 (d, J=8.8 Hz, 2H), 7.09-7.02 (m, 3H), 6.99-6.94 (m, 1H), 6.80-6.56 (m 2H), 4.58-4.43 (m, 1H), 4.41-4.34 (m, 2H), 4.09-4.02 (m, 5H), 3.75-3.67 (m, 6H), 3.64-3.56 (m, 4H), 3.46 (s, 4H), 2.98-2.86 (m, 5H), 2.43 (s, 2H), 1.84-1.70 (m, 4H), 1.19 (t, J=6.8 Hz, 3H), 1.04 (s, 6H). LCMS (ESI): R_{T}=1.276 min, m/z. found 1118.8 [M-CF₃COOH+H]⁺.

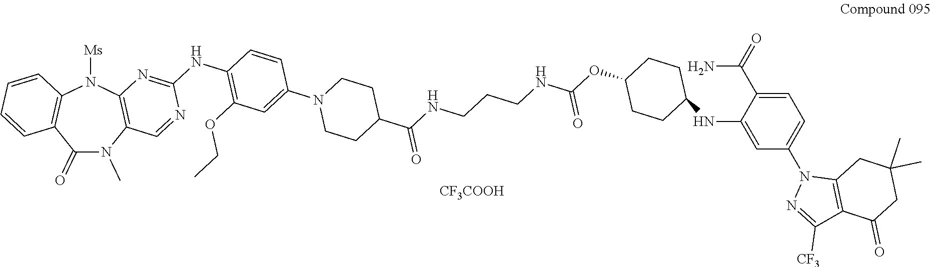 [SMILES: CCOc1cc(N2CCC(C(=O)NCCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(S(C)(=O)=O)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl (3-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamido)propyl)carbamate, trifluoroacetic acid. ¹H NMR (400 MHz, DMSO-d₆): δ 8.85 (s, 1H), 8.64 (s, 1H), 8.43 (s, 1H), 7.99 (s, 1H), 7.86 (s, 1H), 7.81-7.73 (m, 2H), 7.59 (d, J=4.0 Hz, 2H), 7.50-7.35 (m, 2H), 7.10-6.70 (m, 5H), 4.57-4.48 (m, 1H), 4.09-4.02 (m, 3H), 3.73-3.63 (m, 7H), 3.49-3.42 (m, 4H), 3.09-3.03 (m, 2H), 3.00-2.95 (m, 4H), 2.45 (s, 2H), 2.36-2.31 (m, 1H), 2.06-1.78 (m, 8H), 1.54-1.19 (m, 9H), 1.03 (s, 6H). LCMS (ESI): R_{T}=1.509 min, m/z. found 1112.9 [M-CF₃COOH+H]⁺.

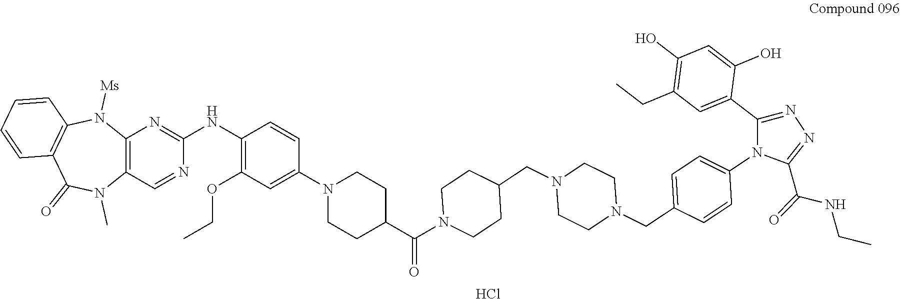 [SMILES: CCNC(=O)c1nnc(-c2cc(CC)c(O)cc2O)n1-c1ccc(CN2CCN(CC3CCN(C(=O)C4CCN(c5ccc(Nc6ncc7c(n6)N(S(C)(=O)=O)c6ccccc6C(=O)N7C)c(OCC)c5)CC4)CC3)CC2)cc1 ; Cl]

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-N-ethyl-5-(5-ethyl-2,4-dihydroxyphenyl)-4H-1,2,4-triazole-3-carboxamide, hydrochloride. ¹H NMR (400 MHz, DMSO-d₆): δ 10.39 (s, 1H), 9.73 (s, 1H), 8.99-8.93 (m, 1H), 8.75 (s, 1H), 8.60 (s, 1H), 7.74 (d, J=7.6 Hz, 1H), 7.58 (d, J=3.6 Hz, 2H), 7.45-7.25 (m, 8H), 6.60-6.47 (m, 3H), 6.32 (s, 1H), 5.05-5.01 (m, 1H), 4.42-4.33 (m, 1H), 4.05-3.96 (m, 3H), 3.71-3.60 (m, 6H), 3.49 (s, 2H), 3.45 (s, 3H), 3.19-3.15 (m, 2H), 3.05-2.99 (m, 1H), 2.79-2.71 (m, 3H), 2.42-2.31 (m, 7H), 2.24-2.11 (m, 4H), 2.03-1.95 (m, 1H), 1.77-1.66 (m, 6H), 1.28-1.14 (m, 8H), 1.07-1.01 (m, 4H), 0.86-0.81 (m, 4H). LCMS (ESI): R_{T}=1.134 min, m/z. found 1095.8 [M−HCl+H]⁺.

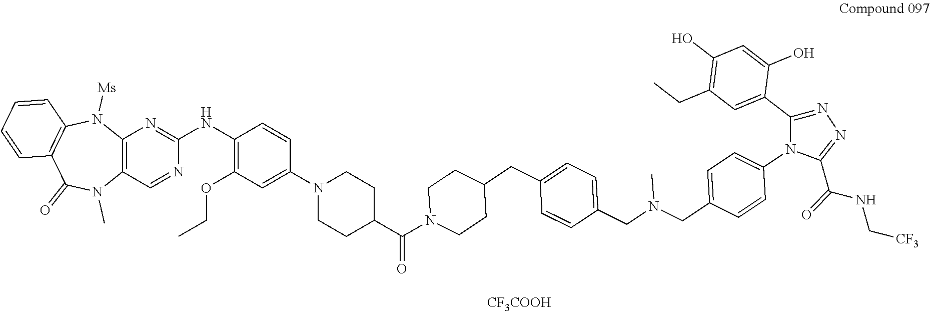 [SMILES: CCOc1cc(N2CCC(C(=O)N3CCC(Cc4ccc(CN(C)Cc5ccc(-n6c(C(=O)NCC(F)(F)F)nnc6-c6cc(CC)c(O)cc6O)cc5)cc4)CC3)CC2)ccc1Nc1ncc2c(n1)N(S(C)(=O)=O)c1ccccc1C(=O)N2C ; "CF3COOH"]

4-(4-(((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)benzyl)(methyl)amino)methyl)phenyl)-5-(5-ethyl-2,4-dihydroxyphenyl)-N-(2,2,2-trifluoroethyl)-4H-1,2,4-triazole-3-carboxamide, trifluoroacetic acid. ¹H NMR (400 MHz, DMSO-d₆): δ 10.03-9.65 (m, 3H), 8.88 (s, 1H), 8.65 (s, 1H), 7.76 (d, J=7.6 Hz, 1H), 7.76-7.29 (m, 12H), 6.96-6.72 (m, 3H), 6.29 (s, 1H), 4.52-4.18 (m, 5H), 4.11-3.92 (m, 5H), 3.76-3.67 (m, 5H), 3.47 (s, 4H), 3.16-2.79 (m, 4H), 2.61-2.55 (m, 4H), 2.29 (q, J=7.4 Hz, 2H), 1.91-1.71 (m, 5H), 1.70-1.53 (m, 2H), 1.32-1.08 (m, 5H), 1.04-0.90 (m, 4H). LCMS (ESI): R_{T}=1.255 min, m/z. found 1184.7 [M-CF₃COOH+H]⁺.

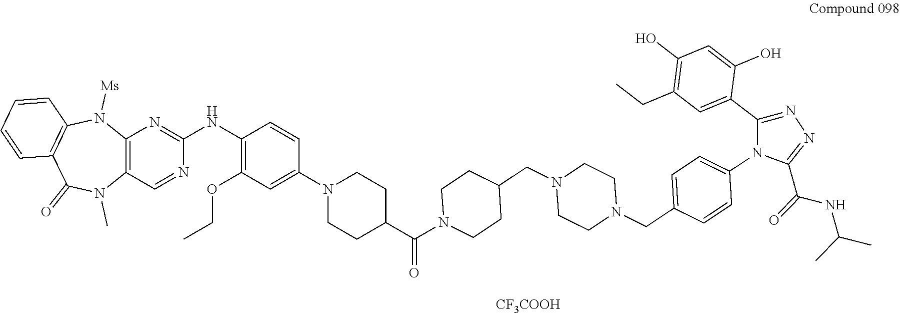 [SMILES: CCOc1cc(N2CCC(C(=O)N3CCC(CN4CCN(Cc5ccc(-n6c(C(=O)NC(C)C)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-isopropyl-4H-1,2,4-triazole-3-carboxamide, trifluoroacetic acid. ¹H NMR (400 MHz, DMSO-d₆): δ 10.34 (d, J=20.0 Hz, 1H), 9.73 (s, 1H), 8.77 (d, J=11.6 Hz, 2H), 8.60 (s, 1H), 7.74 (d, J=8.0 Hz, 1H), 7.59-7.24 (m, 8H), 6.59-6.47 (m, 3H), 6.32 (s, 1H), 4.38 (d, J=12.0 Hz, 1H), 4.19 (q, J=6.8 Hz, 1H), 4.07-3.87 (m, 4H), 3.69 (d, J=10.0 Hz, 5H), 3.53-3.47 (m, 2H), 3.45 (s, 4H), 3.10-2.95 (m, 1H), 2.81-2.68 (m, 3H), 2.43-2.28 (m, 6H), 2.22 (q, J=7.4 Hz, 2H), 2.12 (d, J=6.8 Hz, 2H), 2.04-1.94 (m, 1H), 1.81-1.62 (m, 7H), 1.23-1.07 (m, 10H), 0.89-0.80 (m, 4H). LCMS (ESI): R_{T}=1.155 min, m/z. found 1109.8 [M-CF₃COOH+H]⁺.

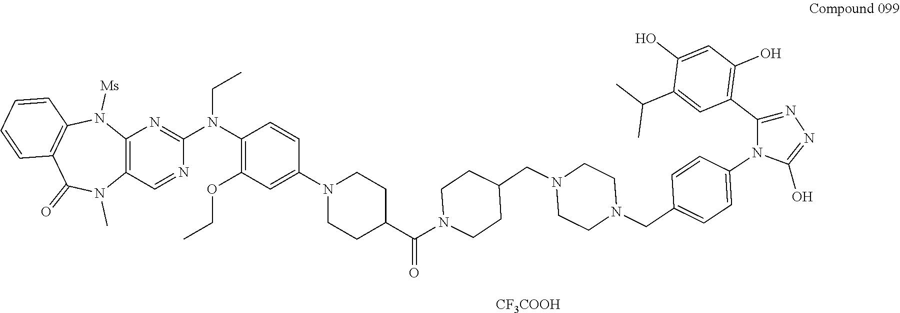 [SMILES: CCOc1cc(N2CCC(C(=O)N3CCC(CN4CCN(Cc5ccc(-n6c(O)nnc6-c6cc(C(C)C)c(O)cc6O)cc5)CC4)CC3)CC2)ccc1N(CC)c1ncc2c(n1)N(S(C)(=O)=O)c1ccccc1C(=O)N2C ; "CF3COOH"]

2-((4-(4-(4-((4-(4-(3-(2,4-dihydroxy-5-isopropylphenyl)-5-hydroxy-4H-1,2,4-triazol-4-yl)benzyl)piperazin-1-yl)methyl)piperidine-1-carbonyl)piperidin-1-yl)-2-ethoxyphenyl)(ethyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.95 (s, 1H), 9.62 (s, 1H), 9.38 (s, 1H), 8.79-8.37 (m, 1H), 7.20 (s, 1H), 7.56-7.35 (m, 4H), 7.21 (d, J=8.8 Hz, 2H), 7.13-6.97 (m, 1H), 6.82 (s, 1H), 6.73-6.47 (m, 2H), 6.26 (s, 1H), 4.43-4.36 (m, 1H), 4.09-3.93 (m, 12H), 3.68-3.64 (m, 3H), 3.44 (s, 4H), 3.21 (s, 3H), 3.09-2.83 (m, 9H), 2.63-2.54 (m, 2H), 2.06-1.89 (m, 1H), 1.82-1.61 (m, 6H), 1.19-1.04 (m, 6H), 0.98 (d, J=6.8 Hz, 7H). LCMS (ESI): R_{T}=5.067 min, m/z. found 1083.3 [M-CF₃COOH+H]⁺.

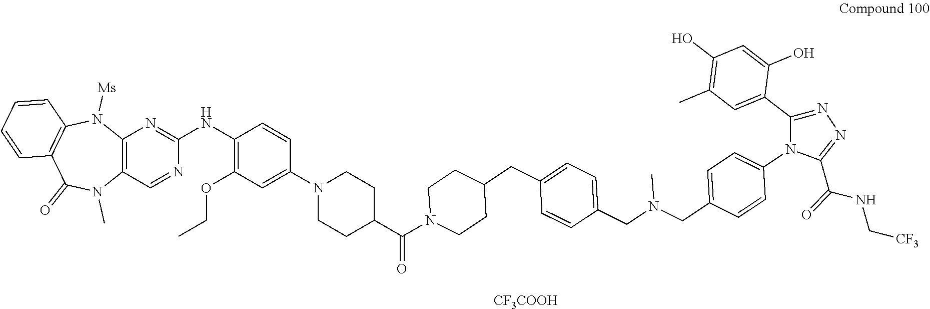 [SMILES: CCOc1cc(N2CCC(C(=O)N3CCC(Cc4ccc(CN(C)Cc5ccc(-n6c(C(=O)NCC(F)(F)F)nnc6-c6cc(C)c(O)cc6O)cc5)cc4)CC3)CC2)ccc1Nc1ncc2c(n1)N(S(C)(=O)=O)c1ccccc1C(=O)N2C ; "CF3COOH"]

5-(2,4-dihydroxy-5-methylphenyl)-4-(4-(((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)benzyl)(methyl)amino)methyl)phenyl)-N-(2,2,2-trifluoroethyl)-4H-1,2,4-triazole-3-carboxamide, trifluoroacetic acid. ¹H NMR (400 MHz, DMSO-d₆): δ 10.04 (s, 1H), 9.70-9.58 (m, 2H), 8.74 (s, 1H), 8.60 (s, 1H), 7.74 (d, J=7.6 Hz, 1H), 7.59-7.13 (m, 13H), 6.65-6.46 (m, 3H), 6.30 (s, 1H), 5.45-5.11 (m, 1H), 4.46-4.36 (m, 1H), 4.00-3.93 (m, 5H), 3.71-3.61 (m, 4H), 3.68 (s, 2H), 3.51-3.45 (m, 4H), 2.99-2.92 (m, 1H), 2.75-2.67 (m, 3H), 2.07-1.97 (m, 4H), 1.80-1.52 (m, 10H), 1.24 (s, 3H), 1.19-1.13 (m, 3H), 0.97-0.83 (m, 2H). LCMS (ESI): R_{T}=1.245 min, m/z. found 1172.1 [M-CF₃COOH+H]⁺.

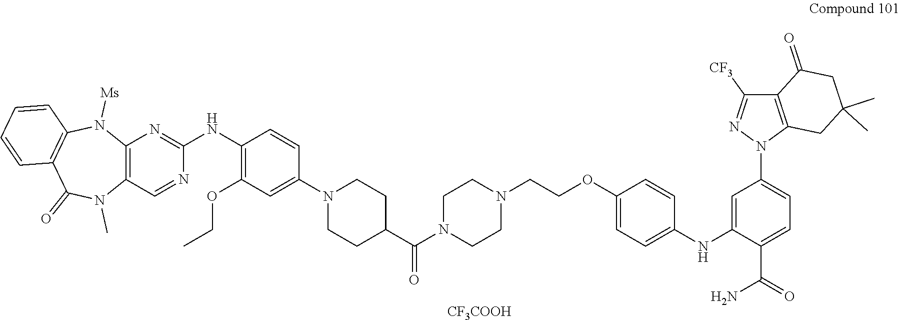 [SMILES: CCOc1cc(N2CCC(C(=O)N3CCN(CCOc4ccc(Nc5cc(-n6nc(C(F)(F)F)c7c6CC(C)(C)CC7=O)ccc5C(N)=O)cc4)CC3)CC2)ccc1Nc1ncc2c(n1)N(S(C)(=O)=O)c1ccccc1C(=O)N2C ; "CF3COOH"]

4-(6,6-dimethyl-4-oxo-3-(trifluoromethyl)-4,5,6,7-tetrahydro-1H-indazol-1-yl)-2-((4-(2-(4-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperazin-1-yl)ethoxy)phenyl)amino)benzamide, trifluoroacetic acid. ¹H NMR (400 MHz, DMSO-d₆): δ 10.16 (s, 1H), 8.38 (s, 1H), 8.27-8.20 (m, 2H), 8.12-8.04 (m, 1H), 7.92 (d, J=8.8 Hz, 1H), 7.71-7.50 (m, 3H), 7.31-6.96 (m, 10H), 4.37 (s, 2H), 4.17-4.11 (m, 3H), 3.86-3.76 (m, 7H), 3.65-3.55 (m, 6H), 3.40 (s, 3H), 3.33 (s, 3H), 3.22-3.11 (m, 2H), 3.00-2.94 (m, 3H), 2.43 (s, 2H), 1.89 (s, 3H), 1.35 (t, J=6.8 Hz, 3H), 1.04 (s, 6H). LCMS (ESI): R_{T} 1.302 min, m/z. found 1154.8 [M-CF₃COOH+H]⁺.

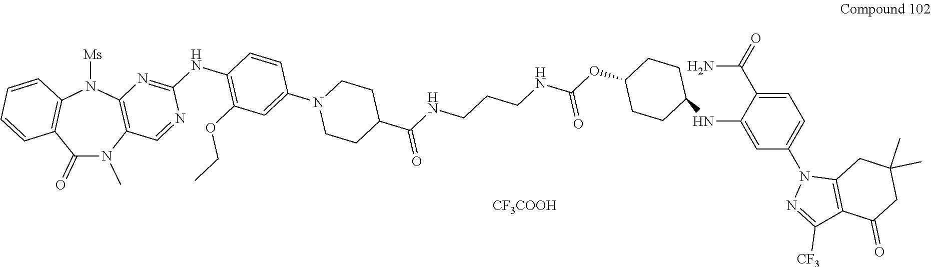 [SMILES: CCOc1cc(N2CCC(C(=O)NCCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(S(C)(=O)=O)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl (3-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamido)propyl)carbamate, trifluoroacetic acid. ¹H NMR (400 MHz, DMSO-d₆): δ 8.39 (s, 2H), 8.22-7.96 (m, 4H), 7.81-7.68 (m, 2H), 7.54-7.50 (m, 1H), 7.37-7.07 (m, 6H), 6.90 (s, 1H), 6.75-6.72 (m, 1H), 4.53 (s, 1H), 4.18-4.12 (m, 2H), 3.69-3.65 (m, 3H), 3.58-3.40 (m, 5H), 3.34-3.24 (m, 5H), 3.11-2.98 (m, 6H), 2.45 (s, 3H), 2.06-1.93 (m, 7H), 1.57-1.48 (m, 4H), 1.38-1.23 (m, 5H), 1.04 (s, 6H). LCMS (ESI): R_{T}=1.519 min, m/z. found 1048.9 [M-CF₃COOH+H]⁺.

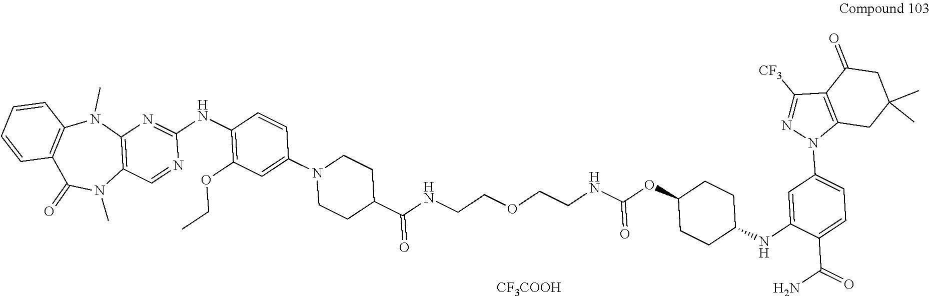 [SMILES: CCOc1cc(N2CCC(C(=O)NCCOCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(C)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl (2-(2-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamido)ethoxy)ethyl)carbamate, trifluoroacetic acid. ¹H NMR (400 MHz, DMSO-d₆): δ 8.54-8.33 (m, 2H), 8.24-7.96 (m, 4H), 7.83-7.67 (m, 2H), 7.55-6.87 (m, 8H), 6.76-6.69 (m, 1H), 4.59-4.49 (m, 1H), 4.18-4.09 (m, 2H), 3.67 (s, 3H), 3.44-3.38 (m, 8H), 3.32 (s, 3H), 3.27-3.20 (m, 3H), 3.17-3.09 (m, 3H), 2.97 (s, 2H), 2.44 (s, 3H), 2.08-1.87 (m, 8H), 1.56-1.43 (m, 2H), 1.38-1.21 (m, 7H), 1.03 (s, 6H). LCMS (ESI): R_{T}=1.506 min, m/z. found 1078.8 [M-CF₃COOH+H]⁺.

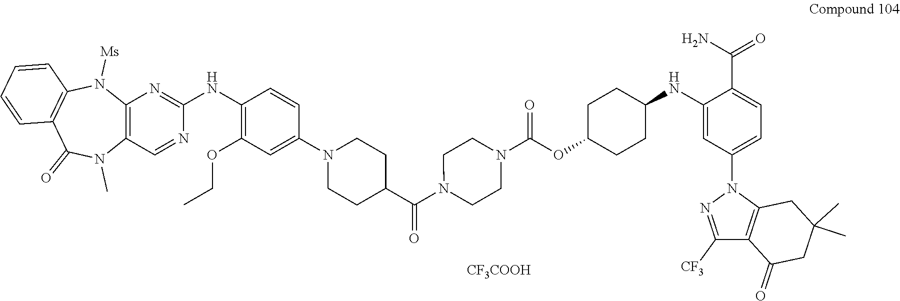 [SMILES: CCOc1cc(N2CCC(C(=O)N3CCN(C(=O)O[C@H]4CC[C@H](Nc5cc(-n6nc(C(F)(F)F)c7c6CC(C)(C)CC7=O)ccc5C(N)=O)CC4)CC3)CC2)ccc1Nc1ncc2c(n1)N(S(C)(=O)=O)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl 4-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperazine-1-carboxylate, trifluoroacetic acid. ¹H NMR (400 MHz, DMSO-d₆): δ 8.36-8.34 (m, 2H), 8.29-7.96 (m, 3H), 7.85-7.67 (m, 2H), 7.56-7.48 (m, 1H), 7.37 (s, 1H), 7.29-7.17 (m, 2H), 7.15-6.95 (m, 2H), 6.89 (d, J=2.0 Hz 1H), 6.74 (dd, J=8.4, 1.6 Hz, 1H), 4.68-4.58 (m, 1H), 4.20-4.12 (m, 2H), 3.68 (s, 4H), 3.52-3.48 (m, 5H), 3.46-3.43 (m, 2H), 3.41-3.37 (m, 5H), 3.33 (s, 4H), 2.99 (s, 3H), 2.45 (s, 2H), 2.06-1.88 (m, 8H), 1.61-1.51 (m, 2H), 1.42-1.33 (m, 5H), 1.04 (s, 6H). LCMS (ESI): R_{T}=1.594 min, m/z. found 1060.9 [M-CF₃COOH+H]⁺.

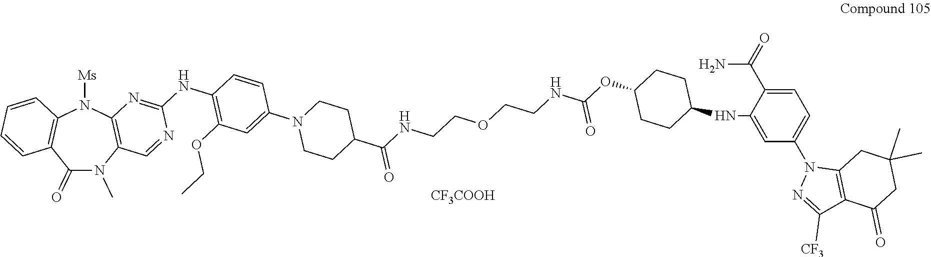 [SMILES: CCOc1cc(N2CCC(C(=O)NCCOCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(S(C)(=O)=O)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl (2-(2-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamido)ethoxy)ethyl)carbamate, trifluoroacetic acid. ¹H NMR (400 MHz, DMSO-d₆): δ 8.83 (s, 1H), 8.64 (s, 1H), 8.43 (s, 1H), 8.05-7.89 (m, 2H), 7.82-7.72 (m, 2H), 7.59 (d, J=3.6 Hz, 2H), 7.52-7.31 (m, 3H), 7.11-7.04 (m, 1H), 6.95-6.67 (m, 4H), 4.58-4.49 (m, 1H), 4.10-4.01 (m, 3H), 3.72-3.65 (m, 6H), 3.48-3.37 (m, 9H), 3.25-3.09 (m, 4H), 3.03-2.85 (m, 4H), 2.44 (s, 3H), 2.08-1.79 (m, 8H), 1.55-1.17 (m, 8H), 1.03 (s, 6H). LCMS (ESI): R_{T}=1.508 min, m/z. found 1142.9 [M-CF₃COOH+H]⁺.

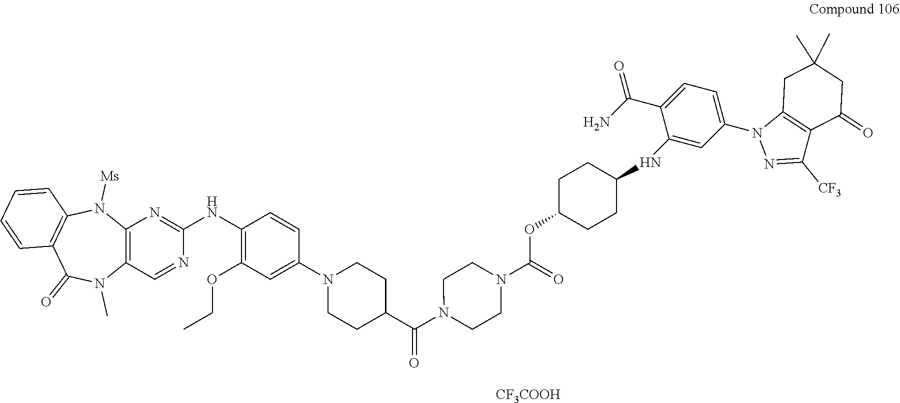 [SMILES: CCOc1cc(N2CCC(C(=O)N3CCN(C(=O)O[C@H]4CC[C@H](Nc5cc(-n6nc(C(F)(F)F)c7c6CC(C)(C)CC7=O)ccc5C(N)=O)CC4)CC3)CC2)ccc1Nc1ncc2c(n1)N(S(C)(=O)=O)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl 4-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperazine-1-carboxylate, trifluoroacetic acid. ¹H NMR (400 MHz, DMSO-d₆): δ 8.89 (s, 1H), 8.66 (s, 1H), 8.49 (s, 1H), 8.00 (s, 1H), 7.81-7.75 (m, 2H), 7.60-7.31 (m, 5H), 6.95-6.73 (m, 4H), 4.65-4.60 (m, 1H), 4.10-4.04 (m, 3H), 3.69 (d, J=8.8 Hz, 4H), 3.60-3.33 (m, 13H), 3.25-3.09 (m, 1H), 3.02-2.87 (m, 3H), 2.45 (s, 2H), 2.07-1.78 (m, 8H), 1.56 (dd, J=20.6, 10.3 Hz, 2H), 1.37 (dd, J=21.2, 10.7 Hz, 2H), 1.22 (t, J=6.8 Hz, 3H), 1.04 (s, 6H). LCMS (ESI): R_{T}=1.590 min, m/z. found 1124.9 [M-CF₃COOH+H]⁺.

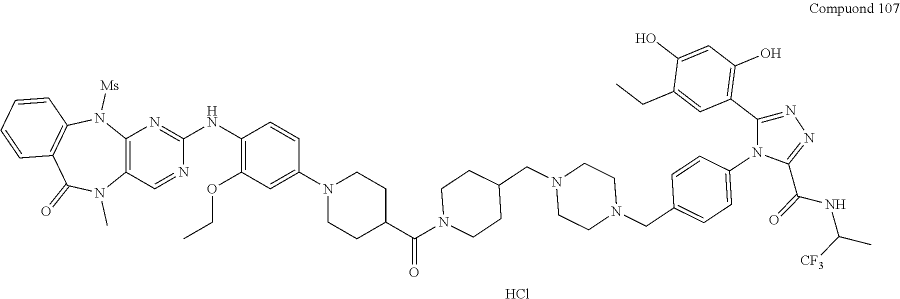 [SMILES: CCOc1cc(N2CCC(C(=O)N3CCC(CN4CCN(Cc5ccc(-n6c(C(=O)NC(C)C(F)(F)F)nnc6-c6cc(CC)c(O)cc6O)cc5)CC4)CC3)CC2)ccc1Nc1ncc2c(n1)N(S(C)(=O)=O)c1ccccc1C(=O)N2C ; Cl]

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, hydrochloride. ¹H NMR (400 MHz, DMSO-d₆): δ 11.58 (s, 1H), 9.80 (s, 1H), 9.66 (d, J=9.0 Hz, 1H), 9.09 (s, 1H), 8.72 (s, 1H), 7.99-7.27 (m, 12H), 6.80 (s, 1H), 6.37 (s, 1H), 4.66 (m, 1H), 4.40 (m, 2H), 4.17-4.12 (m, 4H), 3.72 (s, 7H), 3.60 (s, 4H), 3.48 (s, 5H), 3.11 (s, 4H), 2.61 (t, J=11.6 Hz, 1H), 2.20 (m, 10H), 1.40-1.24 (m, 6H), 1.22-1.03 (m, 2H), 0.95 (t, J=7.5 Hz, 3H). LCMS (ESI): R_{T} 1.140 min, m/z. found 1164.3 [M−HCl+H]⁺.

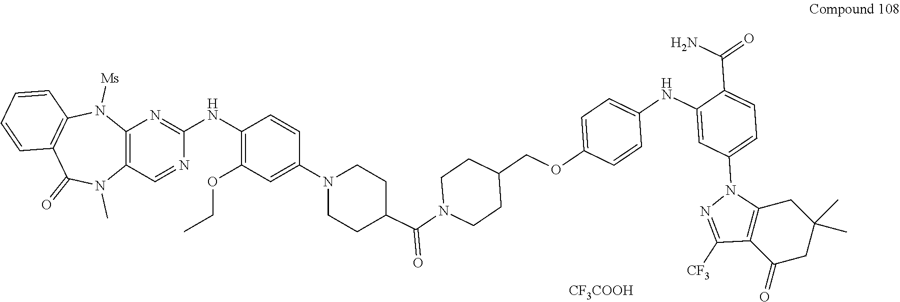 [SMILES: CCOc1cc(N2CCC(C(=O)N3CCC(COc4ccc(Nc5cc(-n6nc(C(F)(F)F)c7c6CC(C)(C)CC7=O)ccc5C(N)=O)cc4)CC3)CC2)ccc1Nc1ncc2c(n1)N(S(C)(=O)=O)c1ccccc1C(=O)N2C ; "CF3COOH"]

4-(6,6-dimethyl-4-oxo-3-(trifluoromethyl)-4,5,6,7-tetrahydro-1H-indazol-1-yl)-2-((4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methoxy)phenyl)amino)benzamide, trifluoroacetic acid. ¹H NMR (400 MHz, DMSO-d₆): δ 10.10 (s, 1H), 8.40 (s, 1H), 8.30-8.11 (m, 3H), 7.91 (d, J=8.4 Hz, 1H), 7.71-7.50 (m, 3H), 7.27-6.93 (m, 10H), 4.47 (d, J=12.4 Hz, 1H), 4.17 (q, J=6.8 Hz, 2H), 4.04 (d, J=12.4 Hz, 1H), 3.86 (d, J=6.0 Hz, 2H), 3.70-3.64 (m, 2H), 3.40 (s, 3H), 3.34 (s, 3H), 3.16-2.02 (m, 2H), 2.91 (s, 2H), 2.69-2.56 (m, 1H), 2.52-2.43 (m, 4H), 2.08-1.79 (m, 7H), 1.38 (t, J=6.8 Hz, 3H), 1.29-1.14 (m, 2H), 1.04 (s, 6H). LCMS (ESI): R_{T}=1.493 min, m/z. found 1040.5 [M-CF₃COOH+H]⁺.

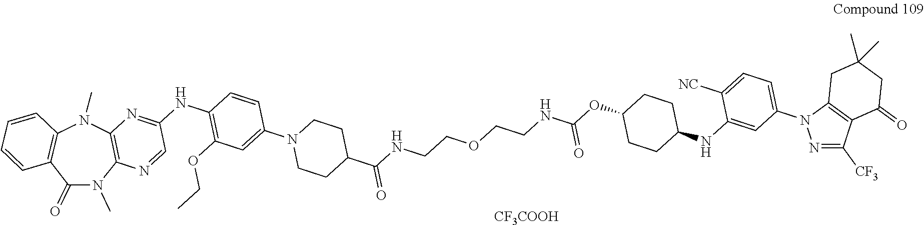 [SMILES: CCOc1cc(N2CCC(C(=O)NCCOCCNC(=O)O[C@H]3CC[C@H](Nc4cc(-n5nc(C(F)(F)F)c6c5CC(C)(C)CC6=O)ccc4C#N)CC3)CC2)ccc1Nc1cnc2c(n1)N(C)c1ccccc1C(=O)N2C ; "CF3COOH"]

(1R,4R)-4-((2-cyano-5-(6,6-dimethyl-4-oxo-3-(trifluoromethyl)-4,5,6,7-tetrahydro-1H-indazol-1-yl)phenyl)amino)cyclohexyl (2-(2-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamido)ethoxy)ethyl)carbamate, trifluoroacetic acid. ¹H NMR (400 MHz, DMSO-d₆): δ 8.37 (s, 1H), 8.17-8.01 (m, 3H), 7.69 (dd, J=7.9, 2.0 Hz, 2H), 7.54-7.49 (m, 1H), 7.26-6.86 (m, 7H), 6.18 (d, J=7.6 Hz, 1H), 4.52-4.43 (m, 1H), 4.14 (q, J=6.8 Hz, 2H), 3.67-3.64 (m, 3H), 3.42-3.39 (m, 6H), 3.33 (s, 3H), 3.23 (dd, J=11.2, 5.6 Hz, 3H), 3.14 (dd, J=11.6, 6.0 Hz, 2H), 2.98 (s, 2H), 2.55-2.38 (m, 5H), 2.08-1.80 (m, 8H), 1.59-1.41 (m, 4H), 1.35 (t, J=6.8 Hz, 3H), 1.03 (s, 6H). LCMS (ESI): R_{T}=1.458 min, m/z. found 1061.5 [M-CF₃COOH+H]⁺.

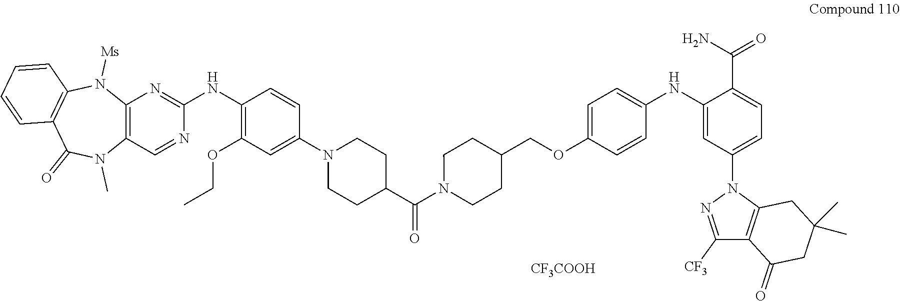 [SMILES: CCOc1cc(N2CCC(C(=O)N3CCC(COc4ccc(Nc5cc(-n6nc(C(F)(F)F)c7c6CC(C)(C)CC7=O)ccc5C(N)=O)cc4)CC3)CC2)ccc1Nc1ncc2c(n1)N(S(C)(=O)=O)c1ccccc1C(=O)N2C ; "CF3COOH"]

4-(6,6-dimethyl-4-oxo-3-(trifluoromethyl)-4,5,6,7-tetrahydro-1H-indazol-1-yl)-2-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methoxy)phenyl)amino)benzamide, trifluoroacetic acid. ¹H NMR (400 MHz, DMSO-d₆): δ 10.09 (s, 1H), 8.92 (s, 1H), 8.67 (s, 1H), 8.20 (s, 1H), 7.90 (d, J=8.4 Hz, 1H), 7.76 (d, J=7.6 Hz, 1H), 7.61-7.45 (m, 5H), 7.23 (d, J=8.8 Hz, 2H), 7.03-6.83 (m, 6H), 4.48-4.44 (m, 1H), 4.12-4.01 (m, 3H), 3.86 (d, J=6.0 Hz, 2H), 3.71-3.64 (m, 5H), 3.30-3.08 (m, 2H), 2.96-2.90 (m, 3H), 2.64-2.57 (m, 1H), 2.51-2.43 (m, 5H), 2.08-1.84 (m, 8H), 1.27-1.15 (m, 6H), 1.03 (s, 6H). LCMS (ESI): R_{T}=1.497 min, m/z. found 1104.4 [M-CF₃COOH+H]⁺.

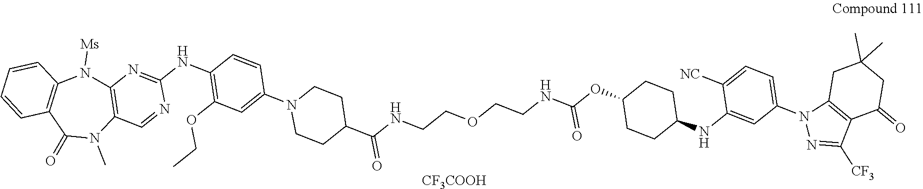 [SMILES: CCOc1cc(N2CCC(C(=O)NCCOCCNC(=O)O[C@H]3CC[C@H](Nc4cc(-n5nc(C(F)(F)F)c6c5CC(C)(C)CC6=O)ccc4C#N)CC3)CC2)ccc1Nc1ncc2c(n1)N(S(C)(=O)=O)c1ccccc1C(=O)N2C ; "CF3COOH"]

(1R,4R)-4-((2-((12-azanylidene)-l3-methyl)-5-(6,6-dimethyl-4-oxo-3-(trifluoromethyl)-4,5,6,7-tetrahydro-1H-indazol-1-yl)phenyl)amino)cyclohexyl (2-(2-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamido)ethoxy)ethyl)carbamate, trifluoroacetic acid. ¹H NMR (400 MHz, DMSO-d₆): δ 8.86 (s, 1H), 8.64 (s, 1H), 7.97 (d, J=5.2 Hz, 1H), 7.77-7.44 (m, 6H), 7.10-6.86 (m, 5H), 6.18 (s, 1H), 4.47 (s, 1H), 4.08-4.01 (m, 2H), 3.70-3.63 (m, 6H), 3.41-3.40 (m, 4H), 3.29-3.20 (m, 2H), 3.15-3.11 (m, 2H), 3.02-2.98 (m, 4H), 2.51-2.34 (m, 6H), 2.08-1.83 (m, 8H), 1.52-1.43 (m, 4H), 1.24-1.18 (m, 4H), 1.03 (s, 6H). LCMS (ESI): R_{T} 1.492 min, m/z. found 1125.5 [M-CF₃COOH+H]⁺.

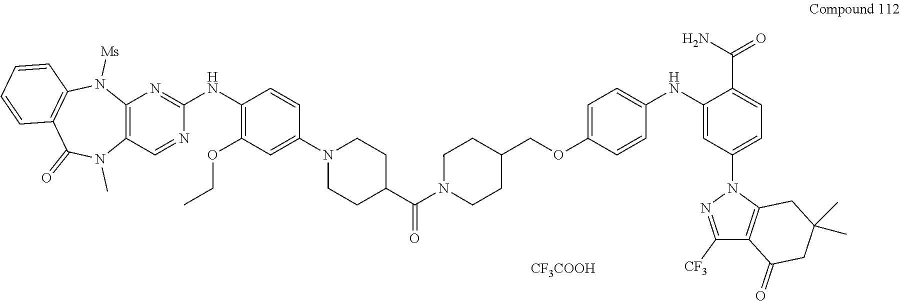 [SMILES: CCOc1cc(N2CCC(C(=O)N3CCC(COc4ccc(Nc5cc(-n6nc(C(F)(F)F)c7c6CC(C)(C)CC7=O)ccc5C(N)=O)cc4)CC3)CC2)ccc1Nc1ncc2c(n1)N(S(C)(=O)=O)c1ccccc1C(=O)N2C ; "CF3COOH"]

4-(6,6-dimethyl-4-oxo-3-(trifluoromethyl)-4,5,6,7-tetrahydro-1H-indazol-1-yl)-2-((4-((1-(1-(3-ethoxy-4-(ethyl(5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methoxy)phenyl)amino)benzamide, trifluoroacetic acid. ¹H NMR (400 MHz, DMSO-d₆): δ 10.10 (s, 1H), 8.86 (s, 1H), 8.19 (s, 1H), 7.90 (d, J=8.4 Hz, 1H), 7.71-7.41 (m, 5H), 7.23 (d, J=8.8 Hz, 2H), 7.03-6.93 (m, 5H), 6.62-6.45 (m, 2H), 4.44 (s, 1H), 4.06-3.74 (m, 11H), 3.45-3.40 (m, 3H), 3.22 (s, 2H), 3.12-3.00 (m, 1H), 2.90-2.73 (m, 5H), 2.59-2.50 (m, 1H), 2.43 (s, 2H), 2.03-1.95 (m, 1H), 1.86-1.68 (m, 6H), 1.23-1.10 (m, 8H), 1.08 (s, 6H). LCMS (ESI): R_{T}=1.907 min, m/z. found 1132.3 [M-CF₃COOH+H]⁺.

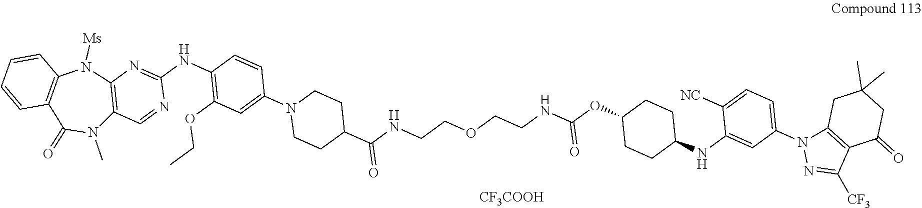 [SMILES: CCOc1cc(N2CCC(C(=O)NCCOCCNC(=O)O[C@H]3CC[C@H](Nc4cc(-n5nc(C(F)(F)F)c6c5CC(C)(C)CC6=O)ccc4C#N)CC3)CC2)ccc1Nc1ncc2c(n1)N(S(C)(=O)=O)c1ccccc1C(=O)N2C ; "CF3COOH"]

(1R,4R)-4-((2-((12-azanylidene)-l3-methyl)-5-(6,6-dimethyl-4-oxo-3-(trifluoromethyl)-4,5,6,7-tetrahydro-1H-indazol-1-yl)phenyl)amino)cyclohexyl (2-(2-(1-(3-ethoxy-4-(ethyl(5-methyl-11-(methylsulfonyl)-6-oxo-6,11-dihydro-5H-benzo[e]pyrimido[5,4-b][1,4]diazepin-2-yl)amino)phenyl)piperidine-4-carboxamido)ethoxy)ethyl)carbamate, trifluoroacetic acid. ¹H NMR (400 MHz, DMSO-d₆): δ 8.65 (s, 1H), 8.45 (s, 1H), 7.88-7.87 (m, 1H), 7.71-7.68 (m, 3H), 7.48-7.42 (m, 3H), 7.08-6.87 (m, 4H), 6.62-6.60 (m, 2H), 6.19-6.17 (m, 1H), 4.48-4.46 (m, 1H), 4.00-3.71 (m, 6H), 3.53-3.38 (m, 8H), 3.22-2.98 (m, 8H), 2.72-2.70 (m, 2H), 2.44 (s, 2H), 2.33-2.32 (m, 1H), 1.96-1.95 (m, 4H), 1.71-1.44 (m, 9H), 1.23-1.08 (m, 5H), 1.02 (s, 6H). LCMS (ESI): R_{T} 1.860 min, m/z. found 1153.3 [M-CF₃COOH+H]⁺.

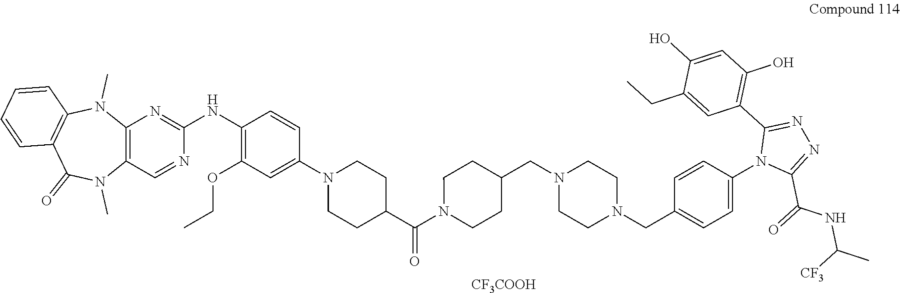 [SMILES: CCOc1cc(N2CCC(C(=O)N3CCC(CN4CCN(Cc5ccc(-n6c(C(=O)NC(C)C(F)(F)F)nnc6-c6cc(CC)c(O)cc6O)cc5)CC4)CC3)CC2)ccc1Nc1ncc2c(n1)N(C)c1ccccc1C(=O)N2C ; "CF3COOH"]

4-(4-((4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, trifluoroacetic acid. ¹H NMR (400 MHz, DMSO-d₆): δ 10.21 (s, 1H), 9.70 (s, 1H), 9.58-9.56 (m, 1H), 8.31 (s, 1H), 7.86 (s, 1H), 7.80-7.78 (m, 1H), 7.68-7.66 (m, 1H), 7.51-7.47 (m, 1H), 7.34-7.14 (m, 6H), 6.61 (s, 2H), 6.51-6.48 (m, 1H), 6.31 (s, 1H), 4.66-4.64 (m, 1H), 4.39-4.36 (m, 1H), 4.10-3.95 (m, 3H), 3.67-3.64 (m, 2H), 3.48 (s, 2H), 3.38 (s, 3H), 3.30-3.28 (m, 5H), 3.02-2.98 (m, 1H), 2.87-2.68 (m, 3H), 2.38-2.33 (m, 6H), 2.25-2.21 (m, 2H), 2.12-2.11 (m, 2H), 1.77-1.68 (m, 7H), 1.33-1.28 (m, 7H), 0.88-0.84 (m, 5H). LCMS (ESI): R_{T} 1.140 min, m/z. found 1100.3 [M-CF₃COOH+H]⁺.

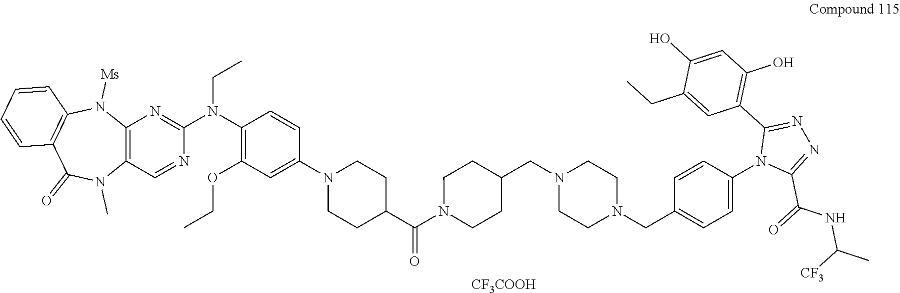 [SMILES: CCOc1cc(N2CCC(C(=O)N3CCC(CN4CCN(Cc5ccc(-n6c(C(=O)NC(C)C(F)(F)F)nnc6-c6cc(CC)c(O)cc6O)cc5)CC4)CC3)CC2)ccc1N(CC)c1ncc2c(n1)N(S(C)(=O)=O)c1ccccc1C(=O)N2C ; "CF3COOH"]

4-(4-((4-((1-(1-(3-ethoxy-4-(ethyl(5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, trifluoroacetic acid. ¹H NMR (400 MHz, DMSO-d₆): δ 10.20 (s, 1H), 9.70 (s, 1H), 9.58-9.56 (m, 1H), 8.66 (s, 1H), 7.72-7.68 (m, 2H), 7.49-7.41 (m, 3H), 7.32-7.25 (m, 4H), 7.05-6.98 (m, 1H), 6.62-6.59 (m, 3H), 6.31 (s, 1H), 4.66-4.64 (m, 1H), 4.37-4.36 (m, 1H), 3.97-3.75 (m, 9H), 3.48-3.41 (m, 6H), 3.30-3.22 (m, 2H), 3.05-2.95 (m, 1H), 2.82-2.76 (m, 3H), 2.45-2.38 (m, 5H), 2.27-2.21 (m, 5H), 1.77-1.71 (m, 8H), 1.14-1.07 (m, 5H), 0.88-0.73 (m, 5H). LCMS (ESI): R_{T}=1.229 min, m/z. found 1190.1 [M-CF₃COOH—H]⁻.

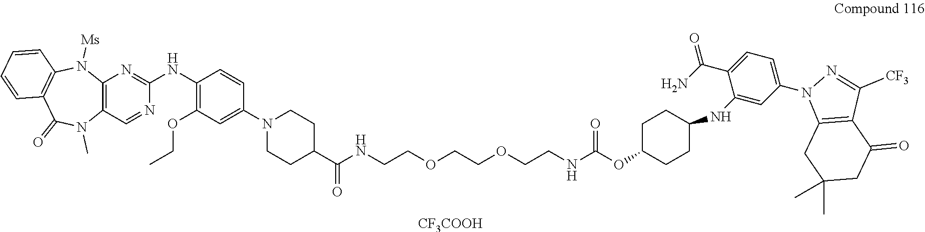 [SMILES: CCOc1cc(N2CCC(C(=O)NCCOCCOCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(S(C)(=O)=O)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl (2-(2-(2-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamido)ethoxy)ethoxy)ethyl)carbamate, trifluoroacetic acid. ¹H NMR (400 MHz, DMSO-d₆): δ 8.76 (s, 1H), 8.60 (s, 1H), 8.43 (d, J=7.2 Hz, 1H), 8.07-7.70 (m, 4H), 7.62-7.54 (m, 2H), 7.48-7.22 (m, 3H), 7.13-7.06 (m, 1H), 6.89 (d, J=1.6 Hz, 1H), 6.73 (dd, J=8.4, 1.6 Hz, 1H), 6.59 (d, J=2.0 Hz, 1H), 6.51-6.45 (m, 1H), 4.58-4.48 (m, 1H), 4.07-3.94 (m, 2H), 3.75-3.63 (m, 5H), 3.50 (s, 4H), 3.45 (s, 3H), 3.42-3.38 (m, 4H), 3.32-3.30 (m, 1H), 3.25-3.17 (m, 2H), 3.14-3.08 (m, 2H), 2.98 (s, 2H), 2.69-2.59 (m, 2H), 2.44 (s, 2H), 2.34-2.22 (m, 1H), 2.07-1.87 (m, 4H), 1.78-1.60 (m, 4H), 1.54-1.43 (m, 2H), 1.38-1.25 (m, 2H), 1.15 (t, J=6.8 Hz, 3H), 1.03 (s, 6H). LCMS (ESI): R_{T}=1.415 min, m/z. found 1187.0 [M-CF₃COOH+H]⁺.

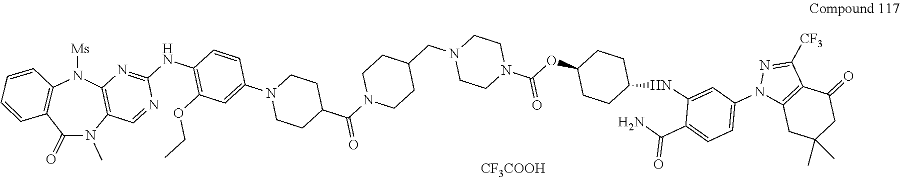 [SMILES: CCOc1cc(N2CCC(C(=O)N3CCC(CN4CCN(C(=O)O[C@H]5CC[C@H](Nc6cc(-n7nc(C(F)(F)F)c8c7CC(C)(C)CC8=O)ccc6C(N)=O)CC5)CC4)CC3)CC2)ccc1Nc1ncc2c(n1)N(S(C)(=O)=O)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl 4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazine-1-carboxylate, trifluoroacetic acid. ¹H NMR (400 MHz, DMSO-d₆): δ 9.47 (s, 1H), 8.85 (s, 1H), 8.63 (s, 1H), 8.50 (s, 1H), 8.03 (s, 1H), 7.83-7.73 (m, 2H), 7.59 (d, J=3.6 Hz, 2H), 7.51-7.30 (m, 3H), 6.89 (d, J=1.6 Hz, 1H), 6.78-6.62 (m, 2H), 4.68-4.60 (m, 1H), 4.45-4.37 (m, 1H), 4.07-4.00 (m, 6H), 3.74-3.66 (m, 6H), 3.54-3.44 (m, 7H), 3.30-3.18 (m, 2H), 3.06-2.97 (m, 6H), 2.93-2.81 (m, 2H), 2.64-2.54 (m, 1H), 2.45 (s, 2H), 2.08-1.91 (m, 5H), 1.82-1.68 (m, 6H), 1.61-1.51 (m, 2H), 1.44-1.33 (m, 2H), 1.21-1.15 (m, 3H), 1.04 (s, 6H). LCMS (ESI): R_{T}=1.345 min, m/z. found 1222.6 [M-CF₃COOH+H]⁺.

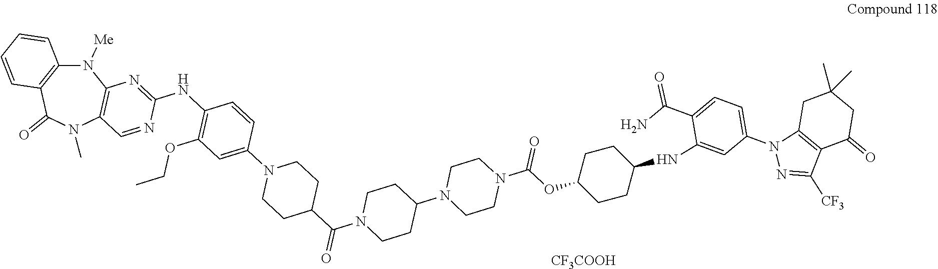 [SMILES: CCOc1cc(N2CCC(C(=O)N3CCC(N4CCN(C(=O)O[C@H]5CC[C@H](Nc6cc(-n7nc(C(F)(F)F)c8c7CC(C)(C)CC8=O)ccc6C(N)=O)CC5)CC4)CC3)CC2)ccc1Nc1ncc2c(n1)N(C)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl 4-(1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)piperazine-1-carboxylate, trifluoroacetic acid. ¹H NMR (400 MHz, DMSO-d₆): δ 8.76 (s, 1H), 8.60 (s, 1H), 8.48 (d, J=7.6 Hz, 1H), 8.00 (s, 1H), 7.82-7.72 (m, 2H), 7.617-0.54 (m, 2H), 7.50-7.23 (m, 3H), 6.88 (d, J=2.0 Hz, 1H), 6.73 (dd, J=8.4, 1.6 Hz, 1H), 6.59 (d, J=1.6 Hz, 1H), 6.48 (dd, J=8.8, 2.0 Hz, 1H), 4.63-4.53 (m, 1H), 4.46-4.37 (m, 1H), 4.09-3.96 (m, 3H), 3.75-3.63 (m, 5H), 3.45 (s, 4H), 3.33-3.31 (m, 6H), 3.08-2.92 (m, 3H), 2.84-2.68 (m, 3H), 2.45 (s, 7H), 2.06-1.88 (m, 4H), 1.84-1.62 (m, 6H), 1.57-1.47 (m, 2H), 1.40-1.28 (m, 3H), 1.15 (t, J=6.8 Hz, 3H), 1.03 (s, 6H). LCMS (ESI): R_{T}=1.318 min, m/z. found 1208.6 [M-CF₃COOH+H]⁺.

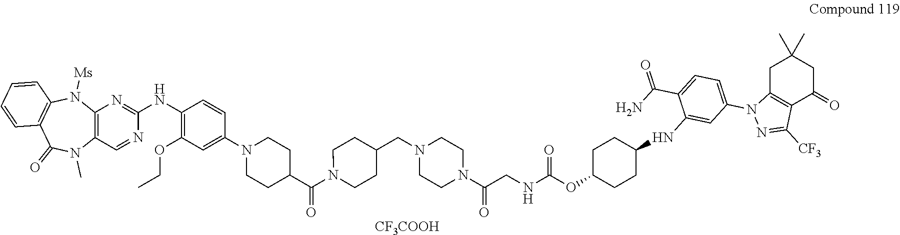 [SMILES: CCOc1cc(N2CCC(C(=O)N3CCC(CN4CCN(C(=O)CNC(=O)O[C@H]5CC[C@H](Nc6cc(-n7nc(C(F)(F)F)c8c7CC(C)(C)CC8=O)ccc6C(N)=O)CC5)CC4)CC3)CC2)ccc1Nc1ncc2c(n1)N(S(C)(=O)=O)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl (2-(4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)-2-oxoethyl)carbamate, trifluoroacetic acid. ¹H NMR (400 MHz, DMSO-d₆): δ 8.70 (s, 1H), 8.60 (s, 1H), 8.42 (d, J=7.6 Hz, 1H), 7.97 (s, 1H), 7.82-7.72 (m, 2H), 7.58 (d, J=4.0 Hz, 2H), 7.48-7.42 (m, 1H), 7.36-7.22 (m, 2H), 7.03-6.96 (m, 1H), 6.89 (s, 1H), 6.72 (d, J=8.0 Hz, 1H), 6.59 (s, 1H), 6.49 (d, J=8.4 Hz, 1H), 4.58-4.50 (m, 1H), 4.43-4.33 (m, 1H), 4.07-3.94 (m, 3H), 3.85-3.78 (m, 2H), 3.73-3.62 (m, 5H), 3.50-3.36 (m, 8H), 3.08-2.94 (m, 3H), 2.82-2.68 (m, 3H), 2.59-2.52 (m, 1H), 2.45 (s, 2H), 2.38-2.26 (m, 4H), 2.17-1.90 (m, 6H), 1.81-1.64 (m, 7H), 1.56-1.46 (m, 2H), 1.39-1.29 (m, 2H), 1.18-1.13 (m, 3H), 1.04 (s, 6H), 0.98-0.80 (m, 2H). LCMS (ESI): R_{T}=1.298 min, m/z. found 1279.6 [M-CF₃COOH+H]⁺.

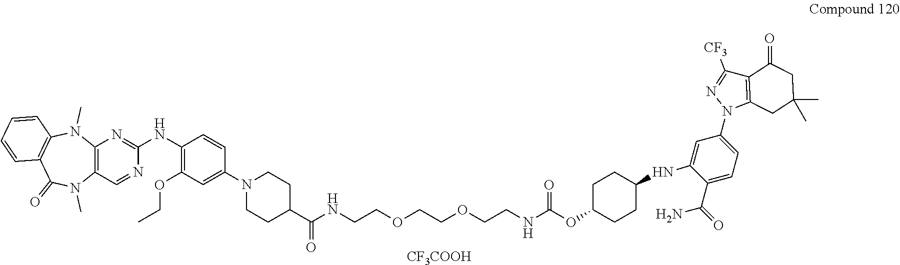 [SMILES: CCOc1cc(N2CCC(C(=O)NCCOCCOCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(C)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl (2-(2-(2-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamido)ethoxy)ethoxy)ethyl)carbamate, trifluoroacetic acid. ¹H NMR (400 MHz, DMSO-d₆): δ 8.42 (d, J=7.2 Hz, 1H), 8.30 (s, 1H), 7.97 (s, 1H), 7.85-7.66 (m, 4H), 7.67 (d, J=8.6 Hz, 1H), 7.48 (t, J=8.4 Hz, 1H), 7.31 (s, 1H), 7.23-7.13 (m, 2H), 7.04 (t, J=6.0 Hz, 1H), 6.88 (s, 1H), 6.75-6.69 (m, 1H), 6.61 (d, J=2.0 Hz, 1H), 6.53-6.46 (m, 1H), 4.53 (s, 1H), 4.07 (q, J=7.2 Hz, 2H), 3.69-3.60 (m, 2H), 3.50 (s, 4H), 3.44-3.36 (m, 8H), 3.28 (s, 3H), 3.23-3.18 (m, 2H), 3.15-3.09 (m, 2H), 2.97 (s, 2H), 2.68-2.57 (m, 2H), 2.44 (s, 2H), 2.30-2.21 (m, 1H), 2.09-1.88 (m, 4H), 1.80-1.62 (m, 4H), 1.54-1.42 (m, 2H), 1.36-1.27 (m, 5H), 1.03 (s, 6H). LCMS (ESI): R_{T}=1.450 min, m/z. found 1123.4 [M-CF₃COOH+H]⁺.

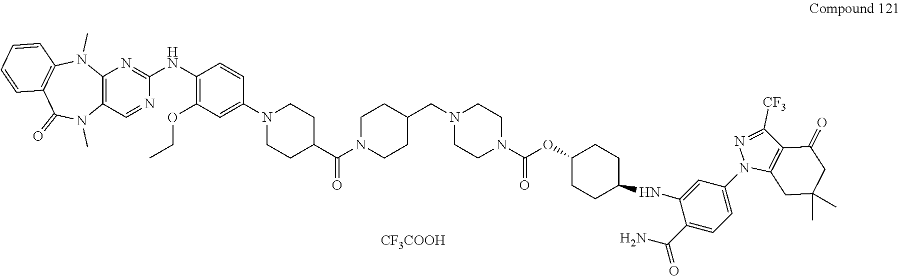 [SMILES: CCOc1cc(N2CCC(C(=O)N3CCC(CN4CCN(C(=O)O[C@H]5CC[C@H](Nc6cc(-n7nc(C(F)(F)F)c8c7CC(C)(C)CC8=O)ccc6C(N)=O)CC5)CC4)CC3)CC2)ccc1Nc1ncc2c(n1)N(C)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl 4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperazine-1-carboxylate, trifluoroacetic acid. ¹H NMR (400 MHz, DMSO-d₆): δ 9.44 (s, 1H), 8.49 (s, 1H), 8.35 (s, 1H), 8.02 (d, J=30.8 Hz, 3H), 7.80 (d, J=8.4 Hz, 1H), 7.69 (d, J=8.8 Hz, 1H), 7.54-7.48 (m, 1H), 7.34 (s, 1H), 7.25-7.16 (m, 2H), 6.92-6.71 (m, 3H), 4.64 (s, 1H), 4.41 (d, J=12.8 Hz, 1H), 4.14-4.09 (m, 2H), 4.05-4.00 (m, 2H), 3.70-3.67 (m, 3H), 3.49 (s, 3H), 3.39 (s, 3H), 3.31-3.20 (m, 6H), 3.15-2.88 (m, 10H), 2.65-2.55 (m, 1H), 2.45 (s, 2H), 2.16-1.93 (m, 6H), 1.79 (s, 6H), 1.62-1.53 (m, 2H), 1.43-1.24 (m, 5H), 1.04 (s, 6H). LCMS (ESI): R_{T}1.250 min, m/z. found 1158.3[M-CF₃COOH+H]⁺.

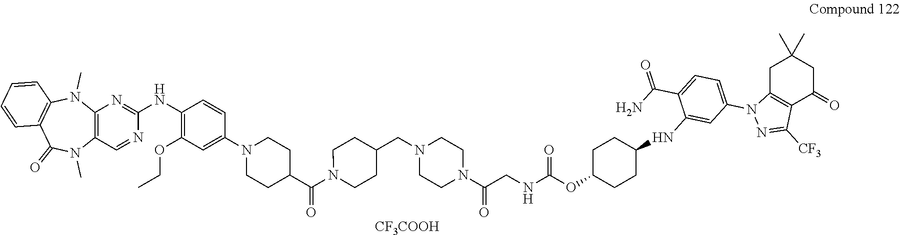 [SMILES: CCOc1cc(N2CCC(C(=O)N3CCC(CN4CCN(C(=O)CNC(=O)O[C@H]5CC[C@H](Nc6cc(-n7nc(C(F)(F)F)c8c7CC(C)(C)CC8=O)ccc6C(N)=O)CC5)CC4)CC3)CC2)ccc1Nc1ncc2c(n1)N(C)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl (2-(4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperazin-1-yl)-2-oxoethyl)carbamate, trifluoroacetic acid. ¹H NMR (400 MHz, DMSO-d₆): δ 8.43 (d, J=7.2 Hz, 1H), 8.32 (s, 1H), 8.00 (s, 1H), 7.86 (s, 1H), 7.79 (d, J=8.4 Hz, 2H), 7.67 (dd, J=7.7, 1.6 Hz, 1H), 7.51-7.45 (m, 1H), 7.35 (s, 1H), 7.24-7.14 (m, 2H), 7.04 (t, J=5.6 Hz, 1H), 6.90 (s, 1H), 6.73 (dd, J=9.2, 1.6 Hz, 1H), 6.61 (d, J=2.2 Hz, 1H), 6.50 (dd, J=8.8, 2.4 Hz, 1H), 4.59-4.48 (m, 1H), 4.39 (d, J=13.6 Hz, 1H), 4.10-3.94 (m, 3H), 3.81 (d, J=5.2 Hz, 2H), 3.66 (d, J=12.0 Hz, 2H), 3.47-3.38 (m, 8H), 3.28 (s, 4H), 3.07-2.96 (m, 3H), 2.79-2.67 (m, 3H), 2.45 (s, 2H), 2.31 (d, J=16.4 Hz, 4H), 2.17-1.90 (m, 6H), 1.82-1.63 (m, 8H), 1.57-1.47 (m, 2H), 1.37-1.28 (m, 5H), 1.04 (s, 7H). LCMS (ESI): R_{T}=1.330 min, m/z. found 1215.7 [M-CF₃COOH+H]⁺.

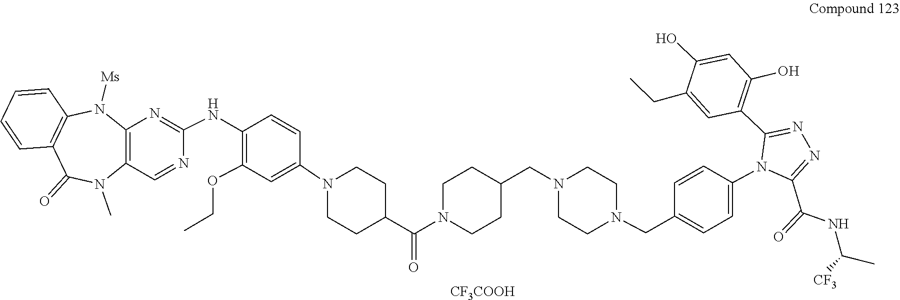 [SMILES: CCOc1cc(N2CCC(C(=O)N3CCC(CN4CCN(Cc5ccc(-n6c(C(=O)N[C@H](C)C(F)(F)F)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

(R)-4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, trifluoroacetic acid. ¹H NMR (400 MHz, DMSO-d₆): δ 9.73-9.61 (m, 2H), 8.85 (s, 1H), 8.64 (s, 1H), 7.75 (d, J=7.6 Hz, 1H), 7.59 (d, J=3.6 Hz, 2H), 7.49-7.32 (m, 6H), 6.84-6.63 (m, 2H), 6.30 (s, 1H), 4.68-4.60 (m, 1H), 4.44-4.36 (m, 2H), 4.33-4.14 (m, 6H), 4.08-0.399 (m, 4H), 3.69 (s, 5H), 3.46 (s, 3H), 3.20-2.77 (m, 10H), 2.61-2.56 (m, 1H), 2.28 (q, J=7.6 Hz, 2H), 2.08-1.95 (m, 1H), 1.85-1.67 (m, 6H), 1.34 (d, J=7.0 Hz, 3H), 1.19 (t, J=6.8 Hz, 3H), 1.14-0.98 (m, 2H), 0.90 (t, J=7.5 Hz, 3H). LCMS (ESI): R_{T} 1.100 min, m/z. found 1164.3 [M-CF₃COOH+H]⁺.

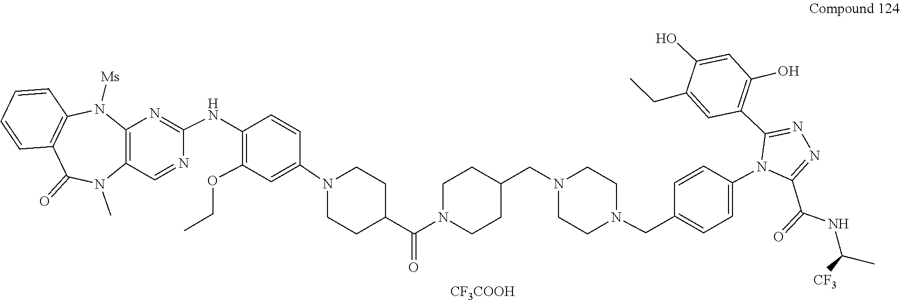 [SMILES: CCOc1cc(N2CCC(C(=O)N3CCC(CN4CCN(Cc5ccc(-n6c(C(=O)N[C@@H](C)C(F)(F)F)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

(S)-4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, trifluoroacetic acid. ¹H NMR (400 MHz, DMSO-d₆): δ 9.73-9.61 (m, 2H), 8.85 (s, 1H), 8.64 (s, 1H), 7.75 (d, J=7.6 Hz, 1H), 7.59 (d, J=3.6 Hz, 2H), 7.49-7.32 (m, 6H), 6.84-6.63 (m, 2H), 6.30 (s, 1H), 4.68-4.60 (m, 1H), 4.44-4.36 (m, 2H), 4.33-4.14 (m, 6H), 4.08-0.399 (m, 4H), 3.69 (s, 5H), 3.46 (s, 3H), 3.20-2.77 (m, 10H), 2.61-2.56 (m, 1H), 2.28 (q, J=7.6 Hz, 2H), 2.08-1.95 (m, 1H), 1.85-1.67 (m, 6H), 1.34 (d, J=7.0 Hz, 3H), 1.19 (t, J=6.8 Hz, 3H), 1.14-0.98 (m, 2H), 0.90 (t, J=7.5 Hz, 3H). LCMS (ESI): R_{T}=1.100 min, m/z. found 1164.3 [M-CF₃COOH+H]⁺.

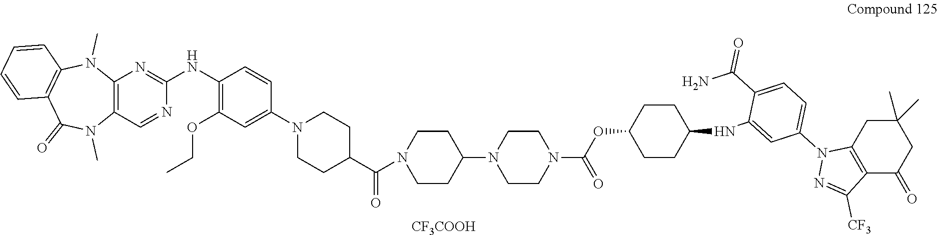 [SMILES: CCOc1cc(N2CCC(C(=O)N3CCC(N4CCN(C(=O)O[C@H]5CC[C@H](Nc6cc(-n7nc(C(F)(F)F)c8c7CC(C)(C)CC8=O)ccc6C(N)=O)CC5)CC4)CC3)CC2)ccc1Nc1ncc2c(n1)N(C)c1ccccc1C(=O)N2C ; "CF3COOH"]

(1R,4R)-4-((2-carbamoyl-5-(6,6-dimethyl-4-oxo-3-(trifluoromethyl)-4,5,6,7-tetrahydro-1H-indazol-1-yl)phenyl)amino)cyclohexyl 4-(1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)piperazine-1-carboxylate, trifluoroacetic acid. ¹H NMR (400 MHz, DMSO-d₆): δ 8.48 (d, J=8.0 Hz, 1H), 8.32 (s, 1H), 8.00 (s, 1H), 7.87-7.77 (m, 3H), 7.67 (dd, J=7.8, 2.0 Hz, 1H), 7.51-7.47 m, 1H), 7.35 (s, 1H), 7.24-7.14 (m, 2H), 6.88 (d, J=1.6 Hz, 1H), 6.73 (dd, J=8.4, 1.6 Hz, 1H), 6.61 (d, J=2.4 Hz, 1H), 6.49 (dd, J=8.8, 2.0 Hz, 1H), 4.58 (s, 1H), 4.42 (d, J=13.0 Hz, 1H), 4.13-3.99 (m, 3H), 3.66 (d, J=12.4 Hz, 2H), 3.52-3.43 (m, 1H), 3.38 (s, 4H), 3.30 (s, 2H), 3.28 (s, 4H), 3.03-2.93 (m, 3H), 2.81-2.65 (m, 3H), 2.45 (s, 7H), 2.02-1.87 (m, 4H), 1.84-1.62 (m, 6H), 1.58-1.47 (m, 2H), 1.38-1.18 (m, 7H), 1.03 (s, 6H), 0.98-0.91 (m, 1H). LCMS (ESI): R_{T}=1.180 min, m/z. found 1144.4 [M-CF₃COOH+H]⁺.

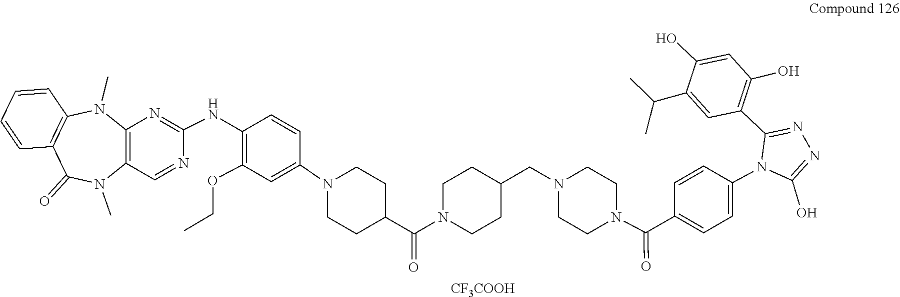 [SMILES: CCOc1cc(N2CCC(C(=O)N3CCC(CN4CCN(C(=O)c5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; "CF3COOH"]

2-((4-(4-(4-((4-(4-(3-(2,4-dihydroxy-5-isopropylphenyl)-5-hydroxy-4H-1,2,4-triazol-4-yl)benzoyl)piperazin-1-yl)methyl)piperidine-1-carbonyl)piperidin-1-yl)-2-ethoxyphenyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.97 (s, 1H), 9.60 (s, 1H), 9.37 (s, 1H), 8.31 (s, 1H), 7.86-7.78 (m, 2H), 7.78-7.66 (m, 1H), 7.49-7.38 (m, 3H), 7.24-7.14 (m, 4H), 6.88 (s, 1H), 6.61-6.60 (m, 1H), 6.50-6.48 (m, 1H), 6.26 (s, 1H), 4.40-4.37 (m, 1H), 4.08-4.05 (m, 3H), 3.67-3.61 (m, 4H), 3.28 (m, 4H), 3.02-2.99 (m, 2H), 2.74-2.70 (m, 3H), 2.51-2.31 (m, 4H), 2.16 (m, 2H), 1.79-1.64 (m, 7H), 1.31-1.24 (m, 4H), 1.01-1.00 (m, 7H), 0.98-0.85 (m, 1H). LCMS (ESI): R_{T}=1.040 min, m/z. found 1005.3 [M-CF₃COOH+H]⁺.

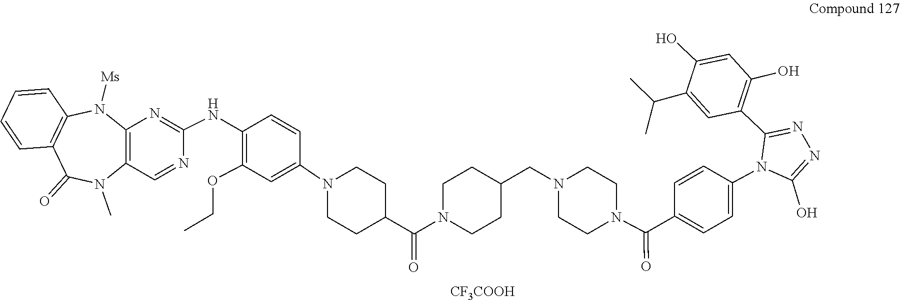 [SMILES: CCOc1cc(N2CCC(C(=O)N3CCC(CN4CCN(C(=O)c5ccc(-n6c(O)nnc6-c6cc(C(C)C)c(O)cc6O)cc5)CC4)CC3)CC2)ccc1Nc1ncc2c(n1)N(S(C)(=O)=O)c1ccccc1C(=O)N2C ; "CF3COOH"]

2-((4-(4-(4-((4-(4-(3-(2,4-dihydroxy-5-isopropylphenyl)-5-hydroxy-4H-1,2,4-triazol-4-yl)benzoyl)piperazin-1-yl)methyl)piperidine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.97 (s, 1H), 9.60 (s, 1H), 9.37 (s, 1H), 8.74 (s, 1H), 8.60 (s, 1H), 7.74 (d, J=7.7 Hz, 1H), 7.58 (d, J=3.5 Hz, 2H), 7.49-7.33 (m, 3H), 7.24 (m, 8.7 Hz, 3H), 6.87 (s, 1H), 6.59 (s, 1H), 6.48 (d, J=8.6 Hz, 1H), 6.25 (s, 1H), 4.37 (s, 1H), 4.07-3.92 (m, 3H), 3.68 (s, 7H), 3.45 (s, 3H), 3.31-3.23 (m, 3H), 3.07-2.96 (m, 2H), 2.75 (s, 3H), 2.33 (s, 4H), 2.16 (s, 2H), 1.71 (m, 7H), 1.15 (t, J=6.9 Hz, 3H), 1.00 (d, J=6.9 Hz, 6H), 0.96-0.78 (m, 2H). LCMS (ESI): R_{T}=1.020 min, m/z. found 1069.3 [M-CF₃COOH+H]⁺.

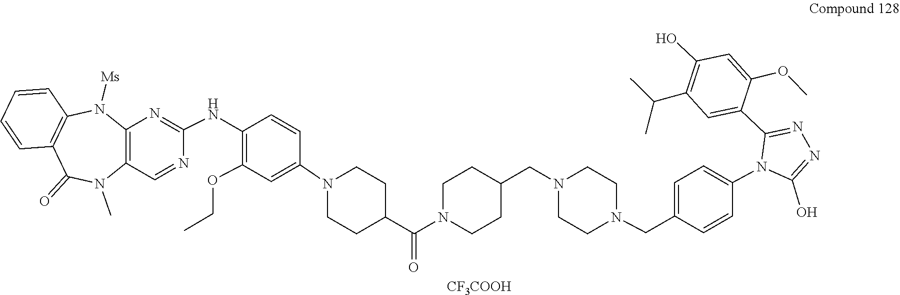 [SMILES: CCOc1cc(N2CCC(C(=O)N3CCC(CN4CCN(Cc5ccc(-n6c(O)nnc6-c6cc(C(C)C)c(O)cc6OC)cc5)CC4)CC3)CC2)ccc1Nc1ncc2c(n1)N(S(C)(=O)=O)c1ccccc1C(=O)N2C ; "CF3COOH"]

2-((2-ethoxy-4-(4-(4-((4-(4-(3-hydroxy-5-(4-hydroxy-5-isopropyl-2-methoxyphenyl)-4H-1,2,4-triazol-4-yl)benzyl)piperazin-1-yl)methyl)piperidine-1-carbonyl)piperidin-1-yl)phenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆): δ 11.95 (s, 1H), 9.83 (s, 1H), 8.82 (s, 1H), 8.63 (s, 1H), 7.75 (d, J=7.8 Hz, 1H), 7.59 (d, J=3.7 Hz, 2H), 7.51-7.31 (m, 4H), 7.11 (d, J=6.1 Hz, 3H), 6.69 (m, 2H), 6.30 (s, 1H), 4.39 (d, J=12.4 Hz, 1H), 4.10-4.00 (m, 8H), 3.69 (s, 7H), 3.46 (s, 3H), 3.21 (s, 3H), 3.12-2.84 (m, 10H), 1.95 (s, 1H), 1.74 (d, J=8.7 Hz, 6H), 1.28-1.03 (m, 12H). LCMS (ESI): R_{T}=1.040 min, m/z. found 1069.3 [M-CF₃COOH+H]⁺.

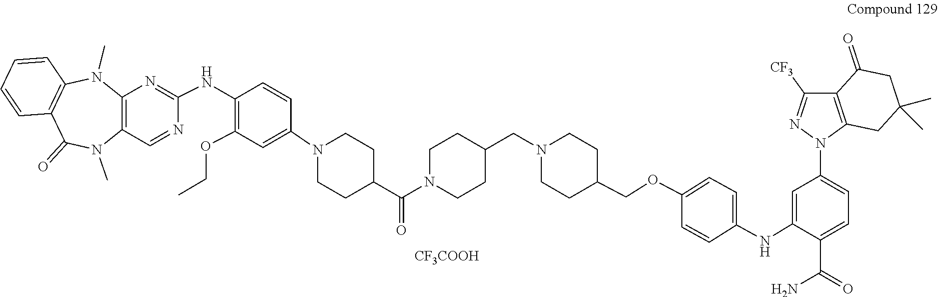 [SMILES: CCOc1cc(N2CCC(C(=O)N3CCC(CN4CCC(COc5ccc(Nc6cc(-n7nc(C(F)(F)F)c8c7CC(C)(C)CC8=O)ccc6C(N)=O)cc5)CC4)CC3)CC2)ccc1Nc1ncc2c(n1)N(C)c1ccccc1C(=O)N2C ; "CF3COOH"]

4-(6,6-dimethyl-4-oxo-3-(trifluoromethyl)-4,5,6,7-tetrahydro-1H-indazol-1-yl)-2-((4-((1-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperidin-4-yl)methoxy)phenyl)amino)benzamide, trifluoroacetic acid. ¹H NMR (400 MHz, DMSO-d₆): δ 10.11 (s, 1H), 9.02 (s, 1H), 8.37 (s, 1H), 8.21-8.02 (m, 3H), 7.91 (d, J=8.8 Hz, 1H), 7.70-7.49 (m, 3H), 7.26-7.16 (m, 4H), 7.03-6.93 (m, 5H), 4.52-4.36 (m, 2H), 4.16-4.10 (m, 3H), 4.05-3.97 (m, 2H), 3.87 (d, J=5.2 Hz, 2H), 3.69-3.56 (m, 4H), 3.39-3.32 (m, 7H), 3.15-3.03 (m, 2H), 3.01-2.87 (m, 6H), 2.61-2.53 (m, 1H), 2.43 (s, 2H), 2.17-1.95 (m, 3H), 1.94-1.70 (m, 6H), 1.68-1.57 (m, 2H), 1.35 (t, J=6.8 Hz, 3H), 1.03 (s, 8H). LCMS (ESI): R_{T}=1.176 min, m/z. found 1137.3 [M-CF₃COOH+H]⁺.

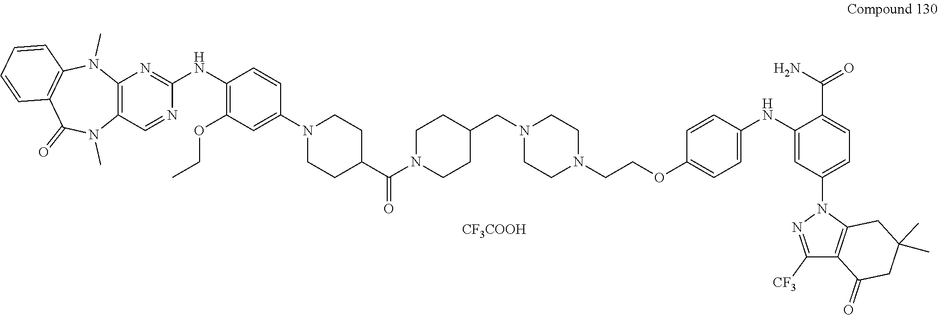 [SMILES: CCOc1cc(N2CCC(C(=O)N3CCC(CN4CCN(CCOc5ccc(Nc6cc(-n7nc(C(F)(F)F)c8c7CC(C)(C)CC8=O)ccc6C(N)=O)cc5)CC4)CC3)CC2)ccc1Nc1ncc2c(n1)N(C)c1ccccc1C(=O)N2C ; "CF3COOH"]

4-(6,6-dimethyl-4-oxo-3-(trifluoromethyl)-4,5,6,7-tetrahydro-1H-indazol-1-yl)-2-((4-(2-(4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperazin-1-yl)ethoxy)phenyl)amino)benzamide, trifluoroacetic acid. ¹H NMR (400 MHz, DMSO-d₆): δ 10.14 (s, 1H), 8.37 (s, 1H), 8.25-8.00 (m, 3H), 7.91 (d, J=8.8 Hz, 1H), 7.70-7.49 (m, 3H), 7.28-7.16 (m, 4H), 7.06-6.94 (m, 7H), 4.82-4.51 (m, 5H), 4.42-4.39 (m, 2H), 4.25 (s, 2H), 4.16-4.11 (m, 2H), 3.99 (d, J=2.0 Hz, 1H), 3.68-3.64 (m, 2H), 3.40-3.32 (m, 10H), 3.24-3.03 (m, 3H), 2.97-2.89 (m, 4H), 2.61-2.54 (m, 1H), 2.43 (s, 3H), 2.02-1.63 (m, 8H), 1.35 (t, J=6.8 Hz, 3H), 1.04 (s, 7H). LCMS (ESI): R_{T}=1.151 min, m/z. found 1152.3 [M-CF₃COOH+H]⁺.

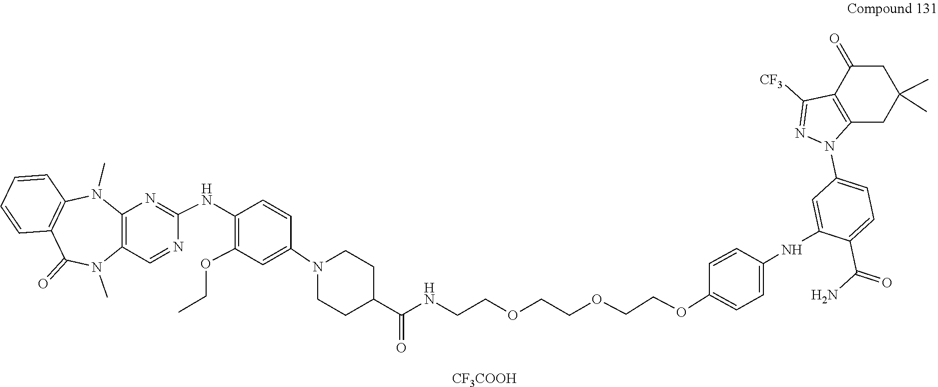 [SMILES: CCOc1cc(N2CCC(C(=O)NCCOCCOCCOc3ccc(Nc4cc(-n5nc(C(F)(F)F)c6c5CC(C)(C)CC6=O)ccc4C(N)=O)cc3)CC2)ccc1Nc1ncc2c(n1)N(C)c1ccccc1C(=O)N2C ; "CF3COOH"]

N-(2-(2-(2-(4-((2-carbamoyl-5-(6,6-dimethyl-4-oxo-3-(trifluoromethyl)-4,5,6,7-tetrahydro-1H-indazol-1-yl)phenyl)amino)phenoxy)ethoxy)ethoxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 10.12 (s, 1H), 8.38 (s, 1H), 8.20-8.06 (m, 4H), 7.90 (d, J=8.4 Hz, 1H), 7.70-7.49 (m, 3H), 7.26-6.93 (m, 10H), 4.16-4.07 (m, 4H), 3.76-3.55 (m, 8H), 3.46-3.22 (m, 12H), 2.90 (s, 2H), 2.42 (s, 3H), 1.91 (s, 4H), 1.35 (t, J=6.8 Hz, 3H), 1.03 (s, 7H). LCMS (ESI): R_{T}=1.443 min, m/z. found 1074.3 [M-CF₃COOH+H]⁺.

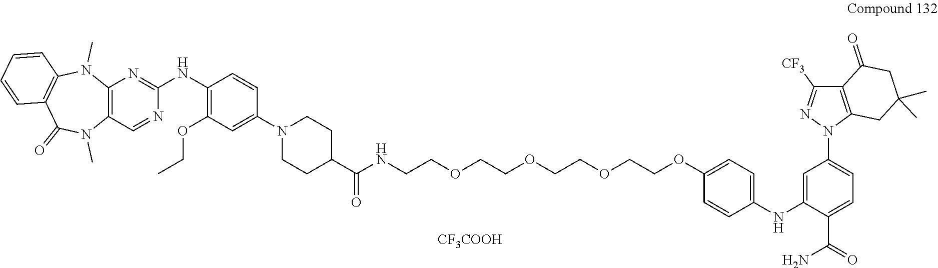 [SMILES: CCOc1cc(N2CCC(C(=O)NCCOCCOCCOCCOc3ccc(Nc4cc(-n5nc(C(F)(F)F)c6c5CC(C)(C)CC6=O)ccc4C(N)=O)cc3)CC2)ccc1Nc1ncc2c(n1)N(C)c1ccccc1C(=O)N2C ; "CF3COOH"]

N-(2-(2-(2-(2-(4-((2-carbamoyl-5-(6,6-dimethyl-4-oxo-3-(trifluoromethyl)-4,5,6,7-tetrahydro-1H-indazol-1-yl)phenyl)amino)phenoxy)ethoxy)ethoxy)ethoxy)ethyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆): δ 10.12 (s, 1H), 8.37 (s, 1H), 8.20-8.05 (m, 4H), 7.90 (d, J=8.4 Hz, 1H), 7.70-7.49 (m, 3H), 7.26-6.93 (m, 10H), 4.16-4.08 (m, 4H), 3.75-3.53 (m, 12H), 3.44-3.21 (m, 12H), 2.90 (s, 2H), 2.42 (s, 3H), 1.91 (s, 4H), 1.35 (t, J=6.8 Hz, 3H), 1.03 (s, 7H). LCMS (ESI): R_{T}=1.443 min, m/z. found 1118.3 [M-CF₃COOH+H]⁺.

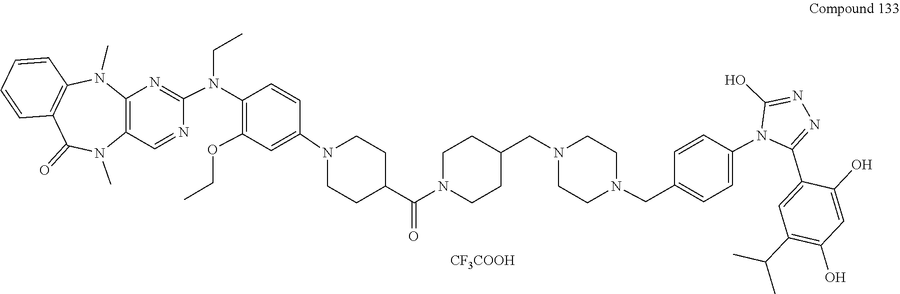 [SMILES: CCOc1cc(N2CCC(C(=O)N3CCC(CN4CCN(Cc5ccc(-n6c(O)nnc6-c6cc(C(C)C)c(O)cc6O)cc5)CC4)CC3)CC2)ccc1N(CC)c1ncc2c(n1)N(C)c1ccccc1C(=O)N2C ; "CF3COOH"]

2-((4-(4-(4-((4-(4-(3-(2,4-dihydroxy-5-isopropylphenyl)-5-hydroxy-4H-1,2,4-triazol-4-yl)benzyl)piperazin-1-yl)methyl)piperidine-1-carbonyl)piperidin-1-yl)-2-ethoxyphenyl)(ethyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 8.12 (s, 1H), 7.65 (d, J=6.8 Hz, 1H), 7.48 (s, 1H), 7.36-7.24 (m, 3H), 7.19-7.09 (m, 4H), 6.93 (s, 1H), 6.69-6.55 (m, 2H), 6.48 (s, 1H), 6.25 (s, 1H), 4.42-4.34 (m, 1H), 4.05-3.86 (m, 5H), 3.78-3.68 (m, 3H), 3.48-3.39 (m, 7H), 3.08-2.88 (m, 2H), 2.82-2.70 (m, 4H), 2.44-2.25 (m, 7H), 2.16-2.07 (m, 2H), 1.82-1.60 (m, 8H), 1.23-0.96 (m, 8H), 0.87 (d, J=6.8 Hz, 8H). LCMS (ESI): R_{T}=1.266 min, m/z. found 1019.9 [M-CF₃COOH+H]⁺.

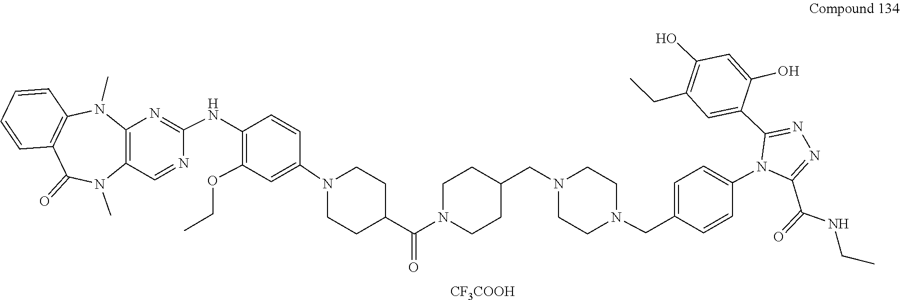 [SMILES: CCNC(=O)c1nnc(-c2cc(CC)c(O)cc2O)n1-c1ccc(CN2CCN(CC3CCN(C(=O)C4CCN(c5ccc(Nc6ncc7c(n6)N(C)c6ccccc6C(=O)N7C)c(OCC)c5)CC4)CC3)CC2)cc1 ; "CF3COOH"]

4-(4-((4-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperazin-1-yl)methyl)phenyl)-N-ethyl-5-(5-ethyl-2,4-dihydroxyphenyl)-4H-1,2,4-triazole-3-carboxamide, trifluoroacetic acid. ¹H NMR (400 MHz, DMSO-d₆): δ 10.38 (s, 1H), 9.70 (s, 1H), 8.99-8.92 (m, 1H), 8.31 (s, 1H), 7.86 (s, 1H), 7.79 (d, J=8.8 Hz, 1H), 7.67 (dd, J=8.0, 1.6 Hz, 1H), 7.53-7.46 (m, 1H), 7.34 (d, J=8.0 Hz, 2H), 7.29-7.21 (m, 3H), 7.19-7.12 (m, 1H), 6.61 (d, J=2.4 Hz, 1H), 6.54 (s, 1H), 6.52-6.47 (m, 1H), 6.32 (s, 1H), 4.42-4.34 (m, 1H), 4.11-4.04 (m, 2H), 4.01-3.93 (m, 1H), 3.70-3.61 (m, 2H), 3.49 (s, 2H), 3.38 (s, 3H), 3.30-3.25 (m, 5H), 3.21-3.13 (m, 2H), 3.08-2.95 (m, 1H), 2.78-2.65 (m, 3H), 2.59-2.51 (m, 2H), 2.44-2.29 (m, 7H), 2.24-2.18 (m, 2H), 2.16-2.09 (m, 2H), 1.80-1.64 (m, 7H), 1.32-1.27 (m, 3H), 1.04 (t, J=7.2 Hz, 3H), 0.85-0.81 (m, 3H). LCMS (ESI): R_{T}1.051 min, m/z. found 1032.3 [M-CF₃COOH+H]⁺.

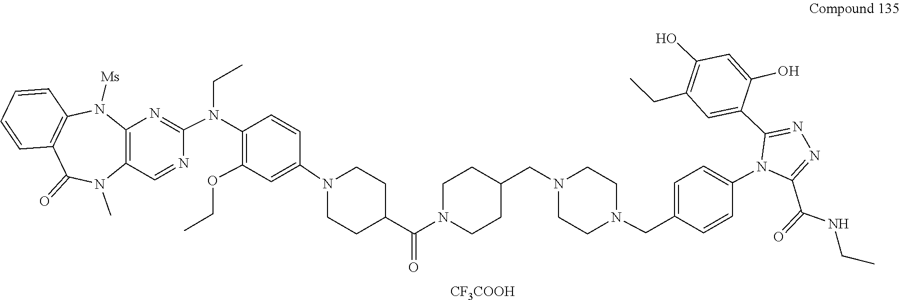 [SMILES: CCNC(=O)c1nnc(-c2cc(CC)c(O)cc2O)n1-c1ccc(CN2CCN(CC3CCN(C(=O)C4CCN(c5ccc(N(CC)c6ncc7c(n6)N(S(C)(=O)=O)c6ccccc6C(=O)N7C)c(OCC)c5)CC4)CC3)CC2)cc1 ; "CF3COOH"]

4-(4-((4-((1-(1-(3-ethoxy-4-(ethyl(5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-N-ethyl-5-(5-ethyl-2,4-dihydroxyphenyl)-4H-1,2,4-triazole-3-carboxamide, trifluoroacetic acid. ¹H NMR (400 MHz, DMSO-d₆): δ 9.71 (s, 1H), 9.04-8.96 (m, 1H), 8.74-8.44 (m, 1H), 7.72 (s, 1H), 7.65-7.20 (m, 8H), 7.16-6.94 (m, 1H), 6.72-6.52 (m, 3H), 6.31 (s, 1H), 4.42-4.38 (m, 1H), 4.02-3.74 (m, 10H), 3.44 (s, 5H), 3.24-3.13 (m, 5H), 3.12-2.76 (m, 11H), 2.63-2.53 (m, 2H), 2.29-2.22 (m, 2H), 2.11-1.91 (m, 1H), 1.82-1.62 (m, 7H), 1.14-1.03 (m, 9H), 0.90-0.84 (m, 3H). LCMS (ESI): R_{T}=1.138 min, m/z. found 1124.3 [M-CF₃COOH+H]⁺.

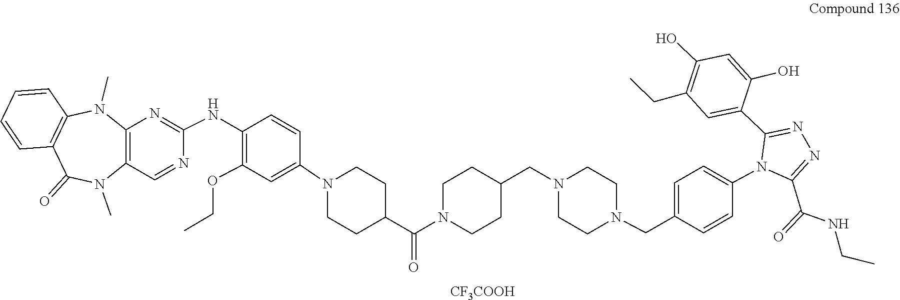 [SMILES: CCNC(=O)c1nnc(-c2cc(CC)c(O)cc2O)n1-c1ccc(CN2CCN(CC3CCN(C(=O)C4CCN(c5ccc(Nc6ncc7c(n6)N(C)c6ccccc6C(=O)N7C)c(OCC)c5)CC4)CC3)CC2)cc1 ; "CF3COOH"]

4-(4-((4-((1-(1-(4-((5,11-dimethyl-6-oxo-6,11-dihydro-5H-benzo[e]pyrimido[5,4-b][1,4]diazepin-2-yl)(ethyl)amino)-3-ethoxyphenyl)piperidine-4-carbonyl)piperidin-4-yl)methyl)piperazin-1-yl)methyl)phenyl)-N-ethyl-5-(5-ethyl-2,4-dihydroxyphenyl)-4H-1,2,4-triazole-3-carboxamide, trifluoroacetic acid. ¹H NMR (400 MHz, DMSO-d₆): δ 9.73 (s, 1H), 9.00 (t, J=5.6 Hz, 1H), 8.17 (s, 1H), 7.67 (d, J=7.2 Hz, 1H), 7.59-7.33 (m, 6H), 7.27-7.13 (m, 2H), 7.06 (d, J=7.2 Hz, 1H), 6.74 (s, 1H), 6.63 (s, 2H), 6.31 (s, 1H), 4.40 (d, J=13.6 Hz, 1H), 4.13-3.66 (m, 11H), 3.34 (s, 5H), 3.21-3.14 (m, 3H), 3.12-2.80 (m, 12H), 2.63-2.54 (m, 2H), 2.26 (q, J=7.4 Hz, 2H), 2.00 (s, 1H), 1.85-1.68 (m, 7H), 1.18-1.04 (m, 9H), 0.87 (t, J=7.4 Hz, 3H). LCMS (ESI): R_{T}=1.160 min, m/z. found 1060.4 [M-CF₃COOH+H]⁺.

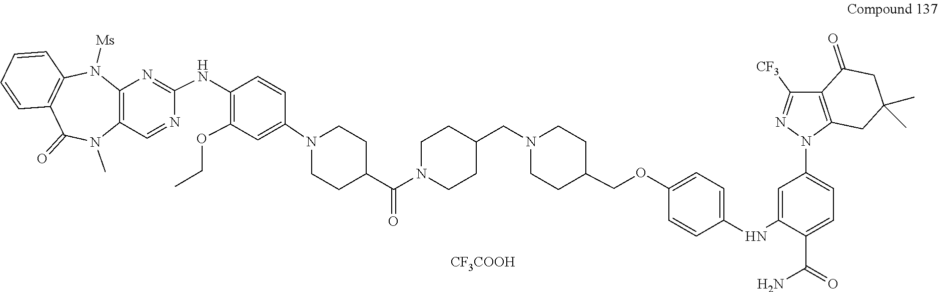 [SMILES: CCOc1cc(N2CCC(C(=O)N3CCC(CN4CCC(COc5ccc(Nc6cc(-n7nc(C(F)(F)F)c8c7CC(C)(C)CC8=O)ccc6C(N)=O)cc5)CC4)CC3)CC2)ccc1Nc1ncc2c(n1)N(S(C)(=O)=O)c1ccccc1C(=O)N2C ; "CF3COOH"]

4-(6,6-dimethyl-4-oxo-3-(trifluoromethyl)-4,5,6,7-tetrahydro-1H-indazol-1-yl)-2-((4-((1-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperidin-4yl)methoxy)phenyl)amino)benzamide, trifluoroacetic acid. ¹H NMR (400 MHz, DMSO-d₆): δ 10.12 (s, 1H), 9.01 (s, 1H), 8.85 (s, 1H), 8.64 (s, 1H), 8.21 (s, 1H), 7.91 (d, J=8.4 Hz, 1H), 7.76 (d, J=7.8 Hz, 1H), 7.65-7.53 (m, 3H), 7.51-7.38 (m, 2H), 7.25 (d, J=8.8 Hz, 2H), 7.05-6.93 (m, 4H), 6.83-6.59 (m, 2H), 4.42 (d, J=14.0 Hz, 1H), 4.09-3.99 (m, 4H), 3.87 (d, J=5.6 Hz, 2H), 3.70 (s, 4H), 3.57 (s, 2H), 3.46 (s, 3H), 3.32-3.21 (m, 1H), 3.16-3.03 (m, 2H), 3.04-2.96 (m, 3H), 2.92 (s, 3H), 2.68-2.55 (m, 1H), 2.43 (s, 4H), 2.15-1.94 (m, 4H), 1.86-1.70 (m, 5H), 1.67-1.56 (m, 2H), 1.23-1.13 (m, 4H), 1.03 (s, 6H). LCMS (ESI): R_{T}=1.300 min, m/z. found 1201.6 [M-CF₃COOH+H]⁺.

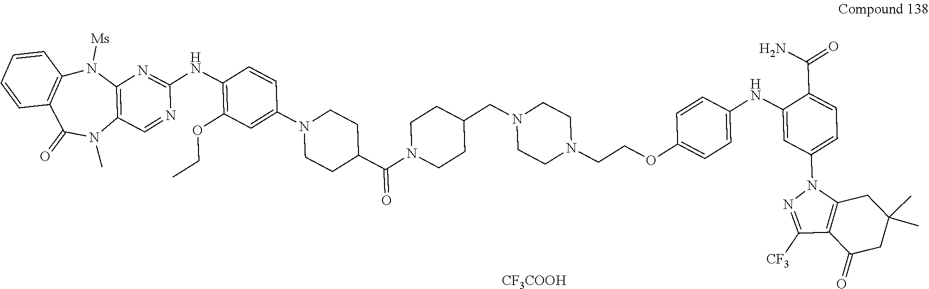 [SMILES: CCOc1cc(N2CCC(C(=O)N3CCC(CN4CCN(CCOc5ccc(Nc6cc(-n7nc(C(F)(F)F)c8c7CC(C)(C)CC8=O)ccc6C(N)=O)cc5)CC4)CC3)CC2)ccc1Nc1ncc2c(n1)N(S(C)(=O)=O)c1ccccc1C(=O)N2C ; "CF3COOH"]

4-(6,6-dimethyl-4-oxo-3-(trifluoromethyl)-4,5,6,7-tetrahydro-1H-indazol-1-yl)-2-((4-(2-(4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)ethoxy)phenyl)amino)benzamide, trifluoroacetic acid. ¹H NMR (400 MHz, DMSO-d₆): δ 10.15 (s, 1H), 8.90 (s, 1H), 8.65 (s, 1H), 8.22 (s, 1H), 7.91 (d, J=8.5 Hz, 1H), 7.76 (d, J=7.7 Hz, 1H), 7.69-7.39 (m, 5H), 7.27 (d, J=8.8 Hz, 2H), 7.08-6.72 (m, 6H), 4.41 (d, J=12.6 Hz, 1H), 4.26 (s, 2H), 4.05 (m, 4H), 3.70 (s, 5H), 3.47 (s, 3H), 3.34 (s, 5H), 3.09 (m, 5H), 2.93 (s, 7H), 2.57 (t, J=12.3 Hz, 1H), 2.43 (s, 2H), 1.99 (s, 1H), 1.79 (s, 6H), 1.26-1.09 (m, 4H), 1.04 (s, 7H). LCMS (ESI): R_{T}1.270 min, m/z. found 1216.6 [M-CF₃COOH+H]⁺.

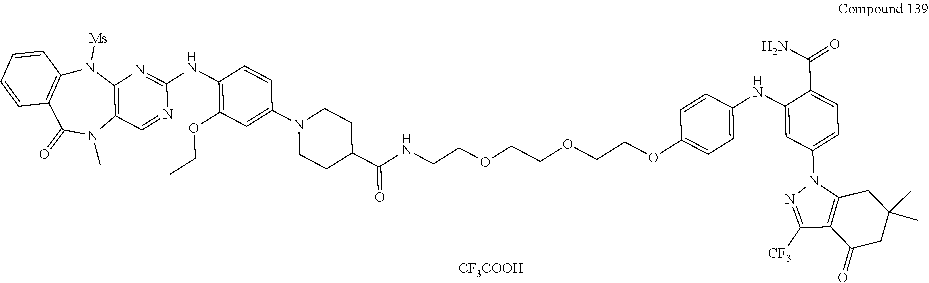 [SMILES: CCOc1cc(N2CCC(C(=O)NCCOCCOCCOc3ccc(Nc4cc(-n5nc(C(F)(F)F)c6c5CC(C)(C)CC6=O)ccc4C(N)=O)cc3)CC2)ccc1Nc1ncc2c(n1)N(S(C)(=O)=O)c1ccccc1C(=O)N2C ; "CF3COOH"]

N-(2-(2-(2-(4-((2-carbamoyl-5-(6,6-dimethyl-4-oxo-3-(trifluoromethyl)-4,5,6,7-tetrahydro-1H-indazol-1-yl)phenyl)amino)phenoxy)ethoxy)ethoxy)ethyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 10.11 (s, 1H), 8.86 (s, 1H), 8.64 (s, 1H), 8.20 (s, 1H), 7.99 (s, 1H), 7.90 (d, J=8.5 Hz, 1H), 7.76 (d, J=7.7 Hz, 1H), 7.59 (d, J=3.8 Hz, 3H), 7.47 (m, 1H), 7.23 (d, J=8.8 Hz, 2H), 7.05-6.92 (m, 4H), 6.86-6.71 (m, 1H), 4.11-4.00 (m, 7H), 3.79-3.63 (m, 8H), 3.62-3.53 (m, 4H), 3.48-3.40 (m, 5H), 3.26-3.20 (m, 2H), 2.90 (s, 2H), 2.42 (s, 3H), 1.83 (s, 4H), 1.21 (m, 3H), 1.03 (s, 6H). LCMS (ESI): R_{T}=1.555 min, m/z. found 1138.6 [M-CF₃COOH+H]⁺.

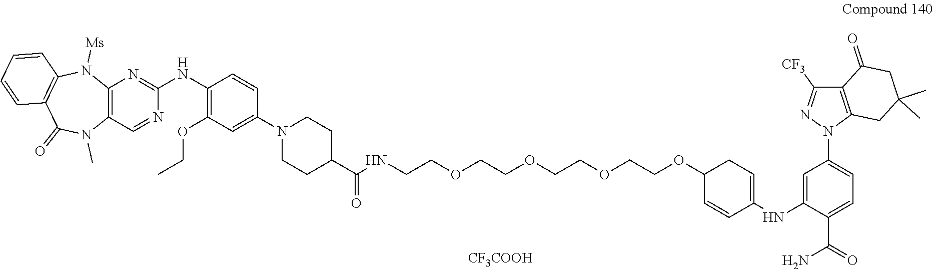 [SMILES: CCOc1cc(N2CCC(C(=O)NCCOCCOCCOCCOC3C=CC(Nc4cc(-n5nc(C(F)(F)F)c6c5CC(C)(C)CC6=O)ccc4C(N)=O)=CC3)CC2)ccc1Nc1ncc2c(n1)N(S(C)(=O)=O)c1ccccc1C(=O)N2C ; "CF3COOH"]

N-(2-(2-(2-(2-(4-((2-carbamoyl-5-(6,6-dimethyl-4-oxo-3-(trifluoromethyl)-4,5,6,7-tetrahydro-1H-indazol-1-yl)phenyl)amino)phenoxy)ethoxy)ethoxy)ethoxy)ethyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 10.11 (s, 1H), 8.88 (s, 1H), 8.65 (s, 1H), 8.20 (s, 1H), 7.99 (s, 1H), 7.90 (d, J=8.5 Hz, 1H), 7.76 (d, J=7.7 Hz, 1H), 7.68-7.40 (m, 5H), 7.23 (d, J=8.8 Hz, 2H), 7.05-6.69 (m, 6H), 4.11-4.03 (m, 4H), 3.79-3.64 (m, 7H), 3.62-3.37 (m, 14H), 3.29-3.19 (m, 2H), 3.02 (s, 1H), 2.91 (s, 2H), 2.42 (s, 3H), 1.83 (s, 4H), 1.20 (t, J=6.7 Hz, 3H), 1.03 (s, 6H). LCMS (ESI): R_{T}=1.555 min, m/z. found 1182.5 [M-CF₃COOH+H]⁺.

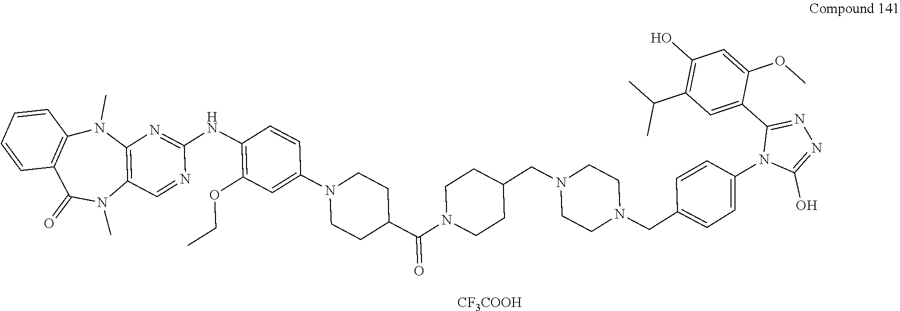 [SMILES: CCOc1cc(N2CCC(C(=O)N3CCC(CN4CCN(Cc5ccc(-n6c(O)nnc6-c6cc(C(C)C)c(O)cc6OC)cc5)CC4)CC3)CC2)ccc1Nc1ncc2c(n1)N(C)c1ccccc1C(=O)N2C ; "CF3COOH"]

2-((2-ethoxy-4-(4-(4-((4-(4-(3-hydroxy-5-(4-hydroxy-5-isopropyl-2-methoxyphenyl)-4H-1,2,4-triazol-4-yl)benzyl)piperazin-1-yl)methyl)piperidine-1-carbonyl)piperidin-1-yl)phenyl)(ethyl)amino)-5,11-dimethyl-5,11-dihydro-6H-benzo[e]pyrimido[5,4-b][1,4]diazepin-6-one, trifluoroacetic acid. ¹H NMR (400 MHz, DMSO-d₆): δ 11.90 (s, 1H), 9.76 (s, 1H), 8.08 (s, 1H), 7.65 (d, J=7.6 Hz, 1H), 7.52-7.39 (m, 1H), 7.26-6.89 (m, 8H), 6.57-6.46 (m, 2H), 6.28 (s, 1H), 4.39-4.35 (m, 1H), 4.01-3.71 (m, 6H), 3.41 (s, 2H), 3.32-3.23 (m, 5H), 3.17 (s, 3H), 3.11-2.97 (m, 2H), 2.82-2.76 (m, 4H), 2.67-2.51 (m, 4H), 2.33 (s, 7H), 2.25-2.10 (m, 2H), 2.02-1.92 (m, 1H), 1.75-1.67 (m, 7H), 1.09-1.00 (m, 10H), 0.98-0.87 (m, 3H). LCMS (ESI): R_{T}=5.062 min, m/z. found 1033.3 [M-CF₃COOH+H]⁺.

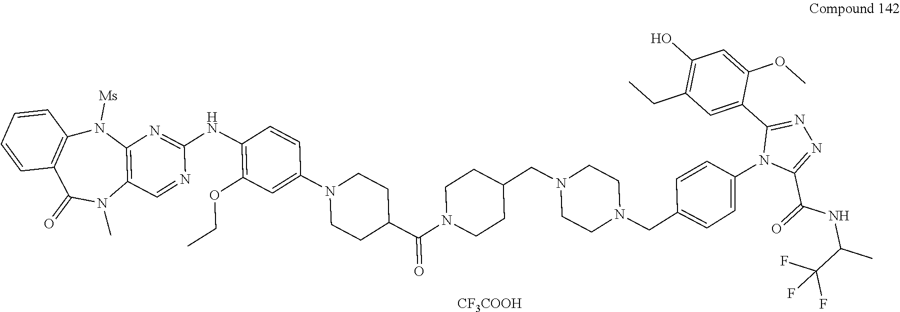 [SMILES: CCOc1cc(N2CCC(C(=O)N3CCC(CN4CCN(Cc5ccc(-n6c(C(=O)NC(C)C(F)(F)F)nnc6-c6cc(CC)c(O)cc6OC)cc5)CC4)CC3)CC2)ccc1Nc1ncc2c(n1)N(S(C)(=O)=O)c1ccccc1C(=O)N2C ; "CF3COOH"]

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-4-hydroxy-2-methoxyphenyl)-N-(1,1,1-trifluoropropan-2-yl)-4H-1,2,4-triazole-3-carboxamide, trifluoroacetic acid. ¹H NMR (400 MHz, DMSO-d₆): δ 9.87 (s, 1H), 9.64 (d, J=8.8 Hz, 1H), 8.86 (s, 1H), 8.64 (s, 1H), 7.75 (d, J=7.6 Hz, 1H), 7.60 (d, J=3.6 Hz, 2H), 7.47-7.45 (m, 2H), 7.35 (s, 2H), 7.20-7.00 (m, 4H), 6.82-6.68 (m, 1H), 6.31 (s, 1H), 4.62-4.55 (m, 2H), 4.38-4.26 (m, 2H), 4.06-3.96 (m, 3H), 3.70 (s, 6H), 3.46 (s, 3H), 3.30 (s, 3H), 3.21-2.58 (m, 11H), 2.45-2.41 (m, 5H), 2.11-1.52 (m, 8H), 1.35 (d, J=7.2 Hz, 3H), 1.21-1.17 (m, 5H), 1.08-1.04 (m, 3H). LCMS (ESI): R_{T}=1.085 min, m/z. found 1178.3 [M-CF₃COOH+H]⁺.

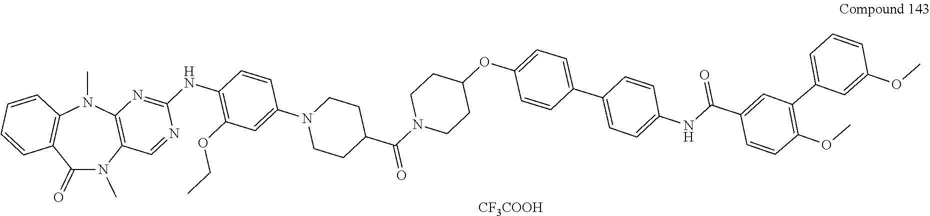 [SMILES: CCOc1cc(N2CCC(C(=O)N3CCC(Oc4ccc(-c5ccc(NC(=O)c6ccc(OC)c(-c7cccc(OC)c7)c6)cc5)cc4)CC3)CC2)ccc1Nc1ncc2c(n1)N(C)c1ccccc1C(=O)N2C ; "CF3COOH"]

N-(4′-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)oxy)-[1,1′-biphenyl]-4-yl)-3′,6-dimethoxy-[1,1′-biphenyl]-3-carboxamide, trifluoroacetic acid. ¹H NMR (400 MHz, DMSO-d₆): δ 10.22 (s, 1H), 8.39 (s, 1H), 8.25-7.95 (m, 4H), 7.85 (d, J=8.4 Hz, 2H), 7.73-7.49 (m, 6H), 7.40-6.95 (m, 11H), 4.75-4.66 (m, 2H), 4.19-4.11 (m, 2H), 3.94-3.78 (m, 8H), 3.71-3.63 (m, 2H), 3.54-3.45 (m, 1H), 3.40 (s, 4H), 3.33 (s, 4H), 3.09-2.95 (m, 1H), 2.10-1.85 (m, 6H), 1.74-1.50 (m, 2H), 1.37 (t, J=6.8 Hz, 3H), 1.27-1.21 (m, 1H). LCMS (ESI): R_{T} 1.778 min, m/z. found 993.1 [M-CF₃COOH+H]⁺.

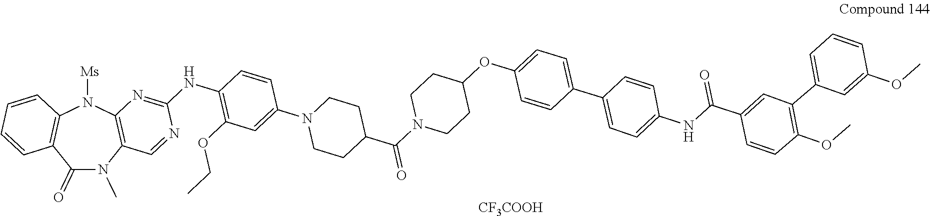 [SMILES: CCOc1cc(N2CCC(C(=O)N3CCC(Oc4ccc(-c5ccc(NC(=O)c6ccc(OC)c(-c7cccc(OC)c7)c6)cc5)cc4)CC3)CC2)ccc1Nc1ncc2c(n1)N(S(C)(=O)=O)c1ccccc1C(=O)N2C ; "CF3COOH"]

N-(4′-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)oxy)-[1,1′-biphenyl]-4-yl)-3′,6-dimethoxy-[1,1′-biphenyl]-3-carboxamide, trifluoroacetic acid. ¹H NMR (400 MHz, DMSO-d₆): δ 10.21 (s, 1H), 8.88 (s, 1H), 8.65 (s, 1H), 8.05-7.96 (m, 2H), 7.88-7.73 (m, 3H), 7.65-7.58 (m, 6H), 7.49-6.94 (m, 9H), 4.74-4.66 (m, 1H), 4.08-4.03 (m, 2H), 3.91-3.78 (m, 14H), 3.73-3.67 (m, 5H), 3.52-3.43 (m, 4H), 3.34-3.26 (m, 1H), 2.98-2.88 (m, 1H), 2.10-1.51 (m, 8H), 1.25-1.15 (m, 3H). LCMS (ESI): R_{T}=1.752 min, m/z. found 1057.1 [M-CF₃COOH+H]⁺.

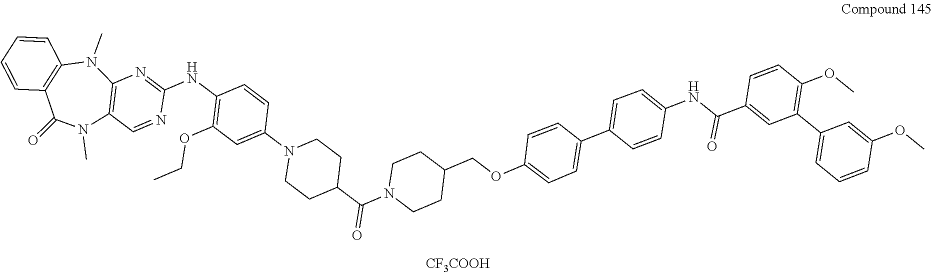 [SMILES: CCOc1cc(N2CCC(C(=O)N3CCC(COc4ccc(-c5ccc(NC(=O)c6ccc(OC)c(-c7cccc(OC)c7)c6)cc5)cc4)CC3)CC2)ccc1Nc1ncc2c(n1)N(C)c1ccccc1C(=O)N2C ; "CF3COOH"]

N-(4′-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methoxy)-[1,1′-biphenyl]-4-yl)-3′,6-dimethoxy-[1,1′-biphenyl]-3-carboxamide, trifluoroacetic acid. ¹H NMR (400 MHz, DMSO-d₆): δ 10.21 (s, 1H), 8.37 (s, 1H), 8.14 (s, 1H), 8.04-7.97 (m, 2H), 7.84 (d, J=8.8 Hz, 2H), 7.70-7.49 (m, 6H), 7.42-6.90 (m, 11H), 4.51-4.43 (m, 1H), 4.20-4.10 (m, 4H), 4.10-4.00 (m, 3H), 3.94-3.89 (m, 3H), 3.87 (s, 3H), 3.80 (s, 3H), 3.71-3.63 (m, 3H), 3.39 (s, 3H), 3.32 (s, 3H), 3.16-2.95 (m, 2H), 2.68-2.57 (m, 1H), 2.12-2.00 (m, 1H), 1.96-1.76 (m, 5H), 1.40-1.32 (m, 3H), 1.27-1.13 (m, 2H). LCMS (ESI): R_{T}=1.858 min, m/z. found 1008.1 [M-CF₃COOH+H]⁺.

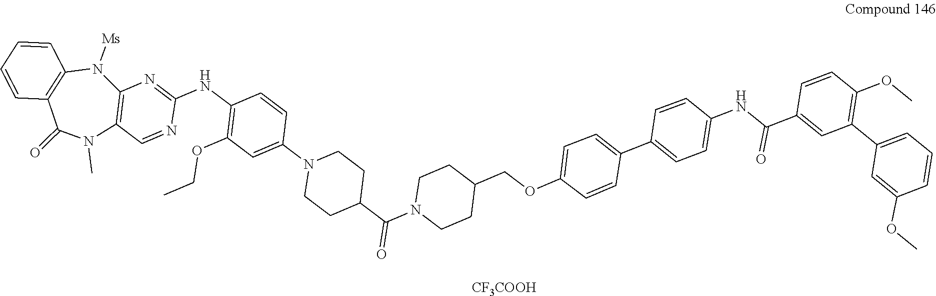 [SMILES: CCOc1cc(N2CCC(C(=O)N3CCC(COc4ccc(-c5ccc(NC(=O)c6ccc(OC)c(-c7cccc(OC)c7)c6)cc5)cc4)CC3)CC2)ccc1Nc1ncc2c(n1)N(S(C)(=O)=O)c1ccccc1C(=O)N2C ; "CF3COOH"]

N-(4′-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methoxy)-[1,1′-biphenyl]-4-yl)-3′,6-dimethoxy-[1,1′-biphenyl]-3-carboxamide, trifluoroacetic acid. ¹H NMR (400 MHz, DMSO-d₆): δ 10.21 (s, 1H), 8.89 (s, 1H), 8.65 (s, 1H), 8.06-7.97 (m, 2H), 7.87-7.74 (m, 3H), 7.64-7.57 (m, 6H), 7.53-6.88 (m, 10H), 4.52-4.44 (m, 1H), 4.11-4.03 (m, 3H), 3.93-3.89 (m, 2H), 3.87 (s, 4H), 3.80 (s, 5H), 3.71 (s, 5H), 3.47 (s, 3H), 3.17-3.07 (m, 1H), 2.96-2.89 (m, 1H), 2.65-2.57 (m, 1H), 2.11-2.02 (m, 1H), 1.93-1.74 (m, 6H), 1.27-1.14 (m, 6H). LCMS (ESI): R_{T}=1.763 min, m/z. found 1071.2 [M-CF₃COOH+H]⁺.

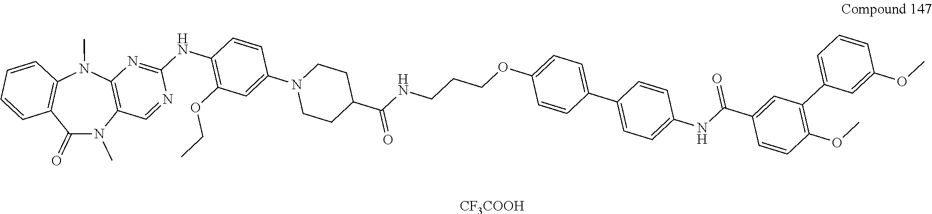 [SMILES: CCOc1cc(N2CCC(C(=O)NCCCOc3ccc(-c4ccc(NC(=O)c5ccc(OC)c(-c6cccc(OC)c6)c5)cc4)cc3)CC2)ccc1Nc1ncc2c(n1)N(C)c1ccccc1C(=O)N2C ; "CF3COOH"]

N-(3-((4′-(3′,6-dimethoxy-[1,1′-biphenyl]-3-carboxamido)-[1,1′-biphenyl]-4-yl)oxy)propyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆) δ 10.21 (s, 1H), 8.37 (s, 1H), 8.23-7.92 (m, 5H), 7.84 (d, J=8.6 Hz, 2H), 7.68 (d, J=7.8 Hz, 1H), 7.61 (d, J=8.6 Hz, 4H), 7.51 (t, J=7.7 Hz, 1H), 7.37 (t, J=7.9 Hz, 1H), 7.26 (dd, J=8.6, 5.0 Hz, 2H), 7.21-7.05 (m, 4H), 7.04-6.91 (m, 4H), 5.17-4.24 (m, 3H), 4.18-4.07 (m, 2H), 4.07-3.98 (m, 2H), 3.83 (d, J=24.8 Hz, 6H), 3.67 (d, J=11.7 Hz, 2H), 3.39 (s, 3H), 3.32 (s, 3H), 3.29-3.20 (m, 3H), 1.98-1.77 (m, 6H), 1.35 (t, J=6.8 Hz, 3H). LCMS (ESI): R_{T}=1.637 min, m/z. found 967.2 [M-CF₃COOH+H]⁺.

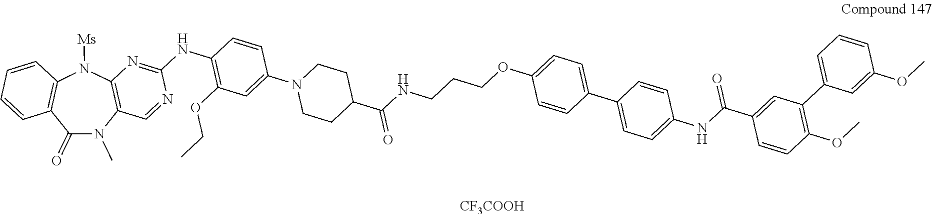 [SMILES: CCOc1cc(N2CCC(C(=O)NCCCOc3ccc(-c4ccc(NC(=O)c5ccc(OC)c(-c6cccc(OC)c6)c5)cc4)cc3)CC2)ccc1Nc1ncc2c(n1)N(S(C)(=O)=O)c1ccccc1C(=O)N2C ; "CF3COOH"]

N-(3-((4′-(3′,6-dimethoxy-[1,1′-biphenyl]-3-carboxamido)-[1,1′-biphenyl]-4-yl)oxy)propyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆) δ 10.21 (s, 1H), 8.92 (s, 1H), 8.66 (s, 1H), 8.11-7.96 (m, 3H), 7.84 (d, J=8.6 Hz, 2H), 7.76 (d, J=7.9 Hz, 1H), 7.61 (d, J=8.4 Hz, 7H), 7.54-7.41 (m, 2H), 7.37 (t, J=8.1 Hz, 1H), 7.26 (d, J=8.5 Hz, 1H), 7.16-6.92 (m, 6H), 4.18-4.01 (m, 9H), 3.83 (d, J=24.8 Hz, 6H), 3.73-3.66 (m, 4H), 3.47 (s, 3H), 3.29-3.23 (m, 2H), 1.99-1.77 (m, 6H), 1.27-1.17 (m, 3H). LCMS (ESI): R_{T}=1.750 min, m/z. found 1032.0 [M-CF₃COOH+H]⁺.

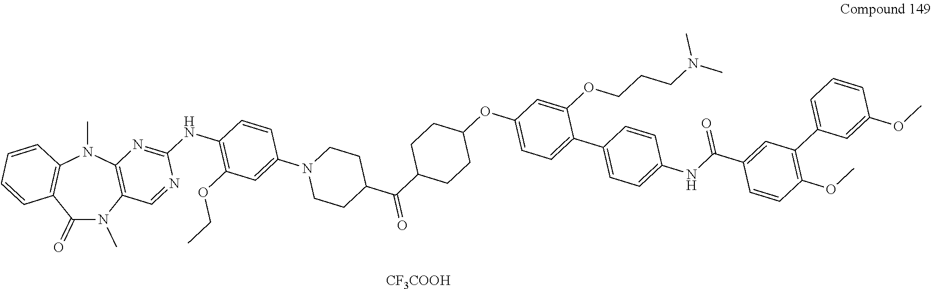 [SMILES: CCOc1cc(N2CCC(C(=O)C3CCC(Oc4ccc(-c5ccc(NC(=O)c6ccc(OC)c(-c7cccc(OC)c7)c6)cc5)c(OCCCN(C)C)c4)CC3)CC2)ccc1Nc1ncc2c(n1)N(C)c1ccccc1C(=O)N2C ; "CF3COOH"]

N-(4′-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)oxy)-2′-(3-(dimethylamino)propoxy)-[1,1′-biphenyl]-4-yl)-3′,6-dimethoxy-[1,1′-biphenyl]-3-carboxamide, trifluoroacetic acid. ¹H NMR (400 MHz, DMSO-d₆) δ 10.21 (s, 1H), 9.40 (s, 1H), 8.36 (s, 1H), 8.17-7.94 (m, 4H), 7.80 (d, J=7.9 Hz, 2H), 7.69 (d, J=7.6 Hz, 1H), 7.55-7.09 (m, 11H), 6.97 (d, J=8.7 Hz, 1H), 6.78-6.67 (m, 2H), 4.71 (s, 1H), 4.16-4.03 (m, 7H), 3.84 (d, J=25.2 Hz, 9H), 3.71-3.65 (m, 2H), 3.53-3.45 (m, 1H), 3.39 (s, 3H), 3.31 (s, 4H), 3.15-3.09 (m, 2H), 2.99-2.90 (m, 1H), 2.77 (d, J=4.1 Hz, 6H), 2.11-2.01 (m, 3H), 1.98-1.92 (m, 1H), 1.83 (s, 3H), 1.72-1.63 (m, 1H), 1.56 (s, 1H), 1.39-1.31 (m, 3H). LCMS (ESI): R_{T}=1.390 min, m/z. found 1095.1 [M-CF₃COOH+H]⁺.

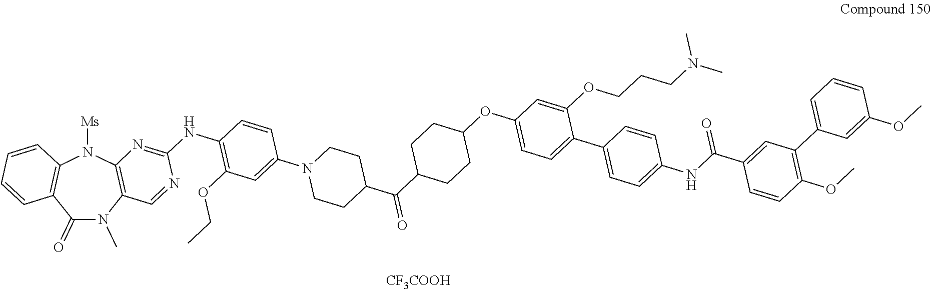 [SMILES: CCOc1cc(N2CCC(C(=O)C3CCC(Oc4ccc(-c5ccc(NC(=O)c6ccc(OC)c(-c7cccc(OC)c7)c6)cc5)c(OCCCN(C)C)c4)CC3)CC2)ccc1Nc1ncc2c(n1)N(S(C)(=O)=O)c1ccccc1C(=O)N2C ; "CF3COOH"]

N-(2′-(3-(dimethylamino)propoxy)-4′-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)oxy)-[1,1′-biphenyl]-4-yl)-3′,6-dimethoxy-[1,1′-biphenyl]-3-carboxamide, trifluoroacetic acid. ¹H NMR (400 MHz, DMSO-d₆) δ 10.21 (s, 1H), 9.43 (s, 1H), 8.85 (s, 1H), 8.63 (s, 1H), 8.05-7.94 (m, 2H), 7.78 (dd, J=18.6, 7.7 Hz, 3H), 7.60 (s, 2H), 7.46 (d, J=8.2 Hz, 3H), 7.38 (t, J=7.7 Hz, 2H), 7.29-7.24 (m, 2H), 7.14-7.08 (m, 2H), 6.96 (d, J=8.4 Hz, 1H), 6.80-6.59 (m, 4H), 4.71 (s, 1H), 4.09-3.99 (m, 5H), 3.84 (d, J=25.2 Hz, 8H), 3.75-3.64 (m, 6H), 3.46 (s, 4H), 3.31 (s, 1H), 3.16-3.07 (m, 2H), 2.91 (s, 1H), 2.77 (d, J=4.3 Hz, 6H), 2.09-1.91 (m, 5H), 1.78 (s, 4H), 1.70-1.63 (m, 1H), 1.56 (s, 1H), 1.22-1.15 (m, 3H). LCMS (ESI): R_{T} 1.390 min, m/z. found 1158.8 [M-CF₃COOH+H]⁺.

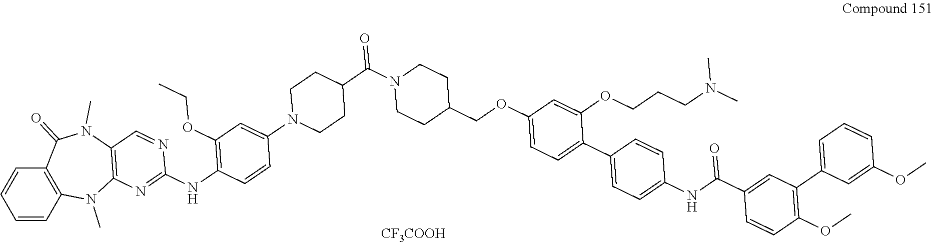 [SMILES: CCOc1cc(N2CCC(C(=O)N3CCC(COc4ccc(-c5ccc(NC(=O)c6ccc(OC)c(-c7cccc(OC)c7)c6)cc5)c(OCCCN(C)C)c4)CC3)CC2)ccc1Nc1ncc2c(n1)N(C)c1ccccc1C(=O)N2C ; "CF3COOH"]

N-(4′-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methoxy)-2′-(3-(dimethylamino)propoxy)-[1,1′-biphenyl]-4-yl)-3′,6-dimethoxy-[1,1′-biphenyl]-3-carboxamide, trifluoroacetic acid. ¹H NMR (400 MHz, DMSO-d₆) δ 10.21 (s, 1H), 9.44 (s, 1H), 8.38 (s, 1H), 8.19 (s, 2H), 8.02 (dd, J=8.6, 2.2 Hz, 1H), 7.97 (d, J=2.3 Hz, 1H), 7.80 (d, J=8.7 Hz, 2H), 7.69 (dd, J=7.8, 1.6 Hz, 1H), 7.55-7.49 (m, 1H), 7.45 (d, J=8.6 Hz, 2H), 7.38 (t, J=7.9 Hz, 1H), 7.29-7.23 (m, 3H), 7.19 (t, J=7.4 Hz, 1H), 7.15-7.06 (m, 3H), 6.98-6.94 (m, 1H), 6.69-6.62 (m, 2H), 4.48 (d, J=12.7 Hz, 1H), 4.15 (q, J=6.9 Hz, 2H), 4.05 (t, J=5.8 Hz, 3H), 3.91 (d, J=6.1 Hz, 2H), 3.87 (s, 3H), 3.81 (s, 3H), 3.67 (d, J=10.7 Hz, 2H), 3.40 (s, 3H), 3.33 (s, 3H), 3.16-3.07 (m, 3H), 3.00 (s, 1H), 2.77 (d, J=4.9 Hz, 6H), 2.63 (t, J=12.8 Hz, 1H), 2.11-2.01 (m, 3H), 1.95-1.78 (m, 6H), 1.42-1.05 (m, 7H). LCMS (ESI): R_{T}=1.280 min, m/z. found 1108.4 [M-CF₃COOH+H]⁺.

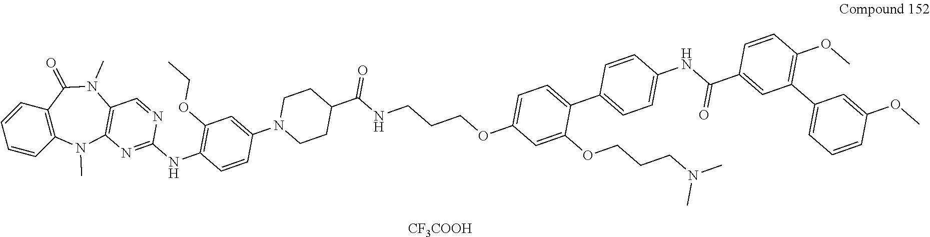 [SMILES: CCOc1cc(N2CCC(C(=O)NCCCOc3ccc(-c4ccc(NC(=O)c5ccc(OC)c(-c6cccc(OC)c6)c5)cc4)c(OCCCN(C)C)c3)CC2)ccc1Nc1ncc2c(n1)N(C)c1ccccc1C(=O)N2C ; "CF3COOH"]

N-(3-((4′-(3′,6-dimethoxy-[1,1′-biphenyl]-3-carboxamido)-2-(3-(dimethylamino)propoxy)-[1,1′-biphenyl]-4-yl)oxy)propyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. 1H NMR (400 MHz, DMSO-d₆) δ 10.21 (s, 1H), 9.44 (s, 1H), 8.37 (s, 1H), 8.21-7.93 (m, 5H), 7.80 (d, J=8.7 Hz, 2H), 7.69 (dd, J=7.7, 1.6 Hz, 1H), 7.54-7.08 (m, 11H), 7.02-6.82 (m, 2H), 6.69-6.61 (m, 2H), 4.17-4.00 (m, 6H), 3.84 (d, J=25.1 Hz, 6H), 3.67 (d, J=11.7 Hz, 2H), 3.39 (s, 3H), 3.32 (s, 3H), 3.28-3.24 (m, 2H), 3.17-3.03 (m, 3H), 2.76 (d, J=4.8 Hz, 6H), 2.43-2.35 (m, 1H), 2.10-2.02 (m, 2H), 1.95-1.79 (m, 6H), 1.38-1.21 (m, 4H). LCMS (ESI): RT=1.240 min, m/z. found 1068.4 [M+CF₃COOH+H]⁺.

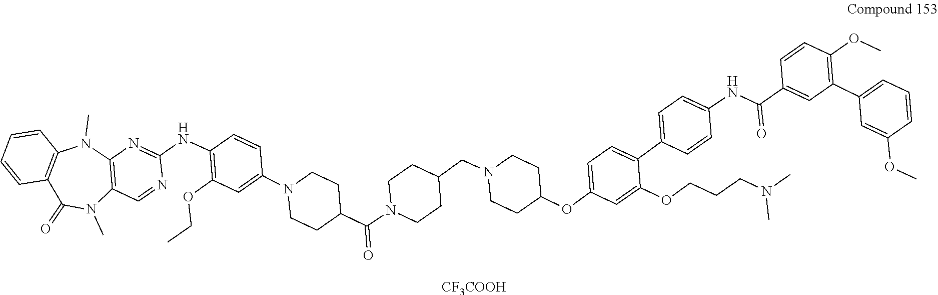 [SMILES: CCOc1cc(N2CCC(C(=O)N3CCC(CN4CCC(Oc5ccc(-c6ccc(NC(=O)c7ccc(OC)c(-c8cccc(OC)c8)c7)cc6)c(OCCCN(C)C)c5)CC4)CC3)CC2)ccc1Nc1ncc2c(n1)N(C)c1ccccc1C(=O)N2C ; "CF3COOH"]

N-(4′-((1-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperidin-4-yl)oxy)-2′-(3-(dimethylamino)propoxy)-[1,1′-biphenyl]-4-yl)-3′,6-dimethoxy-[1,1′-biphenyl]-3-carboxamide, trifluoroacetic acid. ¹H NMR (400 MHz, DMSO-d₆) δ 10.74 (s, 1H), 10.23 (s, 1H), 9.57 (s, 1H), 8.92 (s, 1H), 8.35 (s, 1H), 8.07-7.95 (m, 3H), 7.82 (d, J=8.7 Hz, 2H), 7.68 (dd, J=7.7, 1.6 Hz, 1H), 7.54-7.34 (m, 5H), 7.29-7.07 (m, 5H), 6.96 (dd, J=8.2, 2.0 Hz, 1H), 6.85-6.64 (m, 2H), 5.76 (s, 1H), 5.21-5.12 (m, 2H), 4.43-4.26 (m, 5H), 4.13-4.00 (m, 7H), 3.86 (s, 3H), 3.80 (s, 3H), 3.70-3.60 (m, 2H), 3.39 (s, 3H), 3.30 (s, 3H), 3.19-3.02 (m, 7H), 2.95-2.85 (m, 1H), 2.78 (d, J=4.7 Hz, 6H), 2.66-2.50 (m, 2H), 2.22-2.02 (m, 3H), 1.94-1.68 (m, 6H), 1.32 (t, J=6.8 Hz, 3H). LCMS (ESI): RT=1.330 min, m/z. found 1192.2 [M+CF₃COOH+H]⁺.

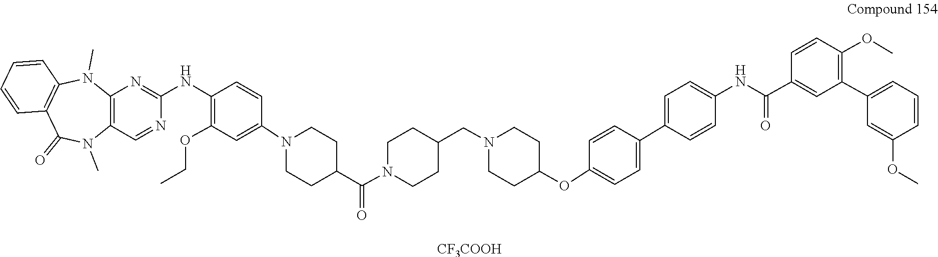 [SMILES: CCOc1cc(N2CCC(C(=O)N3CCC(CN4CCC(Oc5ccc(-c6ccc(NC(=O)c7ccc(OC)c(-c8cccc(OC)c8)c7)cc6)cc5)CC4)CC3)CC2)ccc1Nc1ncc2c(n1)N(C)c1ccccc1C(=O)N2C ; "CF3COOH"]

N-(4′-((1-((1-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperidin-4-yl)methyl)piperidin-4-yl)oxy)-[1,1′-biphenyl]-4-yl)-3′,6-dimethoxy-[1,1′-biphenyl]-3-carboxamide, trifluoroacetic acid. ¹H NMR (400 MHz, CD₃OD) δ 8.56 (d, J=8.8 Hz, 1H), 8.33 (s, 1H), 8.03-7.91 (m, 2H), 7.76 (d, J=8.1 Hz, 3H), 7.63-7.48 (m, 6H), 7.37-7.04 (m, 12H), 6.91 (d, J=9.2 Hz, 1H), 4.62 (d, J=12.9 Hz, 1H), 4.30-4.16 (m, 3H), 3.90 (s, 3H), 3.83 (s, 4H), 3.78-3.66 (m, 5H), 3.52-3.31 (m, 8H), 3.25-3.09 (m, 6H), 2.80-2.69 (m, 1H), 2.31-1.90 (m, 13H), 1.52 (t, J=6.8 Hz, 3H). LCMS (ESI): RT=1.340 min, m/z. found 1090.4 [M+CF₃COOH+H]⁺.

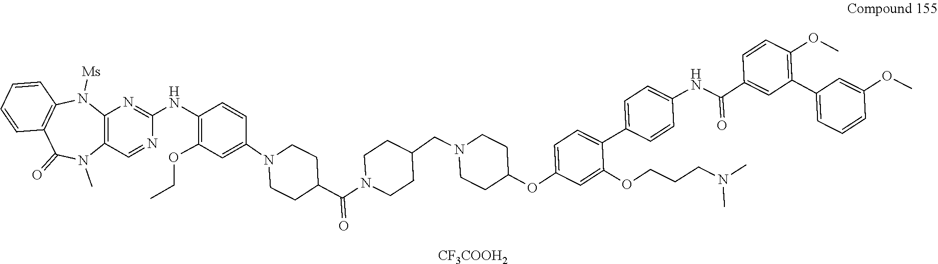

N-(2′-(3-(dimethylamino)propoxy)-4′-((1-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperidin-4-yl)oxy)-[1,1′-biphenyl]-4-yl)-3′,6-dimethoxy-[1,1′-biphenyl]-3-carboxamide, trifluoroacetic acid ¹H NMR (400 MHz, CD₃OD) δ 8.56 (d, J=8.8 Hz, 1H), 8.33 (s, 1H), 8.03-7.91 (m, 2H), 7.76 (d, J=8.1 Hz, 3H), 7.63-7.48 (m, 6H), 7.37-7.04 (m, 12H), 6.91 (d, J=9.2 Hz, 1H), 4.62 (d, J=12.9 Hz, 1H), 4.30-4.16 (m, 3H), 3.90 (s, 3H), 3.83 (s, 4H), 3.78-3.66 (m, 5H), 3.52-3.31 (m, 8H), 3.25-3.09 (m, 6H), 2.80-2.69 (m, 1H), 2.31-1.90 (m, 13H), 1.52 (t, J=6.8 Hz, 3H). LCMS (ESI): RT=1.304 min, m/z. found 1254.0 [M+CF₃COOH+H]⁺.

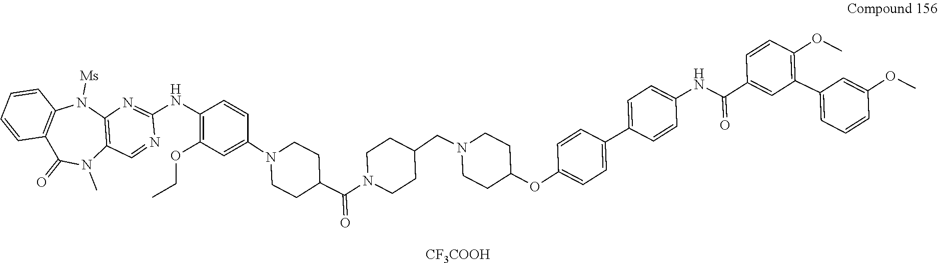 [SMILES: CCOc1cc(N2CCC(C(=O)N3CCC(CN4CCC(Oc5ccc(-c6ccc(NC(=O)c7ccc(OC)c(-c8cccc(OC)c8)c7)cc6)cc5)CC4)CC3)CC2)ccc1Nc1ncc2c(n1)N(S(C)(=O)=O)c1ccccc1C(=O)N2C ; "CF3COOH"]

N-(4′-((1-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperidin-4-yl)oxy)-[1,1′-biphenyl]-4-yl)-3′,6-dimethoxy-[1,1′-biphenyl]-3-carboxamide, trifluoroacetic acid. ¹H NMR (400 MHz, DMSO-d₆) δ 10.72 (s, 1H), 10.22 (s, 1H), 9.57 (s, 1H), 8.86 (d, J=40.0 Hz, 2H), 8.62 (s, 1H), 8.05-7.95 (m, 2H), 7.83-7.74 (m, 3H), 7.60-7.24 (m, 9H), 7.15-7.07 (m, 2H), 6.97-6.05 (m, 1H), 6.71-6.56 (m, 2H), 5.76 (s, 1H), 5.21-5.12 (m, 2H), 4.45-4.29 (m, 4H), 4.06-3.99 (m, 7H), 3.86 (s, 3H), 3.80 (s, 3H), 3.69 (s, 4H), 3.46 (s, 3H), 3.19-3.01 (m, 7H), 2.88-2.74 (m, 9H), 2.62-2.50 (m, 2H), 2.20-2.04 (m, 3H), 1.95-1.67 (m, 6H), 1.17 (t, J=6.8 Hz, 3H). LCMS (ESI): RT=1.285 min, m/z. found 1256.5 [M+CF₃COOH+H]⁺.

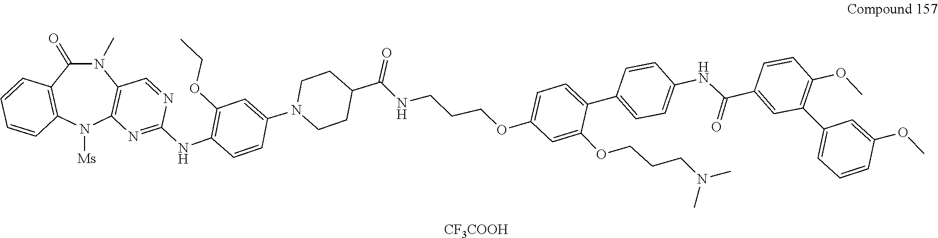 [SMILES: CCOc1cc(N2CCC(C(=O)NCCCOc3ccc(-c4ccc(NC(=O)c5ccc(OC)c(-c6cccc(OC)c6)c5)cc4)c(OCCCN(C)C)c3)CC2)ccc1Nc1ncc2c(n1)N(S(C)(=O)=O)c1ccccc1C(=O)N2C ; "CF3COOH"]

N-(3-((4′-(3′,6-dimethoxy-[1,1′-biphenyl]-3-carboxamido)-2-(3-(dimethylamino)propoxy)-[1,1′-biphenyl]-4-yl)oxy)propyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆): δ 10.20 (s, 1H), 9.42 (s, 1H), 8.79 (s, 1H), 8.62 (s, 1H), 8.04-7.94 (m, 3H), 7.83-7.72 (m, 3H), 7.58 (d, J=3.4 Hz, 2H), 7.45 (d, J=8.8 Hz, 3H), 7.25 (t, J=7.6 Hz, 7H), 7.19-7.15 (m, 6H), 7.00-6.94 (m, 1H), 6.70-6.63 (m, 3H), 4.09-3.99 (m, 6H), 3.87 (s, 3H), 3.80 (s, 3H), 3.72-3.66 (m, 5H), 3.27-3.22 (m, 2H), 3.14-3.06 (m, 2H), 2.76 (d, J=4.7 Hz, 6H), 2.30 (s, 6H), 2.12-2.04 (m, 2H), 1.92-1.85 (m, 2H), 1.81-1.75 (m, 2H), 1.23 (s, 1H), 1.20-1.12 (m, 3H). LCMS (ESI): R_{T}=1.350 min, m/z. found 1132.7 [M-CF₃COOH+H]⁺.

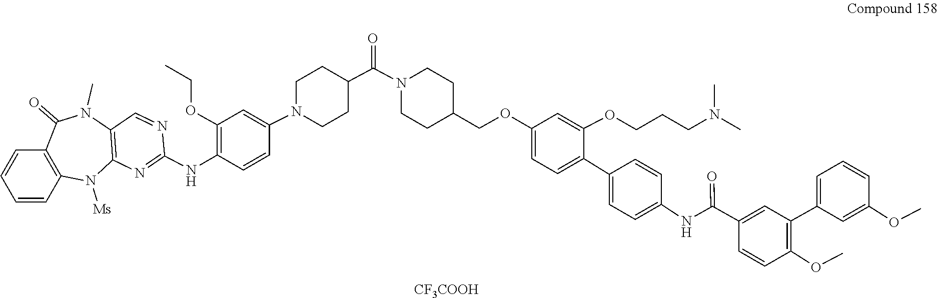 [SMILES: CCOc1cc(N2CCC(C(=O)N3CCC(COc4ccc(-c5ccc(NC(=O)c6ccc(OC)c(-c7cccc(OC)c7)c6)cc5)c(OCCCN(C)C)c4)CC3)CC2)ccc1Nc1ncc2c(n1)N(S(C)(=O)=O)c1ccccc1C(=O)N2C ; "CF3COOH"]

N-(2′-(3-(dimethylamino)propoxy)-4′-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methoxy)-[1,1′-biphenyl]-4-yl)-3′,6-dimethoxy-[1,1′-biphenyl]-3-carboxamide, trifluoroacetic acid. ¹H NMR (400 MHz, CD₃OD) δ 8.68 (s, 1H), 8.37 (d, J=8.7 Hz, 1H), 7.98 (dd, J=8.6, 2.3 Hz, 1H), 7.93 (d, J=2.3 Hz, 1H), 7.85 (d, J=6.5 Hz, 1H), 7.75 (d, J=8.6 Hz, 2H), 7.66-7.55 (m, 6H), 7.50 (t, J=7.5 Hz, 1H), 7.35-7.02 (m, 8H), 6.92 (d, J=1.7 Hz, 1H), 4.61 (d, J=13.8 Hz, 1H), 4.27-4.13 (m, 3H), 3.90 (s, 3H), 3.83 (s, 3H), 3.80-3.62 (m, 5H), 3.61-3.50 (m, 7H), 3.44-3.33 (m, 2H), 3.25-3.10 (m, 4H), 2.74 (t, J=11.9 Hz, 1H), 2.45-1.84 (m, 12H), 1.47 (t, J=7.0 Hz, 3H), 1.38-1.18 (m, 4H). LCMS (ESI): RT=1.420 min, m/z. found 1171.2 [M-CF₃COOH+H]⁺.

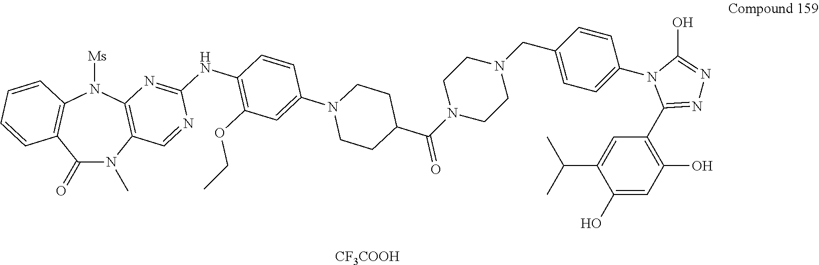 [SMILES: CCOc1cc(N2CCC(C(=O)N3CCN(Cc4ccc(-n5c(O)nnc5-c5cc(C(C)C)c(O)cc5O)cc4)CC3)CC2)ccc1Nc1ncc2c(n1)N(S(C)(=O)=O)c1ccccc1C(=O)N2C ; "CF3COOH"]

2-((4-(4-(4-(4-(3-(2,4-dihydroxy-5-isopropylphenyl)-5-hydroxy-4H-1,2,4-triazol-4-yl)benzyl)piperazine-1-carbonyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, CD₃OD) δ 8.53 (s, 1H), 7.81 (dd, 1H), 7.67-7.56 (m, 2H), 7.54-7.37 (m, 4H), 7.25 (d, J=8.3 Hz, 2H), 6.74 (s, 1H), 6.65 (d, J=2.2 Hz, 1H), 6.58 (dd, J=8.7, 2.4 Hz, 1H), 6.27 (s, 1H), 4.13-3.97 (m, 2H), 3.73-3.54 (m, 13H), 3.09-2.98 (m, 1H), 2.86-2.74 (m, 3H), 2.58-2.39 (m, 4H), 2.25-2.12 (m, 1H), 1.92-1.74 (m, 4H), 1.33-1.28 (m, 7H), 0.98-0.89 (m, 6H). LCMS (ESI): R_{T} 1.160 min, m/z. found 958.5 [M-CF₃COOH+H]⁺.

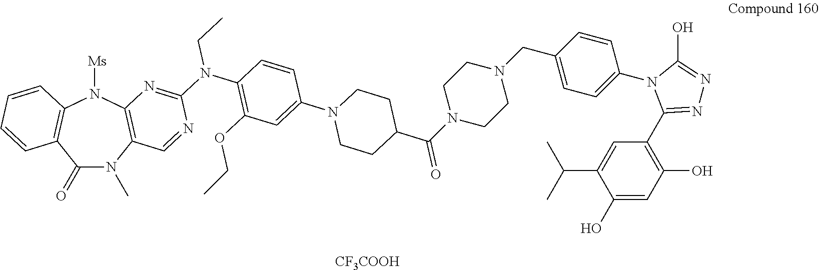 [SMILES: CCOc1cc(N2CCC(C(=O)N3CCN(Cc4ccc(-n5c(O)nnc5-c5cc(C(C)C)c(O)cc5O)cc4)CC3)CC2)ccc1N(CC)c1ncc2c(n1)N(S(C)(=O)=O)c1ccccc1C(=O)N2C ; "CF3COOH"]

2-((4-(4-(4-(4-(3-(2,4-dihydroxy-5-isopropylphenyl)-5-hydroxy-4H-1,2,4-triazol-4-yl)benzyl)piperazine-1-carbonyl)piperidin-1-yl)-2-ethoxyphenyl)(ethyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, CD₃OD) δ 8.53 (s, 1H), 7.81 (dd, 1H), 7.67-7.56 (m, 2H), 7.54-7.37 (m, 4H), 7.25 (d, J=8.3 Hz, 2H), 6.74 (s, 1H), 6.65 (d, J=2.2 Hz, 1H), 6.58 (dd, J=8.7, 2.4 Hz, 1H), 6.27 (s, 1H), 4.13-3.97 (m, 2H), 3.73-3.54 (m, 13H), 3.09-2.98 (m, 1H), 2.86-2.74 (m, 3H), 2.58-2.39 (m, 4H), 2.25-2.12 (m, 1H), 1.92-1.74 (m, 4H), 1.33-1.28 (m, 7H), 0.98-0.89 (m, 6H). LCMS (ESI): R_{T}=1.160 min, m/z. found 985.5 [M-CF₃COOH+H]⁺.

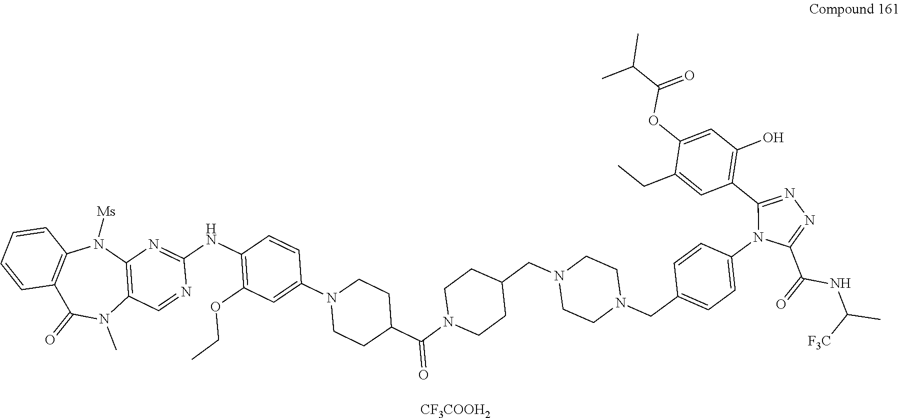

4-(4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-((1,1,1-trifluoropropan-2-yl)carbamoyl)-4H-1,2,4-triazol-3-yl)-2-ethyl-5-hydroxyphenyl isobutyrate, trifluoroacetic acid. ¹H NMR (400 MHz, DMSO-d₆): δ 8.68 (s, 1H), 8.34 (d, J=8.2 Hz, 1H), 7.86 (d, J=6.4 Hz, 1H), 7.65-7.60 (m, 1H), 7.56 (d, J=7.3 Hz, 1H), 7.52-7.45 (m, 3H), 7.36 (d, J=8.3 Hz, 2H), 7.19 (s, 1H), 7.14 (d, J=8.5 Hz, 1H), 7.08 (s, 1H), 6.46 (s, 1H), 4.73-4.63 (m, 1H), 4.58 (d, J=12.4 Hz, 1H), 4.32-4.03 (m, 4H), 3.92 (s, 2H), 3.76 (s, 3H), 3.59 (d, J=13.9 Hz, 9H), 3.20 (d, J=14.0 Hz, 5H), 2.96 (s, 4H), 2.86-2.80 (m, 3H), 2.71 (t, J=11.8 Hz, 2H), 2.34 (q, J=7.5 Hz, 2H), 2.14-2.10 (M, 6H), 2.01-1.80 (m, 3H), 1.47-1.41 (m, 6H), 1.28 (d, J=7.0 Hz, 6H), 0.99 (t, J=7.5 Hz, 3H). LCMS (ESI): RT=1.360 min, m/z. found 618.3 [M/2-CF₃COOH+H]⁺.

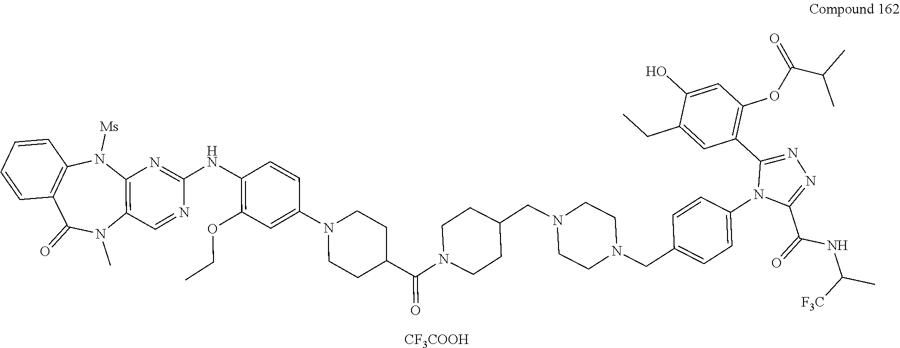 [SMILES: CCOc1cc(N2CCC(C(=O)N3CCC(CN4CCN(Cc5ccc(-n6c(C(=O)NC(C)C(F)(F)F)nnc6-c6cc(CC)c(O)cc6OC(=O)C(C)C)cc5)CC4)CC3)CC2)ccc1Nc1ncc2c(n1)N(S(C)(=O)=O)c1ccccc1C(=O)N2C ; "CF3COOH"]

2-(4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-((1,1,1-trifluoropropan-2-yl)carbamoyl)-4H-1,2,4-triazol-3-yl)-4-ethyl-5-hydroxyphenyl isobutyrate, trifluoroacetic acid. ¹H NMR (400 MHz, DMSO-d₆): δ 8.68 (s, 1H), 8.34 (d, J=8.2 Hz, 1H), 7.86 (d, J=6.4 Hz, 1H), 7.65-7.60 (m, 1H), 7.56 (d, J=7.3 Hz, 1H), 7.52-7.45 (m, 3H), 7.36 (d, J=8.3 Hz, 2H), 7.19 (s, 1H), 7.14 (d, J=8.5 Hz, 1H), 7.08 (s, 1H), 6.46 (s, 1H), 4.73-4.63 (m, 1H), 4.58 (d, J=12.4 Hz, 1H), 4.32-4.03 (m, 4H), 3.92 (s, 2H), 3.76 (s, 3H), 3.59 (d, J=13.9 Hz, 9H), 3.20 (d, J=14.0 Hz, 5H), 2.96 (s, 4H), 2.86-2.80 (m, 3H), 2.71 (t, J=11.8 Hz, 2H), 2.34 (q, J=7.5 Hz, 2H), 2.14-2.10 (M, 6H), 2.01-1.80 (m, 3H), 1.47-1.41 (m, 6H), 1.28 (d, J=7.0 Hz, 6H), 0.99 (t, J=7.5 Hz, 3H). LCMS (ESI): RT=1.360 min, m/z. found 618.3 [M/2-CF₃COOH+H]⁺.

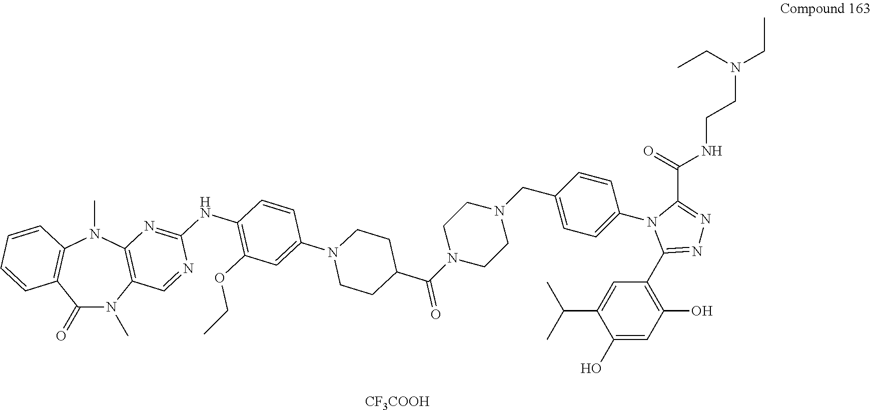 [SMILES: CCOc1cc(N2CCC(C(=O)N3CCN(Cc4ccc(-n5c(C(=O)NCCN(CC)CC)nnc5-c5cc(C(C)C)c(O)cc5O)cc4)CC3)CC2)ccc1Nc1ncc2c(n1)N(C)c1ccccc1C(=O)N2C ; "CF3COOH"]

N-(2-(diethylamino)ethyl)-5-(2,4-dihydroxy-5-isopropylphenyl)-4-(4-((4-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperazin-1-yl)methyl)phenyl)-4H-1,2,4-triazole-3-carboxamide, trifluoroacetic acid. ¹H NMR (400 MHz, DMSO-d₆): δ 9.77 (s, 1H), 9.24 (t, J=6.0 Hz, 2H), 8.34 (s, 1H), 8.03 (s, 1H), 7.92-7.90 (m, 1H), 7.68 (dd, J=8.0, 4.0 Hz, 1H), 7.54-7.47 (m, 3H), 7.44 (d, J=8.0 Hz, 2H), 7.24 (d, J=8.0 Hz, 1H), 7.18 (t, J=6.0 Hz, 1H), 6.80 (s, 1H), 6.73 (s, 1H), 6.31 (s, 1H), 4.35 (s, 1H), 4.10 (q, J=16.0 Hz, 1H), 3.70-3.67 (m, 8H), 3.56-3.52 (m, 4H), 3.39 (s, 3H), 3.30 (s, 3H), 3.22-3.17 (m, 8H), 3.00-2.85 (m, 5H), 1.79 (s, 4H), 1.32 (t, J=6.0 Hz, 3H), 1.19 (t, J=6.0 Hz, 6H), 0.92 (d, J=8.0 Hz, 6H). LCMS (ESI): R_{T}=1.120 min, m/z. found 1021.2 [M-CF₃COOH+H]⁺.

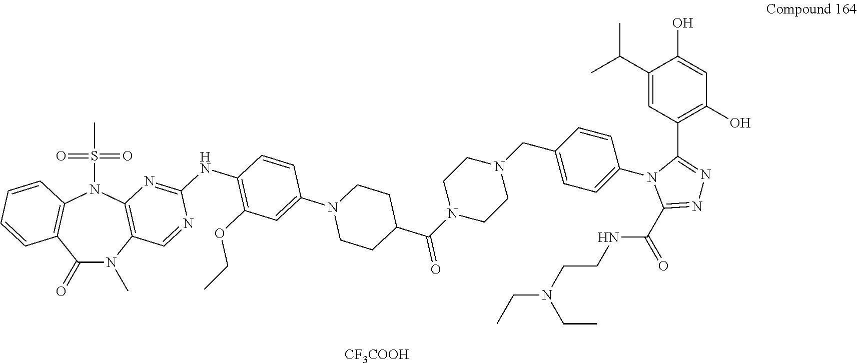 [SMILES: CCOc1cc(N2CCC(C(=O)N3CCN(Cc4ccc(-n5c(C(=O)NCCN(CC)CC)nnc5-c5cc(C(C)C)c(O)cc5O)cc4)CC3)CC2)ccc1Nc1ncc2c(n1)N(S(C)(=O)=O)c1ccccc1C(=O)N2C ; "CF3COOH"]

N-(2-(diethylamino)ethyl)-5-(2,4-dihydroxy-5-isopropylphenyl)-4-(4-((4-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperazin-1-yl)methyl)phenyl)-4H-1,2,4-triazole-3-carboxamide, trifluoroacetic acid. ¹H NMR (400 MHz, DMSO-d₆): δ 9.78 (s, 1H), 9.24 (t, J=5.9 Hz, 2H), 8.78 (s, 1H), 8.62 (s, 1H), 7.75 (d, J=8.0 Hz, 1H), 7.59 (d, J=4.0 Hz, 2H), 7.52 (d, J=8.3 Hz, 2H), 7.45-7.43 (m, 2H), 7.33 (d, J=8.0 Hz, 1H), 6.73 (s, 1H), 6.67 (s, 1H), 6.56 (d, J=8.0 Hz, 1H), 6.31 (s, 1H), 4.34 (s, 1H), 4.07-3.99 (m, 1H), 3.73-3.68 (m, 6H), 3.54 (s, 6H), 3.49 (s, 3H), 3.46 (s, 3H), 3.24-3.16 (m, 8H), 3.00-2.93 (m, 1H), 2.84 (s, 4H), 1.74 (s, 4H), 1.22-1.14 (m, 9H), 0.92 (d, J=8.0 Hz, 6H). LCMS (ESI): R_{T}=1.090 min, m/z. found 1085.3 [M-CF₃COOH+H]⁺.

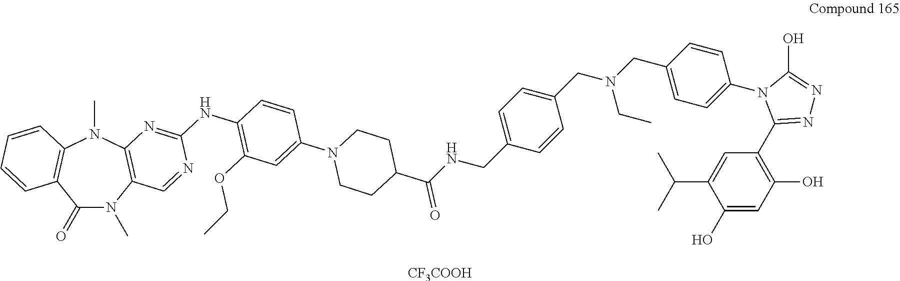 [SMILES: CCOc1cc(N2CCC(C(=O)NCc3ccc(CN(CC)Cc4ccc(-n5c(O)nnc5-c5cc(C(C)C)c(O)cc5O)cc4)cc3)CC2)ccc1Nc1ncc2c(n1)N(C)c1ccccc1C(=O)N2C ; "CF3COOH"]

N-(4-(((4-(3-(2,4-dihydroxy-5-isopropylphenyl)-5-hydroxy-4H-1,2,4-triazol-4-yl)benzyl)(ethyl)amino)methyl)benzyl)-1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carboxamide, trifluoroacetic acid. ¹H NMR (400 MHz, DMSO-d₆) δ 12.00 (s, 1H), 9.83 (s, 1H), 9.67 (s, 1H), 9.37 (s, 1H), 8.56 (t, J=5.8 Hz, 1H), 8.37 (s, 1H), 8.15 (s, 1H), 8.02 (s, 1H), 7.69 (dd, J=7.7, 1.6 Hz, 1H), 7.53-7.49 (m, 3H), 7.45 (d, J=8.1 Hz, 2H), 7.34 (d, J=8.1 Hz, 2H), 7.27-7.24 (m, 3H), 7.18 (t, J=7.5 Hz, 1H), 7.03-6.96 (m, 1H), 6.90 (s, 2H), 6.25 (s, 1H), 4.31 (dd, J=20.5, 8.4 Hz, 6H), 4.14-4.12 (m, 3H), 3.68 (d, J=12.0 Hz, 2H), 3.39 (s, 3H), 3.32 (s, 3H), 3.13 (s, 1H), 3.04-2.91 (m, 3H), 1.94 (s, 4H), 1.35 (t, J=6.9 Hz, 3H), 1.25 (t, J=7.1 Hz, 4H), 1.01 (d, J=6.9 Hz, 6H). LCMS (ESI): R_{T}=1.250 min, m/z. found 972.5 [M-CF₃COOH+H]⁺.

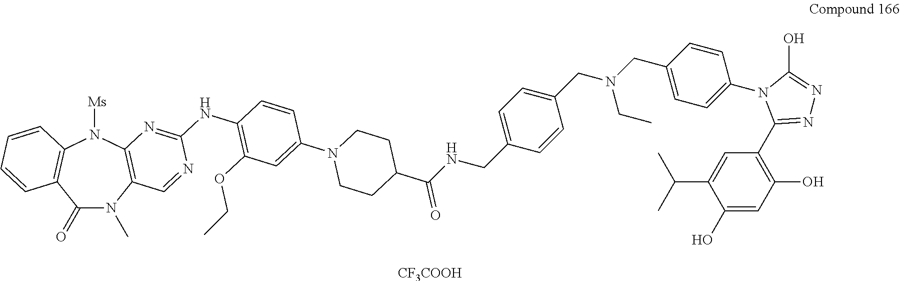 [SMILES: CCOc1cc(N2CCC(C(=O)NCc3ccc(CN(CC)Cc4ccc(-n5c(O)nnc5-c5cc(C(C)C)c(O)cc5O)cc4)cc3)CC2)ccc1Nc1ncc2c(n1)N(S(C)(=O)=O)c1ccccc1C(=O)N2C ; "CF3COOH"]

N-(4-(((4-(3-(2,4-dihydroxy-5-isopropylphenyl)-5-hydroxy-4H-1,2,4-triazol-4-yl)benzyl)(ethyl)amino)methyl)benzyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆) δ 12.00 (s, 1H), 9.70 (d, J=39.7 Hz, 2H), 9.36 (s, 1H), 8.84 (s, 1H), 8.63 (s, 1H), 8.50 (t, J=6.0 Hz, 1H), 7.75 (d, J=7.8 Hz, 1H), 7.59 (d, J=3.7 Hz, 2H), 7.52-7.42 (m, 5H), 7.33 (d, J=8.0 Hz, 2H), 7.26 (d, J=8.2 Hz, 2H), 6.90 (s, 1H), 6.83-6.57 (m, 2H), 6.24 (s, 1H), 4.34-4.26 (m, 5H), 4.06-4.02 (m, 2H), 3.74-3.68 (m, 5H), 3.46 (s, 3H), 3.03-2.84 (m, 5H), 2.45-2.39 (m, 1H), 1.90-1.75 (m, 4H), 1.26-1.23 (m, 4H), 1.18 (t, J=6.7 Hz, 3H), 1.01 (d, J=6.8 Hz, 6H). LCMS (ESI): R_{T} 1.240 min, m/z. found 1036.4 [M-CF₃COOH+H]⁺.

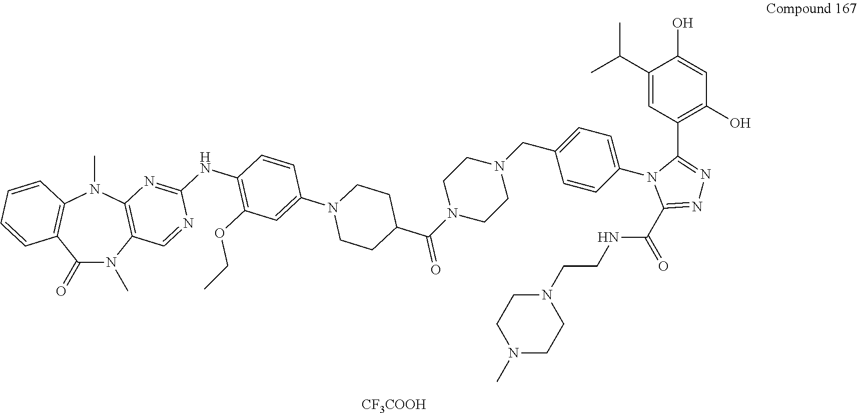 [SMILES: CCOc1cc(N2CCC(C(=O)N3CCN(Cc4ccc(-n5c(C(=O)NCCN6CCN(C)CC6)nnc5-c5cc(C(C)C)c(O)cc5O)cc4)CC3)CC2)ccc1Nc1ncc2c(n1)N(C)c1ccccc1C(=O)N2C ; "CF3COOH"]

5-(2,4-dihydroxy-5-isopropylphenyl)-4-(4-((4-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperazin-1-yl)methyl)phenyl)-N-(2-(4-methylpiperazin-1-yl)ethyl)-4H-1,2,4-triazole-3-carboxamide, trifluoroacetic acid. ¹H NMR (400 MHz, DMSO-d₆) δ 9.79 (s, 1H), 8.99-8.96 (m, 1H), 8.34 (s, 1H), 8.06 (s, 1H), 7.93 (d, J=8.6 Hz, 1H), 7.69 (dd, J=7.8, 1.6 Hz, 1H), 7.59-7.47 (m, 4H), 7.44 (d, J=8.4 Hz, 2H), 7.24 (d, J=8.1 Hz, 1H), 7.18 (t, J=7.4 Hz, 1H), 6.84 (s, 1H), 6.76-6.71 (m, 1H), 6.69 (s, 1H), 6.32 (s, 1H), 4.37 (s, 2H), 4.14-4.08 (m, 3H), 3.70-3.67 (m, 8H), 3.40-3.34 (m, 11H), 3.31 (s, 5H), 2.99-2.92 (m, 4H), 2.78 (s, 3H), 2.75-2.67 (m, 3H), 1.81 (s, 4H), 1.33 (t, J=6.9 Hz, 3H), 0.90 (d, J=6.9 Hz, 6H). LCMS (ESI): R_{T} 1.240 min, m/z. found 1048.2 [M-CF₃COOH+H]⁺.

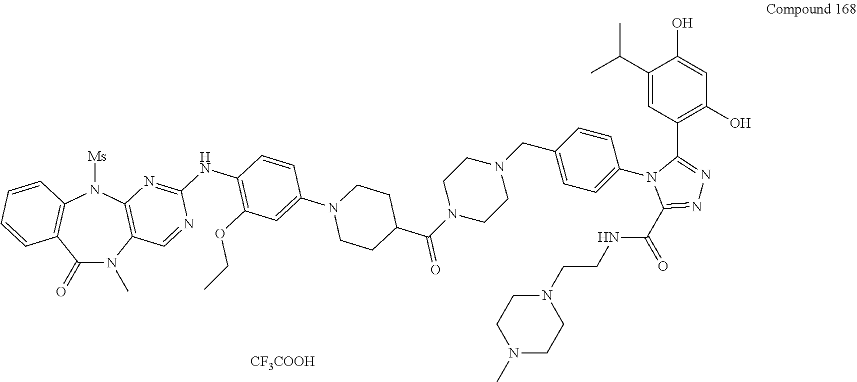 [SMILES: CCOc1cc(N2CCC(C(=O)N3CCN(Cc4ccc(-n5c(C(=O)NCCN6CCN(C)CC6)nnc5-c5cc(C(C)C)c(O)cc5O)cc4)CC3)CC2)ccc1Nc1ncc2c(n1)N(S(C)(=O)=O)c1ccccc1C(=O)N2C ; "CF3COOH"]

2-((4-(4-(2-(4-(4-(3-(2,4-dihydroxy-5-isopropylphenyl)-5-hydroxy-4H-1,2,4-triazol-4-yl)benzyl)piperazin-1-yl)-2-oxoethoxy)piperidin-1-yl)-2-ethoxyphenyl)amino)-5,11-dimethyl-5H-benzo[e]pyrimido[5,4-b][1,4]diazepin-6(11H)-one, trifluoroacetic acid. ¹H NMR (400 MHz, DMSO-d₆): δ 9.86 (s, 1H), 9.07-9.04 (m, 1H), 8.88 (s, 1H), 8.69 (s, 1H), 7.82 (d, J=8.0 Hz, 1H), 7.66-7.60 (m, 4H), 7.55-7.49 (m, 3H), 7.44 (d, J=12.0 Hz, 1H), 6.79-6.76 (m, 2H), 6.68 (d, J=8.0 Hz, 1H), 6.38 (s, 1H), 4.44 (s, 2H), 4.13-4.08 (m, 2H), 3.80-3.75 (m, 8H), 3.52 (s, 3H), 3.46-3.42 (m, 4H), 3.30-3.21 (m, 4H), 3.05-3.01 (m, 1H), 3.00-2.92 (m, 4H), 2.85 (s, 3H), 2.80-2.72 (m, 3H), 1.83-1.80 (m, 4H), 1.25 (t, J=6.0 Hz, 3H), 0.97 (d, J=8.0 Hz, 6H). LCMS (ESI): R_{T}=1.135 min, m/z. found 1112.2 [M-CF₃COOH+H]⁺.

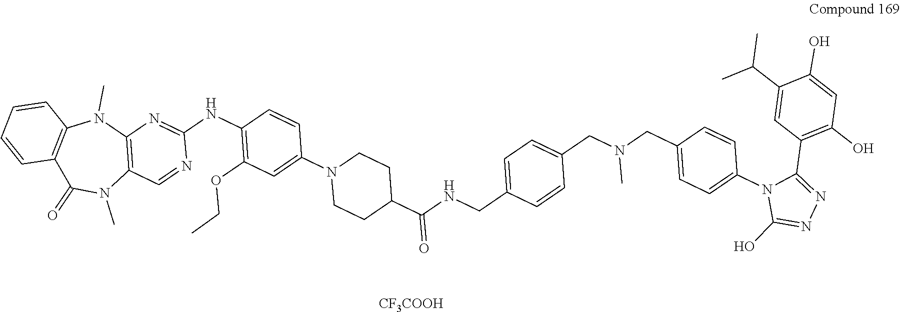 [SMILES: CCOc1cc(N2CCC(C(=O)NCc3ccc(CN(C)Cc4ccc(-n5c(O)nnc5-c5cc(C(C)C)c(O)cc5O)cc4)cc3)CC2)ccc1Nc1ncc2c(n1)N(C)c1ccccc1C(=O)N2C ; "CF3COOH"]

2-((4-(4-(2-(4-(4-(3-(2,4-dihydroxy-5-isopropylphenyl)-5-hydroxy-4H-1,2,4-triazol-4-yl)benzyl)piperazin-1-yl)-2-oxoethoxy)piperidin-1-yl)-2-ethoxyphenyl)amino)-5,11-dimethyl-5H-benzo[e]pyrimido[5,4-b][1,4]diazepin-6(11H)-one, trifluoroacetic acid. ¹H NMR (400 MHz, DMSO-d₆): δ 11.98 (s, 1H), 9.93 (s, 1H), 9.63 (s, 1H), 9.34 (s, 1H), 8.53 (s, 1H), 8.36 (s, 1H), 8.05 (d, J=45.0 Hz, 2H), 7.68 (d, J=7.7 Hz, 1H), 7.48 (dd, J=22.0, 8.1 Hz, 5H), 7.34 (d, J=8.0 Hz, 2H), 7.26 (t, J=8.8 Hz, 3H), 7.18 (t, J=7.5 Hz, 1H), 6.90 (s, 2H), 6.24 (s, 1H), 4.73-4.52 (m, 3H), 4.37 (dd, J=23.7, 9.2 Hz, 6H), 4.15 (dd, J=19.2, 12.3 Hz, 5H), 3.68 (d, J=11.8 Hz, 2H), 3.39 (s, 3H), 3.32 (s, 3H), 3.01 (dd, J=13.8, 6.9 Hz, 1H), 1.91 (s, 4H), 1.34 (t, J=6.9 Hz, 3H), 1.01 (d, J=6.8 Hz, 6H). LCMS (ESI): RT=1.420 min, m/z. found 958.6 [M-CF₃COOH+H]⁺.

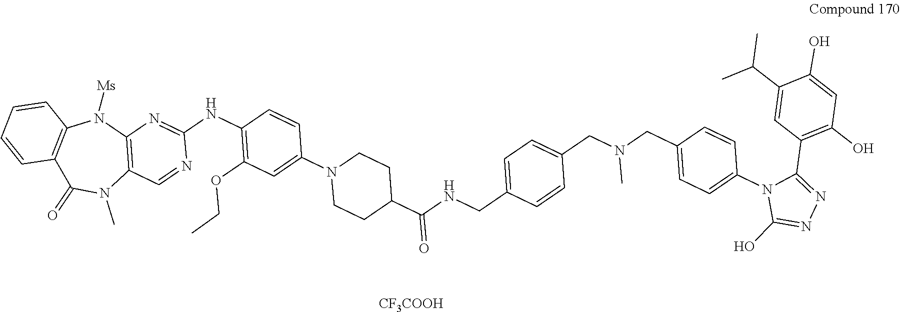 [SMILES: CCOc1cc(N2CCC(C(=O)NCc3ccc(CN(C)Cc4ccc(-n5c(O)nnc5-c5cc(C(C)C)c(O)cc5O)cc4)cc3)CC2)ccc1Nc1ncc2c(n1)N(S(C)(=O)=O)c1ccccc1C(=O)N2C ; "CF3COOH"]

N-(4-(((4-(3-(2,4-dihydroxy-5-isopropylphenyl)-5-hydroxy-4H-1,2,4-triazol-4-yl)benzyl)(methyl)amino)methyl)benzyl)-1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carboxamide, trifluoroacetic acid. ¹H NMR (400 MHz, DMSO-d₆) δ 11.98 (s, 1H), 9.88-9.73 (m, 1H), 9.61 (s, 1H), 9.32 (s, 1H), 8.79 (s, 1H), 8.62 (s, 1H), 8.54-8.38 (m, 1H), 7.75 (d, J=7.8 Hz, 1H), 7.58 (d, J=3.9 Hz, 2H), 7.52-7.43 (m, 5H), 7.30 (dd, J=27.8, 8.2 Hz, 5H), 6.90 (s, 1H), 6.72-6.53 (m, 2H), 6.24 (s, 1H), 4.46-4.30 (m, 5H), 4.26-4.13 (m, 4H), 4.07-3.98 (m, 3H), 3.71 (d, J=17.5 Hz, 5H), 3.46 (s, 3H), 3.06-2.95 (m, 1H), 2.84-2.71 (m, 1H), 2.43-2.35 (m, 1H), 1.88-1.72 (m, 4H), 1.17 (t, J=6.9 Hz, 3H), 1.01 (d, J=6.9 Hz, 6H). LCMS (ESI): R_{T} 1.040 min, m/z. found 1022.3 [M-CF₃COOH+H]⁺.

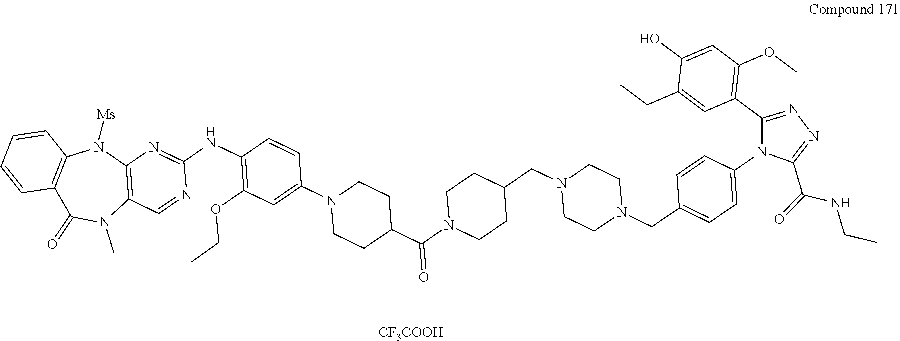 [SMILES: CCNC(=O)c1nnc(-c2cc(CC)c(O)cc2OC)n1-c1ccc(CN2CCN(CC3CCN(C(=O)C4CCN(c5ccc(Nc6ncc7c(n6)N(S(C)(=O)=O)c6ccccc6C(=O)N7C)c(OCC)c5)CC4)CC3)CC2)cc1 ; "CF3COOH"]

4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-N-ethyl-5-(5-ethyl-4-hydroxy-2-methoxyphenyl)-4H-1,2,4-triazole-3-carboxamide, trifluoroacetic acid. ¹H NMR (400 MHz, DMSO-d₆) δ 9.80 (s, 1H), 9.07-9.00 (m, 1H), 8.82 (s, 1H), 8.62 (s, 1H), 7.75 (d, J=7.8 Hz, 1H), 7.59 (d, J=4.0 Hz, 2H), 7.51-7.44 (m, 2H), 7.33 (s, 3H), 7.22-7.06 (m, 3H), 6.77-6.51 m, 1H), 6.28 (s, 1H), 4.44-4.37 (m, 2H), 4.10-3.97 (m, 6H), 3.74-3.64 (m, 6H), 3.46 (s, 3H), 3.30 (s, 3H), 3.23-3.14 (m, 3H), 3.11-2.77 (m, 9H), 2.44-2.33 (m, 4H), 1.82-1.63 (m, 7H), 1.21-1.14 (m, 3H), 1.08-1.03 (m, 8H) LCMS (ESI): R_{T}=1.040 min, m/z. found 1110.3 [M-CF₃COOH+H]⁺.

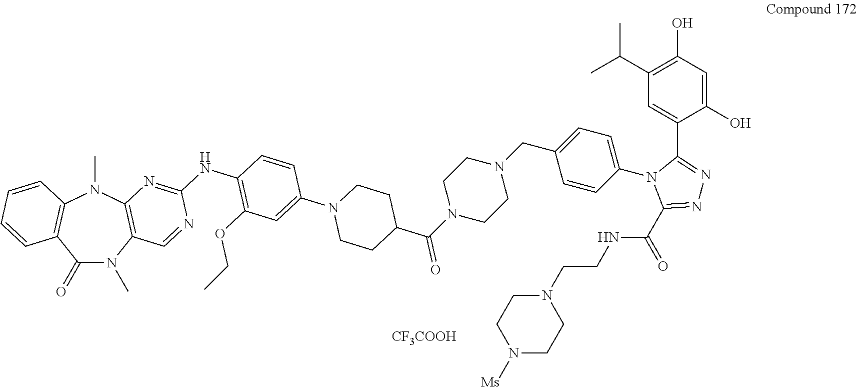 [SMILES: CCOc1cc(N2CCC(C(=O)N3CCN(Cc4ccc(-n5c(C(=O)NCCN6CCN(S(C)(=O)=O)CC6)nnc5-c5cc(C(C)C)c(O)cc5O)cc4)CC3)CC2)ccc1Nc1ncc2c(n1)N(C)c1ccccc1C(=O)N2C ; "CF3COOH"]

5-(2,4-dihydroxy-5-isopropylphenyl)-4-(4-((4-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperazin-1-yl)methyl)phenyl)-N-(2-(4-(methylsulfonyl)piperazin-1-yl)ethyl)-4H-1,2,4-triazole-3-carboxamide, trifluoroacetic acid. ¹H NMR (400 MHz, DMSO-d₆) δ 10.72-10.22 (m, 1H), 9.79 (s, 1H), 9.37-9.23 (m, 1H), 8.36 (s, 1H), 8.11 (s, 1H), 8.03-7.90 (m, 1H), 7.69 (dd, J=7.7, 1.7 Hz, 1H), 7.57-7.42 (m, 5H), 7.28-7.14 (m, 2H), 7.00-6.74 (m, 2H), 6.72 (s, 1H), 6.32 (s, 1H), 4.53-4.28 (m, 3H), 4.12 (q, J=6.9 Hz, 2H), 3.85-3.44 (m, 9H), 3.39 (s, 4H), 3.36-3.07 (m, 13H), 3.06-2.88 (m, 8H), 1.83 (s, 4H), 1.34 (t, J=6.9 Hz, 3H), 0.92 (d, J=6.9 Hz, 6H). LCMS (ESI): R_{T}1.000 min, m/z. found 1111.2 [M-CF₃COOH+H]⁺.

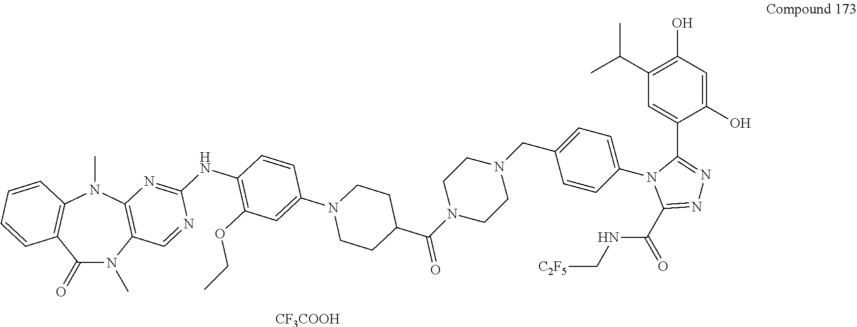

5-(2,4-dihydroxy-5-isopropylphenyl)-4-(4-((4-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperazin-1-yl)methyl)phenyl)-N-(2,2,3,3,3-pentafluoropropyl)-4H-1,2,4-triazole-3-carboxamide, trifluoroacetic acid. ¹H NMR (400 MHz, DMSO-d₆) δ 10.43-9.99 (m, 1H), 9.76 (s, 1H), 9.66 (t, J=6.5 Hz, 1H), 8.36 (s, 1H), 8.12 (s, 1H), 7.97 (s, 1H), 7.69 (dd, J=7.7, 1.6 Hz, 1H), 7.59-7.43 (m, 5H), 7.28-7.14 (m, 2H), 7.04-6.68 (m, 3H), 6.31 (s, 1H), 4.57-4.32 (m, 3H), 4.29-3.94 (m, 5H), 3.69 (d, J=11.7 Hz, 2H), 3.55-3.43 (m, 1H), 3.39 (s, 3H), 3.31 (s, 5H), 3.20-2.85 (m, 7H), 1.83 (s, 4H), 1.34 (t, J=6.9 Hz, 3H), 0.92 (d, J=6.9 Hz, 6H). LCMS (ESI): R_{T}=1.260 min, m/z. found 1053.2 [M-CF₃COOH+H]⁺.

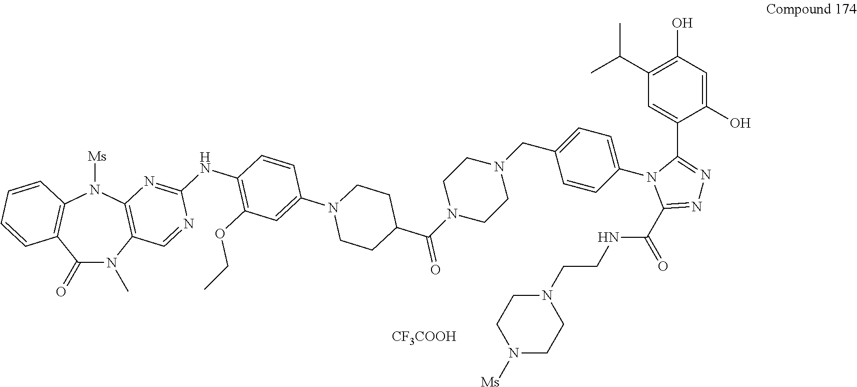 [SMILES: CCOc1cc(N2CCC(C(=O)N3CCN(Cc4ccc(-n5c(C(=O)NCCN6CCN(S(C)(=O)=O)CC6)nnc5-c5cc(C(C)C)c(O)cc5O)cc4)CC3)CC2)ccc1Nc1ncc2c(n1)N(S(C)(=O)=O)c1ccccc1C(=O)N2C ; "CF3COOH"]

5-(2,4-dihydroxy-5-isopropylphenyl)-4-(4-((4-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperazin-1-yl)methyl)phenyl)-N-(2-(4-(methylsulfonyl)piperazin-1-yl)ethyl)-4H-1,2,4-triazole-3-carboxamide, trifluoroacetic acid. ¹H NMR (400 MHz, DMSO-d₆): δ 9.77 (s, 1H), 9.28 (s, 1H), 8.81 (s, 1H), 8.62 (s, 1H), 7.75 (d, J=7.9 Hz, 1H), 7.51 (m, 8H), 7.36 (s, 1H), 6.65-6.63 (m, 3H), 6.31 (s, 1H), 4.38 (s, 4H), 4.11-3.95 (m, 3H), 3.69 (s, 7H), 3.55 (s, 3H), 3.46 (s, 4H), 3.20 (d, J=66.1 Hz, 8H), 3.03 (s, 4H), 2.98-2.87 (m, 6H), 1.75 (s, 4H), 1.18 (t, J=6.8 Hz, 3H), 0.92 (d, J=6.9 Hz, 6H). LCMS (ESI): R_{T}=1.033 min, m/z. found 1175.2 [M-CF₃COOH+H]⁺.

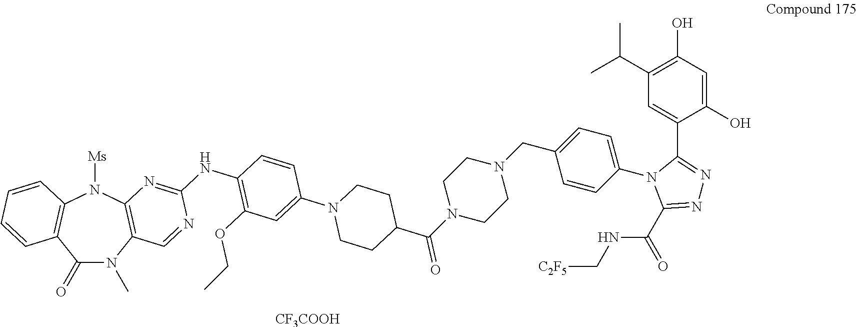

5-(2,4-dihydroxy-5-isopropylphenyl)-4-(4-((4-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperazin-1-yl)methyl)phenyl)-N-(2,2,3,3,3-pentafluoropropyl)-4H-1,2,4-triazole-3-carboxamide, trifluoroacetic acid. ¹H NMR (400 MHz, DMSO-d₆): δ 10.13 (s, 1H), 9.76-9.65 (m, 2H), 8.83 (s, 1H), 8.63 (s, 1H), 7.75 (d, J=7.7 Hz, 1H), 7.57 (dd, J=17.9, 6.0 Hz, 4H), 7.44 (dd, J=19.4, 14.1 Hz, 4H), 6.69-6.66 (m, 3H), 6.31 (s, 1H), 4.39 (s, 3H), 4.31-4.17 (m, 1H), 4.02-3.98 (m, 4H), 3.69 (s, 5H), 3.39 (d, J=56.6 Hz, 6H), 3.13 (s, 1H), 3.06-2.79 (m, 6H), 1.77 (s, 4H), 1.19 (t, J=6.8 Hz, 3H), 0.92 (d, J=6.9 Hz, 6H). LCMS (ESI): R_{T}=1.260 min, m/z. found 1117.1 [M-CF₃COOH+H]⁺.

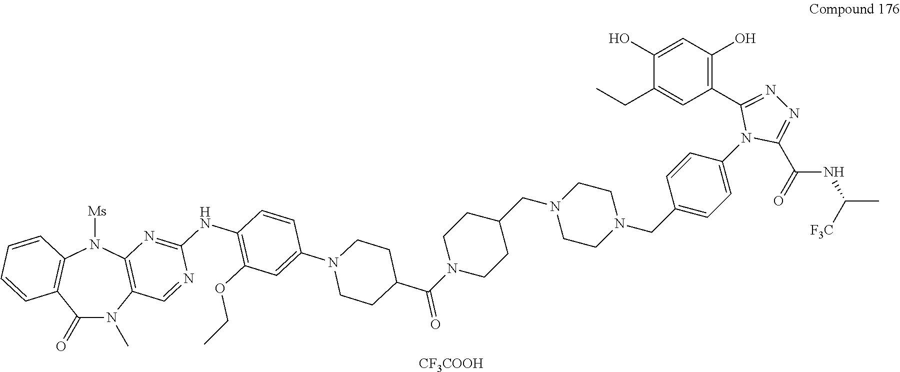 [SMILES: CCOc1cc(N2CCC(C(=O)N3CCC(CN4CCN(Cc5ccc(-n6c(C(=O)N[C@H](C)C(F)(F)F)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

(R)-4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, trifluoroacetic acid. ¹H NMR (400 MHz, DMSO-d₆) δ 9.83-9.54 (m, 2H), 8.86 (s, 1H), 8.65 (s, 1H), 7.76 (d, J=7.6 Hz, 1H), 7.59 (d, J=3.6 Hz, 2H), 7.55-7.38 (m, 4H), 7.34 (d, J=8.2 Hz, 2H), 6.88 (s, 1H), 6.76 (s, 1H), 6.69 (s, 1H), 6.31 (s, 1H), 4.69-4.59 (m, 2H), 4.40 (d, J=13.2 Hz, 1H), 4.13-3.80 (m, 5H), 3.69 (s, 5H), 3.47 (s, 3H), 3.23-2.74 (m, 11H), 2.63-2.50 (m, 4H), 2.28 (q, J=7.5 Hz, 2H), 2.01 (s, 1H), 1.90-1.71 (m, 6H), 1.34 (d, J=7.0 Hz, 3H), 1.21 (t, J=6.9 Hz, 3H), 1.17-0.97 (m, 2H), 0.90 (t, J=7.5 Hz, 3H). LCMS (ESI): R_{T}=1.180 min, m/z. found 1164.3 [M-CF₃COOH+H]⁺.

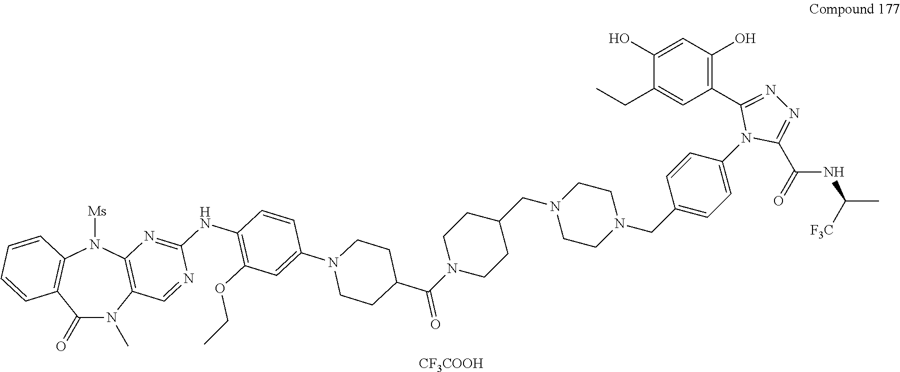 [SMILES: CCOc1cc(N2CCC(C(=O)N3CCC(CN4CCN(Cc5ccc(-n6c(C(=O)N[C@@H](C)C(F)(F)F)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

(S)-4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, trifluoroacetic acid. ¹H NMR (400 MHz, DMSO-d₆) δ 9.93 (s, 1H), 9.60 (d, J=9.1 Hz, 1H), 8.98 (s, 1H), 8.70 (s, 1H), 7.78-7.74 (m, 1H), 7.74-7.29 (m, 10H), 6.77 (s, 1H), 6.47 (s, 1H), 4.71-4.61 (m, 2H), 4.38 (d, J=12.0 Hz, 2H), 4.14-4.07 (m, 4H), 3.71 (s, 5H), 3.65-3.55 (m, 4H), 3.48 (s, 4H), 3.45-3.25 (m, 5H), 3.16-3.02 (m, 3H), 2.71-2.55 (m, 2H), 2.32 (q, J=7.4 Hz, 2H), 2.17-1.98 (m, 3H), 1.95-1.79 (m, 3H), 1.39-1.19 (m, 10H), 1.11-1.01 (m, 1H), 0.95 (t, J=7.5 Hz, 3H). LCMS (ESI): R_{T}=1.180 min, m/z. found 1164.3 [M-CF₃COOH+H]⁺.

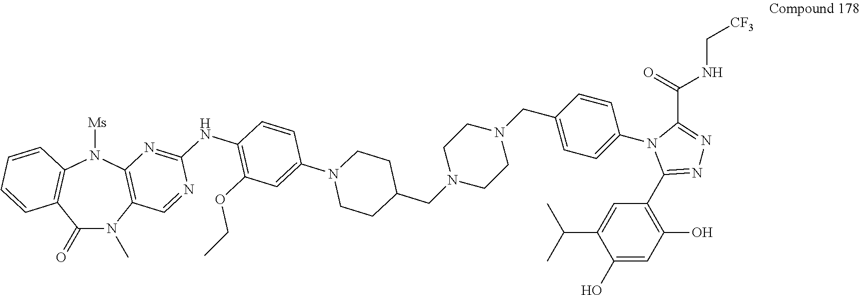 [SMILES: CCOc1cc(N2CCC(CN3CCN(Cc4ccc(-n5c(C(=O)NCC(F)(F)F)nnc5-c5cc(C(C)C)c(O)cc5O)cc4)CC3)CC2)ccc1Nc1ncc2c(n1)N(S(C)(=O)=O)c1ccccc1C(=O)N2C]

5-(2,4-dihydroxy-5-isopropylphenyl)-4-(4-((4-((1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidin-4-yl)methyl)piperazin-1-yl)methyl)phenyl)-N-(2,2,2-trifluoroethyl)-4H-1,2,4-triazole-3-carboxamide. ¹H NMR (400 MHz, DMSO-d₆): δ 9.57 (s, 1H), 8.72 (s, 1H), 8.60 (s, 1H), 7.74 (d, J=7.3 Hz, 1H), 7.61-7.53 (m, 2H), 7.49-7.17 (m, 7H), 6.63-6.44 (m, 3H), 6.24 (s, 1H), 4.04-3.91 (m, 4H), 3.66 (d, J=19.0 Hz, 4H), 3.48 (d, J=19.5 Hz, 6H), 2.95-2.89 (m, 1H), 2.68-2.60 (m, 2H), 2.41 (s, 5H), 2.17 (d, J=7.1 Hz, 2H), 2.05-1.94 (m, 1H), 1.78 (d, J=11.7 Hz, 2H), 1.65 (s, 1H), 1.24 (s, 4H), 1.15 (t, J=6.9 Hz, 4H), 0.93 (d, J=6.7 Hz, 1H), 0.79 (d, J=6.8 Hz, 6H). LCMS (ESI): R_{T}=1.210 min, m/z. found 1053.5 [M+H]⁺.

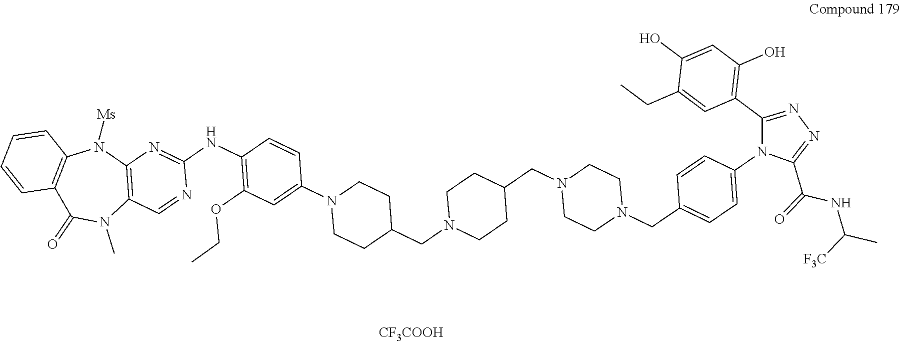 [SMILES: CCOc1cc(N2CCC(CN3CCC(CN4CCN(Cc5ccc(-n6c(C(=O)NC(C)C(F)(F)F)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

4-(4-((4-((1-((1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidin-4-yl)methyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide 2,2,2-trifluoroacetate, trifluoroacetic acid. ¹H NMR (400 MHz, DMSO-d₆): δ 9.80-9.56 (m, 2H), 9.19 (s, 1H), 8.79 (s, 1H), 8.62 (s, 1H), 7.75 (d, J=7.7 Hz, 1H), 7.64-7.28 (m, 9H), 6.64 (d, J=32.5 Hz, 3H), 6.31 (s, 1H), 4.63 (dd, J=15.6, 7.5 Hz, 1H), 4.11-3.94 (m, 3H), 3.69 (s, 6H), 3.57 (s, 2H), 3.46 (s, 3H), 3.17 (s, 2H), 3.02 (s, 4H), 2.94-2.62 (m, 8H), 2.28 (q, J=7.4 Hz, 3H), 1.89 (dd, J=27.5, 12.1 Hz, 7H), 1.40-1.31 (m, 8H), 1.18 (t, J=6.8 Hz, 3H), 0.89 (t, J=7.5 Hz, 3H). LCMS (ESI): R_{T}=1.090 min, m/z. found 1150.6 [M-CF₃COOH+H]⁺.

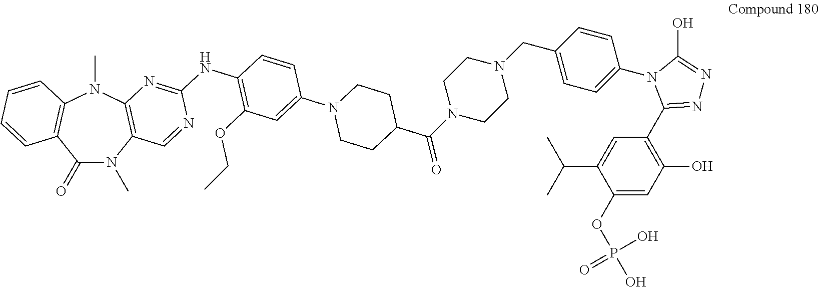 [SMILES: CCOc1cc(N2CCC(C(=O)N3CCN(Cc4ccc(-n5c(O)nnc5-c5cc(C(C)C)c(OP(=O)(O)O)cc5O)cc4)CC3)CC2)ccc1Nc1ncc2c(n1)N(C)c1ccccc1C(=O)N2C]

4-(4-(4-((4-(1-(4-((5,11-dimethyl-6-oxo-6,11-dihydro-5H-benzo[e]pyrimido[5,4-b][1,4]diazepin-2-yl)amino)-3-ethoxyphenyl)piperidine-4-carbonyl)piperazin-1-yl)methyl)phenyl)-5-hydroxy-4H-1,2,4-triazol-3-yl)-5-hydroxy-2-isopropylphenyl dihydrogen phosphate. ¹H NMR (400 MHz, DMSO-d₆): δ 8.53 (s, 1H), 8.33 (s, 1H), 7.75 (d, J=7.9 Hz, 1H), 7.55-7.41 (m, 3H), 7.31-7.24 (m, 3H), 7.22-7.13 (m, 4H), 6.86 (s, 1H), 4.34 (s, 2H), 4.26 (d, J=7.0 Hz, 2H), 3.70 (s, 2H), 3.63-3.56 (m, 2H), 3.52-3.44 (m, 8H), 3.21-3.16 (m, 2H), 3.13-3.11 (m, 1H), 2.98 (s, 2H), 2.05 (s, 5H), 1.52 (t, J=6.9 Hz, 3H), 1.16 (d, J=6.9 Hz, 6H). LCMS (ESI): RT=1.040 min, m/z. found 974.2 [M+H]⁺.

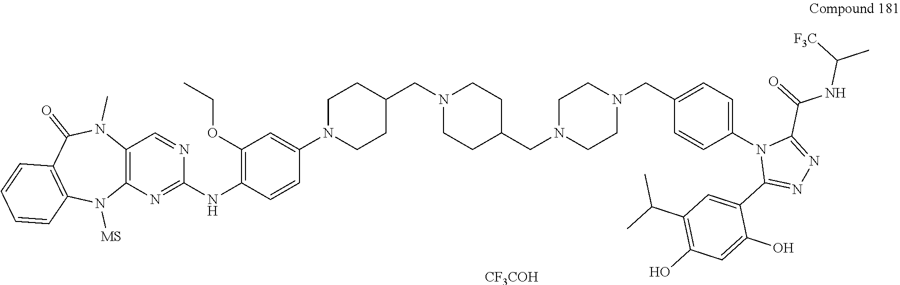

5-(2,4-dihydroxy-5-isopropylphenyl)-4-(4-((4-((1-((1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidin-4-yl)methyl)piperidin-4-yl)methyl)piperazin-1-yl)methyl)phenyl)-N-(1,1,1-trifluoropropan-2-yl)-4H-1,2,4-triazole-3-carboxamide, trifluoroacetic acid. ¹H NMR (400 MHz, DMSO-d₆) δ 9.77 (s, 1H), 9.61 (d, J=8.9 Hz, 1H), 9.20-9.05 (m, 1H), 8.77 (s, 1H), 8.61 (s, 1H), 7.75 (d, J=7.8 Hz, 1H), 7.58 (s, 2H), 7.48-7.32 (m, 5H), 6.66 (d, J=6.4 Hz, 2H), 6.56 (d, J=7.8 Hz, 1H), 6.33 (s, 1H), 5.32 (t, J=4.6 Hz, 1H), 4.70-4.59 (m, 1H), 4.11-3.98 (m, 4H), 3.68 (s, 8H), 3.46 (s, 4H), 3.07-2.73 (m, 12H), 2.02-1.81 (m, 7H), 1.34 (d, J=7.0 Hz, 4H), 1.24 (s, 5H), 1.18 (t, J=6.7 Hz, 3H), 0.87 (d, J=6.0 Hz, 6H). LCMS (ESI): R_{T}=1.105 min, m/z. found 1164.7 [M-CF₃COOH+H]⁺.

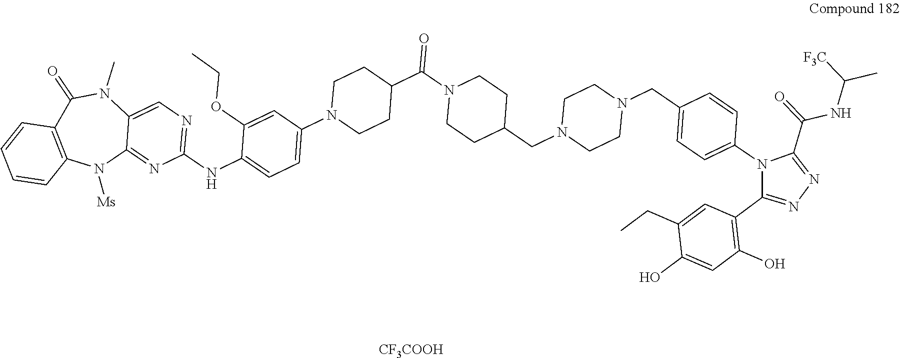 [SMILES: CCOc1cc(N2CCC(C(=O)N3CCC(CN4CCN(Cc5ccc(-n6c(C(=O)NC(C)C(F)(F)F)nnc6-c6cc(CC)c(O)cc6O)cc5)CC4)CC3)CC2)ccc1Nc1ncc2c(n1)N(S(C)(=O)=O)c1ccccc1C(=O)N2C ; "CF3COOH"]

5-(2,4-dihydroxy-5-isopropylphenyl)-4-(4-((4-((1-(1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidine-4-carbonyl)piperidin-4-yl)methyl)piperazin-1-yl)methyl)phenyl)-N-(1,1,1-trifluoropropan-2-yl)-4H-1,2,4-triazole-3-carboxamide, trifluoroacetic acid. ¹H NMR (400 MHz, DMSO-d₆) δ 9.77 (s, 1H), 9.62 (s, 1H), 9.59 (s, 1H), 8.83 (s, 1H), 8.63 (s, 1H), 7.75 (d, J=7.8 Hz, 1H), 7.59 (d, J=3.7 Hz, 3H), 7.54-7.32 (m, 8H), 6.83-6.60 (m, 3H), 6.33 (s, 1H), 4.68-4.59 (m, 1H), 4.40 (d, J=11.9 Hz, 1H), 4.11-3.93 (m, 4H), 3.69 (s, 7H), 3.46 (s, 5H), 2.96-2.88 (m, 4H), 2.04-1.92 (m, 2H), 1.75 (s, 8H), 1.34 (d, J=7.0 Hz, 4H), 1.24 (s, 3H), 1.19 (t, J=6.7 Hz, 5H), 0.87 (d, J=6.8 Hz, 6H). LCMS (ESI): R_{T} 1.210 min, m/z. found 1178.7[M-CF₃COOH+H]⁺.

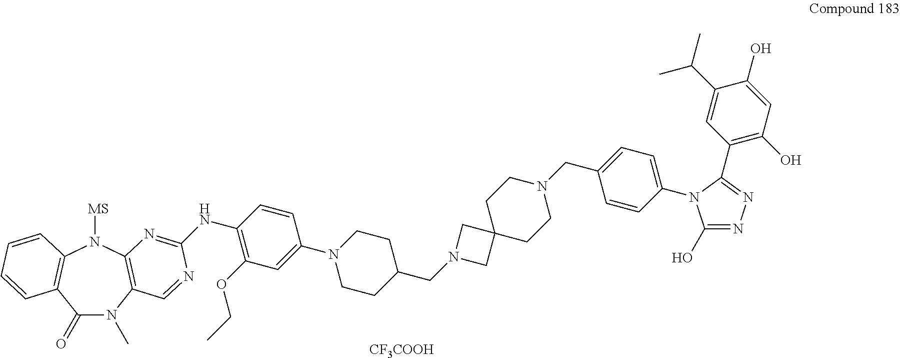

2-((4-(4-((7-(4-(3-(2,4-dihydroxy-5-isopropylphenyl)-5-hydroxy-4H-1,2,4-triazol-4-yl)benzyl)-2,7-diazaspiro[3.5]nonan-2-yl)methyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, DMSO-d₆) δ 11.98 (s, 1H), 10.03 (s, 1H), 9.80-9.60 (m, 2H), 9.37 (s, 1H), 8.74 (s, 1H), 8.60 (s, 1H), 7.75 (d, J=8.2 Hz, 1H), 7.58 (s, 2H), 7.47 (d, J=8.0 Hz, 2H), 7.29 (t, J=9.6 Hz, 2H), 6.84 (s, 1H), 6.62 (s, 1H), 6.52 (d, J=8.3 Hz, 1H), 6.26 (s, 1H), 4.24 (s, 1H), 4.15 (s, 1H), 4.09-3.89 (m, 5H), 3.68 (s, 3H), 3.46 (s, 3H), 3.29-3.17 (m, 4H), 3.07-2.85 (m, 3H), 2.65 (d, J=11.1 Hz, 2H), 2.35-2.16 (m, 3H), 2.09-1.94 (m, 1H), 1.80 (dd, J=42.0, 11.6 Hz, 4H), 1.37-1.21 (m, 4H), 1.17 (t, J=6.9 Hz, 3H), 1.00 (dd, J=6.6, 3.6 Hz, 6H). LCMS (ESI): R_{T}=0.940 min, m/z. found 984.3 [M-CF₃COOH+H]⁺.

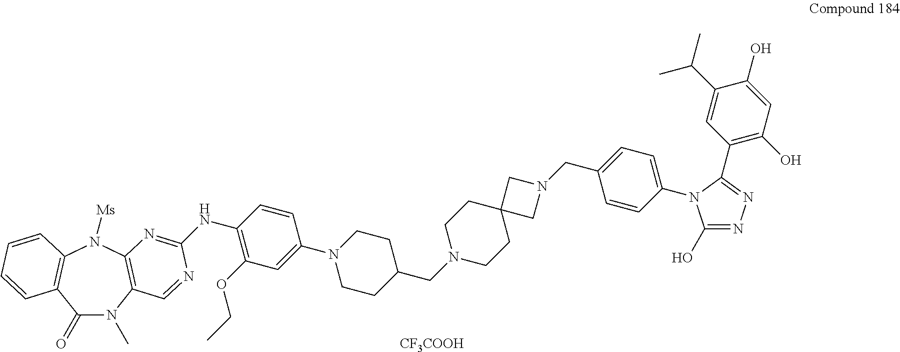 [SMILES: CCOc1cc(N2CCC(CN3CCC4(CC3)CN(Cc3ccc(-n5c(O)nnc5-c5cc(C(C)C)c(O)cc5O)cc3)C4)CC2)ccc1Nc1ncc2c(n1)N(S(C)(=O)=O)c1ccccc1C(=O)N2C ; "CF3COOH"]

2-((4-(4-((2-(4-(3-(2,4-dihydroxy-5-isopropylphenyl)-5-hydroxy-4H-1,2,4-triazol-4-yl)benzyl)-2,7-diazaspiro[3.5]nonan-7-yl)methyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one, trifluoroacetic acid. ¹H NMR (400 MHz, CD₃OD) δ 8.61 (s, 1H), 7.96 (d, J=8.8 Hz, 1H), 7.84 (dd, J=7.7, 1.5 Hz, 1H), 7.64-7.45 (m, 6H), 7.38 (d, J=8.4 Hz, 2H), 6.89 (d, J=4.9 Hz, 2H), 6.83 (d, J=8.2 Hz, 1H), 6.21 (s, 1H), 4.46 (s, 2H), 4.17-4.07 (m, 5H), 3.74-3.67 (m, 2H), 3.56 (d, J=4.3 Hz, 6H), 3.14-3.06 (m, 5H), 2.24-2.14 (m, 3H), 2.06-1.98 (m, 3H), 1.67-1.57 (m, 2H), 1.40-1.27 (m, 9H), 1.03 (d, J=6.9 Hz, 6H). LCMS (ESI): R_{T}=1.150 min, m/z. found 984.5 [M-CF₃COOH+H]⁺.

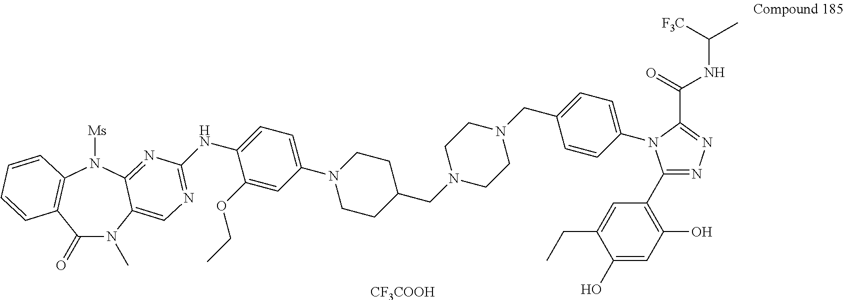 [SMILES: CCOc1cc(N2CCC(CN3CCN(Cc4ccc(-n5c(C(=O)NC(C)C(F)(F)F)nnc5-c5cc(CC)c(O)cc5O)cc4)CC3)CC2)ccc1Nc1ncc2c(n1)N(S(C)(=O)=O)c1ccccc1C(=O)N2C ; "CF3COOH"]

4-(4-((4-((1-(3-ethoxy-4-((5-methyl-11-(methylsulfonyl)-6-oxo-6,11-dihydro-5H-benzo[e]pyrimido[5,4-b][1,4]diazepin-2-yl)amino)phenyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, trifluoroacetic acid. ¹H NMR (400 MHz, DMSO-d₆) δ 9.70 (s, 1H), 9.60 (d, J=9.0 Hz, 1H), 8.77 (s, 1H), 8.62 (s, 1H), 7.75 (d, J=7.4 Hz, 1H), 7.60-7.57 (m, 2H), 7.49-7.29 (m, 7H), 6.68 (s, 2H), 6.59 (s, 1H), 6.31 (s, 1H), 4.67-4.61 (m, 1H), 4.07-3.99 (m, 4H), 3.68 (s, 5H), 3.46 (s, 3H), 2.99 (s, 4H), 2.77 (s, 2H), 2.34-2.24 (m, 3H), 2.03-1.97 (m, 1H), 1.84 (d, J=12.5 Hz, 2H), 1.34 (d, J=7.0 Hz, 5H), 1.24 (s, 3H), 1.18 (t, J=6.9 Hz, 4H), 0.92-0.84 (m, 4H). LCMS (ESI): R_{T}=1.210 min, m/z. found 1053.3 [M-CF₃COOH+H]⁺.

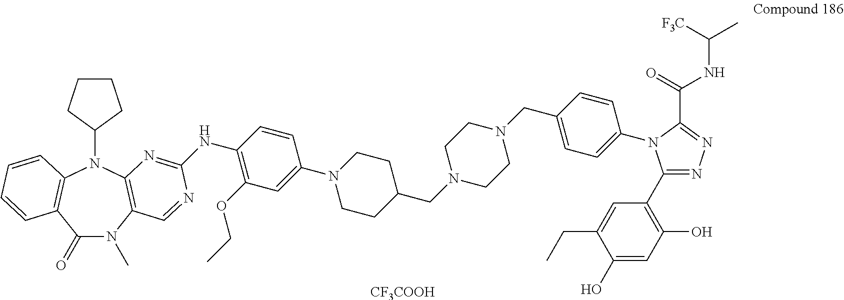 [SMILES: CCOc1cc(N2CCC(CN3CCN(Cc4ccc(-n5c(C(=O)NC(C)C(F)(F)F)nnc5-c5cc(CC)c(O)cc5O)cc4)CC3)CC2)ccc1Nc1ncc2c(n1)N(C1CCCC1)c1ccccc1C(=O)N2C ; "CF3COOH"]

4-(4-((4-((1-(4-((11-cyclopentyl-5-methyl-6-oxo-6,11-dihydro-5H-benzo[e]pyrimido[5,4-b][1,4]diazepin-2-yl)amino)-3-ethoxyphenyl)piperidin-4-yl)methyl)piperazin-1-yl)methyl)phenyl)-5-(5-ethyl-2,4-dihydroxyphenyl)-N-(1,1,1-trifluoropropan-2-yl)-4H-1,2,4-triazole-3-carboxamide, trifluoroacetic acid. ¹H NMR (400 MHz, DMSO-d₆): δ 9.69 (s, 1H), 9.60 (d, J=9.2 Hz, 1H), 8.35 (s, 1H), 7.93 (s, 1H), 7.87-7.73 (m, 1H), 7.57 (d, J=7.7 Hz, 1H), 7.48-7.24 (m, 7H), 7.16 (t, J=7.2 Hz, 1H), 6.68 (s, 3H), 6.31 (s, 1H), 4.63 (s, 2H), 4.07 (d, J=7.5 Hz, 3H), 3.41 (s, 6H), 3.07 (s, 8H), 2.28 (d, J=7.6 Hz, 5H), 2.06 (s, 2H), 1.84 (s, 2H), 1.57 (s, 6H), 1.36-1.26 (m, 9H), 0.89 (t, J=7.5 Hz, 4H). LCMS (ESI): RT=1.330 min, m/z. found 1043.3 [M-CF3COOH+H]⁺.

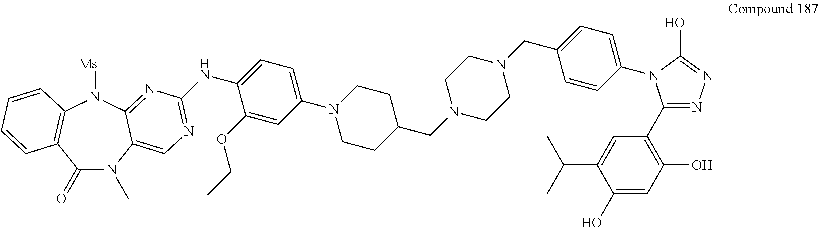 [SMILES: CCOc1cc(N2CCC(CN3CCN(Cc4ccc(-n5c(O)nnc5-c5cc(C(C)C)c(O)cc5O)cc4)CC3)CC2)ccc1Nc1ncc2c(n1)N(S(C)(=O)=O)c1ccccc1C(=O)N2C]

2-((4-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-2-ethoxyphenyl)amino)-5-methyl-11-(methylsulfonyl)-5,11-dihydro-6H-benzo[e]pyrimido[5,4-b][1,4]diazepin-6-one. ¹H NMR (400 MHz, DMSO-d₆): δ 11.92 (s, 1H), 9.60 (s, 1H), 9.41 (s, 1H), 8.72 (s, 1H), 8.60 (s, 1H), 7.74 (d, J=7.5 Hz, 1H), 7.58 (d, J=3.7 Hz, 2H), 7.48-7.41 (m, 1H), 7.27 (dd, J=16.9, 8.5 Hz, 3H), 7.13 (d, J=8.3 Hz, 2H), 6.76 (s, 1H), 6.57 (s, 1H), 6.47 (d, J=8.7 Hz, 1H), 6.27 (s, 1H), 4.07-3.91 (m, 3H), 3.65 (d, J=20.0 Hz, 5H), 3.45 (d, J=4.4 Hz, 6H), 3.03-2.90 (m, 1H), 2.63 (t, J=11.4 Hz, 2H), 2.37 (s, 6H), 2.16 (d, J=6.4 Hz, 2H), 2.03-1.95 (m, 1H), 1.76 (d, J=11.0 Hz, 2H), 1.62 (s, 1H), 1.32 (d, J=15.0 Hz, 2H), 1.15 (t, J=6.9 Hz, 3H), 0.94 (d, J=6.9 Hz, 6H). LCMS (ESI): RT=1.150 min, m/z. found 944.3 [M+H]⁺.

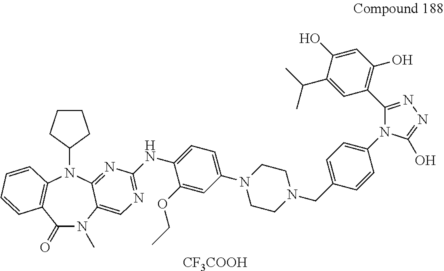 [SMILES: CCOc1cc(N2CCN(Cc3ccc(-n4c(O)nnc4-c4cc(C(C)C)c(O)cc4O)cc3)CC2)ccc1Nc1ncc2c(n1)N(C1CCCC1)c1ccccc1C(=O)N2C ; "CF3COOH"]

11-cyclopentyl-2-((4-(4-(4-(3-(2,4-dihydroxy-5-isopropylphenyl)-5-hydroxy-4H-1,2,4-triazol-4-yl)benzyl)piperazin-1-yl)-2-ethoxyphenyl)amino)-5-methyl-5,11-dihydro-6H-benzo[e]pyrimido[5,4-b][1,4]diazepin-6-one, trifluoroacetic acid. ¹H NMR (400 MHz, DMSO-d₆): δ 11.93 (s, 1H), 9.62 (s, 1H), 8.47 (s, 1H), 8.33 (s, 1H), 7.85 (s, 1H), 7.75 (d, J=8.9 Hz, 1H), 7.56 (d, J=6.0 Hz, 1H), 7.43 (d, J=7.4 Hz, 1H), 7.34 (d, J=8.0 Hz, 1H), 7.26 (d, J=8.6 Hz, 1H), 7.15 (d, J=7.8 Hz, 3H), 6.78 (s, 1H), 6.60 (s, 1H), 6.49 (s, 1H), 6.27 (s, 1H), 4.05 (d, J=5.6 Hz, 2H), 3.52 (s, 2H), 3.40 (s, 3H), 2.33 (s, 1H), 2.08 (s, 4H), 1.56 (s, 5H), 1.29-1.23 (m, 8H), 1.17-1.09 (m, 3H), 0.96 (d, J=6.8 Hz, 6H). LCMS (ESI): RT=1.117 min, m/z. found 837.5[M-CF₃COOH+H]⁺.

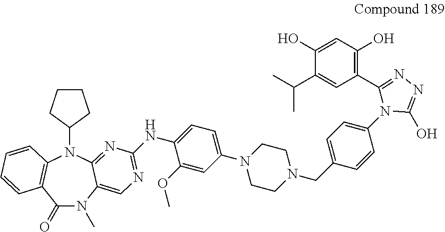 [SMILES: COc1cc(N2CCN(Cc3ccc(-n4c(O)nnc4-c4cc(C(C)C)c(O)cc4O)cc3)CC2)ccc1Nc1ncc2c(n1)N(C1CCCC1)c1ccccc1C(=O)N2C]

2-((4-(4-(2-(4-(4-(3-(2,4-dihydroxy-5-isopropylphenyl)-5-hydroxy-4H-1,2,4-triazol-4-yl)benzyl)piperazin-1-yl)-2-oxoethoxy)piperidin-1-yl)-2-ethoxyphenyl)amino)-5,11-dimethyl-5H-benzo[e]pyrimido[5,4-b][1,4]diazepin-6(11H)-one, trifluoroacetic acid. ¹H NMR (400 MHz, DMSO-d₆) δ 11.92 (s, 1H), 9.59 (s, 1H), 9.40 (s, 1H), 8.32 (s, 1H), 7.93 (s, 1H), 7.69 (d, J=8.8 Hz, 1H), 7.55 (dd, J=7.7, 1.6 Hz, 1H), 7.43 (ddd, J=12.2, 4.9 Hz, 1H), 7.34 (d, J=8.3 Hz, 2H), 7.26 (d, J=8.3 Hz, 1H), 7.15 (t, J=7.6 Hz, 3H), 6.78 (s, 1H), 6.61 (d, J=2.2 Hz, 1H), 6.51-6.45 (m, 1H), 6.28 (s, 1H), 3.78 (s, 3H), 3.52 (s, 2H), 3.40 (s, 3H), 3.12 (s, 4H), 2.99-2.95 (m, 1H), 1.56 (s, 5H), 1.28-1.17 (m, 8H), 0.96 (d, J=6.9 Hz, 6H). LCMS (ESI): R_{T}=1.097 min, m/z. found 823.4 [M-CF₃COOH+H]⁺.

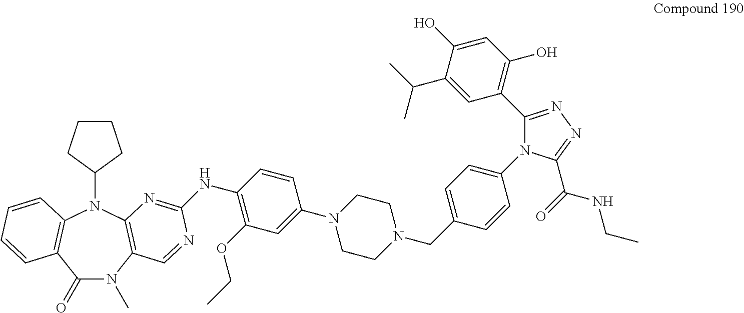 [SMILES: CCNC(=O)c1nnc(-c2cc(C(C)C)c(O)cc2O)n1-c1ccc(CN2CCN(c3ccc(Nc4ncc5c(n4)N(C4CCCC4)c4ccccc4C(=O)N5C)c(OCC)c3)CC2)cc1]

4-(4-((4-(4-((11-cyclopentyl-5-methyl-6-oxo-6,11-dihydro-5H-benzo[e]pyrimido[5,4-b][1,4]diazepin-2-yl)amino)-3-ethoxyphenyl)piperazin-1-yl)methyl)phenyl)-5-(2,4-dihydroxy-5-isopropylphenyl)-N-ethyl-4H-1,2,4-triazole-3-carboxamide. ¹H NMR (400 MHz, DMSO-d₆) δ 8.95 (t, J=5.8 Hz, 1H), 8.34 (s, 1H), 7.86 (s, 1H), 7.76 (d, J=8.7 Hz, 1H), 7.56 (dd, J=7.7, 1.6 Hz, 1H), 7.44 (dd, J=13.4, 5.0 Hz, 3H), 7.29 (dd, J=24.1, 8.3 Hz, 3H), 7.15 (t, J=7.5 Hz, 1H), 6.65-6.57 (m, 2H), 6.50 (dd, J=8.8, 2.2 Hz, 1H), 6.33 (s, 1H), 4.65-4.54 (m, 1H), 4.12-4.00 (m, 2H), 3.58 (s, 2H), 3.40 (s, 4H), 3.23-3.08 (m, 7H), 3.00-2.87 (m, 1H), 2.56 (s, 4H), 2.32-2.22 (m, 1H), 2.12-2.00 (m, 1H), 1.65-1.46 (m, 5H), 1.44-1.33 (m, 1H), 1.27 (t, J=6.9 Hz, 3H), 1.05 (t, J=7.2 Hz, 3H), 0.83 (d, J=6.9 Hz, 6H). LCMS (ESI): R_{T}=1.437 min, m/z. found 892.5 [M+H]⁺.

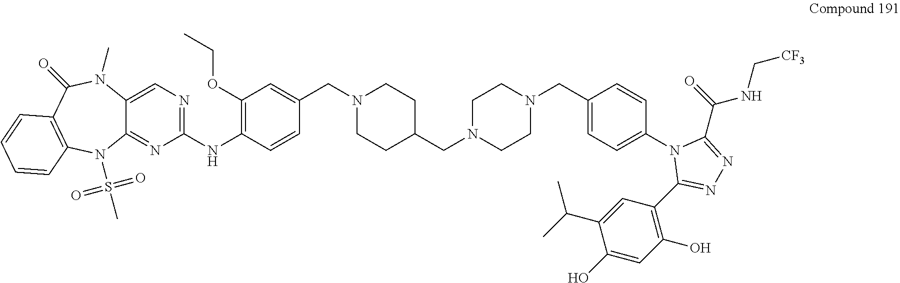 [SMILES: CCOc1cc(CN2CCC(CN3CCN(Cc4ccc(-n5c(C(=O)NCC(F)(F)F)nnc5-c5cc(C(C)C)c(O)cc5O)cc4)CC3)CC2)ccc1Nc1ncc2c(n1)N(S(C)(=O)=O)c1ccccc1C(=O)N2C]

5-(2,4-dihydroxy-5-isopropylphenyl)-4-(4-((4-((1-(3-ethoxy-4-((5-methyl-11-(methylsulfonyl)-6-oxo-6,11-dihydro-5H-benzo[e]pyrimido[5,4-b][1,4]diazepin-2-yl)amino)benzyl)piperidin-4-yl)methyl)piperazin-1-yl)methyl)phenyl)-N-(2,2,2-trifluoroethyl)-4H-1,2,4-triazole-3-carboxamide. ¹H NMR (400 MHz, DMSO-d₆): δ 10.45 (s, 1H), 9.83 (s, 1H), 9.68-9.54 (m, 1H), 8.92 (s, 1H), 8.69 (s, 1H), 7.76 (d, J=7.5 Hz, 2H), 7.70-7.55 (m, 3H), 7.53-7.25 (m, 6H), 6.61 (s, 1H), 6.38 (s, 1H), 4.22-3.89 (m, 6H), 3.70 (s, 4H), 3.48 (s, 7H), 2.98-2.81 (m, 2H), 2.45-2.06 (m, 9H), 1.24 (s, 8H), 0.82 (d, J=6.8 Hz, 7H). LCMS (ESI): R_{T}=1.367 min, m/z. found 1067.5 [M+H]⁺.

### Example 2: Testing Various CHAMP Molecules

### Materials and Methods

### Cell Lines

The following cancer cell lines were employed: A549 human lung carcinoma (ATCC, #CCL-185); BT-474 human breast carcinoma (ATCC, #HTB-20); MDA-MB-231 human breast adenocarcinoma (ATCC, #CRM−HTB-26); MDA-MB-468 human breast adenocarcinoma (ATCC, #HTB-132); MV-4-11 human acute myeloid leukemia (ATCC, #CRL-9591); and U-87 MG human glioblastoma (ATCC, #HTB-14). Cell lines were cultured essentially according to ATCC recommendations.

### HSP90α-Binding Fluorescent Polarization (FP) Assay

Binding of test compounds to HSP90α protein was measured by fluorescent polarization (FP) using the HSP90α (N-terminal) Assay Kit (BPS Bioscience, #50298), following the manufacturer's instructions, except as noted. Fluorescently labeled HSP90-binding compounds, either the provided FITC-geldanamycin (5 nM final concentration) or RNK04010, a triazolone-based HSP90 binding small molecule labeled with BODIPY through a piperazine-phenyl linker (5 nM final concentration) were employed. A 2.5-fold serial dilution of each test compound ranging from 20 μM to 5.2 nM was assayed for binding to HSP90α. After the final step of adding HSP90α protein to each assay well, plates were mixed by brief shaking, incubated at 25° C. for 120 min for FITC-geldanamycin or 300 min for RNK04010, and fluorescence was measured using a PerkinElmer EnVision Plate Reader. Background-subtracted mP values were calculated from raw data and a four-parameter “log[inhibitor] vs. response” curve was fitted and IC50 values (the concentration at which 50% of the maximal inhibition occurs) calculated using GraphPad Prism 7 software.

### MAPK7 Kinase Assay

The inhibition of MAPK7 kinase activity by test compounds was measured by the ADP-Glo Kinase Assay (Promega, #V6930), following the manufacturer's instructions, except as noted. 3-fold serial dilutions of each test compound were prepared ranging from 50 μM to 2.54 nM. Also, as a positive control, a 4-fold serial dilution of the MAPK7 inhibitor, XMD17-109 (MedChemExpress, #HY-15665) was prepared ranging from 10 μM to 0.04 nM. In brief, in 384-well plates, test compounds were added to the kinase reaction mix containing recombinant MAPK7 protein (Carna Biosciences, #04-146; 20 nM final concentration), myelin basic protein (SignalChem Biotech, #M42-54G; 0.1 mg/ml final concentration) and ATP (340 μM final concentration), then mixed by shaking and incubated with ADP-Glo Reagent and Kinase Detection Reagent. Luminescence was measured on a BioTek plate reader. The % inhibition was calculated using following equation and plotted and IC50 values (the concentration at which 50% of the maximal inhibition occurs) calculated using GraphPad Prism 7 software:

$$\% Inhibition = ⌊ \frac{{\overline{Lum}}_{vehicle} - {Lum}_{compd}}{{\overline{Lum}}_{vehicle} - {\overline{Lum}}_{positive}} ⌋ * 100$$

L̅u̅m̅ₚₒₛᵢₜᵢᵥₑ: The average ratio for 10 wells of positive controls (10 μM XMD17-109) across the plate.

L̅u̅m̅_{vehicle}: The average ratio for 10 wells of negative controls (0.5% DMSO) across the plate.

### MAPK7 Western Blot Protein Degradation Assay

U-87 MG human glioblastoma cells were seeded in 6- or 12-well tissue culture plates, and after 1 hr, test compounds were added at various concentrations and incubated at 37° C./5% CO₂ for 48 hr. Cells were then washed with cold PBS, aspirated and cold RIPA buffer containing a protease/phosphatase inhibitor cocktail was added to lyse cells. After centrifugation, the total protein concentrations of cell lysates were determined using the BCA protein assay. Samples were normalized for equivalent protein concentrations, 5×SDS-PAGE loading buffer added and denatured at 100° C. for 10 min. 20 μl of each sample/well was loaded on a SDS-PAGE gel and electrophoresed for 20 min at 80 V, then 120 V for 1.5 hr. Gels were then electroblotted to nitrocellulose membranes using a wet-transfer method at 250 mA for 2.5 hr. Membranes were incubated with blocking buffer for 1 hr and washed 3 times with TBST for 5 min. Membranes were then incubated with anti-MAPK7 (anti-ERK5; Cell Signaling Technology, #12950) and anti-3-Actin (Cell Signaling Technology, #3700) monoclonal antibodies diluted in blocking buffer at 4° C. overnight per the manufacturer's recommendations. After washing 3 times, blots were incubated with appropriately labeled secondary antibodies for 1 hr at room temperature and washed again. Fluorescence imaging and quantitation was performed using a LI-COR Odyssey. Results was analyzed using GraphPad Prism 7 software.

### ERBB2 (HER2) Flow Cytometry Protein Degradation Assay

BT-474 human breast carcinoma cells were plated in 24-well tissue culture plates at 250,000 cells/well and incubated at 37° C./5% CO₂ for 24 hr. Cells were then treated with test compounds at various concentrations and incubated at 37° C./5% CO₂ for 24 hr. To analyze total ERBB2 expression by flow cytometry, cells were detached with trypsin, washed, counted and treated with 10 μl/10⁶ cells PE-conjugated, anti-ERBB2 monoclonal antibody (R&D Systems, #FAB1129P) for 30 min at 25° C. in dark. Cells were then washed, resuspended in 200 μl 1% paraformaldehyde and analyzed by flow cytometry. Compound inhibition of was determined by the following equation and DC50 values (the concentration at which 50% of the maximal degradation of ERBB2 occurs) calculated using GraphPad Prism 7 software:

% Inhibition=100−(D−B)/(S−B)*100%.
S: Fluorescence intensity of cells with antibody
D: Fluorescence intensity of compound-treated cells with antibody
B: Fluorescence intensity of cells without antibody

### Cancer Cell Line Proliferation (CCK-8) Assays

Cells were plated in 96-well tissue culture plates at 4,000 cells/well and incubated at 37° C./5% CO₂ for 24 hr. 3-fold serial dilutions of each test compound were prepared ranging from 20 μM to 1.02 nM. Cells were then treated with test compounds at various concentrations with a final concentration of 0.5% DMSO/well, and then incubated at 37° C./5% CO₂ for 72 hr. 10 μl the cell proliferation detection reagent CCK-8 (Dojindo Molecular Technologies, #CK04) was added to each well and incubated at 37° C./5% CO₂ for 3-4 hr and the absorbance at 450 nm measured using an Perkin Elmer EnVision Plate Reader. Compound inhibition of was determined by the following equation and EC50 values (the concentration at which 50% of the maximal inhibition occurs) calculated using GraphPad Prism 7 software:

% Inhibition=100−(D−B)/(S−B)*100%.
S: Absorbance of cells treated with DMSO
D: Absorbance of compound-treated cells
B: Absorbance of medium with DMSO without cells

### Results

A number of synthetic schemes have been developed to construct various CHAMP molecules designed to degrade MAPK7, which are termed MAPK7-CHAMP molecules. Representative examples are shown, each consisting of a HSP90 binder linked to a MAPK7 binder. Similar chemistry can be applied to other CHAMP molecules not limited to these specific HSP90- and MAPK7-binding moieties.

HSP90α-binding fluorescent polarization (FP) assays measuring competition with the fluorescently labeled HSP90 binders, FITC-geldanamycin or RNK04010 (BODIPY-labeled), were applied to assess the binding capabilities of CHAMP molecules to HSP90. As shown in Table 1, CHAMP molecules containing HSP90-binding moieties documented in the literature were generally in agreement with the published structure activity relationship (SAR).

The incorporation a MAPK7 binder of similar molecular weight to the HSP90 binder into the CHAMPs typically had only minimal impact on the binding of CHAMP molecules to HSP90α in this assay (Table 1). There are a number of reasons: first the co-crystal structures of these moieties with their corresponding proteins are available and allow precise structure-based molecular designs; and secondly, the linker is constructed to provide rigidity with suitable length.

Binding by a variety of CHAMP molecules to MAPK7 was assessed by measuring inhibition of MAPK7 phosphorylation activity in a biochemical kinase assay, as shown in Table 1. CHAMP molecules containing MAPK7-binding moieties documented in the literature were generally in agreement with the published SAR.

The incorporation of a chaperone-binding moiety, such as a HSP90 binder, typically had only minimal impact on the binding of CHAMP molecules to MAPK7 as measured by this assay. There are a number of reasons: first the co-crystal structures of these moieties with their corresponding proteins are available and allow precise structure-based molecular designs; and secondly, the linker is constructed to provide rigidity with suitable length.

Heterobifunctional CHAMP molecules with both a MAPK7-binding moiety and a HSP90-binding moiety are designed to induce targeted protein degradation (TPD) of MAPK7. As shown in Table 2, U-87 MG human glioblastoma cells, which express MAPK7, were treated for 48 hr with various concentrations of CHAMP compounds and degradation of MAPK7 was observed by Western blotting.

CHAMP molecules may include chaperone or chaperone complex binders that have a range of different binding affinities. In different embodiments, it is desirable to use a high-affinity binder, a moderate-affinity binder or a low-affinity binder. Since a HSP90-binding moiety that interacts with the N-terminal ATP-binding pocket of HSP90 may inhibit HSP90 activity and induce the degradation of HSP90 client proteins, some CHAMP molecules may not only induce the degradation of the desired target protein or proteins (which may or may not be HSP90 client proteins), but also simultaneously induce the degradation of HSP90 client proteins. As shown in Table 1, CHAMP compounds also displayed various levels of degradation of HSP90 client protein ERBB2 as assessed by flow cytometry in ERBB2-expressing BT-474 human breast carcinoma cells.

As shown in Table 1, various MAPK7 CHAMP molecules also inhibited the growth and/or survival of a panel of cancer cell lines as measured by a CCK-8 cell line proliferation assay.

TABLE 1
Biochemical and Cell-based Assays of Compounds

| | HSP90α | HSP90α | | | | MDA- | MDA- | | |
|---|---|---|---|---|---|---|---|---|---|
| Comp | binding | binding | MAPK7 | ERBB2 | A549 | MB-231 | MB-468 | MV-4-11 | U87MG |
| # | (BODIPY)¹ | (FITC)² | kinase³ | degradation⁴ | CCK-8⁵ | CCK-8⁶ | CCK-8⁷ | CCK-8⁸ | CCK-8⁹ |
| 001 | | A | | C | | | | B | C |
| 002 | | A | | B | | | | A | B |
| 003 | | B | | B | | | | A | B |
| 004 | B | B | | B | B | A | A | A | A |
| 005 | | B | | B | | | | A | A |
| 006 | | B | | C | | | | B | C |
| 007 | | B | | B | | | | A | B |
| 008 | | B | | | | | | A | B |
| 009 | | B | | | | | | A | B |
| 010 | | B | | C | | C | C | A | B |
| 011 | | B | | | | | | B | C |
| 012 | | B | | | | | | B | C |
| 013 | | B | | B | | B | B | A | B |
| 014 | | B | | | | | | B | B |
| 015 | | B | | | | | | A | B |
| 016 | | B | | | | | | A | B |
| 017 | | B | | | | | | A | C |
| 018 | B | | | B | | | | A | B |
| 019 | B | | | | | | | A | B |
| 020 | B | | | | | | | B | B |
| 021 | B | | | | | | | B | C |
| 022 | B | | | B | | | | A | B |
| 023 | B | | | | | | | A | B |
| 024 | B | | C | B | | | | A | A |
| 025 | B | | | B | | | | A | B |
| 026 | C | | | C | | | | C | C |
| 027 | B | | | C | | | | B | C |
| 028 | B | | | B | | | | B | C |
| 029 | C | | | B | | | | B | C |
| 030 | B | | | B | | | | B | C |
| 031 | C | | | C | | | | C | C |
| 032 | A | | C | B | | | | A | B |
| 033 | | | | | | | | | B |
| 034 | B | | | | | | | A | B |
| 035 | B | | | B | | | | A | B |
| 036 | B | | | B | | | | A | C |
| 037 | C | | | B | | | | B | B |
| 038 | B | | | B | | | | A | C |
| 039 | C | | | B | | | | B | B |
| 040 | B | | | | B | A | A | A | B |
| 041 | A | | | | | | | A | A |
| 042 | A | | C | B | | | | A | A |
| 043 | B | | C | B | | | | A | A |
| 044 | A | | | | | | | A | A |
| 045 | B | | | | | | | A | B |
| 046 | B | | | | | | | A | B |
| 047 | B | | C | B | | | | A | B |
| 048 | B | | | | | | | A | B |
| 049 | B | | | | | A | A | A | B |
| 050 | B | | | | | | | A | B |
| 051 | B | | | | | | | A | A |
| 052 | C | | C | C | C | C | C | C | C |
| 053 | B | | | | | B | A | A | B |
| 054 | B | | C | B | B | B | B | A | B |
| 055 | B | | | | | | | A | B |
| 056 | B | | | B | | | | A | B |
| 057 | B | | | B | | | | A | B |
| 058 | B | | | | | | | A | A |
| 059 | A | | | | | | | A | A |
| 060 | B | | | | | | | A | A |
| 061 | A | | B | B | B | | | A | B |
| 062 | A | | | | | | | A | C |
| 063 | A | | | | | | | A | A |
| 064 | A | | | | | | | A | A |
| 065 | C | | | C | | | | C | C |
| 066 | C | | | C | | | | C | C |
| 067 | C | | | | | | | B | C |
| 068 | C | | | | | | | B | C |
| 069 | B | | | | | | | A | B |
| 070 | B | | | B | | | | A | B |
| 071 | B | | | | | | | B | C |
| 072 | B | | | | | | | A | B |
| 073 | C | | | | | | | C | C |
| 074 | C | | | | | | | B | C |
| 075 | A | | | B | | | | A | A |
| 076 | C | | | | | | | B | C |
| 077 | B | | | | | | | A | B |
| 078 | B | | B | B | B | | | A | B |
| 079 | A | | | | | | | B | C |
| 080 | B | | | | | | | A | C |
| 081 | A | | | | | | | A | B |
| 082 | A | | | | | | | A | A |
| 083 | A | | | | | | | A | A |
| 084 | B | | | | | | | A | B |
| 085 | B | | | | | | | A | B |
| 086 | A | | | | | | | A | B |
| 087 | B | | | | | | | A | B |
| 088 | B | | | | | | | B | C |
| 089 | B | | | | | | | B | C |
| 090 | B | | | | | | | B | C |
| 091 | A | | | | | | | B | C |
| 092 | B | | | | | | | C | C |
| 093 | A | | | | | | | B | C |
| 094 | B | | | | | | | B | C |
| 095 | C | | | | | | | B | C |
| 096 | A | | A | B | | | | A | A |
| 097 | B | | | | | | | A | C |
| 098 | A | | | B | | | | A | A |
| 099 | A | | C | B | | | | A | A |
| 100 | C | | | | | | | B | C |
| 101 | C | | | | | | | C | C |
| 102 | C | | | | | | | B | C |
| 103 | C | | C | C | | | | B | C |
| 104 | C | | | | | | | C | C |
| 105 | C | | C | C | | | | B | C |
| 106 | C | | | | | | | B | C |
| 107 | B | | | | | | | A | |
| 108 | C | | | | | | | C | C |
| 109 | C | | C | | | | | C | C |
| 110 | C | | C | | | | | C | C |
| 111 | C | | C | | | | | C | C |
| 112 | C | | C | | | | | C | C |
| 113 | C | | C | | | | | C | C |
| 114 | B | | | | | | | A | B |
| 115 | B | | C | B | | | | A | B |
| 116 | C | | | | | | | B | C |
| 117 | C | | | | | | | B | C |
| 118 | C | | | | | | | B | C |
| 119 | C | | | | | | | A | C |
| 120 | C | | | | | | | B | C |
| 121 | C | | | | | | | C | C |
| 122 | C | | | | | | | B | C |
| 123 | B | | | B | | | | A | B |
| 124 | B | | | B | | | | A | B |
| 125 | | C | | | | | | C | C |
| 126 | | B | | | | | | A | B |
| 127 | | B | | | | | | B | B |
| 128 | | C | A | | | | | C | C |
| 129 | | B | | | | | | B | C |
| 130 | | C | | | | | | B | C |
| 131 | | C | | | | | | B | C |
| 132 | | C | | | | | | B | C |
| 133 | A | | C | | | | | A | B |
| 134 | A | | | | | | | A | A |
| 135 | A | | C | | | | | A | B |
| 136 | A | | | | | | | A | B |
| 137 | C | | | | | | | B | |
| 138 | C | | | | | | | B | |
| 139 | C | | | | | | | B | |
| 140 | C | | | | | | | B | |
| 141 | C | | C | | | | | C | |
| 142 | | | B | | | | | | |
| 143 | | | | | | | | C | C |
| 144 | | | | | | | | C | C |
| 145 | | | | | | | | C | C |
| 146 | | | | | | | | C | C |
| 147 | | | | | | | | C | C |
| 148 | | | | | | | | C | C |
| 149 | | | | | | | | C | C |
| 150 | | | | | | | | C | C |
| 151 | | | | | | | | | C |
| 152 | | | | | | | | C | C |
| 153 | | | | | | | | C | |
| 154 | | | | | | | | C | |
| 155 | | | | | | | | C | |
| 156 | | | | | | | | C | |
| 157 | | | | | | | | C | |
| 158 | | | | | | | | C | |
| 159 | | | | | | | | A | A |
| 160 | | | | | | | | A | B |
| 161 | | | | | | | | A | B |
| 162 | | | | | | | | B | B |
| 163 | | | | | | | | A | A |
| 164 | | | | | | | | A | A |
| 165 | | | | | | | | A | B |
| 166 | | | | | | | | A | B |
| 167 | | | | | | | | A | A |
| 168 | | | | | | | | A | A |
| 169 | | | | | | | | A | B |
| 170 | | | | | | | | A | A |
| 171 | | | | | | | | C | C |
| 172 | | | | | | | | A | A |
| 173 | | | | | | | | A | A |
| 174 | | | | | | | | A | A |
| 175 | | | | | | | | A | A |
| 176 | | | | | | | | A | B |
| 177 | | | | | | | | A | B |
| 178 | | | | | | | | A | A |
| 179 | | | | | | | | A | A |
| 180 | | | | | | | | A | A |
| 181 | | | | | | | | A | B |
| 182 | | | | | | | | A | B |
| 183 | | | | | | | | A | B |
| 184 | | | | | | | | A | B |
| 185 | | | | | | | | A | B |
| 186 | | | | | | | | B | C |
| 187 | | | | | | | | A | A |
| 188 | | | | | | | | B | C |
| 189 | | | | | | | | B | C |
| 190 | | | | | | | | B | B |
| 191 | | | | | | | | A | A |

¹HSP90α-binding FP (BODIPY) assay: A. IC50 < 100 nM; B. IC50 = 100-1000 nM; C. IC50 > 1000 nM;
²HSP90α-binding FP (FITC) assay: A. IC50 < 100 nM; B. IC50 = 100-1000 nM; C. IC50 > 1000 nM^{;}
³MAPK7 kinase assay: A. IC50 < 100 nM; B. IC50 = 100-1000 nM; C. IC50 > 1000 nM;
⁴ERBB2 flow cytometry protein degradation assay in BT-474 cells: A. DC50 < 100 nM; B. DC50 = 100-1000 nM; C. DC50 > 1000 nM;
⁵A549 CCK-8 proliferation assay: A. EC50 < 100 nM; B. EC50 = 100-1000 nM; C. EC50 > 1000 nM;
⁶MDA-MB-231 CCK-8 proliferation assay: A. EC50 < 100 nM; B. EC50 = 100-1000 nM; C. EC50 > 1000 nM;
⁷MDA-MB-468 CCK-8 proliferation assay: A. EC50 < 100 nM; B. EC50 = 100-1000 nM; C. EC50 > 1000 nM;
⁸MV-4-11 CCK-8 proliferation assay: A. EC50 < 100 nM; B. EC50 = 100-1000 nM; C. EC50 > 1000 nM;
⁹U87 MG CCK-8 proliferation assay: A. EC50 < 100 nM; B. EC50 = 100-1000 nM; C. EC50 > 1000 nM

TABLE 2
MAPK7 Degradation by Compounds

| | MAPK7 | MAPK7 | MAPK7 | MAPK7 |
|---|---|---|---|---|
| Compound | degradation | degradation | degradation | degradation |
| # | (30 nM)¹ | (100 nM)¹ | (300 nM)¹ | (1000 nM)¹ |
| 004 | C | C | | A |
| 005 | C | C | | A |
| 013 | C | C | | A |
| 018 | C | C | | A |
| 021 | C | C | C | C |
| 022 | C | C | B | A |
| 024 | C | B | | A |
| 025 | C | C | | A |
| 032 | C | B | A | A |
| 036 | C | C | B | A |
| 040 | C | B | A | A |
| 043 | C | B | A | A |
| 054 | C | C | A | A |

¹MAPK7 Western blot protein degradation assay in U-87 MG cells:
A. >66% degradation;
B. 33-66% degradation;
C. < 33% degradation

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A compound of the Formula I: 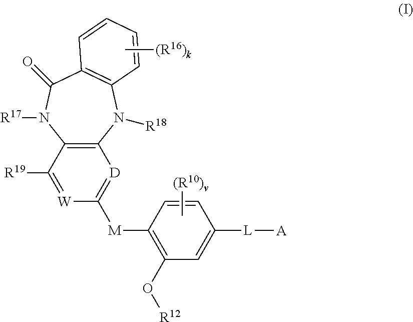
or a pharmaceutically acceptable salt thereof, wherein,
A is a chemical moiety that binds HSP90 protein;
L is a linker;
W and D are each independently N or CR⁹;
R¹⁰, R¹⁶, and R¹⁹ are each independently selected from halo, (C₁-C₆)alkyl, (C₂-C₆)alkenyl, halo(C₂-C₆)alkenyl, (C₂-C₆)alkynyl, —(C₁-C₆)alkylOR^{c}, —(C₁-C₆)alkylN(R^{d})₂, —(C₁-C₆)alkylC(O)OR^{d}, —(C₁-C₆)alkylC(O)N(R^{d})₂, —(C₁-C₆)alkylO(C₁-C₆)alkylN(R^{d})₂, —(C₁-C₆)alkylSOR^{d}, —(C₁-C₆)alkylS(O)₂R^{d}, —(C₁-C₆)alkylSON(R^{d})₂, —(C₁-C₆)alkylSO₂N(R^{d})₂, —(C₁-C₆)alkylcycloalkyl, —(C₁-C₆)alkylheterocyclyl, —(C₁-C₆)alkylheteroaryl, —(C₁-C₆)alkylaryl, —(C₁-C₆)alkoxy, halo(C₁-C₆)alkoxy, CN, aryl, heteroaryl, cycloalkyl, heterocycloalkyl, —C(O)R^{d}, —C(O)OR^{d}, —C(O)N(R^{d})₂, N(R^{d})₂, —C(O)NR^{d}(C₁-C₆)alkylN(R^{d})₂, —NR^{d}(C₁-C₆)alkylN(R^{d})₂, —NR^{d}(C₁-C₆)alkylOR^{d}, —SOR^{d}, —S(O)₂R^{d}, —SON(R^{d})₂, —SO₂N(R^{d})₂, and CN, wherein each aryl, cycloalkyl, heterocyclyl, and heteroaryl alone and in connection with —(C₁-C₆)alkylcycloalkyl, —(C₁-C₆)alkylheterocyclyl, —(C₁-C₆)alkylheteroaryl, —(C₁-C₆)alkylaryl are optionally substituted with 1 to 3 groups selected from R^{e};
M is O, S, or NR¹¹;
R¹¹, R¹⁷, R¹⁸, and R²⁰, are each independently selected from hydrogen, (C₁-C₆)alkyl, and S(O)₂(C₁-C₆)alkyl;
R¹² is hydrogen, (C₁-C₆)alkyl, halo(C₁-C₆)alkyl, —(C₁-C₆)alkylOR^{c}, S(O)₂(C₁-C₆)alkyl, aryl, heteroaryl, cycloalkyl, heterocycloalkyl, C(O)(C₁-C₆)alkyl, or —(C₁-C₆)alkylaryl, wherein each aryl, cycloalkyl, heterocyclyl, and heteroaryl alone and in connection with —(C₁-C₆)alkylaryl are optionally substituted with 1 to 3 groups selected from R^{e};
R^{c} and R^{d} are each independently selected from hydrogen, (C₁-C₆)alkyl, and halo(C₁-C₆)alkyl;
R^{e} is selected from halo, oxo, CN, NO₂, —N(R^{d})₂, —OR, —C(O)OR^{d}, (C₁-C₆)alkyl, —(C₁-C₆)alkylOR^{c}, halo(C₁-C₆)alkyl, (C₁-C₆)alkoxy, halo(C₁-C₆)alkoxy, —(C₁-C₆)alkylC(O)OR^{d}, —(C₁-C₆)alkylC(O)N(R^{d})₂, (C₂-C₆)alkenyl, halo(C₂-C₆)alkenyl, (C₂-C₆)alkynyl, —(C₁-C₆)alkylSR^{d}, —(C₁-C₆)alkylOR^{c}, —(C₁-C₆)alkylN(R^{d})₂, —C(O)N(R^{d})₂, —C(O)NR^{d}C₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, aryl, heteroaryl, cycloalkyl, and heterocycloalkyl; and
k and v are each independently 0, 1, 2, or 3.

2. The compound of claim 1, or a pharmaceutically acceptable salt thereof, wherein A is selected from 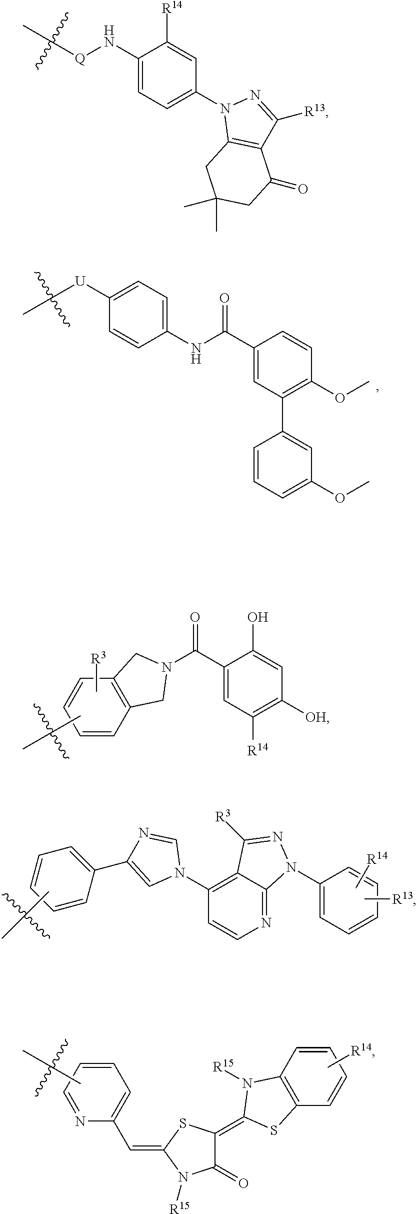 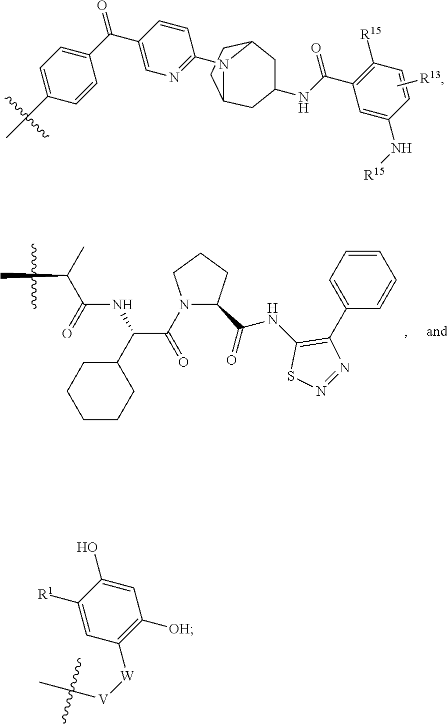
wherein
Q and U are each independently selected from phenyl, heteroaryl, heterocyclyl, and cycloalkyl, each of which being optionally substituted with 1 to 3 groups selected from R²;
R¹³ and R¹⁴ are each independently selected from hydrogen, halo, —CN, (C₁-C₄)alkyl, halo(C₁-C₄)alkyl, and —C(O)NR^{a}R^{b};
R¹⁵ is hydrogen, (C₁-C₄)alkyl, or halo(C₁-C₄)alkyl;
W is 5- or 6-membered heteroaryl optionally substituted with 1 to 3 groups selected from R²;
V is phenyl or 5- to 9-membered heteroaryl optionally substituted with 1 to 3 groups selected from R³;
R¹ is halo, (C₁-C₄)alkyl, halo(C₁-C₄)alkyl, (C₁-C₄)alkoxy, or halo(C₁-C₄)alkoxy;
R² is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{a}, —OR^{a}, —C(O)R^{a}, —C(O)OR^{a}, —C(O)NR^{a}R^{b}, —C(O)NR^{a}(C₁₋₄alkylene)OR^{a}, —C(O)NR^{a}(C₁₋₄alkylene)NR^{a}R^{b}, —C(O)NR^{a}(C₁₋₄alkylene)OR, —NR^{a}R^{b}, —O(C₁₋₄ alkylene)NR^{a}R^{b}, —C₁₋₄alkylNR^{a}R^{b}, —SR^{a}, —S(O)R^{a}, —S(O)₂R^{a}, —S(O)NR^{a}R^{b}, —SO₂NR^{a}R^{b}, —NR^{a}(C₁₋₄ alkyl)OR^{a}, —SH, —S(C₁₋₄alkyl), —NR^{a}(C₁₋₄alkyl)NR^{a}R^{b}, —C₁₋₆alkylC(O)NR^{a}R^{b}, —O(C₁₋₄ alkylene)NR^{a}C(O)(C₁₋₄alkylene)NR^{a}R^{b}, phenyl or 5- to 7-membered heteroaryl, wherein said phenyl and 5- to 7-membered heteroaryl are each optionally and independently substituted with 1 to 3 groups selected from R⁴;
R^{a} and R^{b} are each independently selected from hydrogen and (C₁-C₄)alkyl, wherein said (C₁-C₄)alkyl is optionally substituted with one or more halo or a 3- to 7-membered heterocyclyl, or both; and
R³ and R⁴ are each independently halo, —NR^{a}R^{b}, (C₁-C₄)alkyl, halo(C₁-C₄)alkyl, (C₁-C₄)alkoxy, or halo(C₁-C₄)alkoxy.

3. The compound of claim 1, or a pharmaceutically acceptable salt thereof, wherein A is

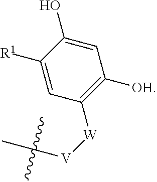

4. (canceled)

5. The compound of claim 3, wherein the compound is of the Formula: 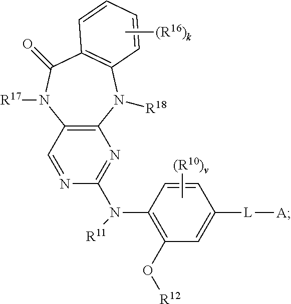
or a pharmaceutically acceptable salt thereof.

6. (canceled)

7. (canceled)

8. The compound of claim 5, or a pharmaceutically acceptable salt thereof, wherein R¹¹ is hydrogen; and R¹⁷ is (C₁-C₆)alkyl.

9. (canceled)

10. (canceled)

11. The compound of claim 5, or a pharmaceutically acceptable salt thereof, wherein R¹² is (C₁-C₆)alkyl.

12. (canceled)

13. The compound of claim 5, or a pharmaceutically acceptable salt thereof, wherein R¹⁸ is (C₁-C₃)alkyl or S(O)₂(C₁-C₃)alkyl.

14. (canceled)

15. The compound of claim 5, or a pharmaceutically acceptable salt thereof, wherein A is selected from 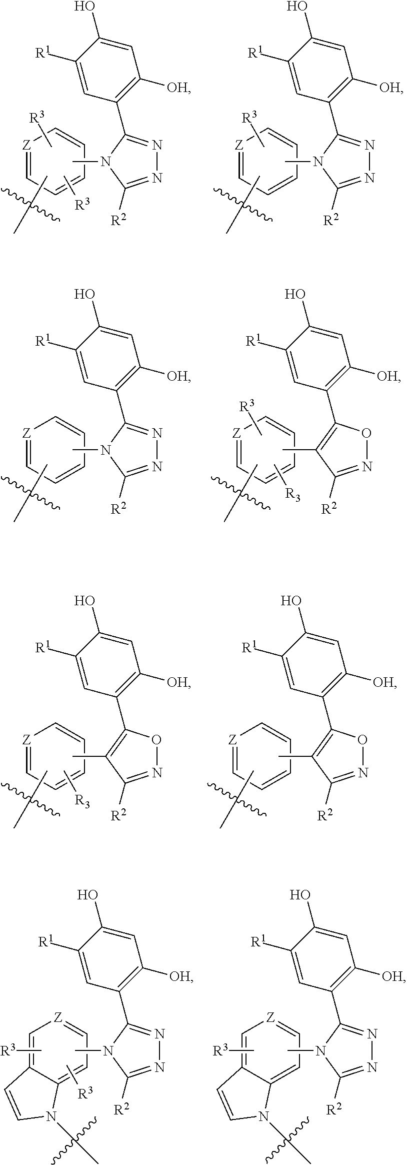 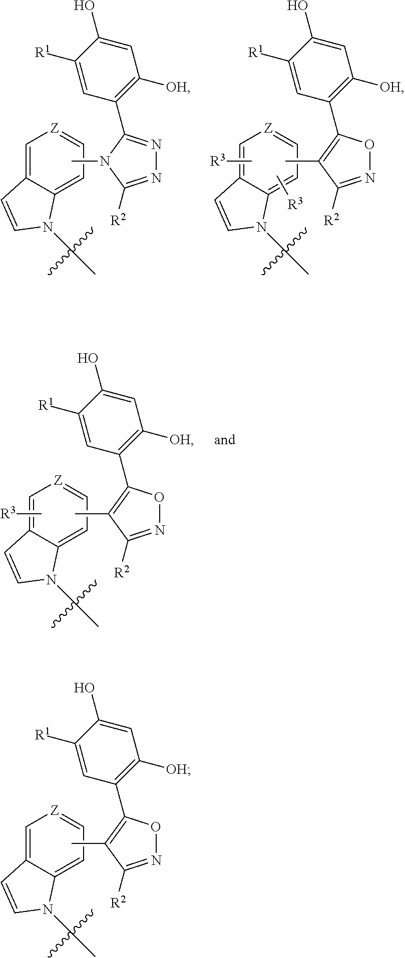
and
Z is N or CH.

16-19. (canceled)

20. The compound of claim 5, or a pharmaceutically acceptable salt thereof, wherein A is

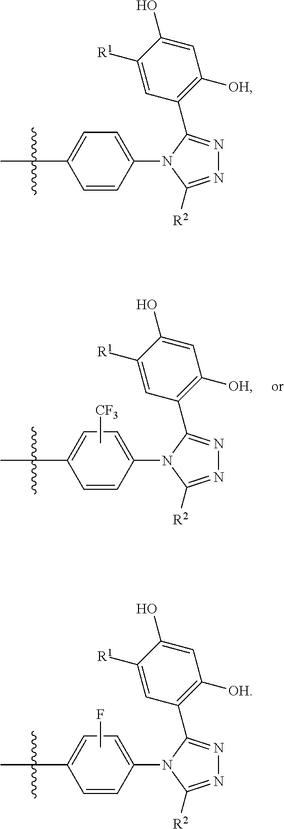

21. The compound of claim 5, or a pharmaceutically acceptable salt thereof, wherein A is

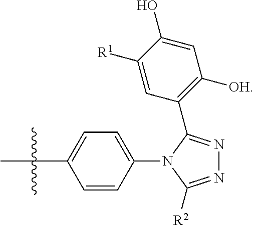

22. The compound of claim 15, or a pharmaceutically acceptable salt thereof, wherein R¹ is halo or (C₁-C₄)alkyl.

23. (canceled)

24. (canceled)

25. The compound of claim 15, or a pharmaceutically acceptable salt thereof, wherein R² is —OR^{a}, —SR^{a}, —C(O)NR^{a}R^{b}, or —C(O)NR^{a}(C₁₋₄alkylene)NR^{a}R^{b}.

26. The compound of claim 25, or a pharmaceutically acceptable salt thereof, wherein R^{a} and R^{b} are each independently selected from hydrogen and (C₁-C₄)alkyl, wherein said (C₁-C₄)alkyl is optionally substituted with 1 to 3 halo or a 6-membered heterocyclyl.

27-29. (canceled)

30. The compound of claim 1, or a pharmaceutically acceptable salt thereof, wherein
L is Het¹-X¹—*, Het¹-X¹-Het²-X²—*, Het¹-O—(CH₂)ₘ—X¹-Het²-X²—*, Het¹-O—(CH₂)ₘX¹—NR^{c}—(CH₂CH₂O)ₙ(CH₂)ₘ-Het²-X²—*, Het¹-X¹—NR^{c}—(CH₂)ₘ—*, Het¹-X¹-Het²-Het³-X²—*, Het¹-X¹—NR^{c}—(CH₂CH₂O)ₙ(CH₂)ₘ—*, Het¹-X¹—NR^{c}—(CH₂CH₂O), Het²-(CH₂)ₘ—X²*, Het¹-X¹—NR^{c}—(CH₂CH₂O)ₙ—*, Het¹-X¹—NR^{c}—(CH₂)ₘ-Het²-X²-Het³-(CH₂)ₘ—*, Het¹-X¹-Het²-(CH₂)ₘ-Het¹- X²—*, Het¹-X¹-Het²-*, Het¹-X¹—NR^{c}—*, Het¹-X¹—NR^{c}—(CH₂)ₘ-Phe-X²-Het²-(CH₂)ₘ—*, Het¹-X¹-Het²-Het³-*, Het¹-X¹-Het²-(CH₂)ₘ-Het³-X²—(CH₂)ₚ—NR^{c}—(CH₂)ₘ—*, Het¹-X¹-Het²-(CH₂)ₘ-Het³-(CH₂)ₘ—O—*, Het¹-X¹-Het²-(CH₂)ₘ-Het³-(CH₂)ₚ—NR^{c}—(CH₂)ₘ—*, Het¹-X¹-Het²-(CH₂CH₂O)ₙ—*, Het¹-X¹—(CH₂)ₘ-Het²-X²—*, —(CH₂CH₂O)ₒ—(CH₂)ₚ-Het¹-X¹-Het²-(CH₂CH₂O)ₙ*, —(CH₂CH₂O)ₙ—(CH₂)ₘ-Het¹-X¹-Het²-X²*, Het¹-X¹-Phe-X²—NR^{c}—X³—*, —(CH₂CH₂O)ₒ—(CH₂)ₚ-Het¹-X¹-Phe- X²—NR^{c}—(CH₂CH₂O)ₙ—*, —(CH₂CH₂O)ₙ—(CH₂)ₘ—NR^{c}-Phe-X¹—*, —(CH₂CH₂O)ₒ—(CH₂)ₚ—NR^{c}-Phe- (CH₂CH₂O)ₙ—*, —(CH₂CH₂O)ₒ—(CH₂)ₚ—NR^{c}—(CH₂CH₂O)ₙ—(CH₂)ₘ—*, (CH₂CH₂O)—(CH₂)ₘ—NR^{c}—(CH₂CH₂O)ₙ—(CH₂)ₘ—C(O)—NR^{d}—(CH₂CH₂O)ₒ—(CH₂)ₚ—*, —(CH₂CH₂O)ₒ—(CH₂)ₚ—NR^{c}—(CH₂CH₂O)ₙ—(CH₂)ₘ-Het¹-X¹-Het²-X²—*, —(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ*, NR^{c}—(CH₂CH₂O)ₙ—(CH₂)ₘ-Phe-NH—X¹-Het¹-X²*, NR^{c}—(CH₂CH₂O)ₙ—(CH₂)ₘ-Phe-NH—X¹-Het¹-X²—(CH₂CH₂O)ₒ*, —(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ—(CH₂)ₚ—*, —(CH₂CH₂O)ₒ—(CH₂)ₚ—NR^{c}—(CH₂CH₂O)ₙ—(CH₂)ₘ- Het¹-X¹—*, —(CH₂CH₂O)ₒ—(CH₂)ₚ—NR^{c}—(CH₂CH₂O)ₙ—(CH₂)ₘ-Het¹-X¹—(CH₂CH₂O)ₙ—*, —(CH₂CH₂O)ₙ—(CH₂)ₘ—NR^{c}—(CH₂)ₘ—C(O)—NR^{d}-Het-X¹-Het²-(CH₂CH₂O)ₒ—(CH₂)ₚ*, or NR^{c}—(CH₂)ₘ—C(O)—NR^{d}—(CH₂)ₘ-Het¹-X¹-Het²-X²*;
* indicates the point of attachment to A;
Het¹, Het², and Het³ are each independently phenyl, a 4- to 6-membered heterocyclyl, 5- to 7-membered heteroaryl, or a 4- to 6-membered cycloalkyl, each of which are optionally substituted with (C₁-C₄)alkyl;
X¹, X², and X³, are each independently C(O) or (CH₂)ᵣ; and
m, n, o, p, q and r are each independently integers selected from 0, 1, 2, 3, 4, 5, and 6.

31. (canceled)

32. The compound of claim 30, or a pharmaceutically acceptable salt thereof, wherein L is Het¹-X¹-Het²-X²—*, Het¹-X¹—NR^{c}—(CH₂)ₘ—*, Het¹-X¹-Het²-Het³-X²—*, or Het¹-X¹-Het²-(CH₂)ₘ-Het³-X²—*.

33. (canceled)

34. (canceled)

35. The compound of claim 32, or a pharmaceutically acceptable salt thereof, wherein Het¹ and Het² are each independently phenyl or a 4- to 6-membered heterocyclyl.

36. (canceled)

37. (canceled)

38. The compound of claim 1, or a pharmaceutically acceptable salt thereof, wherein L is selected from

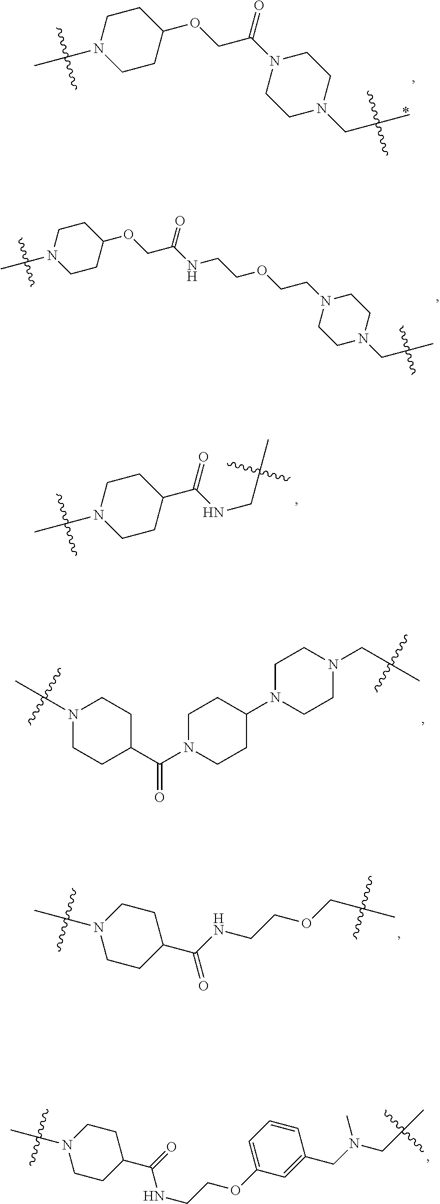

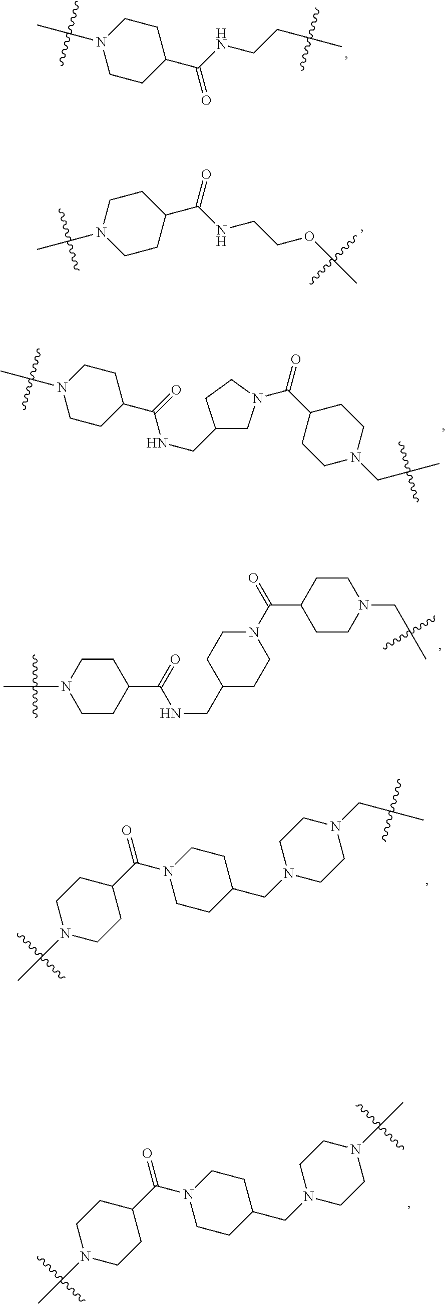 [SMILES: *CCNC(=O)C1CCN(*)CC1 ; *OCCNC(=O)C1CCN(*)CC1 ; *CN1CCC(C(=O)N2CCC(CNC(=O)C3CCN(*)CC3)C2)CC1 ; *CN1CCC(C(=O)N2CCC(CNC(=O)C3CCN(*)CC3)CC2)CC1 ; *CN1CCN(CC2CCN(C(=O)C3CCN(*)CC3)CC2)CC1 ; *N1CCC(C(=O)N2CCC(CN3CCN(*)CC3)CC2)CC1]

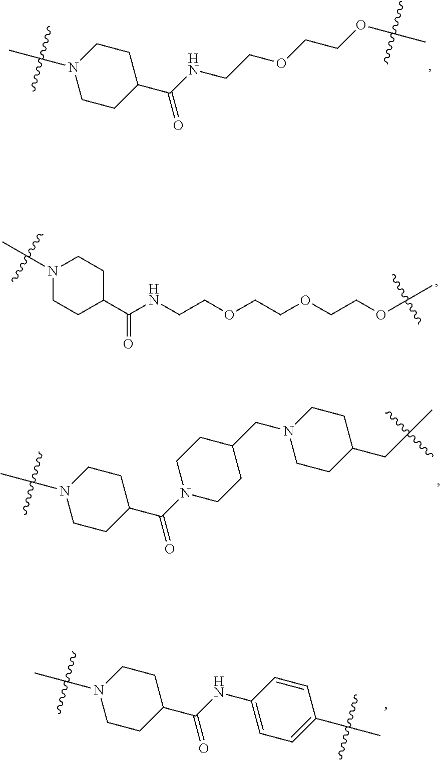 [SMILES: *OCCOCCNC(=O)C1CCN(*)CC1 ; *OCCOCCOCCNC(=O)C1CCN(*)CC1 ; *CC1CCN(CC2CCN(C(=O)C3CCN(*)CC3)CC2)CC1 ; *c1ccc(NC(=O)C2CCN(*)CC2)cc1]

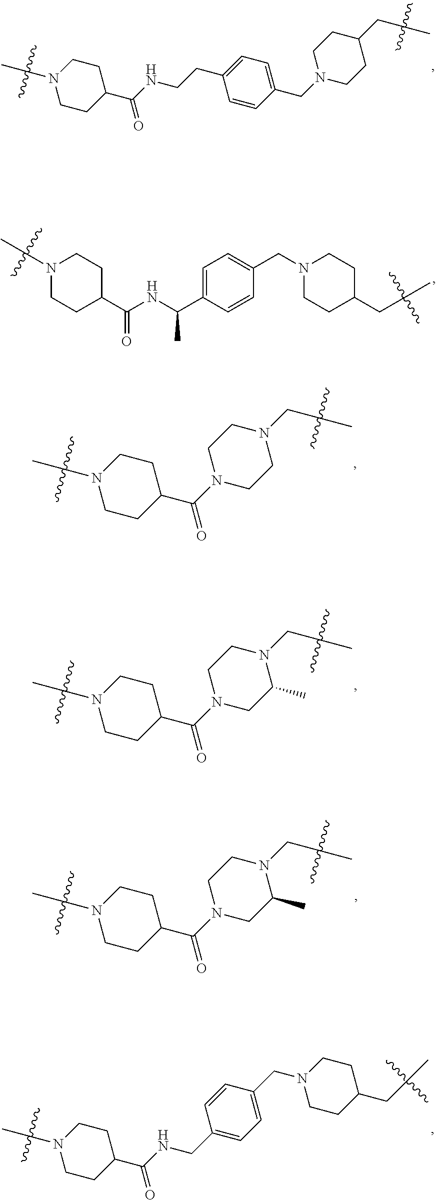 [SMILES: *CC1CCN(Cc2ccc(CCNC(=O)C3CCN(*)CC3)cc2)CC1 ; *CC1CCN(Cc2ccc([C@@H](C)NC(=O)C3CCN(*)CC3)cc2)CC1 ; *CN1CCN(C(=O)C2CCN(*)CC2)CC1 ; *CN1CCN(C(=O)C2CCN(*)CC2)C[C@H]1C ; *CN1CCN(C(=O)C2CCN(*)CC2)C[C@@H]1C ; *CC1CCN(Cc2ccc(CNC(=O)C3CCN(*)CC3)cc2)CC1]

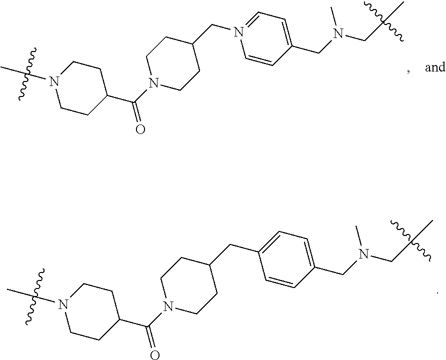

39. (canceled)

40. The compound of claim 1, wherein the compound is selected from the following structural formula: 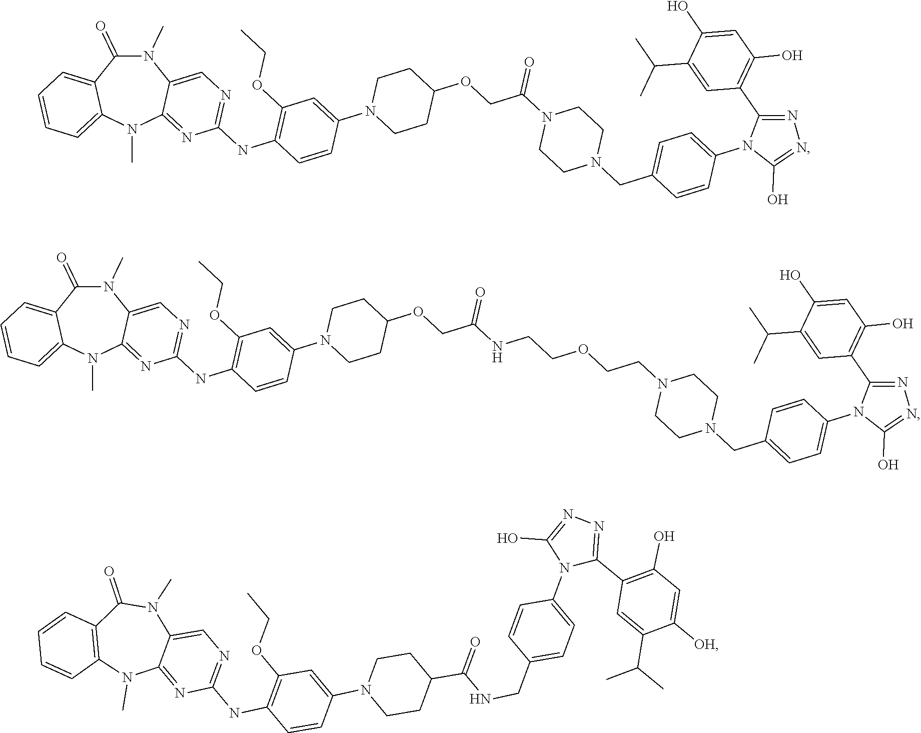 [SMILES: CCOc1cc(N2CCC(OCC(=O)N3CCN(Cc4ccc(-n5c(O)nnc5-c5cc(C(C)C)c(O)cc5O)cc4)CC3)CC2)ccc1Nc1ncc2c(n1)N(C)c1ccccc1C(=O)N2C ; CCOc1cc(N2CCC(OCC(=O)NCCOCCN3CCN(Cc4ccc(-n5c(O)nnc5-c5cc(C(C)C)c(O)cc5O)cc4)CC3)CC2)ccc1Nc1ncc2c(n1)N(C)c1ccccc1C(=O)N2C ; CCOc1cc(N2CCC(C(=O)NCc3ccc(-n4c(O)nnc4-c4cc(C(C)C)c(O)cc4O)cc3)CC2)ccc1Nc1ncc2c(n1)N(C)c1ccccc1C(=O)N2C] 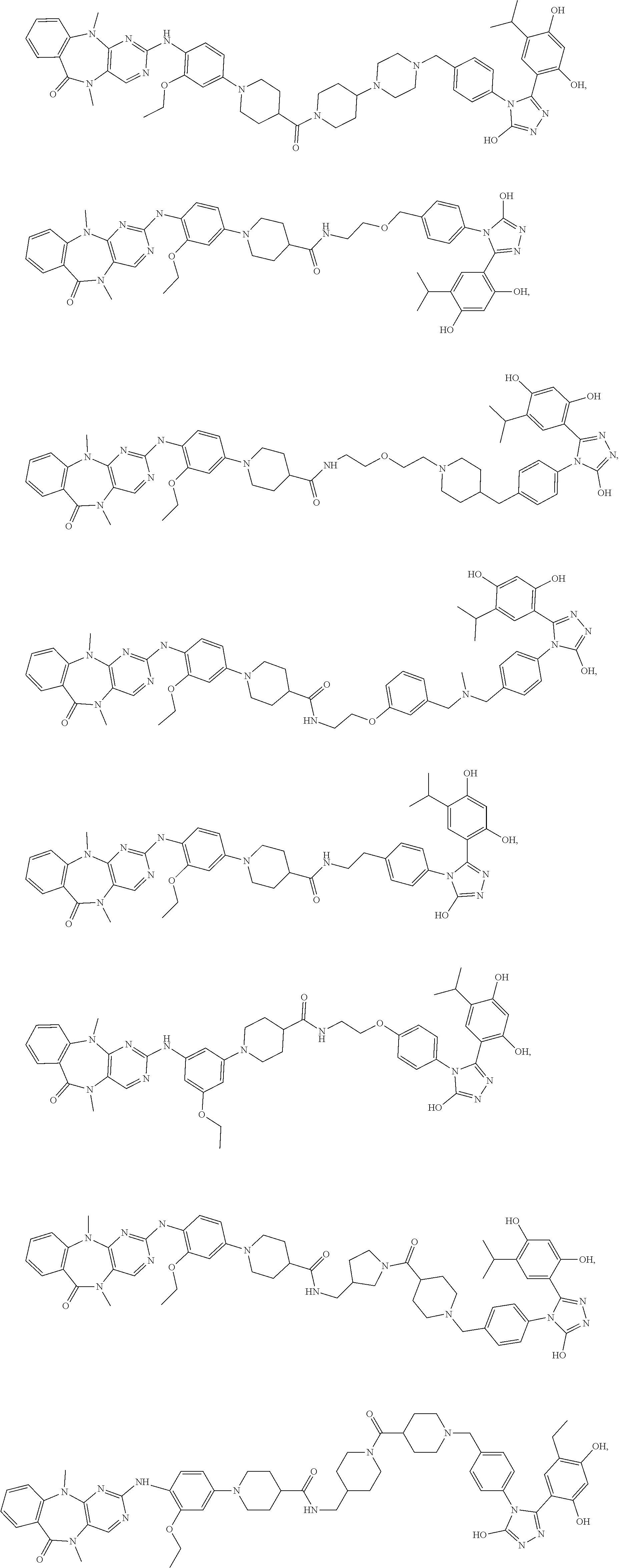 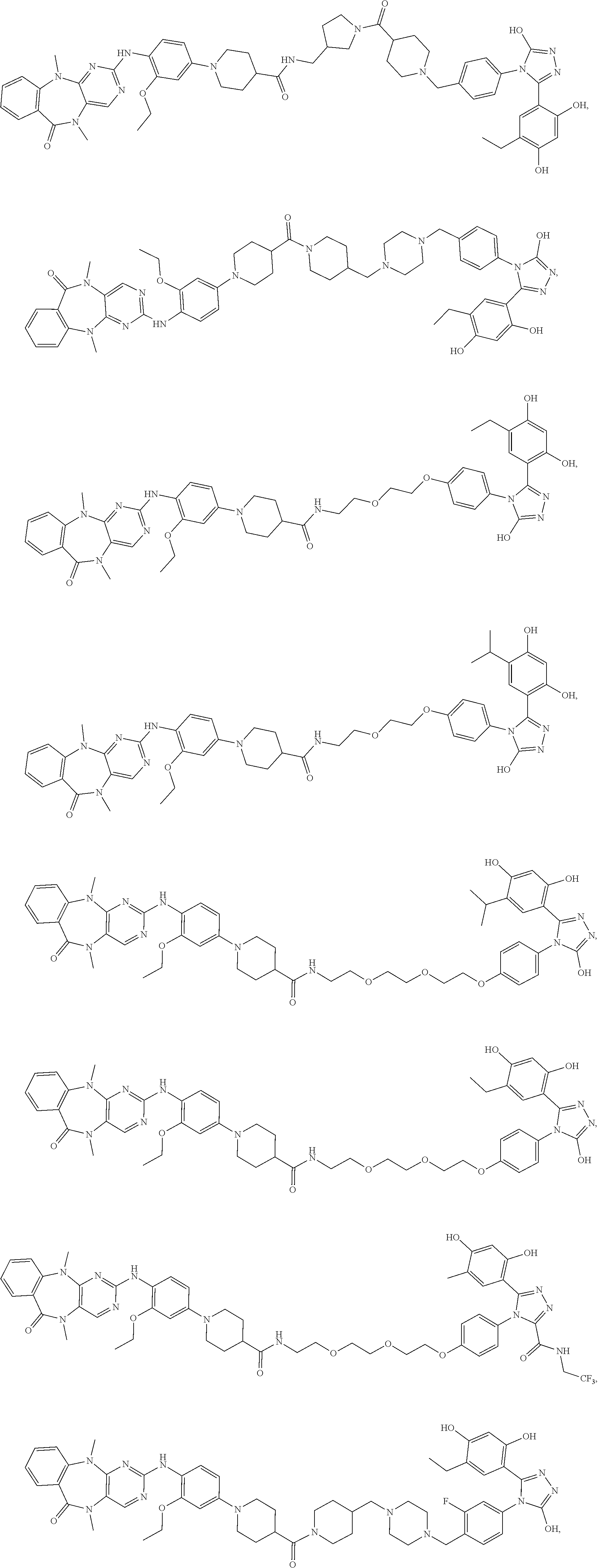 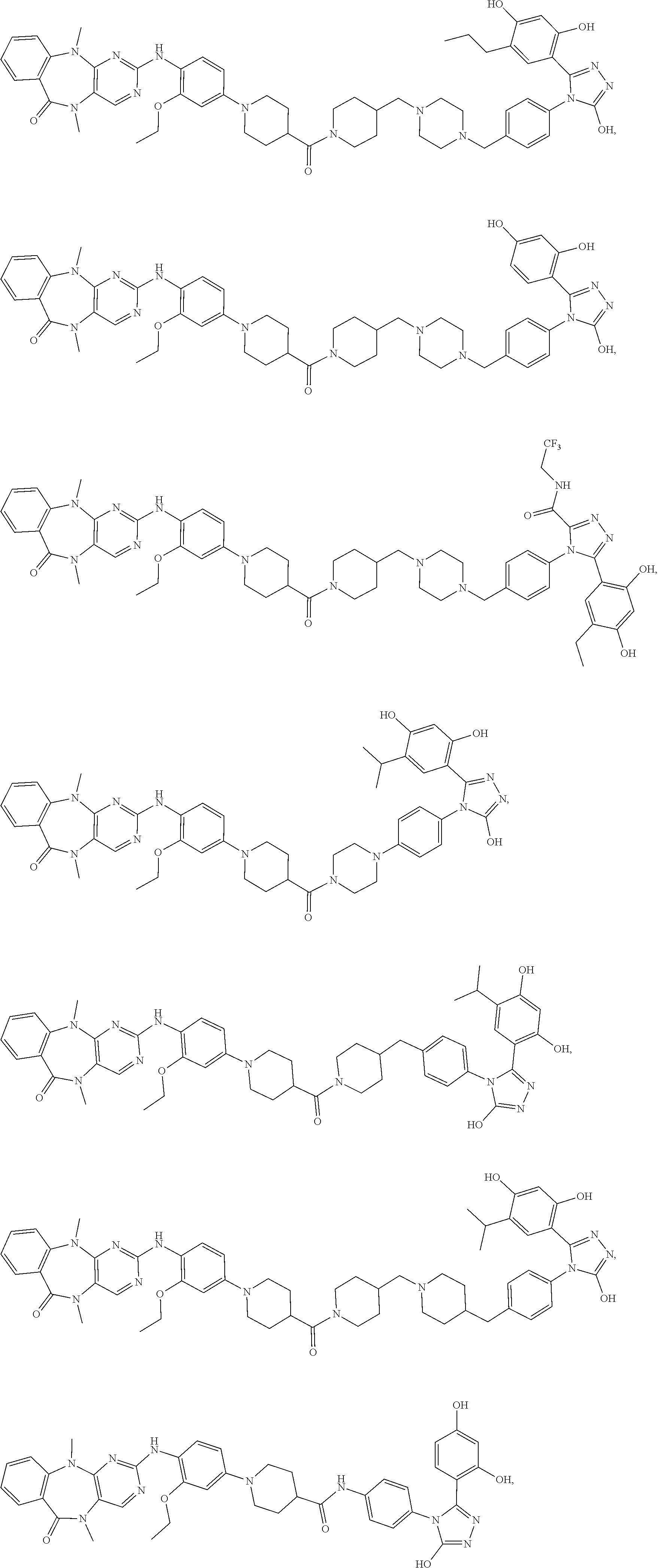 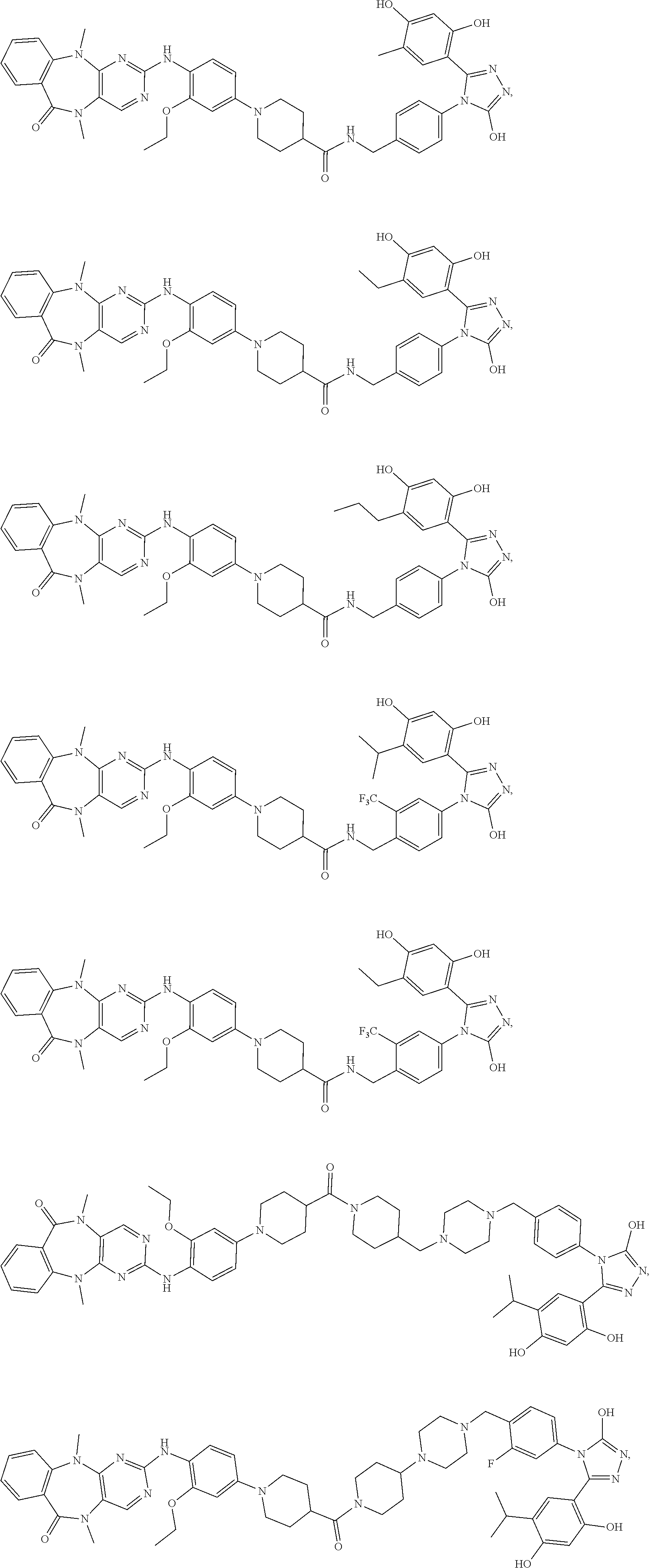 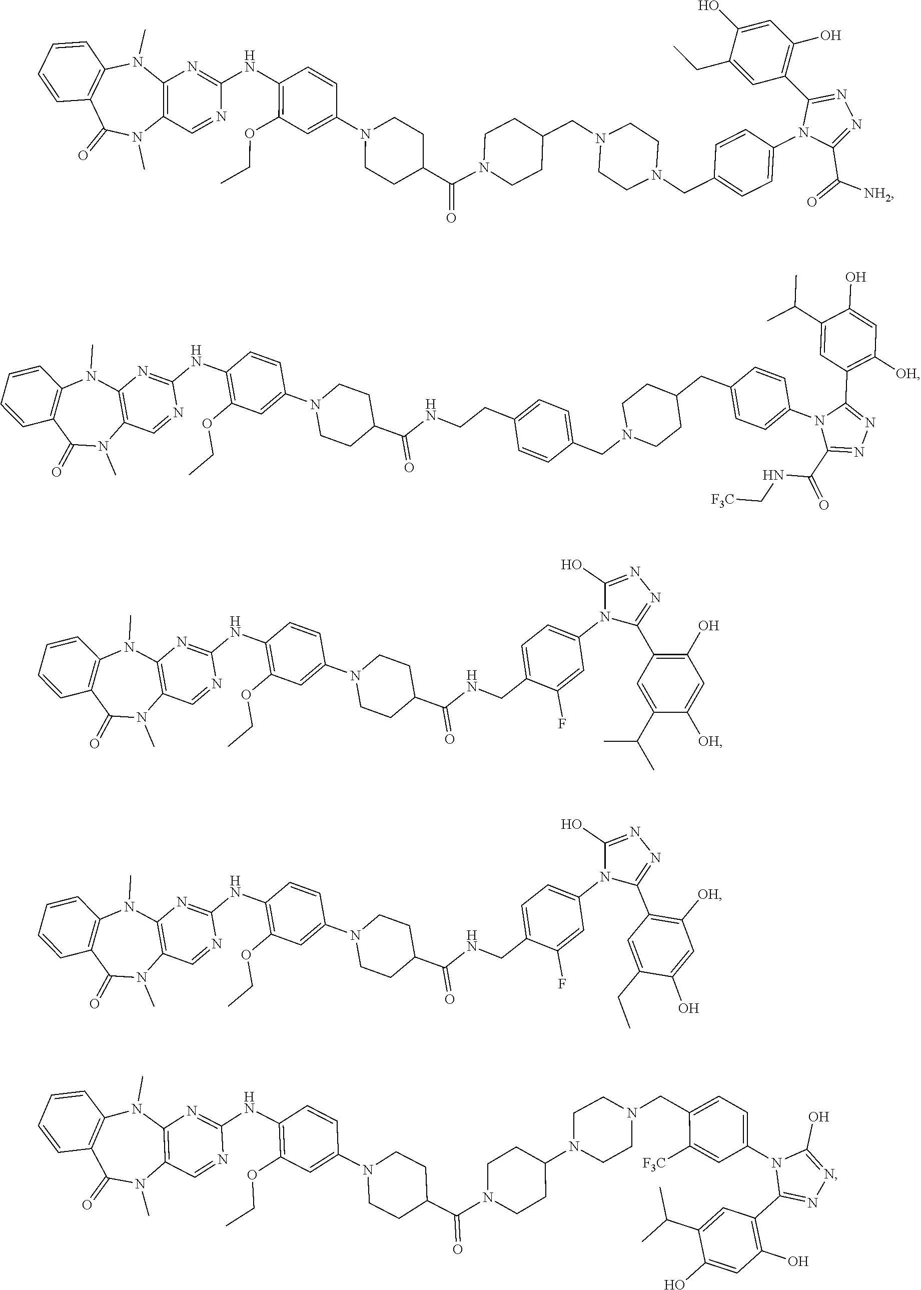 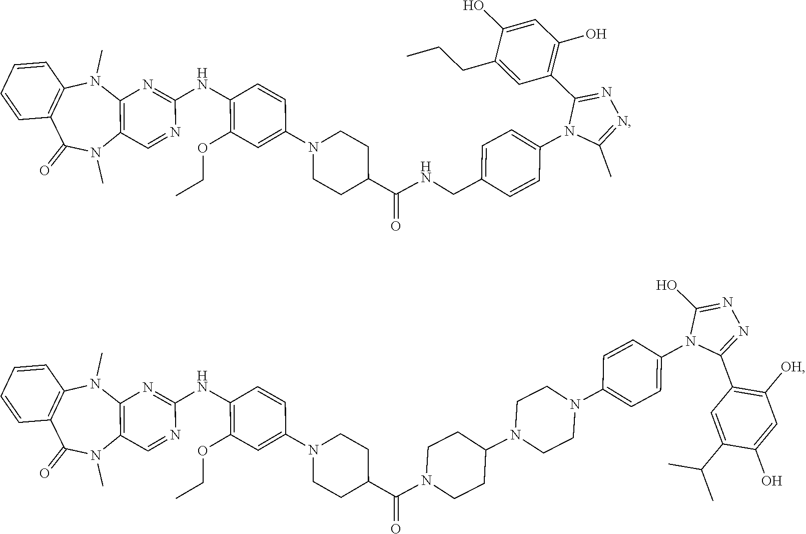 [SMILES: CCCc1cc(-c2nnc(C)n2-c2ccc(CNC(=O)C3CCN(c4ccc(Nc5ncc6c(n5)N(C)c5ccccc5C(=O)N6C)c(OCC)c4)CC3)cc2)c(O)cc1O ; CCOc1cc(N2CCC(C(=O)N3CCC(N4CCN(c5ccc(-n6c(O)nnc6-c6cc(C(C)C)c(O)cc6O)cc5)CC4)CC3)CC2)ccc1Nc1ncc2c(n1)N(C)c1ccccc1C(=O)N2C] 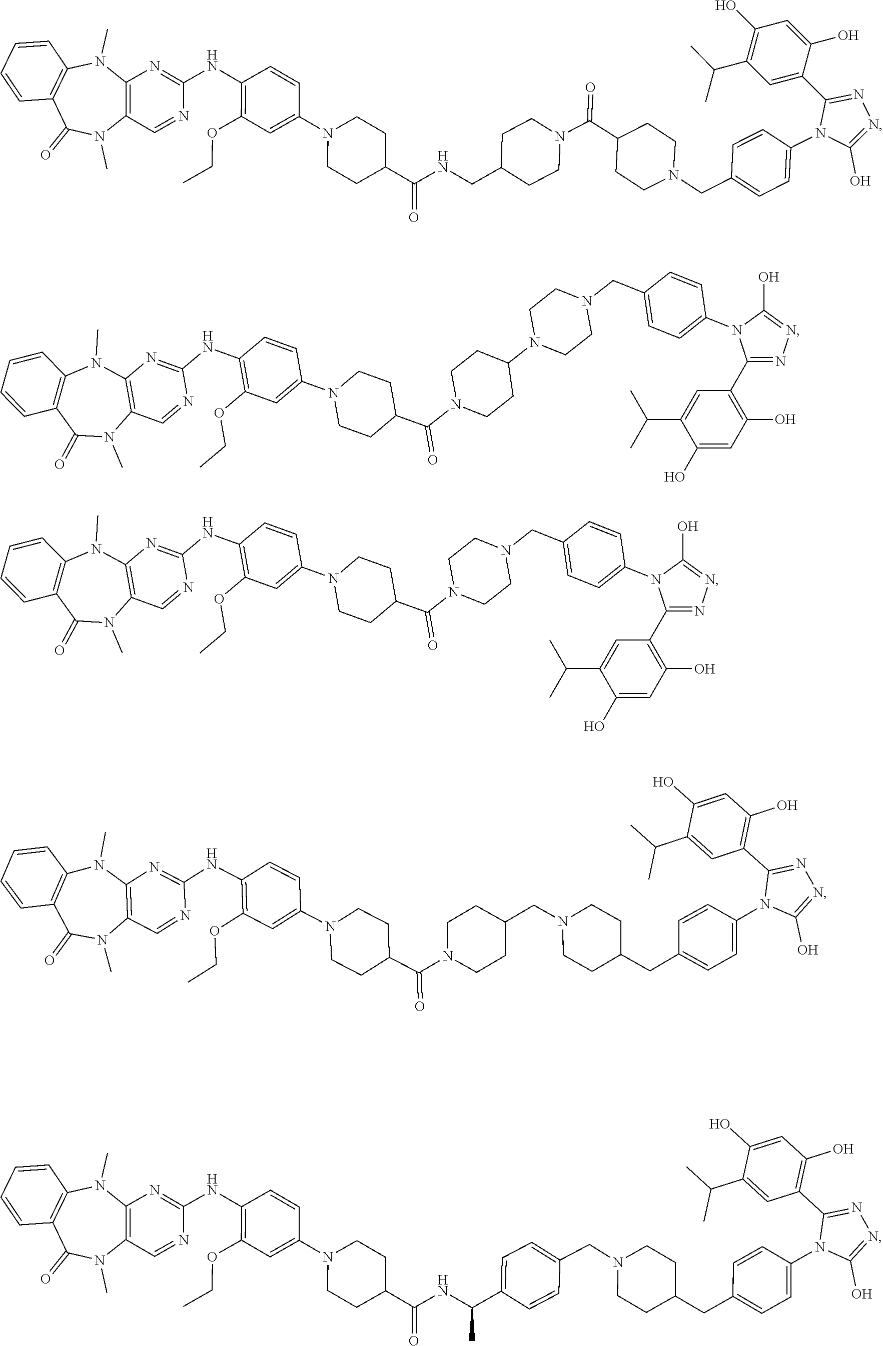 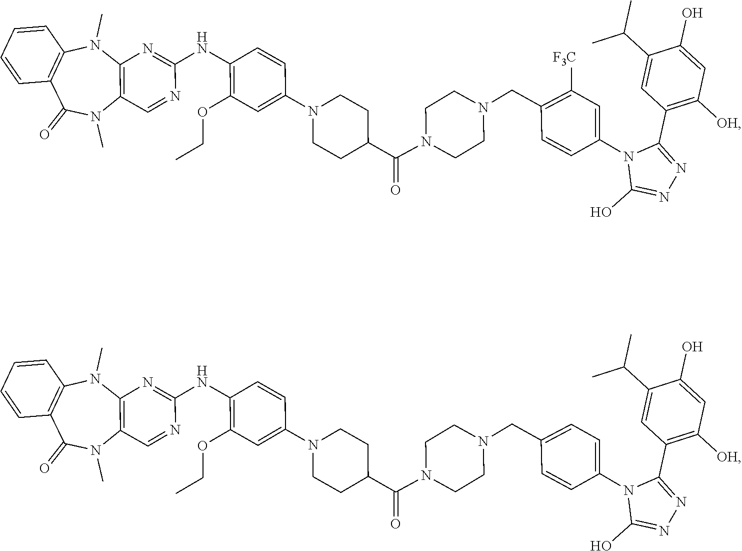 [SMILES: CCOc1cc(N2CCC(C(=O)N3CCN(Cc4ccc(-n5c(O)nnc5-c5cc(C(C)C)c(O)cc5O)cc4C(F)(F)F)CC3)CC2)ccc1Nc1ncc2c(n1)N(C)c1ccccc1C(=O)N2C ; CCOc1cc(N2CCC(C(=O)N3CCN(Cc4ccc(-n5c(O)nnc5-c5cc(C(C)C)c(O)cc5O)cc4)CC3)CC2)ccc1Nc1ncc2c(n1)N(C)c1ccccc1C(=O)N2C] 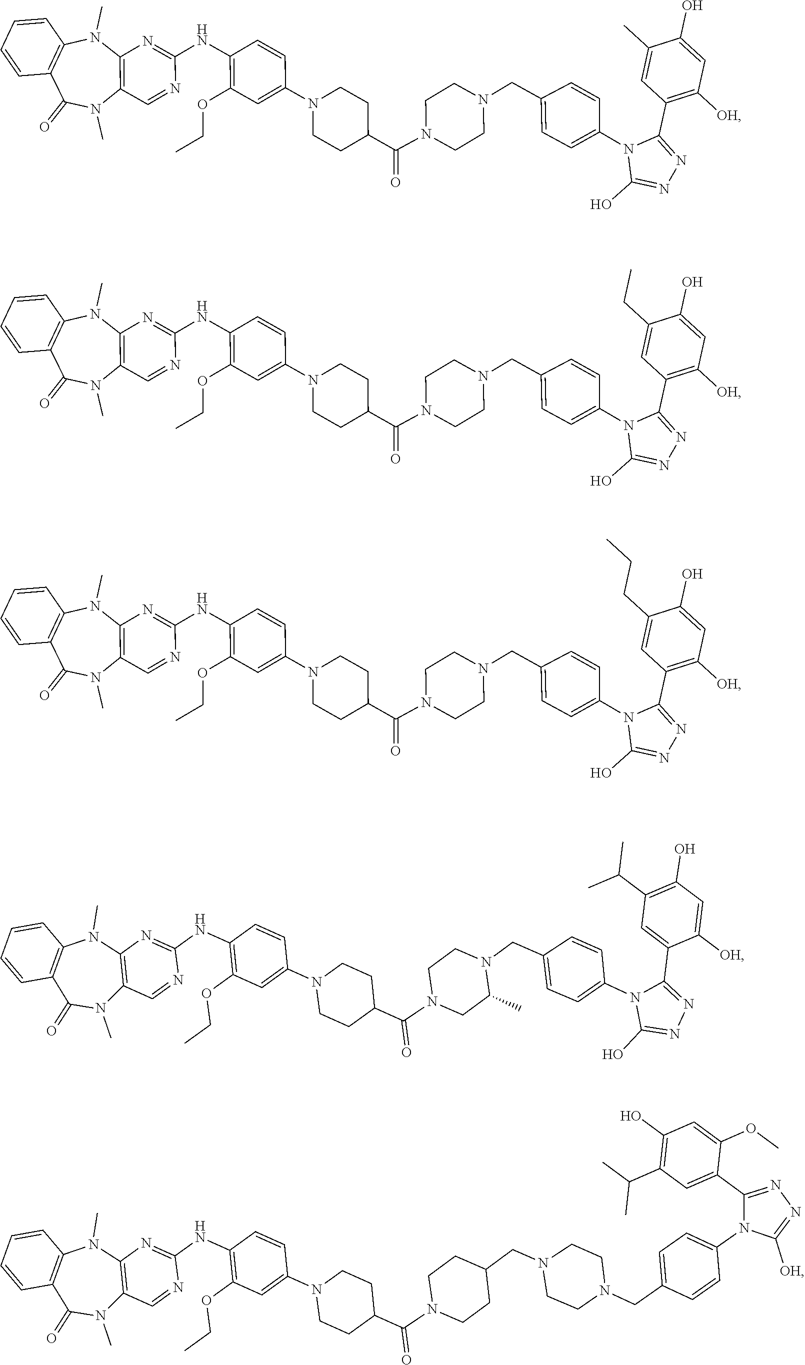 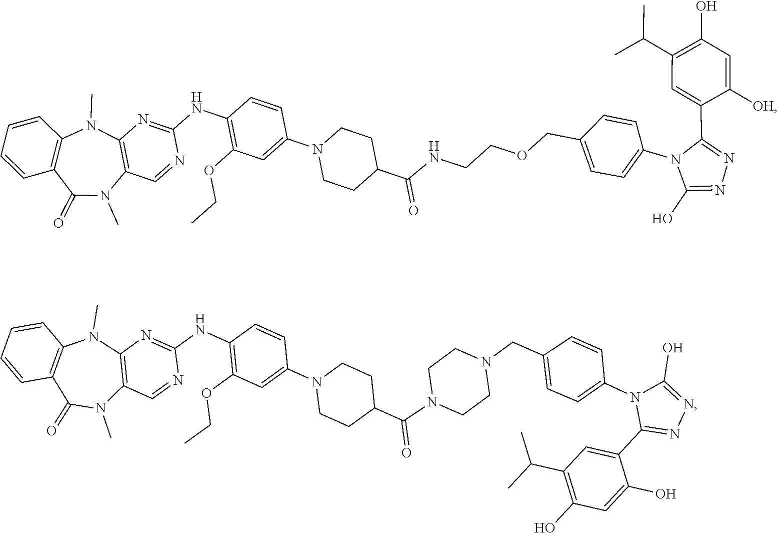 [SMILES: CCOc1cc(N2CCC(C(=O)NCCOCc3ccc(-n4c(O)nnc4-c4cc(C(C)C)c(O)cc4O)cc3)CC2)ccc1Nc1ncc2c(n1)N(C)c1ccccc1C(=O)N2C ; CCOc1cc(N2CCC(C(=O)N3CCN(Cc4ccc(-n5c(O)nnc5-c5cc(C(C)C)c(O)cc5O)cc4)CC3)CC2)ccc1Nc1ncc2c(n1)N(C)c1ccccc1C(=O)N2C] 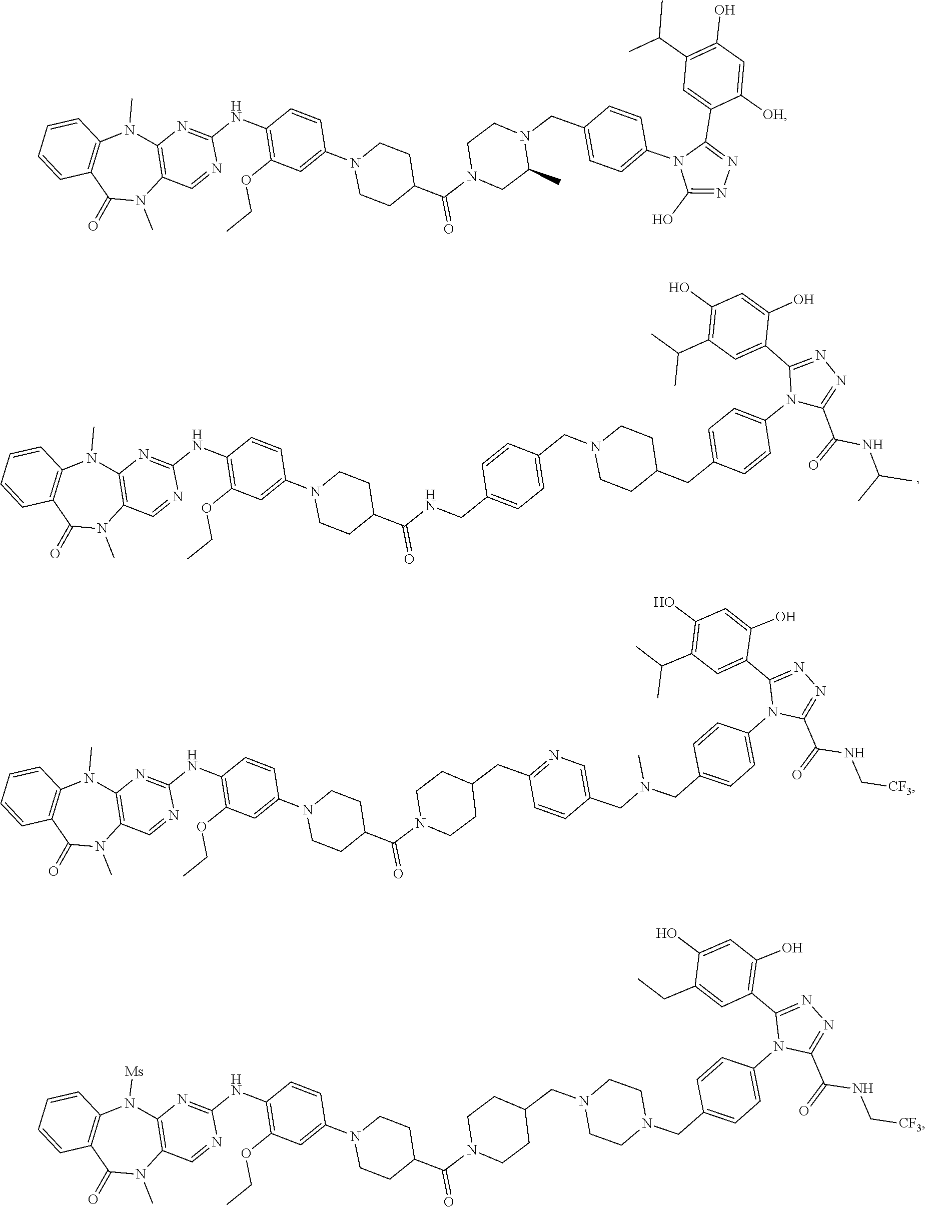 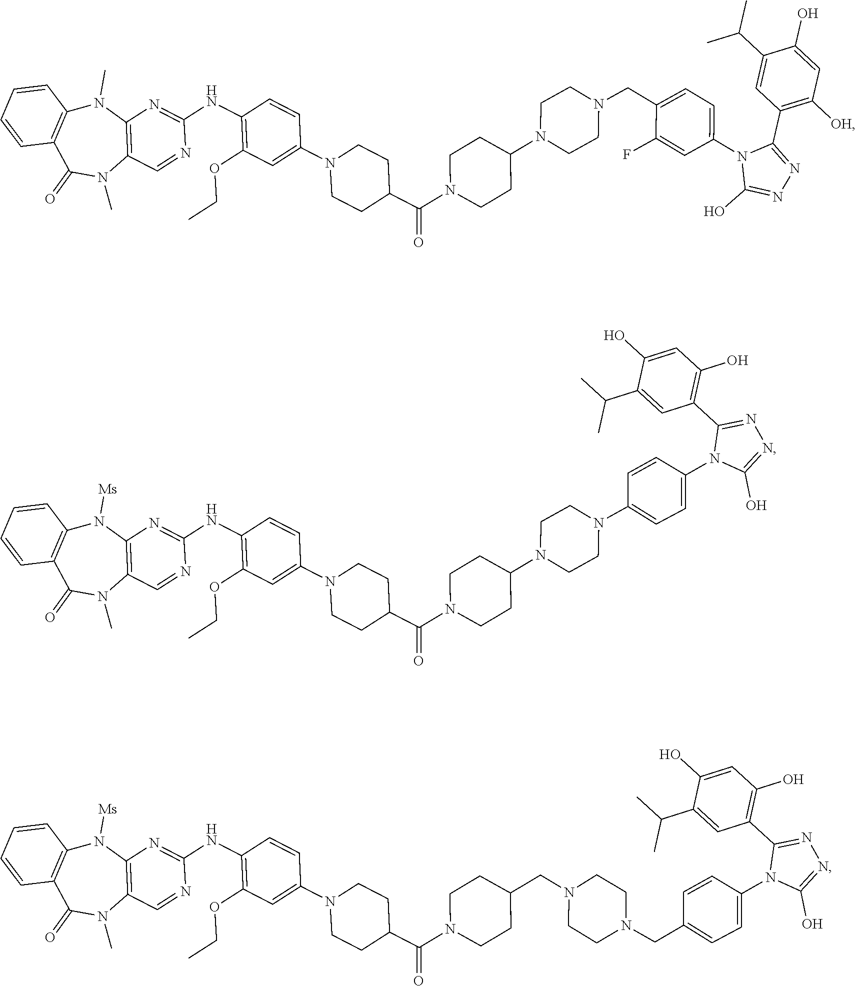 [SMILES: CCOc1cc(N2CCC(C(=O)N3CCC(N4CCN(Cc5ccc(-n6c(O)nnc6-c6cc(C(C)C)c(O)cc6O)cc5F)CC4)CC3)CC2)ccc1Nc1ncc2c(n1)N(C)c1ccccc1C(=O)N2C ; CCOc1cc(N2CCC(C(=O)N3CCC(N4CCN(c5ccc(-n6c(O)nnc6-c6cc(C(C)C)c(O)cc6O)cc5)CC4)CC3)CC2)ccc1Nc1ncc2c(n1)N(S(C)(=O)=O)c1ccccc1C(=O)N2C ; CCOc1cc(N2CCC(C(=O)N3CCC(CN4CCN(Cc5ccc(-n6c(O)nnc6-c6cc(C(C)C)c(O)cc6O)cc5)CC4)CC3)CC2)ccc1Nc1ncc2c(n1)N(S(C)(=O)=O)c1ccccc1C(=O)N2C] 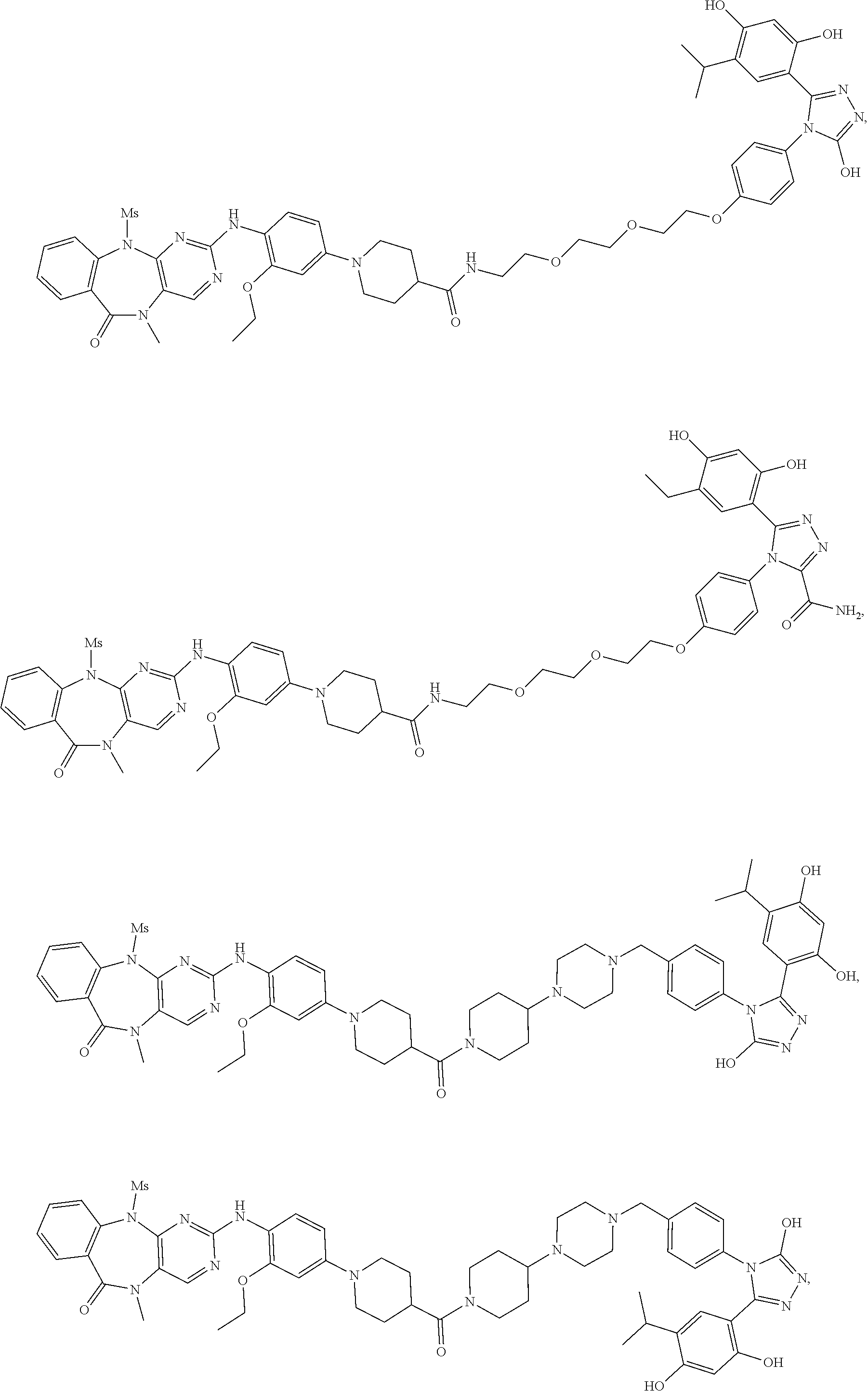 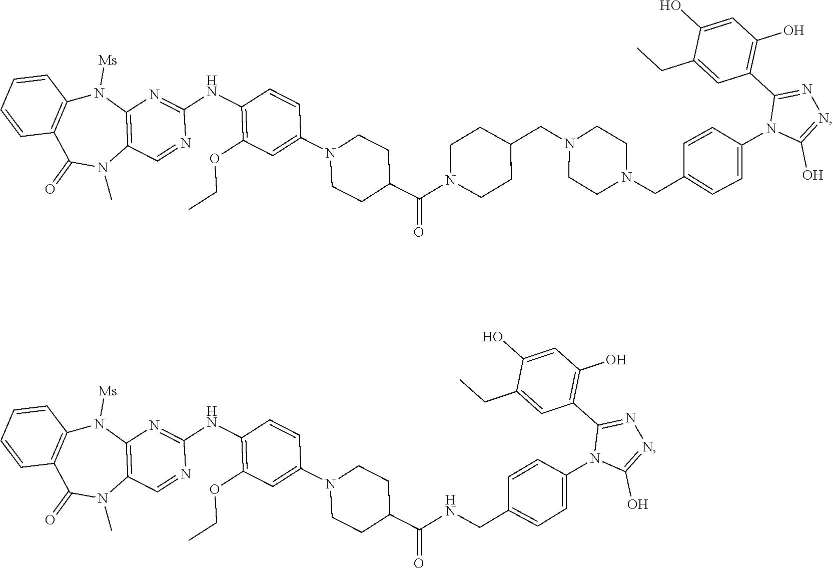 [SMILES: CCOc1cc(N2CCC(C(=O)N3CCC(CN4CCN(Cc5ccc(-n6c(O)nnc6-c6cc(CC)c(O)cc6O)cc5)CC4)CC3)CC2)ccc1Nc1ncc2c(n1)N(S(C)(=O)=O)c1ccccc1C(=O)N2C ; CCOc1cc(N2CCC(C(=O)NCc3ccc(-n4c(O)nnc4-c4cc(CC)c(O)cc4O)cc3)CC2)ccc1Nc1ncc2c(n1)N(S(C)(=O)=O)c1ccccc1C(=O)N2C] 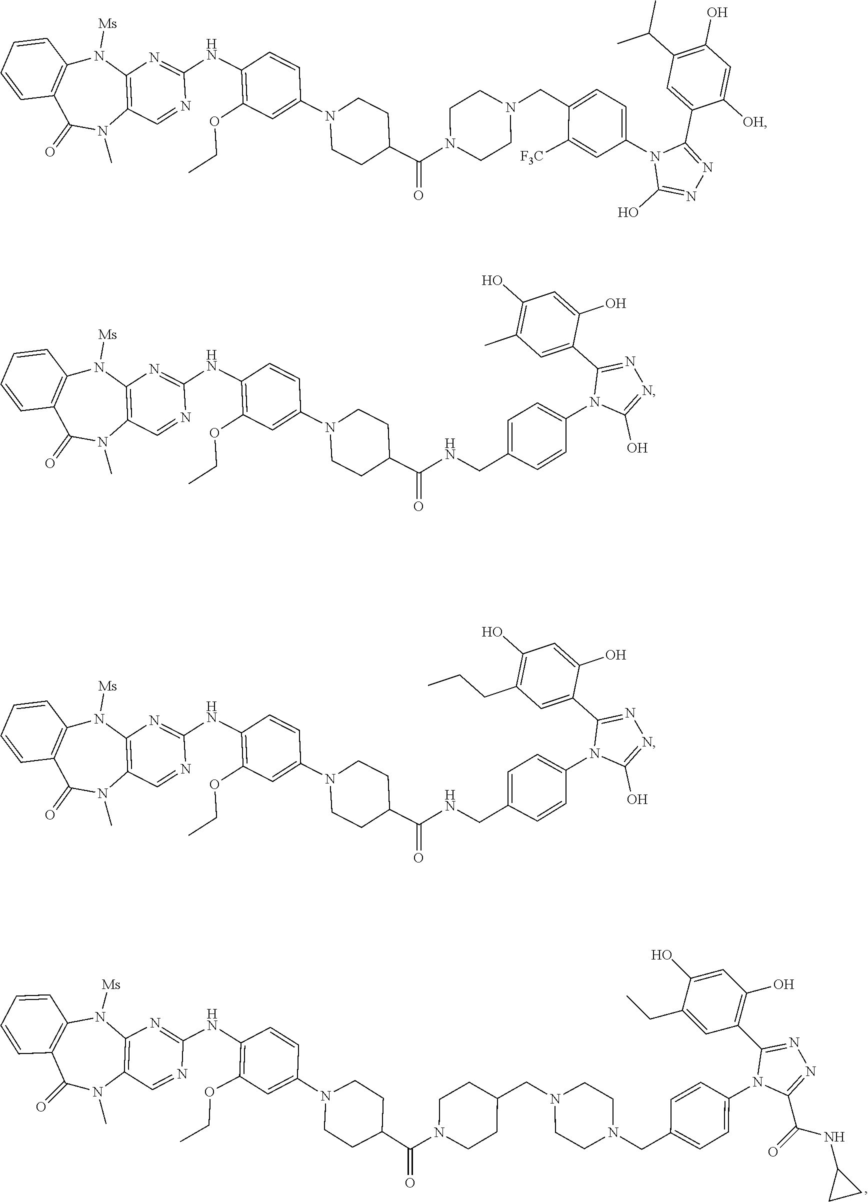 [SMILES: CCOc1cc(N2CCC(C(=O)N3CCN(Cc4ccc(-n5c(O)nnc5-c5cc(C(C)C)c(O)cc5O)cc4C(F)(F)F)CC3)CC2)ccc1Nc1ncc2c(n1)N(S(C)(=O)=O)c1ccccc1C(=O)N2C ; CCOc1cc(N2CCC(C(=O)NCc3ccc(-n4c(O)nnc4-c4cc(C)c(O)cc4O)cc3)CC2)ccc1Nc1ncc2c(n1)N(S(C)(=O)=O)c1ccccc1C(=O)N2C ; CCCc1cc(-c2nnc(O)n2-c2ccc(CNC(=O)C3CCN(c4ccc(Nc5ncc6c(n5)N(S(C)(=O)=O)c5ccccc5C(=O)N6C)c(OCC)c4)CC3)cc2)c(O)cc1O ; CCOc1cc(N2CCC(C(=O)N3CCC(CN4CCN(Cc5ccc(-n6c(C(=O)NC7CC7)nnc6-c6cc(CC)c(O)cc6O)cc5)CC4)CC3)CC2)ccc1Nc1ncc2c(n1)N(S(C)(=O)=O)c1ccccc1C(=O)N2C] 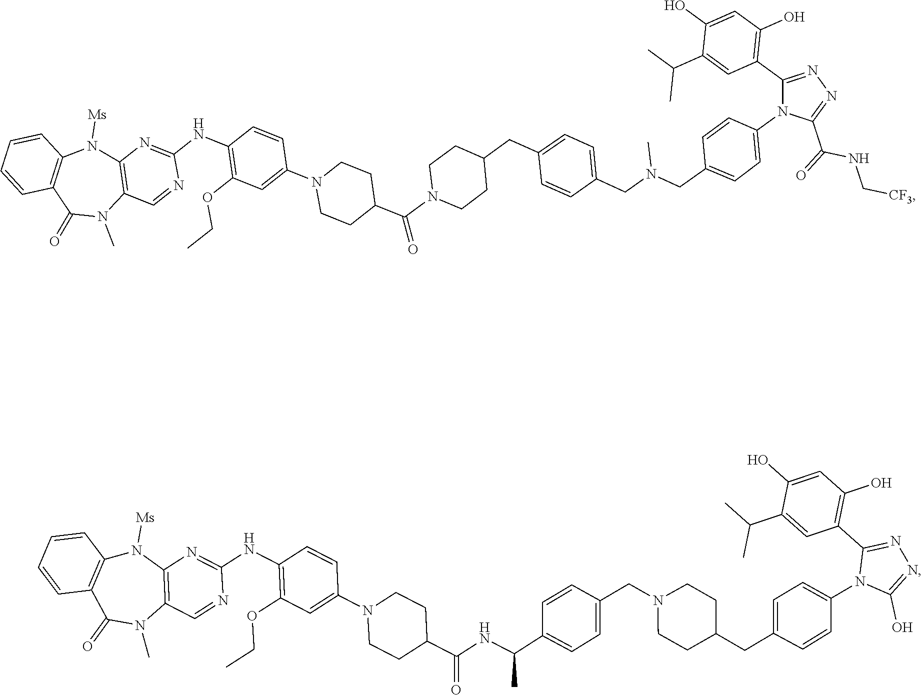 [SMILES: CCOc1cc(N2CCC(C(=O)N3CCC(Cc4ccc(CN(C)Cc5ccc(-n6c(C(=O)NCC(F)(F)F)nnc6-c6cc(C(C)C)c(O)cc6O)cc5)cc4)CC3)CC2)ccc1Nc1ncc2c(n1)N(S(C)(=O)=O)c1ccccc1C(=O)N2C ; CCOc1cc(N2CCC(C(=O)N[C@H](C)c3ccc(CN4CCC(Cc5ccc(-n6c(O)nnc6-c6cc(C(C)C)c(O)cc6O)cc5)CC4)cc3)CC2)ccc1Nc1ncc2c(n1)N(S(C)(=O)=O)c1ccccc1C(=O)N2C] 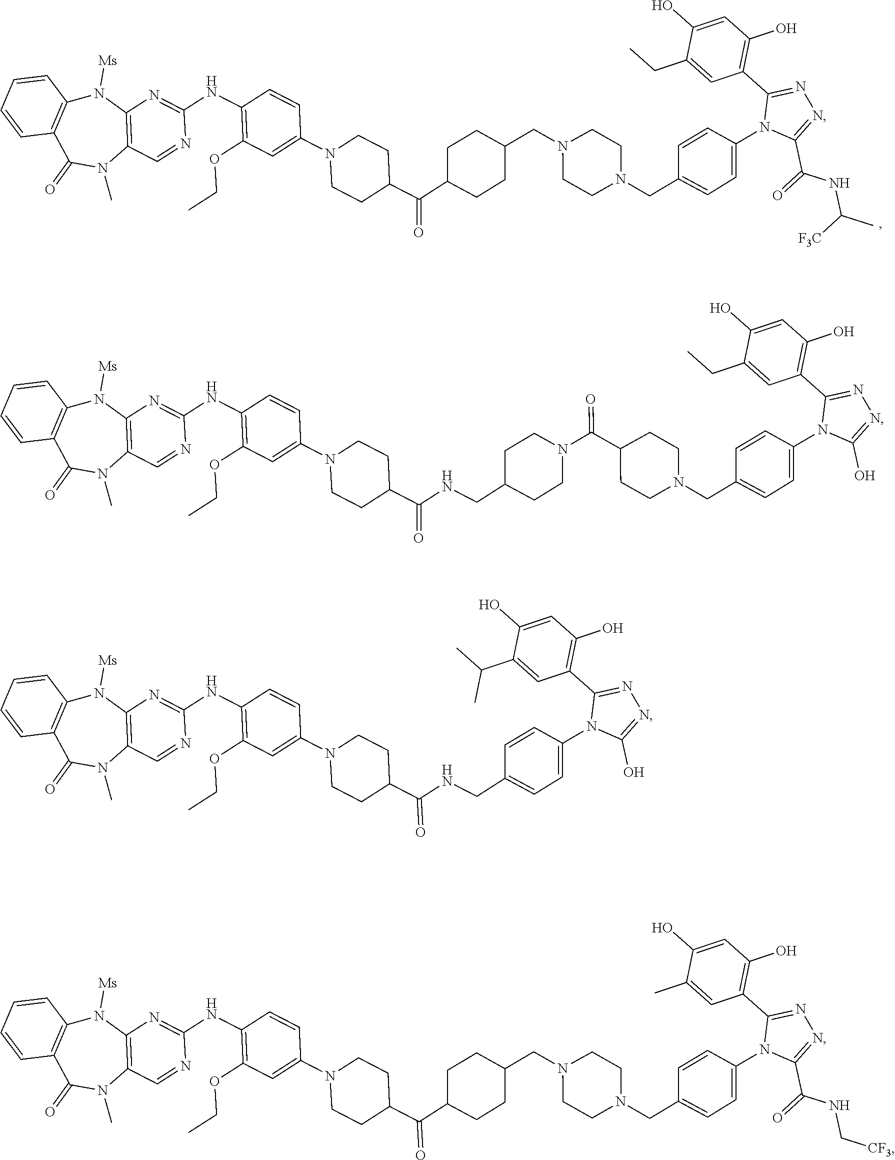 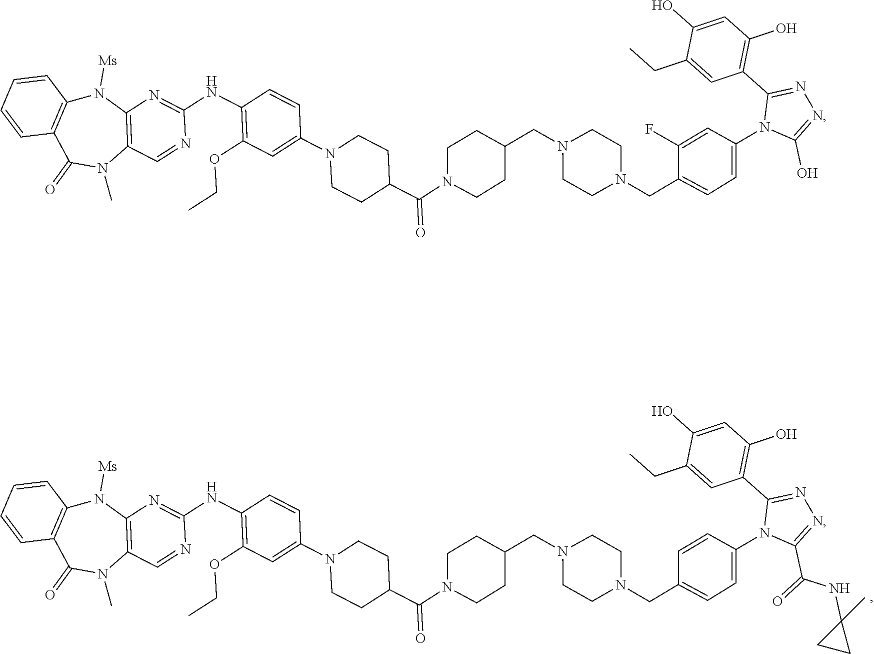 [SMILES: CCOc1cc(N2CCC(C(=O)N3CCC(CN4CCN(Cc5ccc(-n6c(O)nnc6-c6cc(CC)c(O)cc6O)cc5F)CC4)CC3)CC2)ccc1Nc1ncc2c(n1)N(S(C)(=O)=O)c1ccccc1C(=O)N2C ; CCOc1cc(N2CCC(C(=O)N3CCC(CN4CCN(Cc5ccc(-n6c(C(=O)NC7(C)CC7)nnc6-c6cc(CC)c(O)cc6O)cc5)CC4)CC3)CC2)ccc1Nc1ncc2c(n1)N(S(C)(=O)=O)c1ccccc1C(=O)N2C] 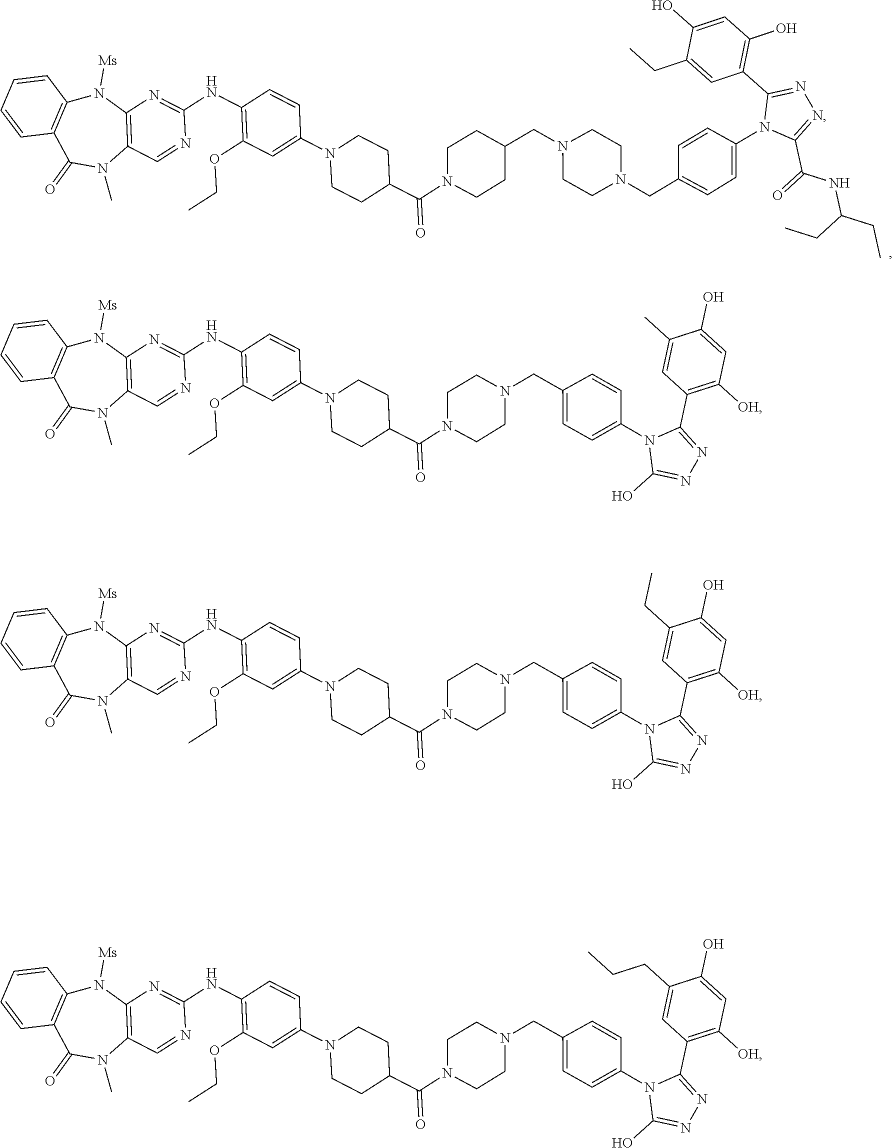 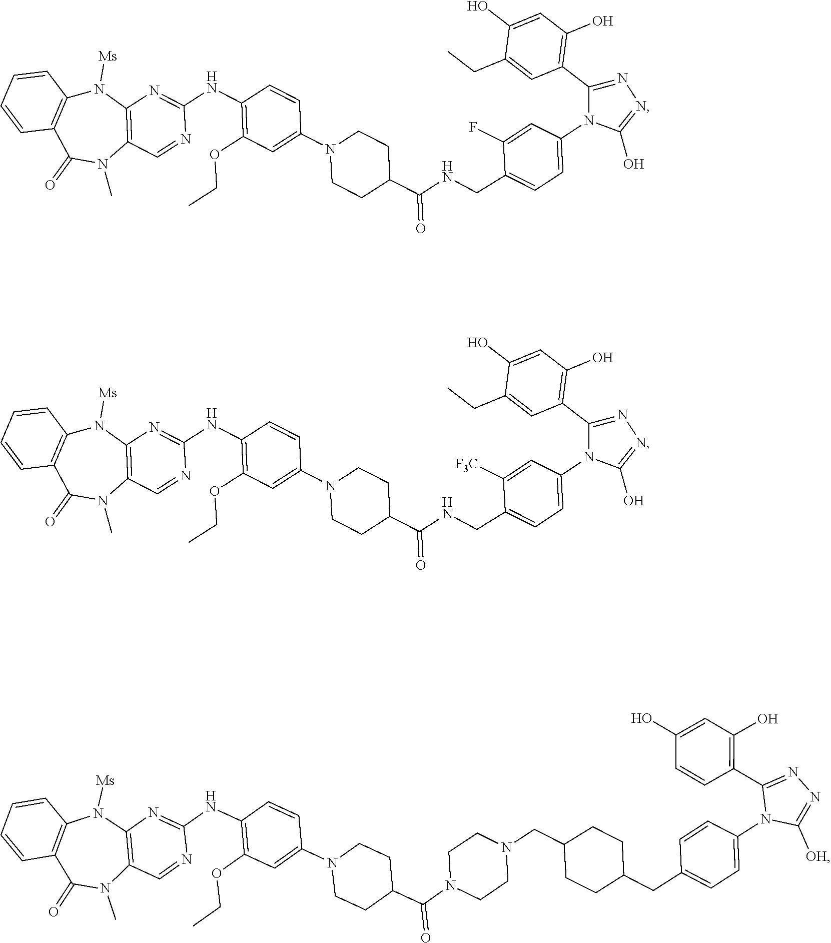 [SMILES: CCOc1cc(N2CCC(C(=O)NCc3ccc(-n4c(O)nnc4-c4cc(CC)c(O)cc4O)cc3F)CC2)ccc1Nc1ncc2c(n1)N(S(C)(=O)=O)c1ccccc1C(=O)N2C ; CCOc1cc(N2CCC(C(=O)NCc3ccc(-n4c(O)nnc4-c4cc(CC)c(O)cc4O)cc3C(F)(F)F)CC2)ccc1Nc1ncc2c(n1)N(S(C)(=O)=O)c1ccccc1C(=O)N2C ; CCOc1cc(N2CCC(C(=O)N3CCN(CC4CCC(Cc5ccc(-n6c(O)nnc6-c6ccc(O)cc6O)cc5)CC4)CC3)CC2)ccc1Nc1ncc2c(n1)N(S(C)(=O)=O)c1ccccc1C(=O)N2C] 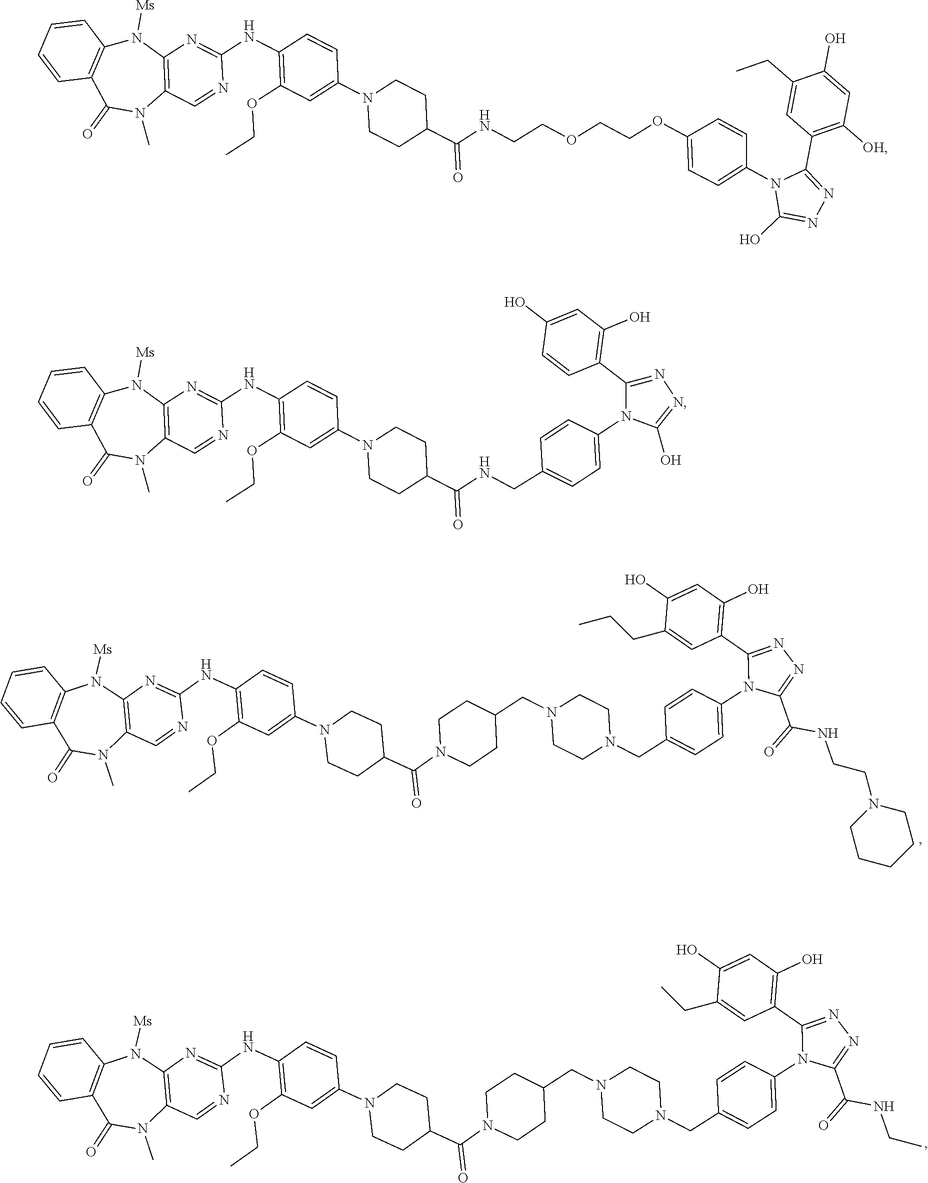 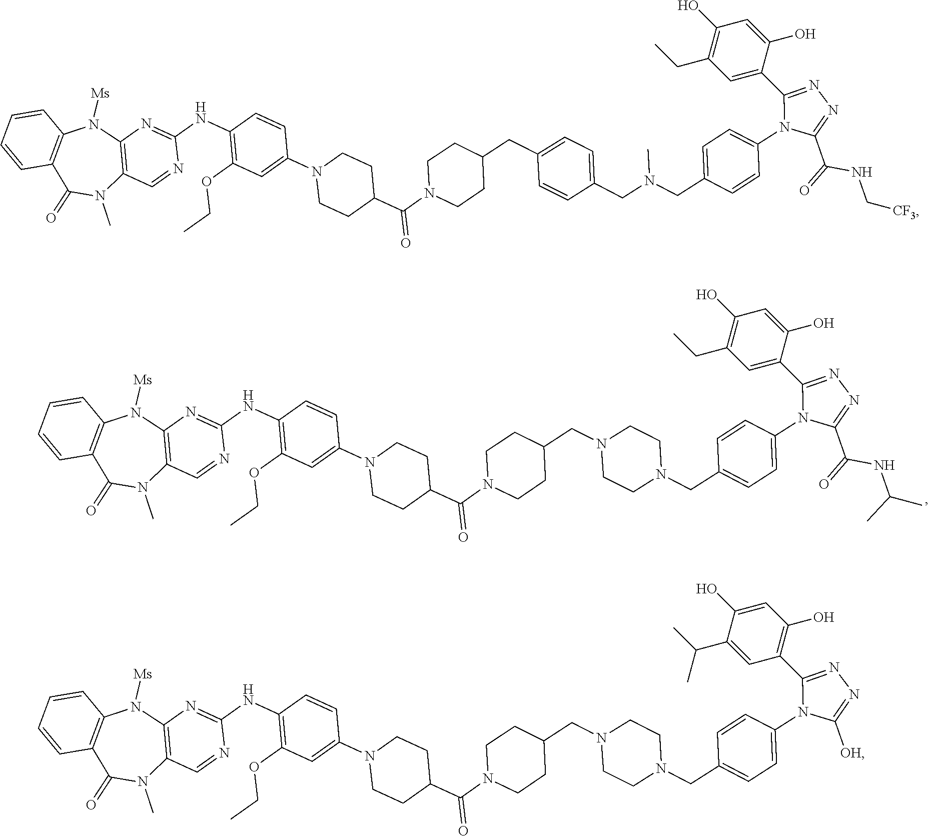 [SMILES: CCOc1cc(N2CCC(C(=O)N3CCC(Cc4ccc(CN(C)Cc5ccc(-n6c(C(=O)NCC(F)(F)F)nnc6-c6cc(CC)c(O)cc6O)cc5)cc4)CC3)CC2)ccc1Nc1ncc2c(n1)N(S(C)(=O)=O)c1ccccc1C(=O)N2C ; CCOc1cc(N2CCC(C(=O)N3CCC(CN4CCN(Cc5ccc(-n6c(C(=O)NC(C)C)nnc6-c6cc(CC)c(O)cc6O)cc5)CC4)CC3)CC2)ccc1Nc1ncc2c(n1)N(S(C)(=O)=O)c1ccccc1C(=O)N2C ; CCOc1cc(N2CCC(C(=O)N3CCC(CN4CCN(Cc5ccc(-n6c(O)nnc6-c6cc(C(C)C)c(O)cc6O)cc5)CC4)CC3)CC2)ccc1Nc1ncc2c(n1)N(S(C)(=O)=O)c1ccccc1C(=O)N2C] 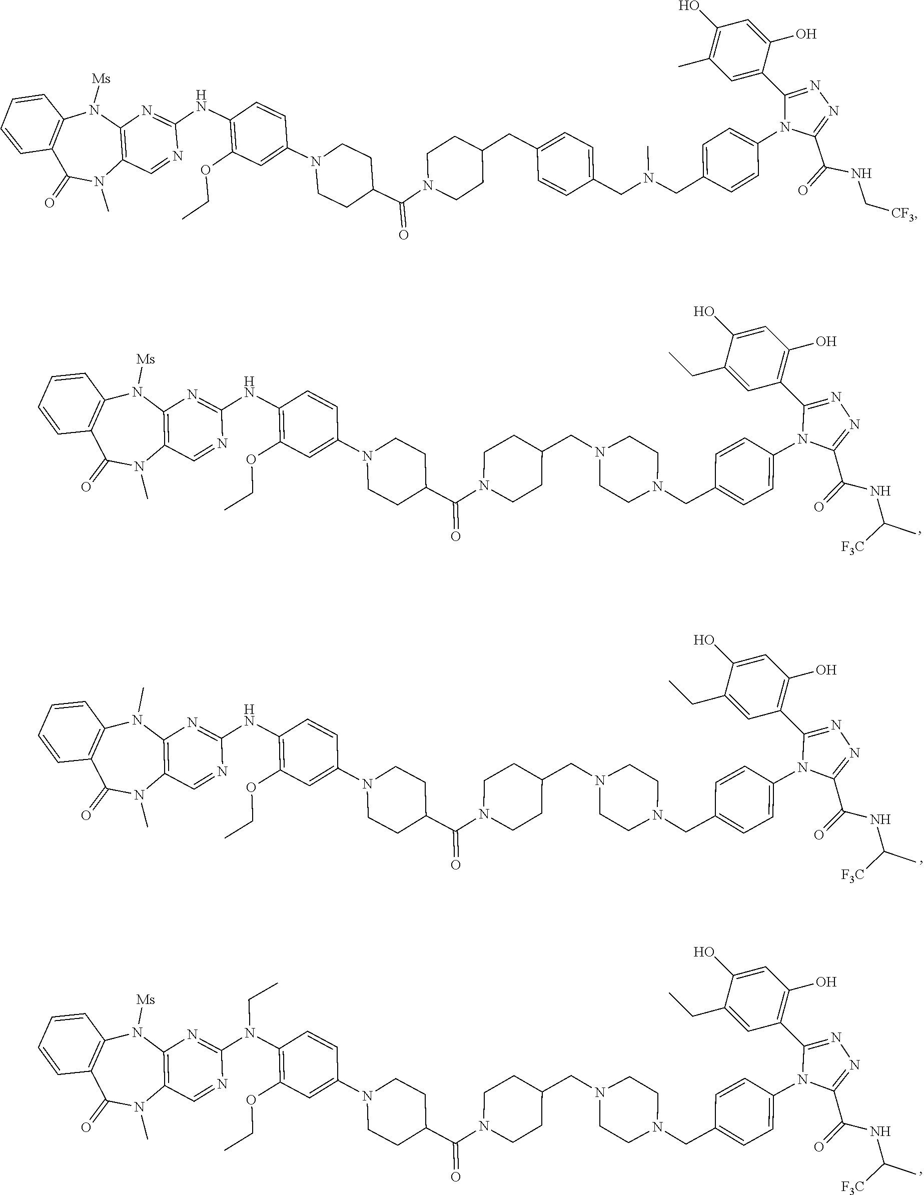 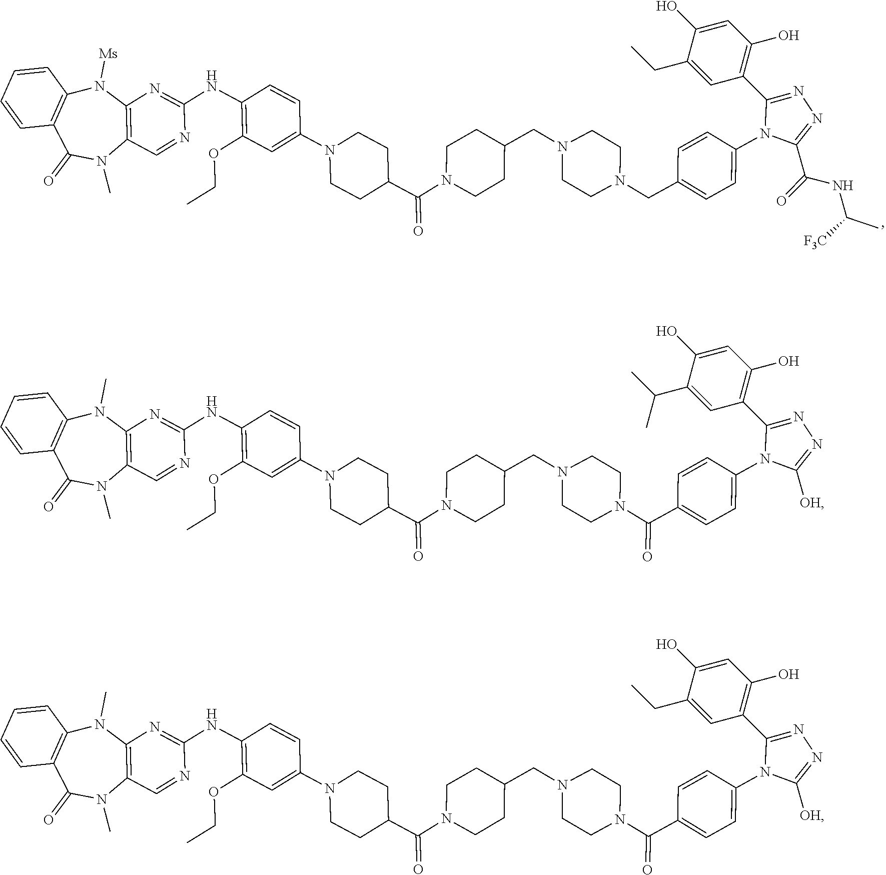 [SMILES: CCOc1cc(N2CCC(C(=O)N3CCC(CN4CCN(Cc5ccc(-n6c(C(=O)N[C@H](C)C(F)(F)F)nnc6-c6cc(CC)c(O)cc6O)cc5)CC4)CC3)CC2)ccc1Nc1ncc2c(n1)N(S(C)(=O)=O)c1ccccc1C(=O)N2C ; CCOc1cc(N2CCC(C(=O)N3CCC(CN4CCN(C(=O)c5ccc(-n6c(O)nnc6-c6cc(C(C)C)c(O)cc6O)cc5)CC4)CC3)CC2)ccc1Nc1ncc2c(n1)N(C)c1ccccc1C(=O)N2C ; CCOc1cc(N2CCC(C(=O)N3CCC(CN4CCN(C(=O)c5ccc(-n6c(O)nnc6-c6cc(CC)c(O)cc6O)cc5)CC4)CC3)CC2)ccc1Nc1ncc2c(n1)N(C)c1ccccc1C(=O)N2C] 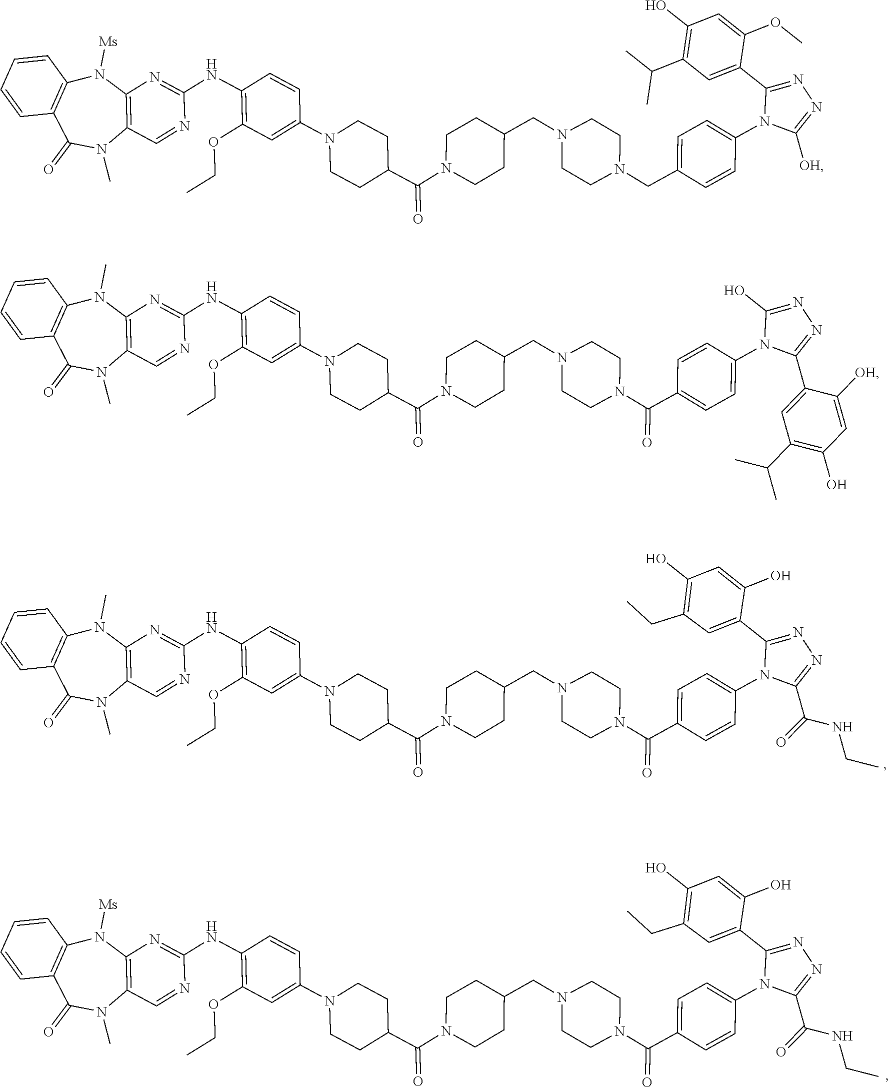 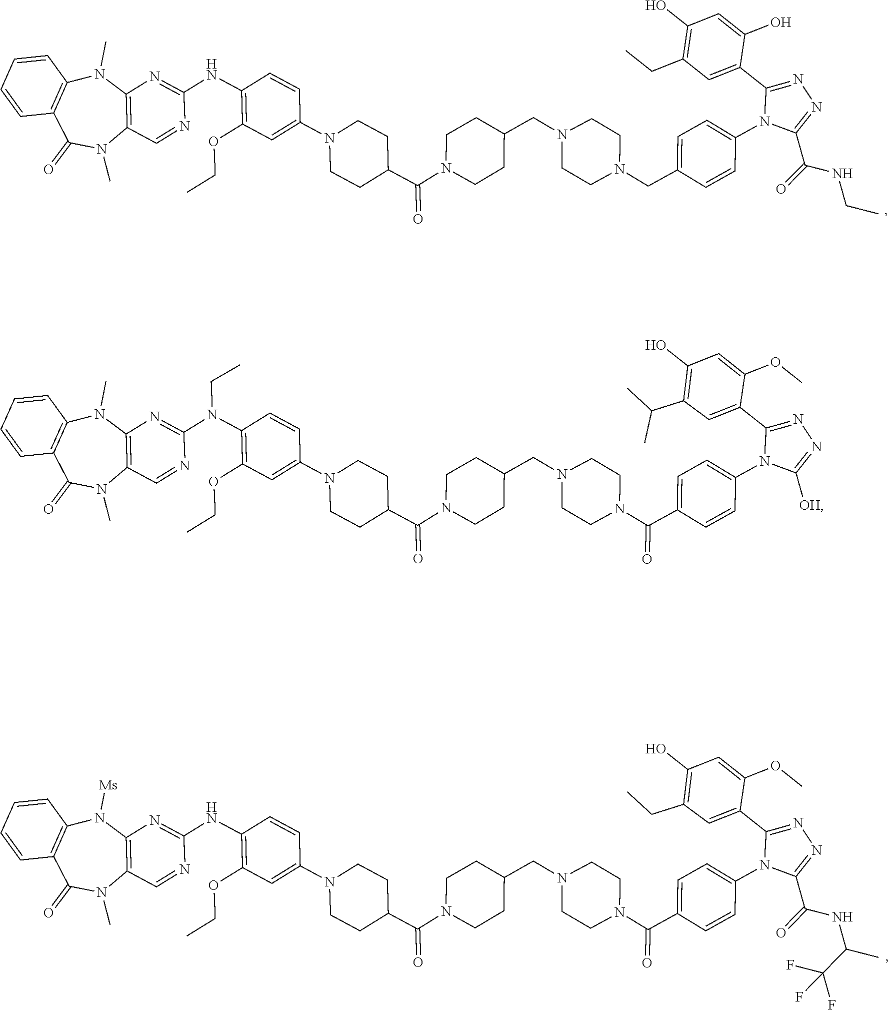 [SMILES: CCNC(=O)c1nnc(-c2cc(CC)c(O)cc2O)n1-c1ccc(CN2CCN(CC3CCN(C(=O)C4CCN(c5ccc(Nc6ncc7c(n6)N(C)c6ccccc6C(=O)N7C)c(OCC)c5)CC4)CC3)CC2)cc1 ; CCOc1cc(N2CCC(C(=O)N3CCC(CN4CCN(C(=O)c5ccc(-n6c(O)nnc6-c6cc(C(C)C)c(O)cc6OC)cc5)CC4)CC3)CC2)ccc1N(CC)c1ncc2c(n1)N(C)c1ccccc1C(=O)N2C ; CCOc1cc(N2CCC(C(=O)N3CCC(CN4CCN(C(=O)c5ccc(-n6c(C(=O)NC(C)C(F)(F)F)nnc6-c6cc(CC)c(O)cc6OC)cc5)CC4)CC3)CC2)ccc1Nc1ncc2c(n1)N(S(C)(=O)=O)c1ccccc1C(=O)N2C] 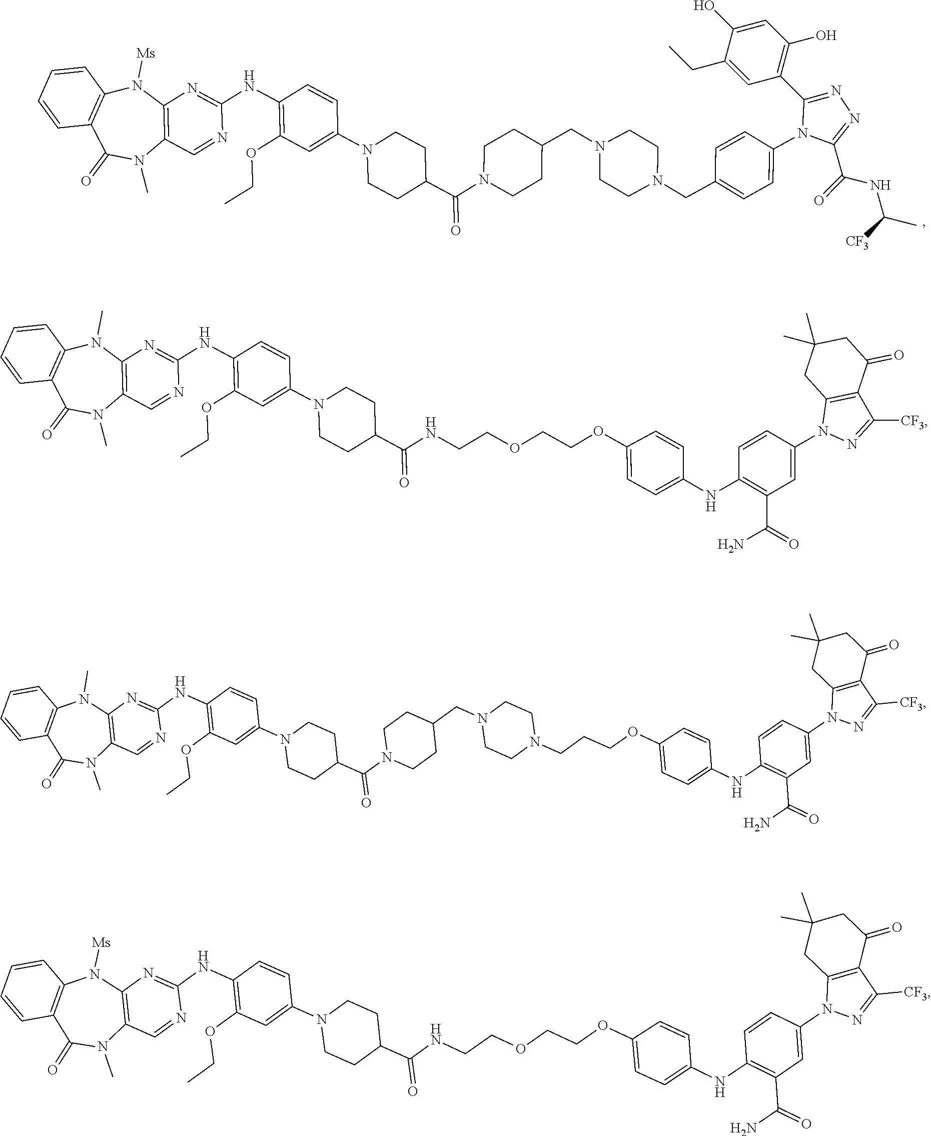 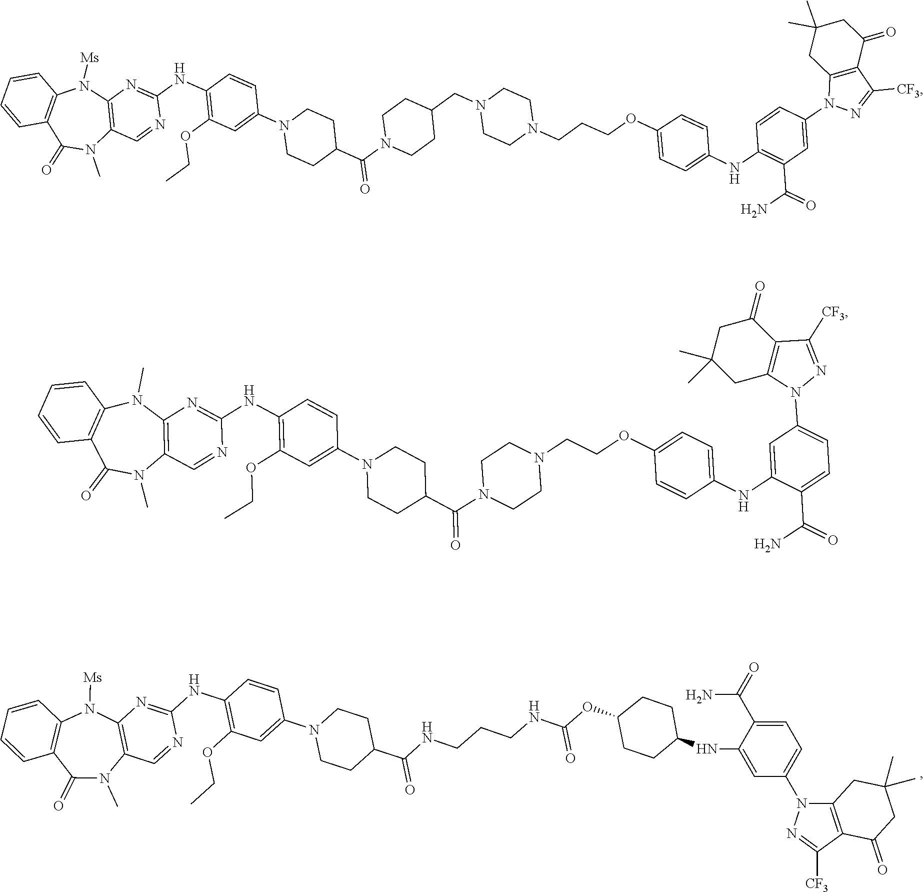 [SMILES: CCOc1cc(N2CCC(C(=O)N3CCC(CN4CCN(CCCOc5ccc(Nc6ccc(-n7nc(C(F)(F)F)c8c7CC(C)(C)CC8=O)cc6C(N)=O)cc5)CC4)CC3)CC2)ccc1Nc1ncc2c(n1)N(S(C)(=O)=O)c1ccccc1C(=O)N2C ; CCOc1cc(N2CCC(C(=O)N3CCN(CCOc4ccc(Nc5cc(-n6nc(C(F)(F)F)c7c6CC(C)(C)CC7=O)ccc5C(N)=O)cc4)CC3)CC2)ccc1Nc1ncc2c(n1)N(C)c1ccccc1C(=O)N2C ; CCOc1cc(N2CCC(C(=O)NCCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(S(C)(=O)=O)c1ccccc1C(=O)N2C] 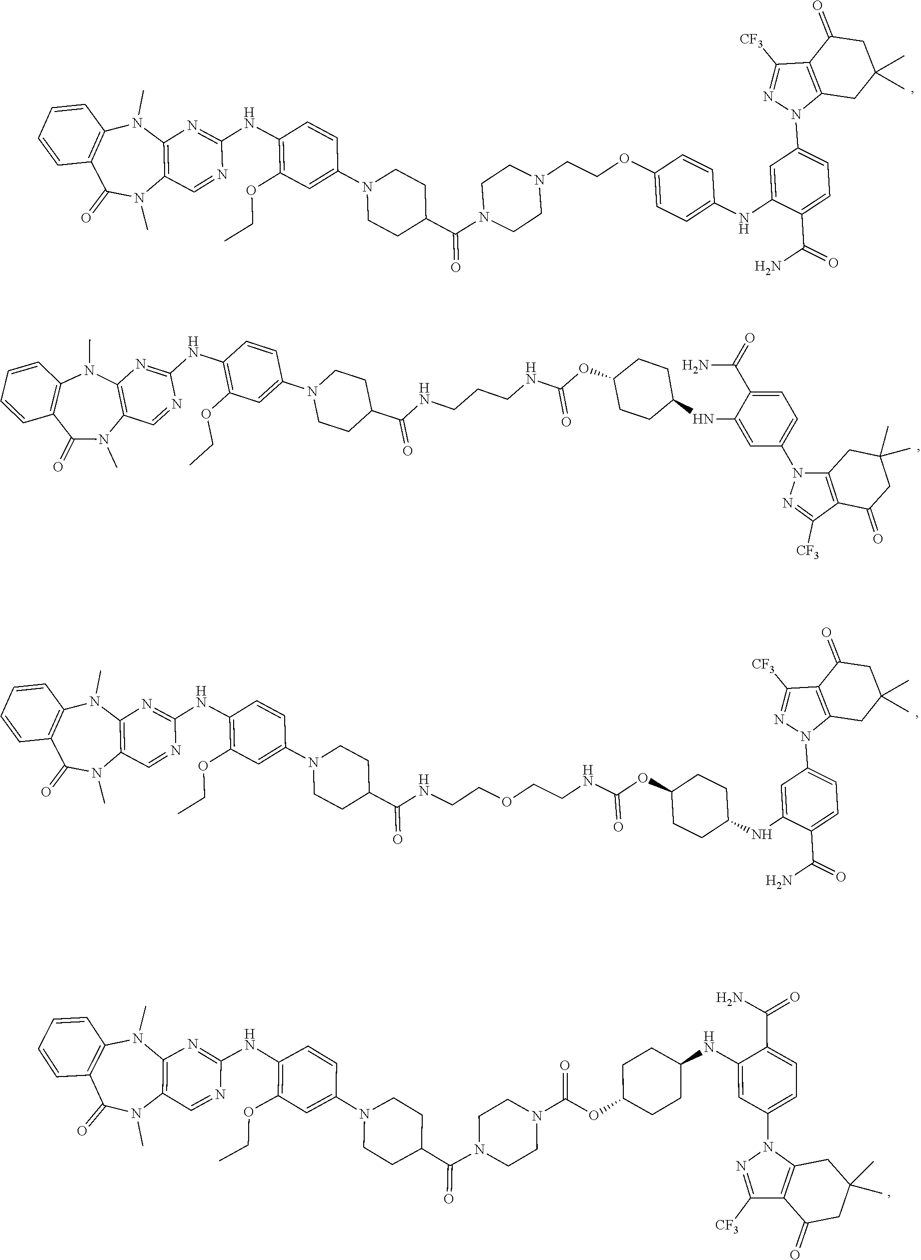 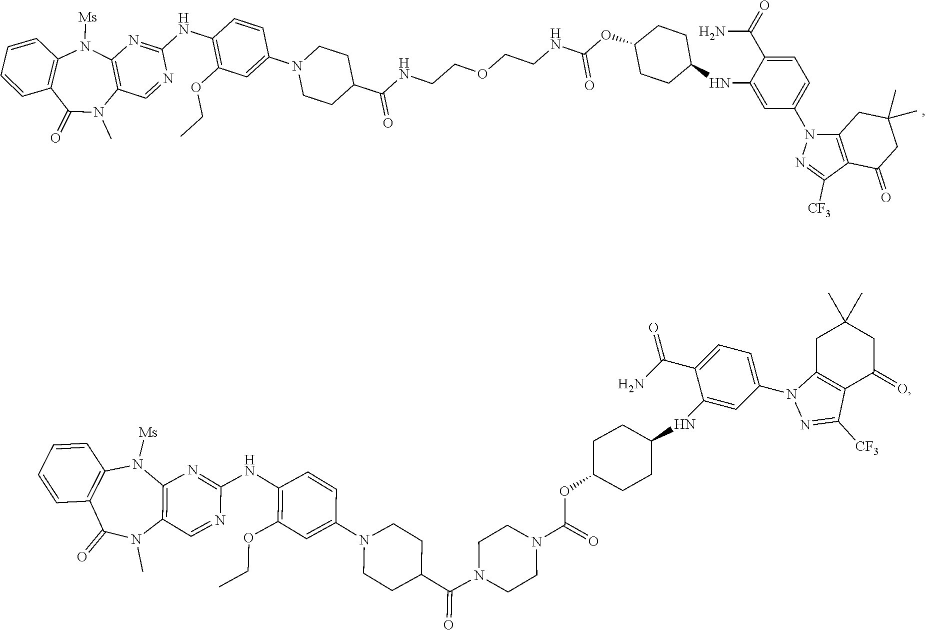 [SMILES: CCOc1cc(N2CCC(C(=O)NCCOCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(S(C)(=O)=O)c1ccccc1C(=O)N2C ; CCOc1cc(N2CCC(C(=O)N3CCN(C(=O)O[C@H]4CC[C@H](Nc5cc(-n6nc(C(F)(F)F)c7c6CC(C)(C)CC7=O)ccc5C(N)=O)CC4)CC3)CC2)ccc1Nc1ncc2c(n1)N(S(C)(=O)=O)c1ccccc1C(=O)N2C] 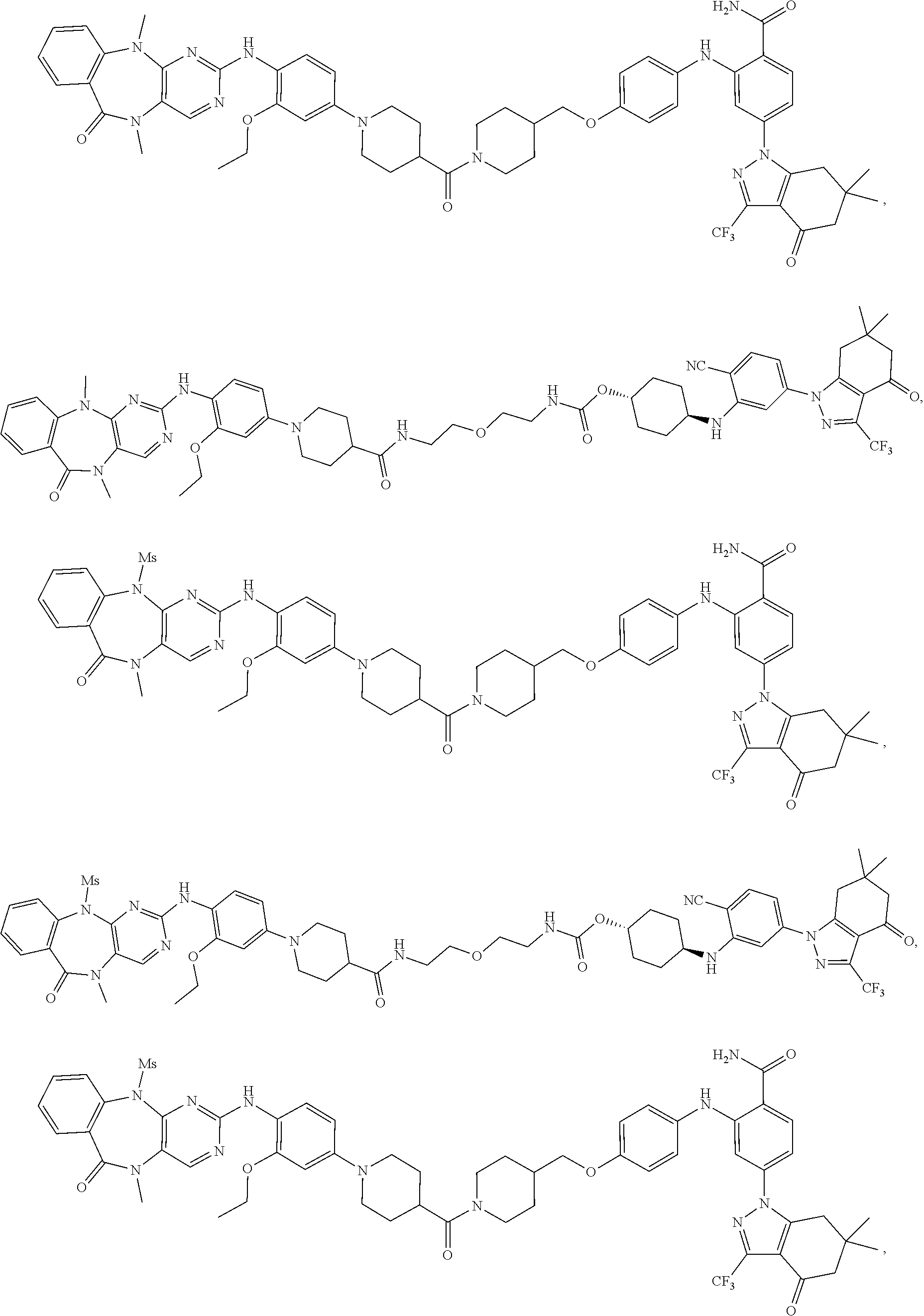 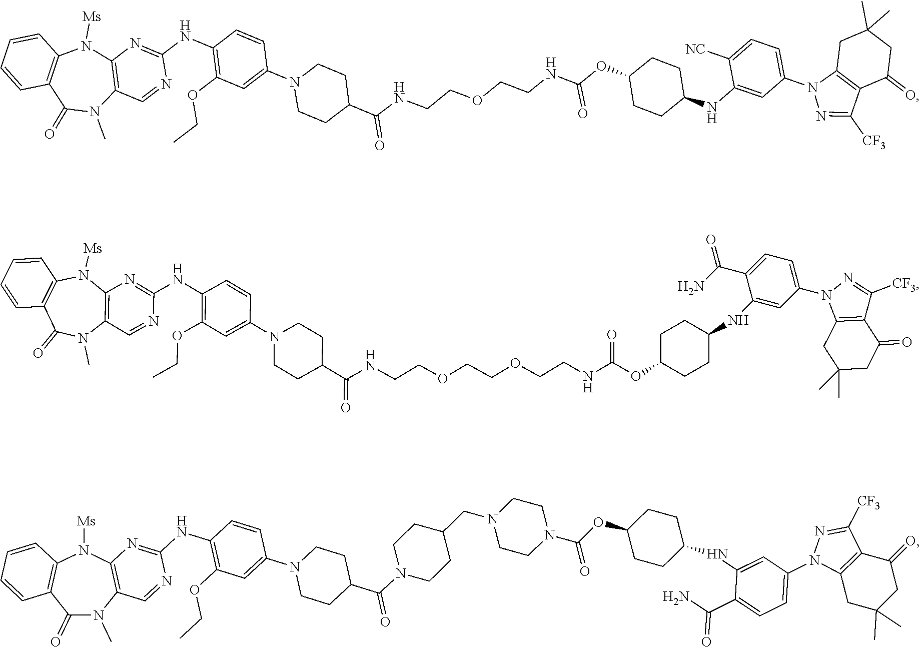 [SMILES: CCOc1cc(N2CCC(C(=O)NCCOCCNC(=O)O[C@H]3CC[C@H](Nc4cc(-n5nc(C(F)(F)F)c6c5CC(C)(C)CC6=O)ccc4C#N)CC3)CC2)ccc1Nc1ncc2c(n1)N(S(C)(=O)=O)c1ccccc1C(=O)N2C ; CCOc1cc(N2CCC(C(=O)NCCOCCOCCNC(=O)O[C@H]3CC[C@H](Nc4cc(-n5nc(C(F)(F)F)c6c5CC(C)(C)CC6=O)ccc4C(N)=O)CC3)CC2)ccc1Nc1ncc2c(n1)N(S(C)(=O)=O)c1ccccc1C(=O)N2C ; CCOc1cc(N2CCC(C(=O)N3CCC(CN4CCN(C(=O)O[C@H]5CC[C@H](Nc6cc(-n7nc(C(F)(F)F)c8c7CC(C)(C)CC8=O)ccc6C(N)=O)CC5)CC4)CC3)CC2)ccc1Nc1ncc2c(n1)N(S(C)(=O)=O)c1ccccc1C(=O)N2C] 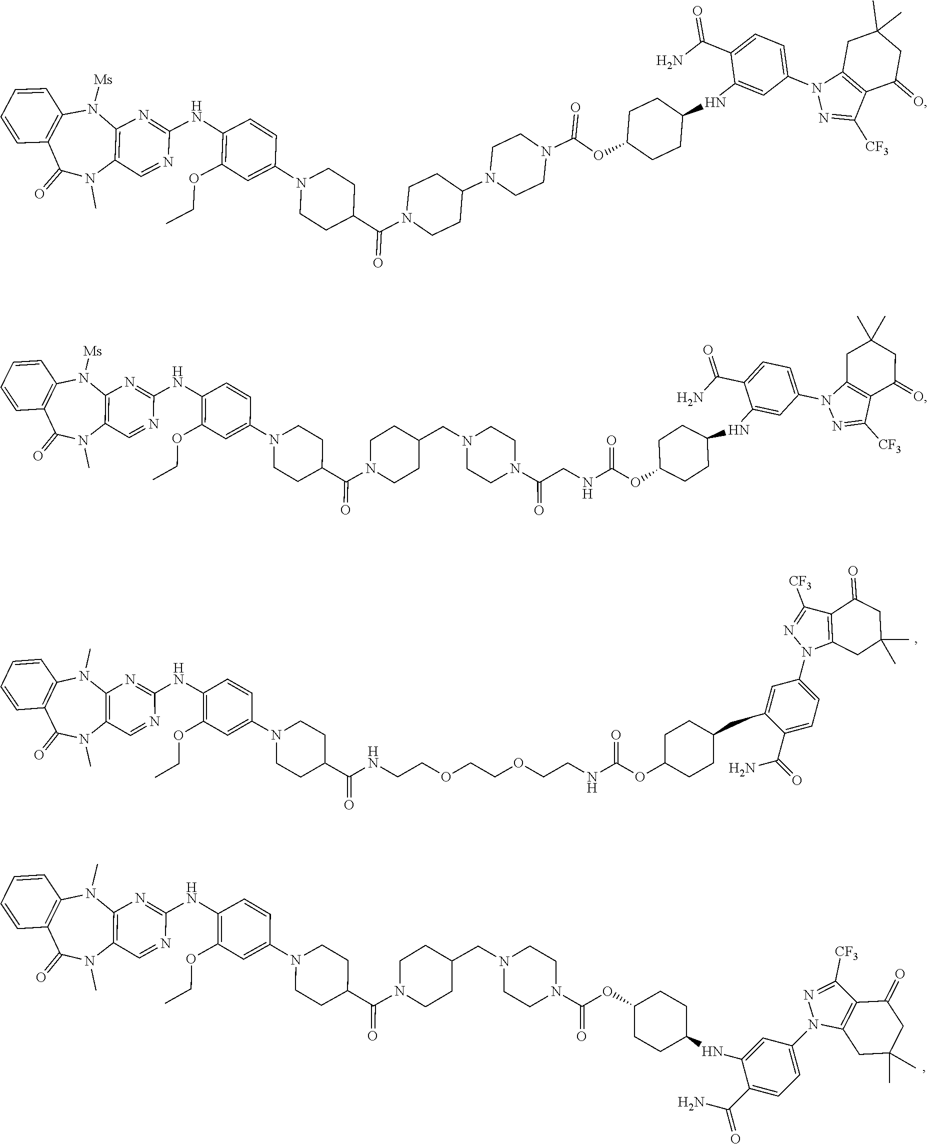 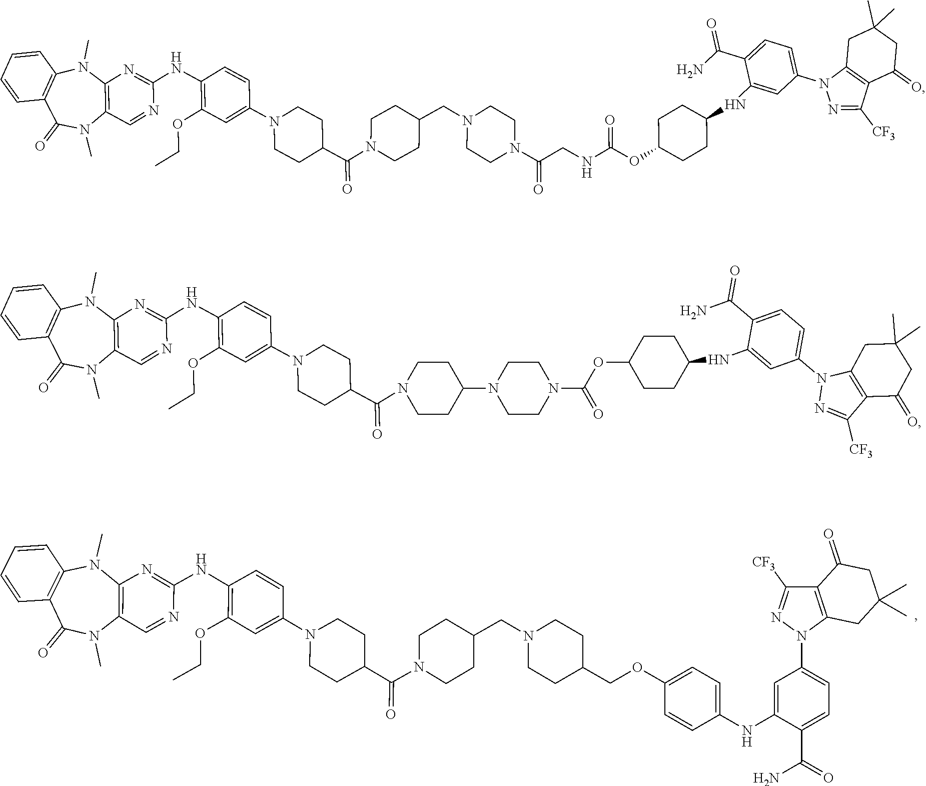 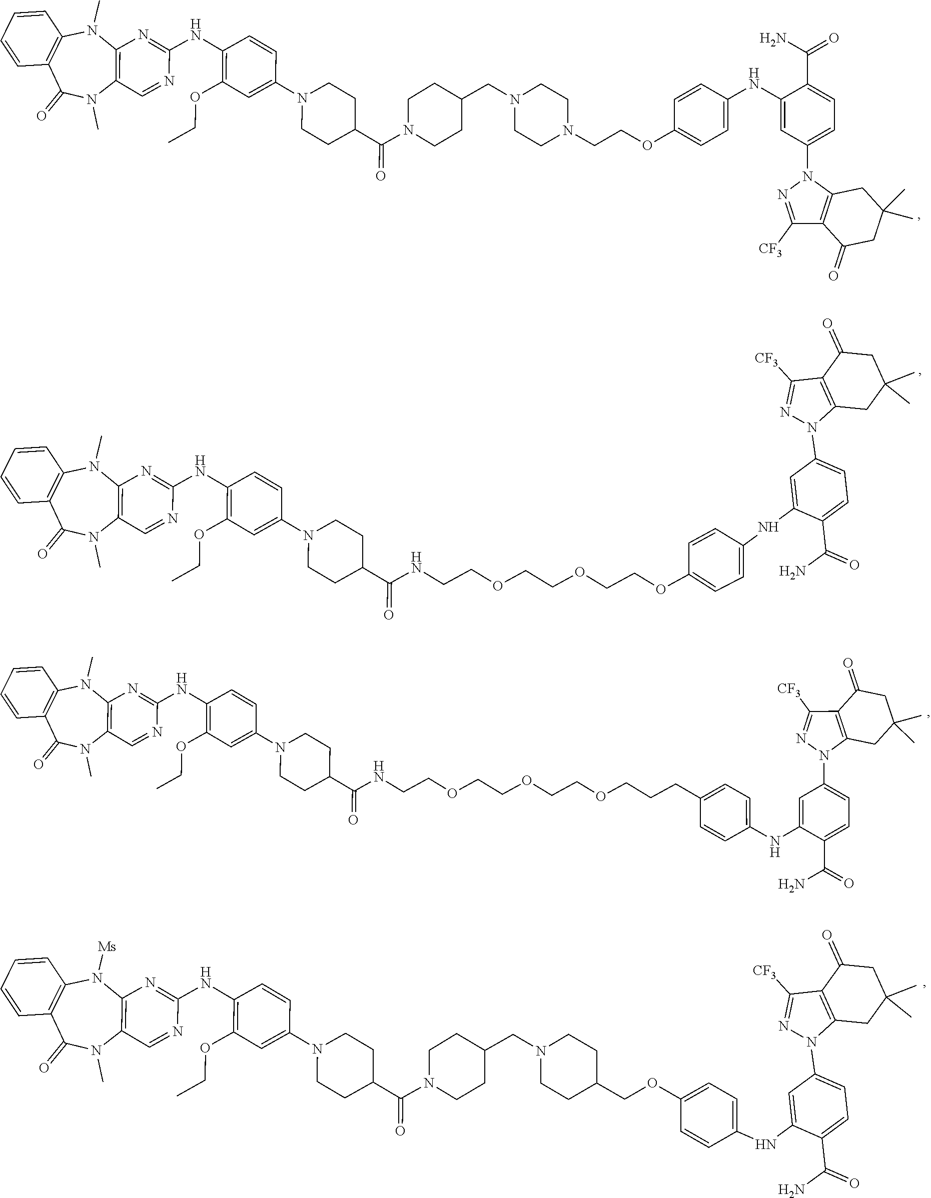 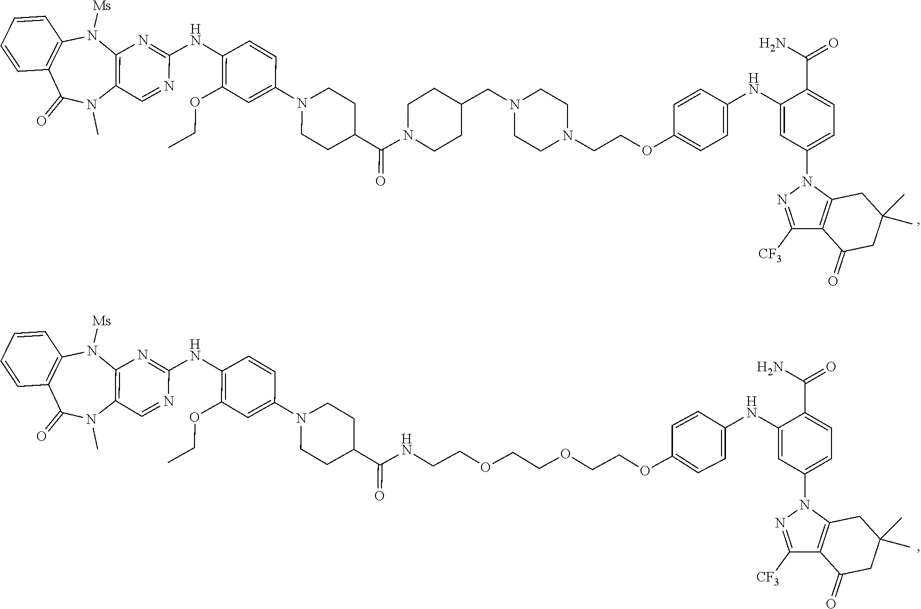 [SMILES: CCOc1cc(N2CCC(C(=O)N3CCC(CN4CCN(CCOc5ccc(Nc6cc(-n7nc(C(F)(F)F)c8c7CC(C)(C)CC8=O)ccc6C(N)=O)cc5)CC4)CC3)CC2)ccc1Nc1ncc2c(n1)N(S(C)(=O)=O)c1ccccc1C(=O)N2C ; CCOc1cc(N2CCC(C(=O)NCCOCCOCCOc3ccc(Nc4cc(-n5nc(C(F)(F)F)c6c5CC(C)(C)CC6=O)ccc4C(N)=O)cc3)CC2)ccc1Nc1ncc2c(n1)N(S(C)(=O)=O)c1ccccc1C(=O)N2C] 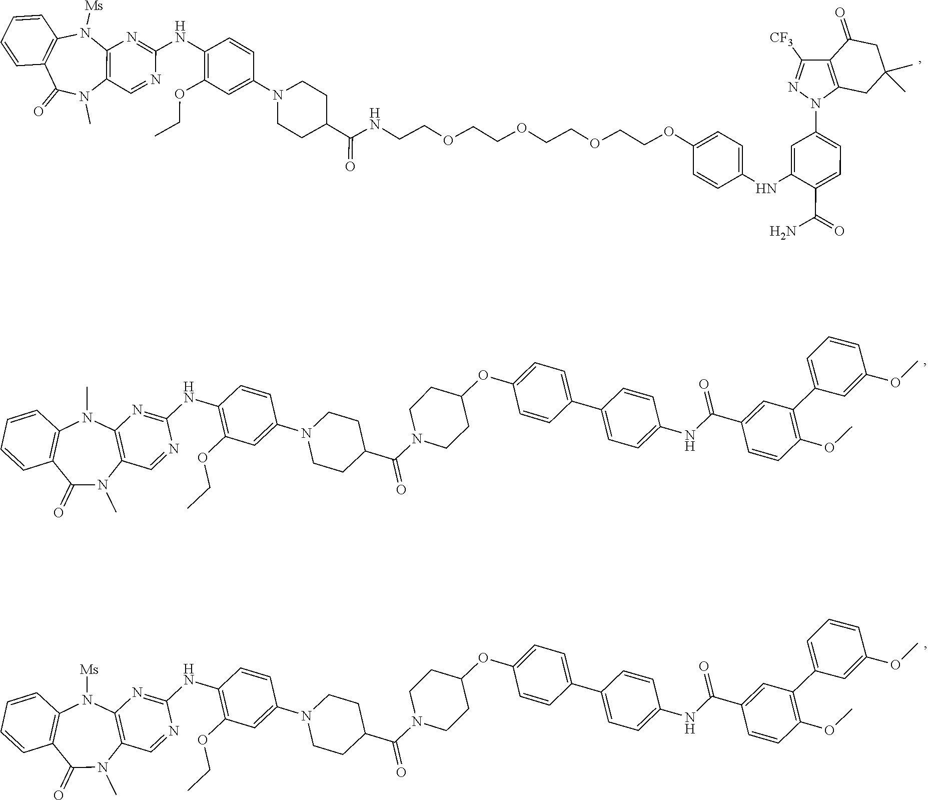 [SMILES: CCOc1cc(N2CCC(C(=O)NCCOCCOCCOCCOc3ccc(Nc4cc(-n5nc(C(F)(F)F)c6c5CC(C)(C)CC6=O)ccc4C(N)=O)cc3)CC2)ccc1Nc1ncc2c(n1)N(S(C)(=O)=O)c1ccccc1C(=O)N2C ; CCOc1cc(N2CCC(C(=O)N3CCC(Oc4ccc(-c5ccc(NC(=O)c6ccc(OC)c(-c7cccc(OC)c7)c6)cc5)cc4)CC3)CC2)ccc1Nc1ncc2c(n1)N(C)c1ccccc1C(=O)N2C ; CCOc1cc(N2CCC(C(=O)N3CCC(Oc4ccc(-c5ccc(NC(=O)c6ccc(OC)c(-c7cccc(OC)c7)c6)cc5)cc4)CC3)CC2)ccc1Nc1ncc2c(n1)N(S(C)(=O)=O)c1ccccc1C(=O)N2C] 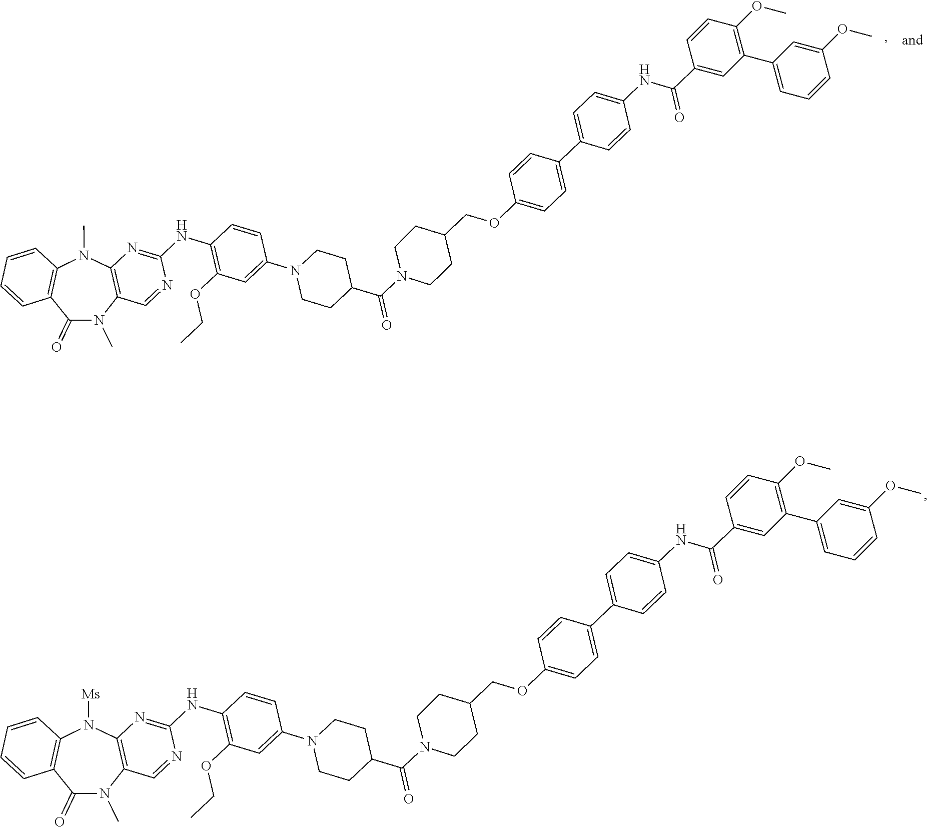 [SMILES: CCOc1cc(N2CCC(C(=O)N3CCC(COc4ccc(-c5ccc(NC(=O)c6ccc(OC)c(-c7cccc(OC)c7)c6)cc5)cc4)CC3)CC2)ccc1Nc1ncc2c(n1)N(C)c1ccccc1C(=O)N2C ; CCOc1cc(N2CCC(C(=O)N3CCC(COc4ccc(-c5ccc(NC(=O)c6ccc(OC)c(-c7cccc(OC)c7)c6)cc5)cc4)CC3)CC2)ccc1Nc1ncc2c(n1)N(S(C)(=O)=O)c1ccccc1C(=O)N2C]
or a pharmaceutically acceptable salt of any of the foregoing.

41. The compound of claim 1, wherein the compound is selected from the following structural formula: 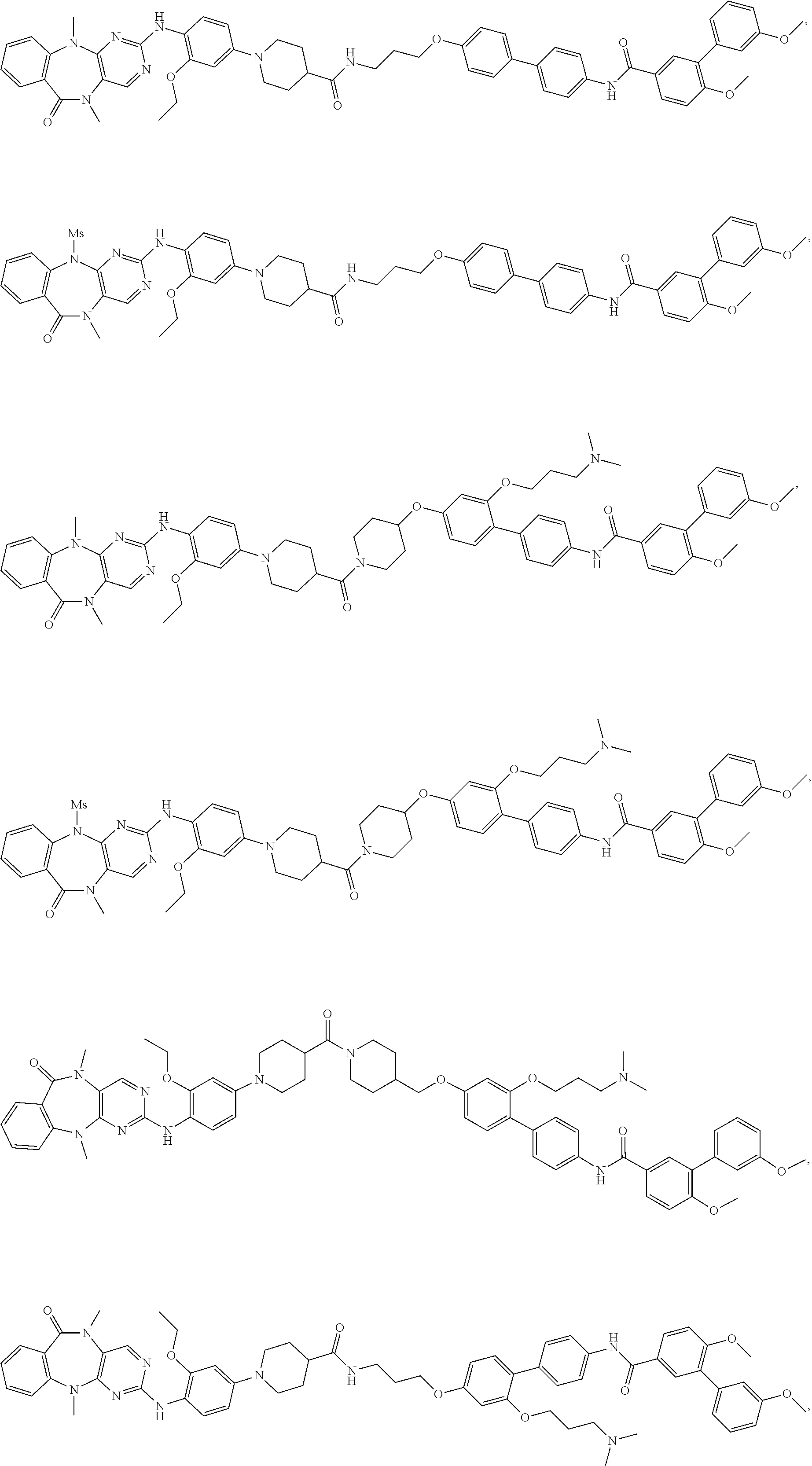 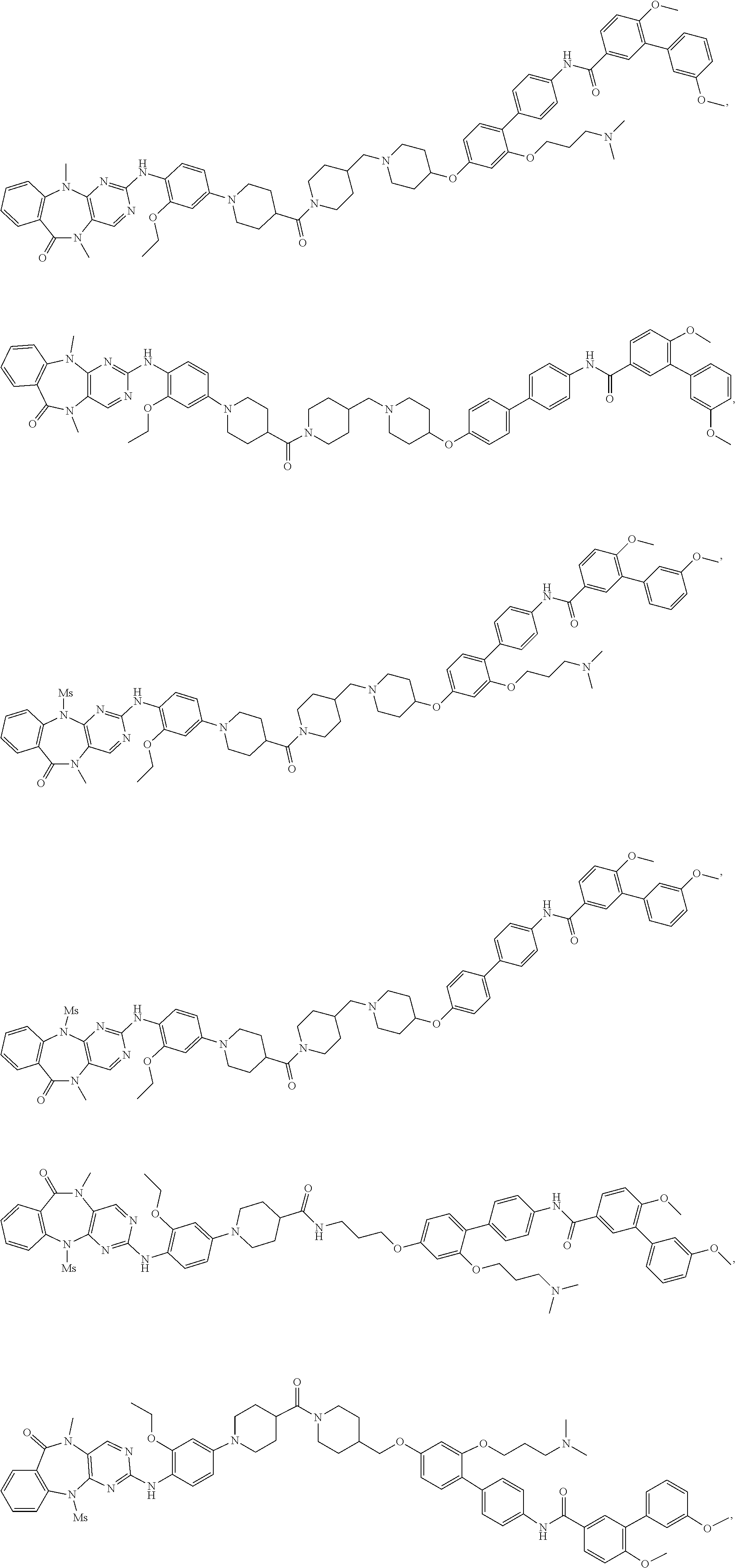 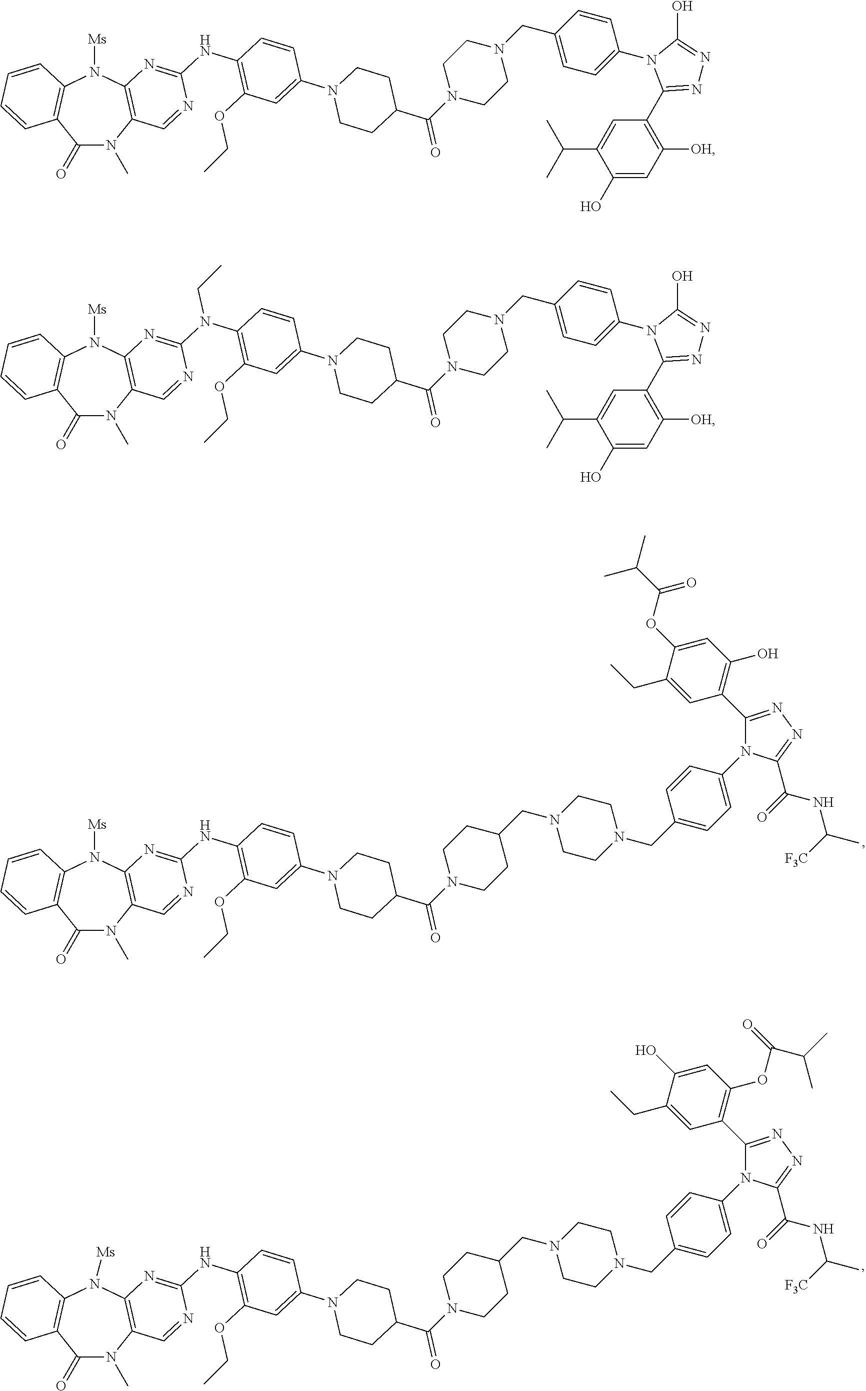 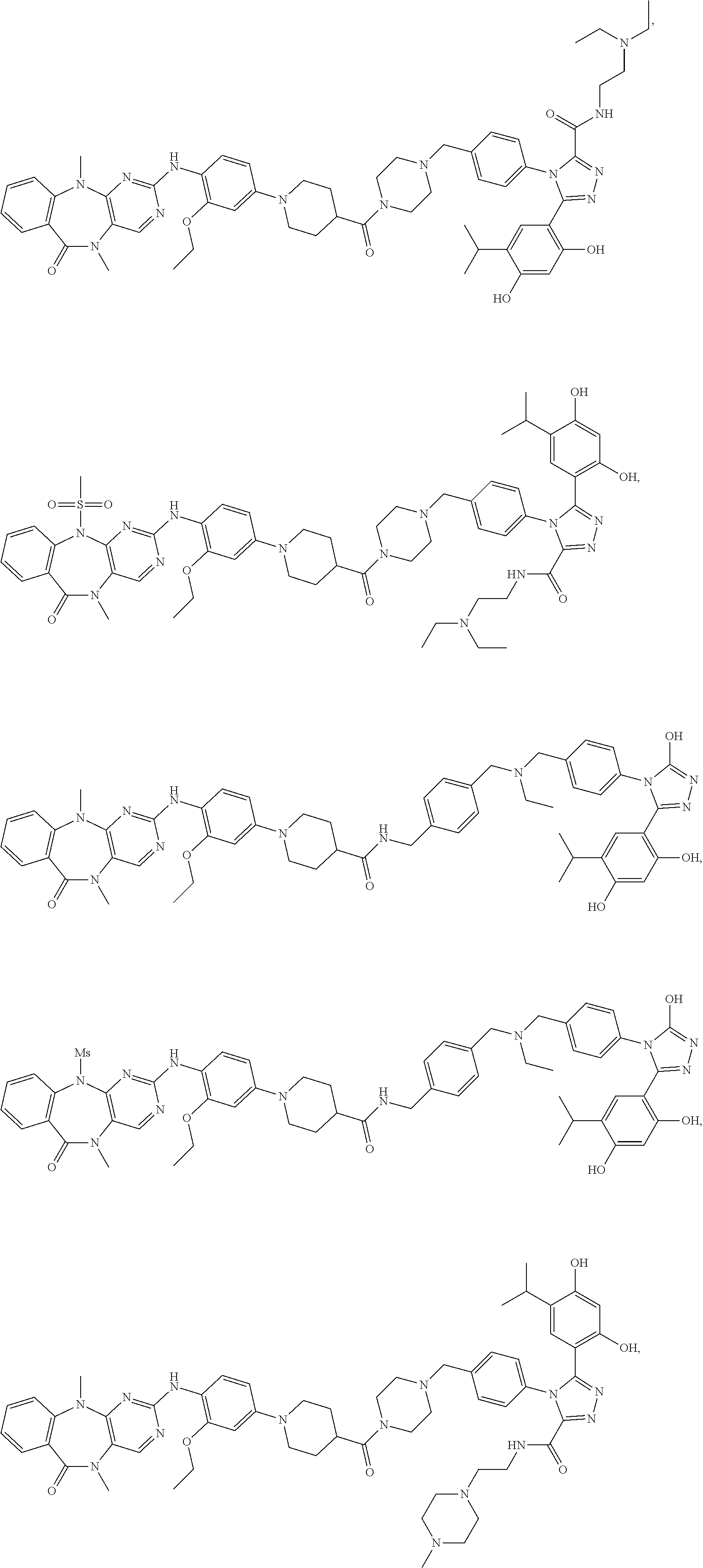 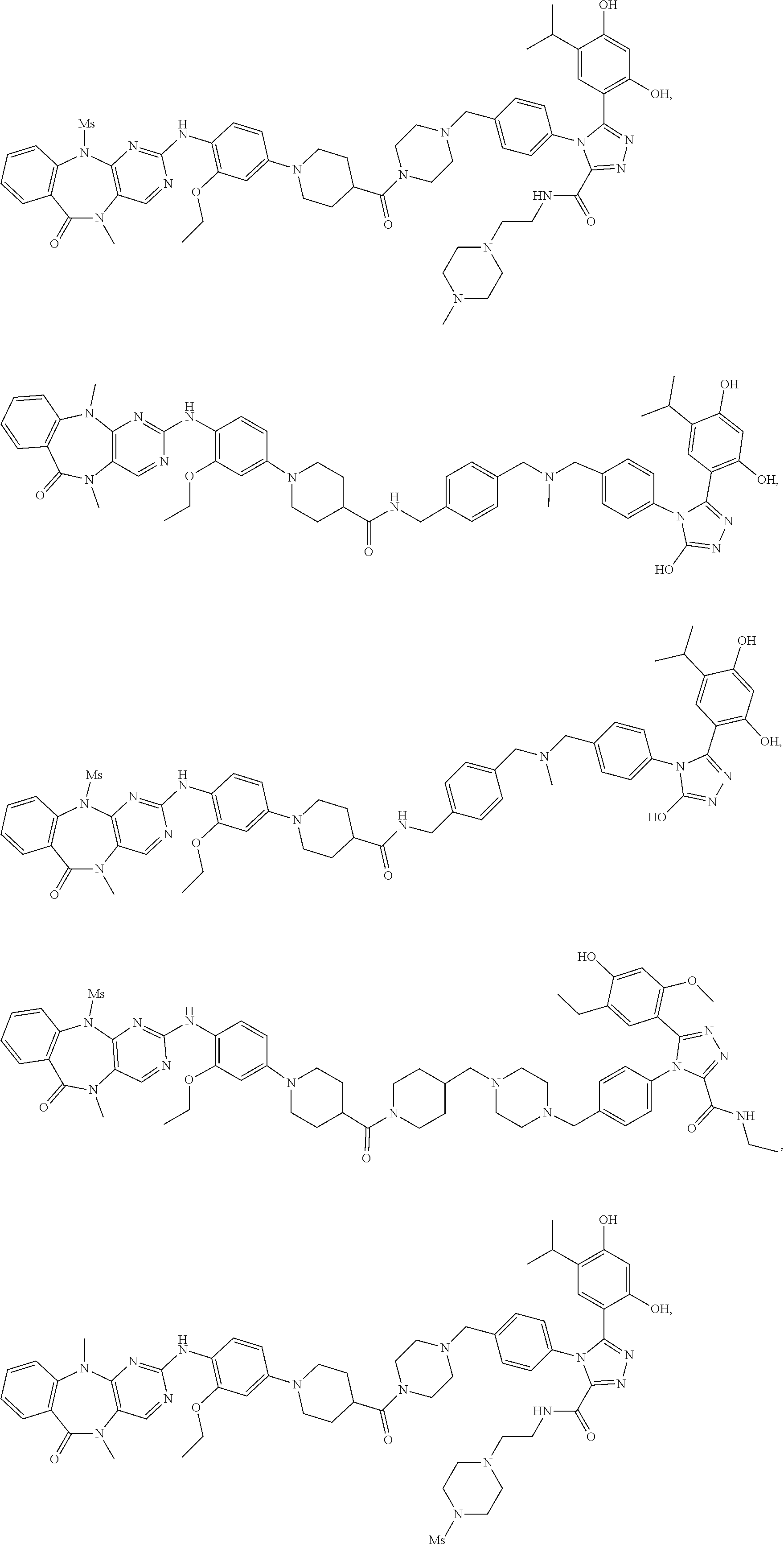 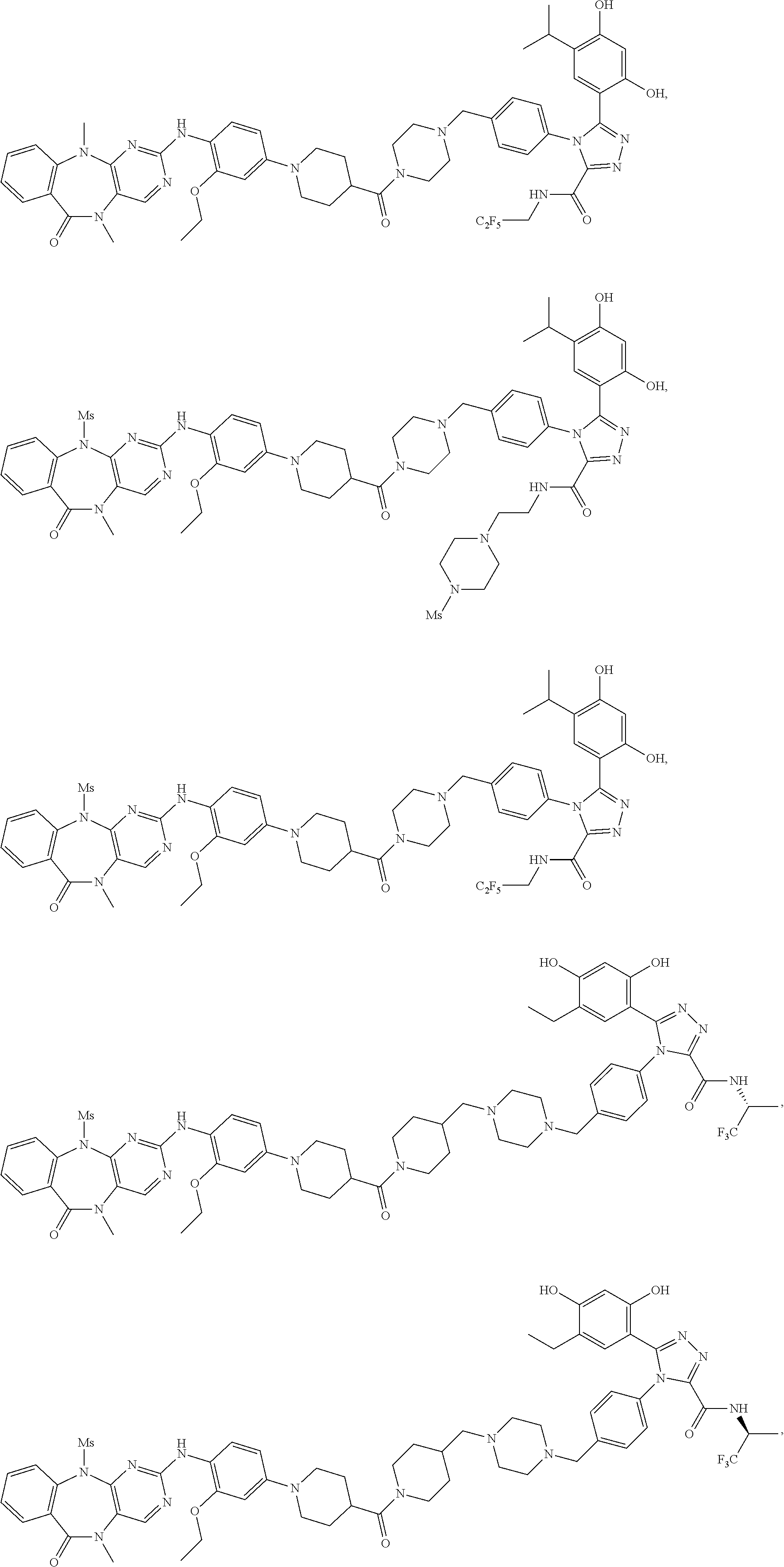 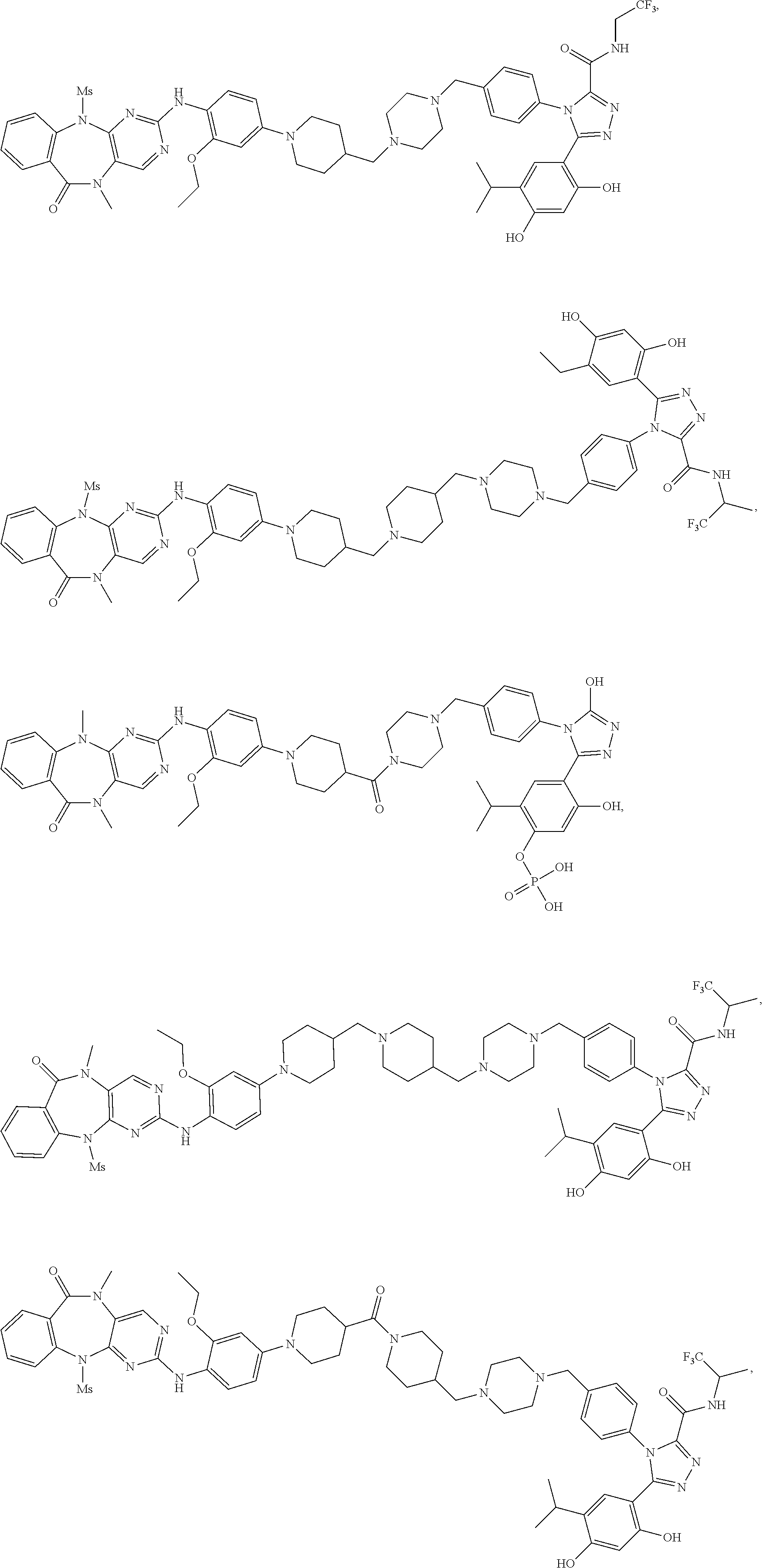 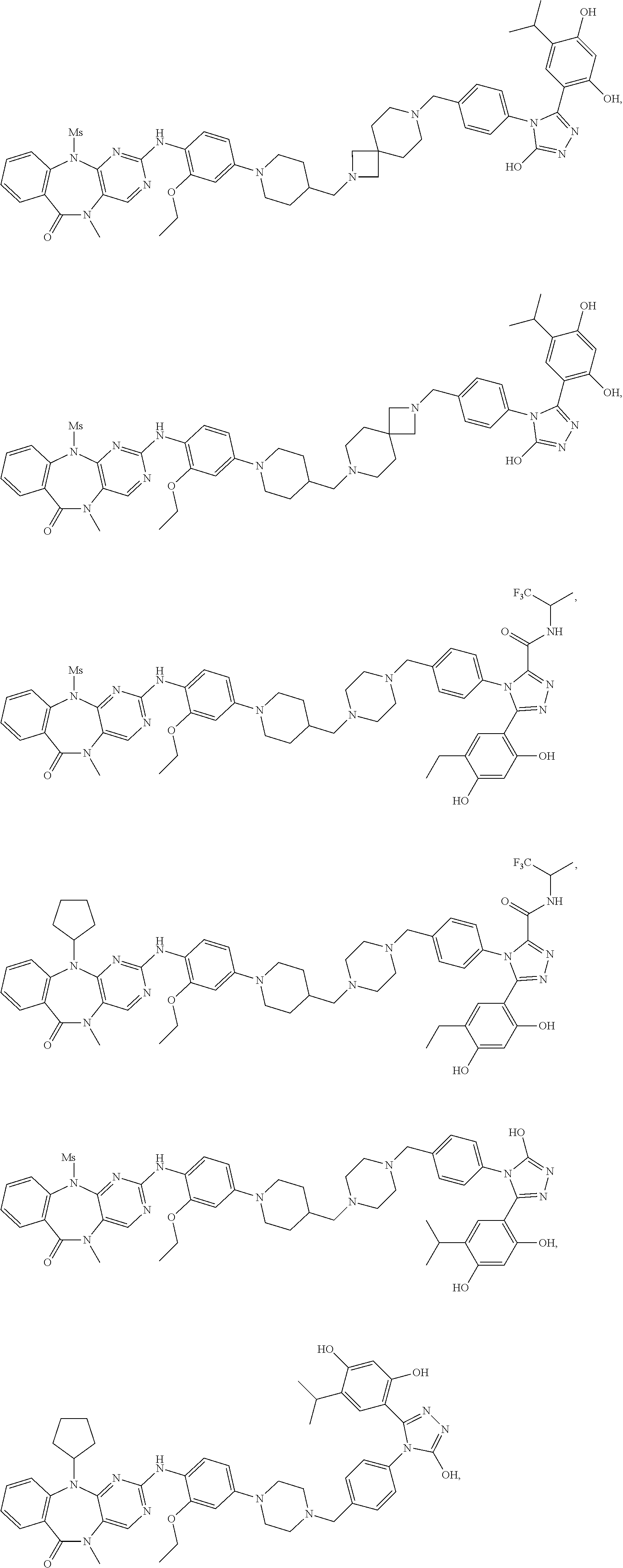 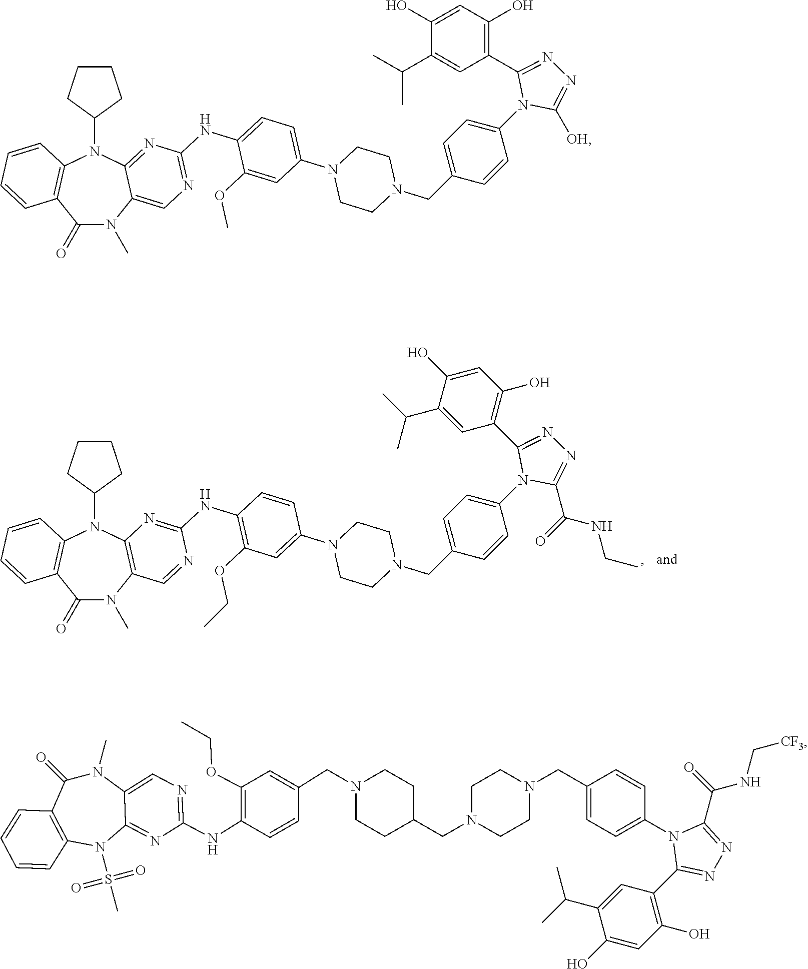 [SMILES: COc1cc(N2CCN(Cc3ccc(-n4c(O)nnc4-c4cc(C(C)C)c(O)cc4O)cc3)CC2)ccc1Nc1ncc2c(n1)N(C1CCCC1)c1ccccc1C(=O)N2C ; CCNC(=O)c1nnc(-c2cc(C(C)C)c(O)cc2O)n1-c1ccc(CN2CCN(c3ccc(Nc4ncc5c(n4)N(C4CCCC4)c4ccccc4C(=O)N5C)c(OCC)c3)CC2)cc1 ; CCOc1cc(CN2CCC(CN3CCN(Cc4ccc(-n5c(C(=O)NCC(F)(F)F)nnc5-c5cc(C(C)C)c(O)cc5O)cc4)CC3)CC2)ccc1Nc1ncc2c(n1)N(S(C)(=O)=O)c1ccccc1C(=O)N2C]
or a pharmaceutically acceptable salt of any of the foregoing.

42. A pharmaceutical composition comprising the compound of claim 1, or a pharmaceutically acceptable salt thereof, and a pharmaceutically acceptable carrier.

43. A method of treating cancer comprising administering to a subject a therapeutically effective amount of a compound of claim 1, or a pharmaceutically acceptable salt thereof.
